SEQUENCE LISTING (515 sequences)

SEQ ID NO: 1 - protein, 162 aa, Homo sapiens
  MISC_FEATURE (1)..(162): IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - DNA, 489 bp, Homo sapiens
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 3 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - DNA, 534 bp, Homo sapiens
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 5 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 6 - DNA, 1127 bp, Homo sapiens
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca

SEQ ID NO: 7 - protein, 136 aa, Homo sapiens
MGLTSQLLPPLFFLLACAGNFVHGHKCDITLQEIIKTLNSLTEQKTLCTELTVTDIFAASKQHACLPTSRHTGTICRPPSARVPSTSTRLQSPERTQLRRKPSAGLRLCSGSSTATMRRTLAAWVRLHSSSTGTSS

SEQ ID NO: 8 - DNA, 411 bp, Homo sapiens
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagcagcatgcatgtctgcccacctccagacacacaggcaccatctgccgccccccatcagcccgtgtcccttccacctcgactcgcctacaaagcccagagagaacacaactgagaaggaaaccttctgcagggctgcgactgtgctccggcagttctacagccaccatgagaaggacactcgctgcctgggtgcgactgcacagcagttccacaggcacaagcagctga

SEQ ID NO: 9 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 10 - DNA, 429 bp, Homo sapiens
ctgaacgatcaggtgctgtttattgatcagggcaaccgcccgctgtttgaagatatgaccgatagcgattgccgcgataacgcgccgcgcaccatttttattattagcatgtataaagatagccagccgcgcggcatggcggtgaccattagcgtgaaatgcgaaaaaattagcaccctgagctgcgaaaacaaaattattagctttaaagaaatgaacccgccggataacattaaagataccaaaagcgatattattttttttcagcgcagcgtgccgggccatgataacaaaatgcagtttgaaagcagcagctatgaaggctattttctggcgtgcgaaaaagaacgcgatctgtttaaactgattctgaaaaaagaagatgaactgggcgatcgcagcattatgtttaccgtgcagaacgaagat

SEQ ID NO: 11 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 75 aa, SARS-CoV-2
MYSFVSEETGTLIVNSVLLFLAFVVFLLVTLAILTALRLCAYCCNIVNVSLVKPSFYVYSRVKNLNSSRVPDLLV

SEQ ID NO: 13 - protein, 222 aa, SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 14 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 15 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 16 - protein, 141 aa, Homo sapiens
MGAPTLPPAWQPFLKDHRISTFKNWPFLEGCACTPERMAEAGFIHCPTENEPDLQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 17 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 18 - protein, 11 aa, Artificial Sequence
  Epitope
TSEKRPFMCAY

SEQ ID NO: 19 - protein, 11 aa, Artificial Sequence
  Epitope
STVTFDGTPSY

SEQ ID NO: 20 - protein, 11 aa, Artificial Sequence
  Epitope
HTTPILCGAQY

SEQ ID NO: 21 - protein, 11 aa, Artificial Sequence
  Epitope
ESQPAIRNQGY

SEQ ID NO: 22 - protein, 11 aa, Artificial Sequence
  Epitope
GSQALLLRTPY

SEQ ID NO: 23 - protein, 11 aa, Artificial Sequence
  Epitope
HSRKHTGEKPY

SEQ ID NO: 24 - protein, 10 aa, Artificial Sequence
  Epitope
FTGTAGACRY

SEQ ID NO: 25 - protein, 10 aa, Artificial Sequence
  Epitope
RTPYSSDNLY

SEQ ID NO: 26 - protein, 10 aa, Artificial Sequence
  Epitope
TTPILCGAQY

SEQ ID NO: 27 - protein, 9 aa, Artificial Sequence
  Epitope
VTFDGTPSY

SEQ ID NO: 28 - protein, 11 aa, Artificial Sequence
  Epitope
LTDVSPEPLQA

SEQ ID NO: 29 - protein, 11 aa, Artificial Sequence
  Epitope
ITDDQLLALLP

SEQ ID NO: 30 - protein, 11 aa, Artificial Sequence
  Epitope
HGTLHLERLAY

SEQ ID NO: 31 - protein, 10 aa, Artificial Sequence
  Epitope
GTLHLERLAY

SEQ ID NO: 32 - protein, 10 aa, Artificial Sequence
  Epitope
CSQLTTLSFY

SEQ ID NO: 33 - protein, 10 aa, Artificial Sequence
  Epitope
LSLQCLQALY

SEQ ID NO: 34 - protein, 9 aa, Artificial Sequence
  Epitope
PTLAKFSPY

SEQ ID NO: 35 - protein, 9 aa, Artificial Sequence
  Epitope
LSNLTHVLY

SEQ ID NO: 36 - protein, 9 aa, Artificial Sequence
  Epitope
WSGNRASLY

SEQ ID NO: 37 - protein, 9 aa, Artificial Sequence
  Epitope
LSHIHASSY

SEQ ID NO: 38 - protein, 11 aa, Artificial Sequence
  Epitope
PTENEPDLAQC

SEQ ID NO: 39 - protein, 11 aa, Artificial Sequence
  Epitope
KLDRERAKNKI

SEQ ID NO: 40 - protein, 11 aa, Artificial Sequence
  Epitope
LKDHRISTFKN

SEQ ID NO: 41 - protein, 11 aa, Artificial Sequence
  Epitope
STFKNWPFLEG

SEQ ID NO: 42 - protein, 11 aa, Artificial Sequence
  Epitope
DDDPIEEHKKH

SEQ ID NO: 43 - protein, 10 aa, Artificial Sequence
  Epitope
PTENEPDLAQ

SEQ ID NO: 44 - protein, 9 aa, Artificial Sequence
  Epitope
PTENEPDLA

SEQ ID NO: 45 - protein, 9 aa, Artificial Sequence
  Epitope
LTLGEFLKL

SEQ ID NO: 46 - protein, 9 aa, Artificial Sequence
  Epitope
LGEFLKLDR

SEQ ID NO: 47 - protein, 9 aa, Artificial Sequence
  Epitope
KLDRERAKN

SEQ ID NO: 48 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 49 - protein, 87 aa, Homo sapiens
MTMYLWLKLLAFGFAFLDTEVFVTGQSPTPSPTGHLQAEEQGSQSKSPNLKSREADSSAFSWWPKAREPLTNHWSKSKSPKAEELGV

SEQ ID NO: 50 - protein, 15 aa, Artificial Sequence
  Viral peptide
MAPTKRKGECPGAAP

SEQ ID NO: 51 - protein, 15 aa, Artificial Sequence
  Viral peptide
KRKGECPGAAPKKPK

SEQ ID NO: 52 - protein, 15 aa, Artificial Sequence
  Viral peptide
ECPGAAPKKPKEPVQ

SEQ ID NO: 53 - protein, 15 aa, Artificial Sequence
  Viral peptide
AAPKKPKEPVQVPKL

SEQ ID NO: 54 - protein, 15 aa, Artificial Sequence
  Viral peptide
KPKEPVQVPKLLIKG

SEQ ID NO: 55 - protein, 15 aa, Artificial Sequence
  Viral peptide
PVQVPKLLIKGGVEV

SEQ ID NO: 56 - protein, 15 aa, Artificial Sequence
  Viral peptide
PKLLIKGGVEVLEVK

SEQ ID NO: 57 - protein, 15 aa, Artificial Sequence
  Viral peptide
IKGGVEVLEVKTGVD

SEQ ID NO: 58 - protein, 15 aa, Artificial Sequence
  Viral peptide
VEVLEVKTGVDAITE

SEQ ID NO: 59 - protein, 15 aa, Artificial Sequence
  Viral peptide
EVKTGVDAITEVECF

SEQ ID NO: 60 - protein, 15 aa, Artificial Sequence
  Viral peptide
GVDAITEVECFLNPE

SEQ ID NO: 61 - protein, 15 aa, Artificial Sequence
  Viral peptide
ITEVECFLNPEMGDP

SEQ ID NO: 62 - protein, 15 aa, Artificial Sequence
  Viral peptide
ECFLNPEMGDPDDNL

SEQ ID NO: 63 - protein, 15 aa, Artificial Sequence
  Viral peptide
NPEMGDPDDNLRGYS

SEQ ID NO: 64 - protein, 15 aa, Artificial Sequence
  Viral peptide
GDPDDNLRGYSQHLS

SEQ ID NO: 65 - protein, 15 aa, Artificial Sequence
  Viral peptide
DNLRGYSQHLSAENA

SEQ ID NO: 66 - protein, 15 aa, Artificial Sequence
  Viral peptide
GYSQHLSAENAFESD

SEQ ID NO: 67 - protein, 15 aa, Artificial Sequence
  Viral peptide
HLSAENAFESDSPDR

SEQ ID NO: 68 - protein, 15 aa, Artificial Sequence
  Viral peptide
ENAFESDSPDRKMLP

SEQ ID NO: 69 - protein, 15 aa, Artificial Sequence
  Viral peptide
ESDSPDRKMLPCYST

SEQ ID NO: 70 - protein, 15 aa, Artificial Sequence
  Viral peptide
PDRKMLPCYSTARIP

SEQ ID NO: 71 - protein, 15 aa, Artificial Sequence
  Viral peptide
MLPCYSTARIPLPNL

SEQ ID NO: 72 - protein, 15 aa, Artificial Sequence
  Viral peptide
YSTARIPLPNLNEDL

SEQ ID NO: 73 - protein, 15 aa, Artificial Sequence
  Viral peptide
RIPLPNLNEDLTCGN

SEQ ID NO: 74 - protein, 15 aa, Artificial Sequence
  Viral peptide
PNLNEDLTCGNLLMW

SEQ ID NO: 75 - protein, 15 aa, Artificial Sequence
  Viral peptide
EDLTCGNLLMWEAVT

SEQ ID NO: 76 - protein, 15 aa, Artificial Sequence
  Viral peptide
CGNLLMWEAVTVKTE

SEQ ID NO: 77 - protein, 15 aa, Artificial Sequence
  Viral peptide
LMWEAVTVKTEVIGI

SEQ ID NO: 78 - protein, 15 aa, Artificial Sequence
  Viral peptide
AVTVKTEVIGITSML

SEQ ID NO: 79 - protein, 15 aa, Artificial Sequence
  Viral peptide
KTEVIGITSMLNLHA

SEQ ID NO: 80 - protein, 15 aa, Artificial Sequence
  Viral peptide
IGITSMLNLHAGSQK

SEQ ID NO: 81 - protein, 15 aa, Artificial Sequence
  Viral peptide
SMLNLHAGSQKVKEN

SEQ ID NO: 82 - protein, 15 aa, Artificial Sequence
  Viral peptide
LHAGSQKVHENGGGK

SEQ ID NO: 83 - protein, 15 aa, Artificial Sequence
  Viral peptide
SQKVHENGGGKPVQG

SEQ ID NO: 84 - protein, 15 aa, Artificial Sequence
  Viral peptide
HENGGGKPVQGSNFH

SEQ ID NO: 85 - protein, 15 aa, Artificial Sequence
  Viral peptide
GGKPVQGSNFHFFAV

SEQ ID NO: 86 - protein, 15 aa, Artificial Sequence
  Viral peptide
VQGSNFHFFAVGGDP

SEQ ID NO: 87 - protein, 15 aa, Artificial Sequence
  Viral peptide
NFHFFAVGGDPLEMQ

SEQ ID NO: 88 - protein, 15 aa, Artificial Sequence
  Viral peptide
FAVGGDPLEMQGVLM

SEQ ID NO: 89 - protein, 15 aa, Artificial Sequence
  Viral peptide
GDPLEMQGVLMNYRT

SEQ ID NO: 90 - protein, 15 aa, Artificial Sequence
  Viral peptide
EMQGVLMNYRTKYPQ

SEQ ID NO: 91 - protein, 15 aa, Artificial Sequence
  Viral peptide
VLMNYRTKYPQGTIT

SEQ ID NO: 92 - protein, 15 aa, Artificial Sequence
  Viral peptide
YRTKYPQGTITPKNP

SEQ ID NO: 93 - protein, 15 aa, Artificial Sequence
  Viral peptide
YPQGTITPKNPTAQS

SEQ ID NO: 94 - protein, 15 aa, Artificial Sequence
  Viral peptide
TITPKNPTAQSQVMN

SEQ ID NO: 95 - protein, 15 aa, Artificial Sequence
  Viral peptide
KNPTAQSQVMNTDHK

SEQ ID NO: 96 - protein, 15 aa, Artificial Sequence
  Viral peptide
AQSQVMNTDHKAYLD

SEQ ID NO: 97 - protein, 15 aa, Artificial Sequence
  Viral peptide
VMNTDHKAYLDKNNA

SEQ ID NO: 98 - protein, 15 aa, Artificial Sequence
  Viral peptide
DHKAYLDKNNAYPVE

SEQ ID NO: 99 - protein, 15 aa, Artificial Sequence
  Viral peptide
YLDKNNAYPVECWIP

SEQ ID NO: 100 - protein, 15 aa, Artificial Sequence
  Viral peptide
NNAYPVECWIPDPSR

SEQ ID NO: 101 - protein, 15 aa, Artificial Sequence
  Viral peptide
PVECWIPDPSRNENT

SEQ ID NO: 102 - protein, 15 aa, Artificial Sequence
  Viral peptide
WIPDPSRNENTRYFG

SEQ ID NO: 103 - protein, 15 aa, Artificial Sequence
  Viral peptide
PSRNENTRYFGTYTG

SEQ ID NO: 104 - protein, 15 aa, Artificial Sequence
  Viral peptide
ENTRYFGTYTGGENV

SEQ ID NO: 105 - protein, 15 aa, Artificial Sequence
  Viral peptide
YFGTYTGGENVPPVL

SEQ ID NO: 106 - protein, 15 aa, Artificial Sequence
  Viral peptide
YTGGENVPPVLHVTN

SEQ ID NO: 107 - protein, 15 aa, Artificial Sequence
  Viral peptide
ENVPPVLHVTNTATT

SEQ ID NO: 108 - protein, 15 aa, Artificial Sequence
  Viral peptide
PVLHVTNTATTVLLD

SEQ ID NO: 109 - protein, 15 aa, Artificial Sequence
  Viral peptide
VINTATTVLLDEQGV

SEQ ID NO: 110 - protein, 15 aa, Artificial Sequence
  Viral peptide
ATTVLLDEQGVGPLC

SEQ ID NO: 111 - protein, 15 aa, Artificial Sequence
  Viral peptide
LLDEQGVGPLCKADS

SEQ ID NO: 112 - protein, 15 aa, Artificial Sequence
  Viral peptide
QGVGPLCKADSLYVS

SEQ ID NO: 113 - protein, 15 aa, Artificial Sequence
  Viral peptide
PLCKADSLYVSAADI

SEQ ID NO: 114 - protein, 15 aa, Artificial Sequence
  Viral peptide
ADSLYVSAADICGLF

SEQ ID NO: 115 - protein, 15 aa, Artificial Sequence
  Viral peptide
YVSAADICGLFTNSS

SEQ ID NO: 116 - protein, 15 aa, Artificial Sequence
  Viral peptide
ADICGLFTNSSGTQQ

SEQ ID NO: 117 - protein, 15 aa, Artificial Sequence
  Viral peptide
GLFTNSSGTQQWRGL

SEQ ID NO: 118 - protein, 15 aa, Artificial Sequence
  Viral peptide
NSSGTQQWRGLARYF

SEQ ID NO: 119 - protein, 15 aa, Artificial Sequence
  Viral peptide
TQQWRGLARYFKIRL

SEQ ID NO: 120 - protein, 15 aa, Artificial Sequence
  Viral peptide
RGLARYFKIRLRKRS

SEQ ID NO: 121 - protein, 15 aa, Artificial Sequence
  Viral peptide
RYFKIRLRKRSVKNP

SEQ ID NO: 122 - protein, 15 aa, Artificial Sequence
  Viral peptide
IRLRKRSVKNPYPIS

SEQ ID NO: 123 - protein, 15 aa, Artificial Sequence
  Viral peptide
KRSVKNPYPISFLLS

SEQ ID NO: 124 - protein, 15 aa, Artificial Sequence
  Viral peptide
KNPYPISFLLSDLIN

SEQ ID NO: 125 - protein, 15 aa, Artificial Sequence
  Viral peptide
PISFLLSDLINRRTQ

SEQ ID NO: 126 - protein, 15 aa, Artificial Sequence
  Viral peptide
LLSDLINRRTQKVDG

SEQ ID NO: 127 - protein, 15 aa, Artificial Sequence
  Viral peptide
LINRRTQKVDGQPMY

SEQ ID NO: 128 - protein, 15 aa, Artificial Sequence
  Viral peptide
RTQKVDGQPMYGMES

SEQ ID NO: 129 - protein, 15 aa, Artificial Sequence
  Viral peptide
VDGQPMYGMESQVEE

SEQ ID NO: 130 - protein, 15 aa, Artificial Sequence
  Viral peptide
PMYGMESQVEEVRVF

SEQ ID NO: 131 - protein, 15 aa, Artificial Sequence
  Viral peptide
MESQVEEVRVFDGTE

SEQ ID NO: 132 - protein, 15 aa, Artificial Sequence
  Viral peptide
VEEVRVFDGTEQLPG

SEQ ID NO: 133 - protein, 15 aa, Artificial Sequence
  Viral peptide
RVFDGTEQLPGDPDM

SEQ ID NO: 134 - protein, 15 aa, Artificial Sequence
  Viral peptide
GTEQLPGDPDMIRYI

SEQ ID NO: 135 - protein, 15 aa, Artificial Sequence
  Viral peptide
LPGDPDMIRYIDRQG

SEQ ID NO: 136 - protein, 15 aa, Artificial Sequence
  Viral peptide
PDMIRYIDRQGQLQT

SEQ ID NO: 137 - protein, 15 aa, Artificial Sequence
  Viral peptide
IRYIDRQGQLQTKMV

SEQ ID NO: 138 - protein, 15 aa, Artificial Sequence
  Viral peptide
MATPSMMPQWAYMHI

SEQ ID NO: 139 - protein, 15 aa, Artificial Sequence
  Viral peptide
SMMPQWAYMHIAGQD

SEQ ID NO: 140 - protein, 15 aa, Artificial Sequence
  Viral peptide
QWAYMHIAGQDASGY

SEQ ID NO: 141 - protein, 15 aa, Artificial Sequence
  Viral peptide
MHIAGQDASGYLSPG

SEQ ID NO: 142 - protein, 15 aa, Artificial Sequence
  Viral peptide
GQDASGYLSPGLVQF

SEQ ID NO: 143 - protein, 15 aa, Artificial Sequence
  Viral peptide
SGYLSPGLVQFARAT

SEQ ID NO: 144 - protein, 15 aa, Artificial Sequence
  Viral peptide
SPGLVQFARATDTYF

SEQ ID NO: 145 - protein, 15 aa, Artificial Sequence
  Viral peptide
VQFARATDTYFSMGN

SEQ ID NO: 146 - protein, 15 aa, Artificial Sequence
  Viral peptide
RATDTYFSMGNKFRN

SEQ ID NO: 147 - protein, 15 aa, Artificial Sequence
  Viral peptide
TYFSMGNKFRNPTVA

SEQ ID NO: 148 - protein, 15 aa, Artificial Sequence
  Viral peptide
MGNKFRNPTVAPFHD

SEQ ID NO: 149 - protein, 15 aa, Artificial Sequence
  Viral peptide
FRNPTVAPTMDVTTD

SEQ ID NO: 150 - protein, 15 aa, Artificial Sequence
  Viral peptide
TVAPTHDVTTDRSQR

SEQ ID NO: 151 - protein, 15 aa, Artificial Sequence
  Viral peptide
THDVTTDRSQRLMLR

SEQ ID NO: 152 - protein, 15 aa, Artificial Sequence
  Viral peptide
TTDRSQRLMLRPVPV

SEQ ID NO: 153 - protein, 15 aa, Artificial Sequence
  Viral peptide
SQRLMLRFVPVDRED

SEQ ID NO: 154 - protein, 15 aa, Artificial Sequence
  Viral peptide
MLRFVPVDREDNTYS

SEQ ID NO: 155 - protein, 15 aa, Artificial Sequence
  Viral peptide
VPVDREDNTYSYKVR

SEQ ID NO: 156 - protein, 15 aa, Artificial Sequence
  Viral peptide
REDNTYSYKVRYTLA

SEQ ID NO: 157 - protein, 15 aa, Artificial Sequence
  Viral peptide
TYSYKVRYTLAVGDN

SEQ ID NO: 158 - protein, 15 aa, Artificial Sequence
  Viral peptide
KVRYTLAVGDNRVLD

SEQ ID NO: 159 - protein, 15 aa, Artificial Sequence
  Viral peptide
TLAVGDNRVLDMAST

SEQ ID NO: 160 - protein, 15 aa, Artificial Sequence
  Viral peptide
GDNRVLDMASTFFDI

SEQ ID NO: 161 - protein, 15 aa, Artificial Sequence
  Viral peptide
VLDMASTFFDIRGVL

SEQ ID NO: 162 - protein, 15 aa, Artificial Sequence
  Viral peptide
ASTFFDIRGVLDRGP

SEQ ID NO: 163 - protein, 15 aa, Artificial Sequence
  Viral peptide
FDIRGVLDRGPSFKP

SEQ ID NO: 164 - protein, 15 aa, Artificial Sequence
  Viral peptide
GVLDRGPSFKPYSGT

SEQ ID NO: 165 - protein, 15 aa, Artificial Sequence
  Viral peptide
RGPSFKPYSGTAYNS

SEQ ID NO: 166 - protein, 15 aa, Artificial Sequence
  Viral peptide
FKPYSGTAYNSLAPK

SEQ ID NO: 167 - protein, 15 aa, Artificial Sequence
  Viral peptide
SGTAYNSLAPKGAPN

SEQ ID NO: 168 - protein, 15 aa, Artificial Sequence
  Viral peptide
YNSLAPKGAPNTSQW

SEQ ID NO: 169 - protein, 15 aa, Artificial Sequence
  Viral peptide
APKGAPNTSQWIVTT

SEQ ID NO: 170 - protein, 15 aa, Artificial Sequence
  Viral peptide
APNTSQWIVTINGDN

SEQ ID NO: 171 - protein, 15 aa, Artificial Sequence
  Viral peptide
SQWIVTINGDNAVIT

SEQ ID NO: 172 - protein, 15 aa, Artificial Sequence
  Viral peptide
VTTNGDNAVTTTTNT

SEQ ID NO: 173 - protein, 15 aa, Artificial Sequence
  Viral peptide
GDNAVTTTTNTFGIA

SEQ ID NO: 174 - protein, 15 aa, Artificial Sequence
  Viral peptide
VITTINTFGIASMKG

SEQ ID NO: 175 - protein, 15 aa, Artificial Sequence
  Viral peptide
TNTFGIASMKGGNIT

SEQ ID NO: 176 - protein, 15 aa, Artificial Sequence
  Viral peptide
GIASMKGGNITKEGL

SEQ ID NO: 177 - protein, 15 aa, Artificial Sequence
  Viral peptide
MKGGNITKEGLQIGK

SEQ ID NO: 178 - protein, 15 aa, Artificial Sequence
  Viral peptide
NITKEGLQIGKDITT

SEQ ID NO: 179 - protein, 15 aa, Artificial Sequence
  Viral peptide
EGLQIGKDITTTEGE

SEQ ID NO: 180 - protein, 15 aa, Artificial Sequence
  Viral peptide
IGKDITTTEGEEKPI

SEQ ID NO: 181 - protein, 15 aa, Artificial Sequence
  Viral peptide
ITTTEGEEKPIYADK

SEQ ID NO: 182 - protein, 15 aa, Artificial Sequence
  Viral peptide
EGEEKPIYADKTYQP

SEQ ID NO: 183 - protein, 15 aa, Artificial Sequence
  Viral peptide
KPIYADKTYQPEPQV

SEQ ID NO: 184 - protein, 15 aa, Artificial Sequence
  Viral peptide
ADKTYQPEPQVGEES

SEQ ID NO: 185 - protein, 15 aa, Artificial Sequence
  Viral peptide
YQPEPQVGEESWTDT

SEQ ID NO: 186 - protein, 15 aa, Artificial Sequence
  Viral peptide
PQVGEESWTDTDGTN

SEQ ID NO: 187 - protein, 15 aa, Artificial Sequence
  Viral peptide
EESWTDTDGTNEKFG

SEQ ID NO: 188 - protein, 15 aa, Artificial Sequence
  Viral peptide
TDTDGTNEKFGGRAL

SEQ ID NO: 189 - protein, 15 aa, Artificial Sequence
  Viral peptide
GTNEKFGGRALKPAT

SEQ ID NO: 190 - protein, 15 aa, Artificial Sequence
  Viral peptide
KFGGRALKPATNMKP

SEQ ID NO: 191 - protein, 15 aa, Artificial Sequence
  Viral peptide
RALKPATNMKPCYGS

SEQ ID NO: 192 - protein, 15 aa, Artificial Sequence
  Viral peptide
PATNMKPCYGSFARP

SEQ ID NO: 193 - protein, 15 aa, Artificial Sequence
  Viral peptide
MKPCYGSFARPTNIK

SEQ ID NO: 194 - protein, 15 aa, Artificial Sequence
  Viral peptide
YGSFARPTNIKGGQA

SEQ ID NO: 195 - protein, 15 aa, Artificial Sequence
  Viral peptide
ARPTNIKGGQAKNRK

SEQ ID NO: 196 - protein, 15 aa, Artificial Sequence
  Viral peptide
NIKGGQAKNRKVKPT

SEQ ID NO: 197 - protein, 15 aa, Artificial Sequence
  Viral peptide
GQAKNRKVKPTTEGG

SEQ ID NO: 198 - protein, 15 aa, Artificial Sequence
  Viral peptide
NRKVKPTTEGGVETE

SEQ ID NO: 199 - protein, 15 aa, Artificial Sequence
  Viral peptide
KPTTEGGVETEEPDI

SEQ ID NO: 200 - protein, 15 aa, Artificial Sequence
  Viral peptide
EGGVETEEPDIDMEF

SEQ ID NO: 201 - protein, 15 aa, Artificial Sequence
  Viral peptide
ETEEPDIDMEFFDGR

SEQ ID NO: 202 - protein, 15 aa, Artificial Sequence
  Viral peptide
PDIDMEFFDGRDAVA

SEQ ID NO: 203 - protein, 15 aa, Artificial Sequence
  Viral peptide
MEFFDGRDAVAGALA

SEQ ID NO: 204 - protein, 15 aa, Artificial Sequence
  Viral peptide
DGRDAVAGALAPEIV

SEQ ID NO: 205 - protein, 15 aa, Artificial Sequence
  Viral peptide
AVAGALAPEIVLYTE

SEQ ID NO: 206 - protein, 15 aa, Artificial Sequence
  Viral peptide
ALAPEIVLYTENVNL

SEQ ID NO: 207 - protein, 15 aa, Artificial Sequence
  Viral peptide
EIVLYTENVNLETPD

SEQ ID NO: 208 - protein, 15 aa, Artificial Sequence
  Viral peptide
YTENVNLETPDSHVV

SEQ ID NO: 209 - protein, 15 aa, Artificial Sequence
  Viral peptide
VNLETPDSHVVYKPE

SEQ ID NO: 210 - protein, 15 aa, Artificial Sequence
  Viral peptide
TPDSHVVYKPETSNN

SEQ ID NO: 211 - protein, 15 aa, Artificial Sequence
  Viral peptide
HVVYKPETSNNSHAN

SEQ ID NO: 212 - protein, 15 aa, Artificial Sequence
  Viral peptide
KPETSNNSHANLGQQ

SEQ ID NO: 213 - protein, 15 aa, Artificial Sequence
  Viral peptide
SNNSHANLGQQAMPN

SEQ ID NO: 214 - protein, 15 aa, Artificial Sequence
  Viral peptide
HANLGQQAMPNRPNY

SEQ ID NO: 215 - protein, 15 aa, Artificial Sequence
  Viral peptide
GQQAMPNRPNYIGPR

SEQ ID NO: 216 - protein, 15 aa, Artificial Sequence
  Viral peptide
MPNRPNYIGFRDNFV

SEQ ID NO: 217 - protein, 15 aa, Artificial Sequence
  Viral peptide
PNYIGFRDNFVGLMY

SEQ ID NO: 218 - protein, 15 aa, Artificial Sequence
  Viral peptide
GFRDNFVGLMYYNST

SEQ ID NO: 219 - protein, 15 aa, Artificial Sequence
  Viral peptide
NFVGLMYYNSTGNMG

SEQ ID NO: 220 - protein, 15 aa, Artificial Sequence
  Viral peptide
LMYYNSTGNMGVLAG

SEQ ID NO: 221 - protein, 15 aa, Artificial Sequence
  Viral peptide
NSTGNMGVLAGQASQ

SEQ ID NO: 222 - protein, 15 aa, Artificial Sequence
  Viral peptide
NMGVLAGQASQLNAV

SEQ ID NO: 223 - protein, 15 aa, Artificial Sequence
  Viral peptide
LAGQASQLNAVVDLQ

SEQ ID NO: 224 - protein, 15 aa, Artificial Sequence
  Viral peptide
ASQLNAVVDLQDRNT

SEQ ID NO: 225 - protein, 15 aa, Artificial Sequence
  Viral peptide
NAVVDLQDRNTELSY

SEQ ID NO: 226 - protein, 15 aa, Artificial Sequence
  Viral peptide
DLQDRNTELSYQLLL

SEQ ID NO: 227 - protein, 15 aa, Artificial Sequence
  Viral peptide
RNTELSYQLLLDSLG

SEQ ID NO: 228 - protein, 15 aa, Artificial Sequence
  Viral peptide
LSYQLLLDSLGDRTR

SEQ ID NO: 229 - protein, 15 aa, Artificial Sequence
  Viral peptide
LLLDSLGDRTRYFSM

SEQ ID NO: 230 - protein, 15 aa, Artificial Sequence
  Viral peptide
SLGDRTRYFSMWNQA

SEQ ID NO: 231 - protein, 15 aa, Artificial Sequence
  Viral peptide
RTRYFSMWNQAVDSY

SEQ ID NO: 232 - protein, 15 aa, Artificial Sequence
  Viral peptide
FSMWNQAVDSYDPDV

SEQ ID NO: 233 - protein, 15 aa, Artificial Sequence
  Viral peptide
NQAVDSYDPDVRIIE

SEQ ID NO: 234 - protein, 15 aa, Artificial Sequence
  Viral peptide
DSYDPDVRIIENHGI

SEQ ID NO: 235 - protein, 15 aa, Artificial Sequence
  Viral peptide
PDVRIIENHGIEDEL

SEQ ID NO: 236 - protein, 15 aa, Artificial Sequence
  Viral peptide
IIENHGIEDELPNYC

SEQ ID NO: 237 - protein, 15 aa, Artificial Sequence
  Viral peptide
HGIEDELPNYCFPLN

SEQ ID NO: 238 - protein, 15 aa, Artificial Sequence
  Viral peptide
DELPNYCFPLNGIGP

SEQ ID NO: 239 - protein, 15 aa, Artificial Sequence
  Viral peptide
NYCFPLNGIGPGHTY

SEQ ID NO: 240 - protein, 15 aa, Artificial Sequence
  Viral peptide
PLNGIGPGHTYQGIK

SEQ ID NO: 241 - protein, 15 aa, Artificial Sequence
  Viral peptide
IGPGHTYQGIKVKTD

SEQ ID NO: 242 - protein, 15 aa, Artificial Sequence
  Viral peptide
HTYQGIKVKTDDTNG

SEQ ID NO: 243 - protein, 15 aa, Artificial Sequence
  Viral peptide
GIKVKTDDTNGWEKD

SEQ ID NO: 244 - protein, 15 aa, Artificial Sequence
  Viral peptide
KTDDTNGWEKDANVA

SEQ ID NO: 245 - protein, 15 aa, Artificial Sequence
  Viral peptide
TNGWEKDANVAPANE

SEQ ID NO: 246 - protein, 15 aa, Artificial Sequence
  Viral peptide
EKDANVAPANEITIG

SEQ ID NO: 247 - protein, 15 aa, Artificial Sequence
  Viral peptide
NVAPANEITIGNNLA

SEQ ID NO: 248 - protein, 15 aa, Artificial Sequence
  Viral peptide
ANEITIGNNLAMEIN

SEQ ID NO: 249 - protein, 15 aa, Artificial Sequence
  Viral peptide
TIGNNLAMEINIQAN

SEQ ID NO: 250 - protein, 15 aa, Artificial Sequence
  Viral peptide
NLAMEINIQANLWRS

SEQ ID NO: 251 - protein, 15 aa, Artificial Sequence
  Viral peptide
EINIQANLWRSFLYS

SEQ ID NO: 252 - protein, 15 aa, Artificial Sequence
  Viral peptide
QANLWRSFLYSNVAL

SEQ ID NO: 253 - protein, 15 aa, Artificial Sequence
  Viral peptide
WRSFLYSNVALYLPD

SEQ ID NO: 254 - protein, 15 aa, Artificial Sequence
  Viral peptide
LYSNVALYLPDVYKY

SEQ ID NO: 255 - protein, 15 aa, Artificial Sequence
  Viral peptide
VALYLPDVYKYTPPN

SEQ ID NO: 256 - protein, 15 aa, Artificial Sequence
  Viral peptide
LPDVYKYTPPNITLP

SEQ ID NO: 257 - protein, 15 aa, Artificial Sequence
  Viral peptide
YKYTPPNITLPTNTN

SEQ ID NO: 258 - protein, 15 aa, Artificial Sequence
  Viral peptide
PPNITLPTNTNTYEY

SEQ ID NO: 259 - protein, 15 aa, Artificial Sequence
  Viral peptide
TLPTNTNTYEYMNGR

SEQ ID NO: 260 - protein, 15 aa, Artificial Sequence
  Viral peptide
NTNTYEYMNGRVVSP

SEQ ID NO: 261 - protein, 15 aa, Artificial Sequence
  Viral peptide
YEYMNGRVVSPSLVD

SEQ ID NO: 262 - protein, 15 aa, Artificial Sequence
  Viral peptide
NGRVVSPSLVDSYIN

SEQ ID NO: 263 - protein, 15 aa, Artificial Sequence
  Viral peptide
VSPSLVDSYINIGAR

SEQ ID NO: 264 - protein, 15 aa, Artificial Sequence
  Viral peptide
LVDSYINIGARWSLD

SEQ ID NO: 265 - protein, 15 aa, Artificial Sequence
  Viral peptide
YINIGARWSLDPMDN

SEQ ID NO: 266 - protein, 15 aa, Artificial Sequence
  Viral peptide
GARWSLDPMDNVNPF

SEQ ID NO: 267 - protein, 15 aa, Artificial Sequence
  Viral peptide
SLDPMDNVNPFNHHR

SEQ ID NO: 268 - protein, 15 aa, Artificial Sequence
  Viral peptide
MDNVNPFNHHRNAGL

SEQ ID NO: 269 - protein, 15 aa, Artificial Sequence
  Viral peptide
NPFNHHRNAGLRYRS

SEQ ID NO: 270 - protein, 15 aa, Artificial Sequence
  Viral peptide
HHRNAGLRYRSMLLG

SEQ ID NO: 271 - protein, 15 aa, Artificial Sequence
  Viral peptide
AGLRYRSMLLGNGRY

SEQ ID NO: 272 - protein, 15 aa, Artificial Sequence
  Viral peptide
YRSMLLGNGRYVPFH

SEQ ID NO: 273 - protein, 15 aa, Artificial Sequence
  Viral peptide
LLGNGRYVPFHIQVP

SEQ ID NO: 274 - protein, 15 aa, Artificial Sequence
  Viral peptide
GRYVPFHIQVPQKFF

SEQ ID NO: 275 - protein, 15 aa, Artificial Sequence
  Viral peptide
PFHIQVPQKFFAVKN

SEQ ID NO: 276 - protein, 15 aa, Artificial Sequence
  Viral peptide
QVPQKFFAVKNLLLL

SEQ ID NO: 277 - protein, 15 aa, Artificial Sequence
  Viral peptide
KFFAVKNLLLLPGSY

SEQ ID NO: 278 - protein, 15 aa, Artificial Sequence
  Viral peptide
VKNLLLLPGSYTYEW

SEQ ID NO: 279 - protein, 15 aa, Artificial Sequence
  Viral peptide
LLLPGSYTYEWNFRK

SEQ ID NO: 280 - protein, 15 aa, Artificial Sequence
  Viral peptide
GSYTYEWNFRKDVNM

SEQ ID NO: 281 - protein, 15 aa, Artificial Sequence
  Viral peptide
YEWNFRKDVNMVLQS

SEQ ID NO: 282 - protein, 15 aa, Artificial Sequence
  Viral peptide
FRKDVNMVLQSSLGN

SEQ ID NO: 283 - protein, 15 aa, Artificial Sequence
  Viral peptide
VNMVLQSSLGNDLRT

SEQ ID NO: 284 - protein, 15 aa, Artificial Sequence
  Viral peptide
LQSSLGNDLRTDGAT

SEQ ID NO: 285 - protein, 15 aa, Artificial Sequence
  Viral peptide
LGNDLRTDGATISFT

SEQ ID NO: 286 - protein, 15 aa, Artificial Sequence
  Viral peptide
LRTDGATISFTSINL

SEQ ID NO: 287 - protein, 15 aa, Artificial Sequence
  Viral peptide
GATISFTSINLYATF

SEQ ID NO: 288 - protein, 15 aa, Artificial Sequence
  Viral peptide
SFTSINLYATFFPMA

SEQ ID NO: 289 - protein, 15 aa, Artificial Sequence
  Viral peptide
INLYATFFPMAKNTA

SEQ ID NO: 290 - protein, 15 aa, Artificial Sequence
  Viral peptide
ATFFPMAKNTASTLE

SEQ ID NO: 291 - protein, 15 aa, Artificial Sequence
  Viral peptide
PMAHNTASTLEAMLR

SEQ ID NO: 292 - protein, 15 aa, Artificial Sequence
  Viral peptide
NTASTLEAMLRNDTN

SEQ ID NO: 293 - protein, 15 aa, Artificial Sequence
  Viral peptide
TLEAMLRNDTNDQSF

SEQ ID NO: 294 - protein, 15 aa, Artificial Sequence
  Viral peptide
MLRNDTNDQSFNDYL

SEQ ID NO: 295 - protein, 15 aa, Artificial Sequence
  Viral peptide
DTNDQSFNDYLSAAN

SEQ ID NO: 296 - protein, 15 aa, Artificial Sequence
  Viral peptide
QSFNDYLSAANMLYP

SEQ ID NO: 297 - protein, 15 aa, Artificial Sequence
  Viral peptide
DYLSAANMLYPIPAN

SEQ ID NO: 298 - protein, 15 aa, Artificial Sequence
  Viral peptide
AANMLYPIPANATNI

SEQ ID NO: 299 - protein, 15 aa, Artificial Sequence
  Viral peptide
LYPIPANATNIPISI

SEQ ID NO: 300 - protein, 15 aa, Artificial Sequence
  Viral peptide
PANATNIPISIPSRN

SEQ ID NO: 301 - protein, 15 aa, Artificial Sequence
  Viral peptide
TNIPISIPSRNWAAF

SEQ ID NO: 302 - protein, 15 aa, Artificial Sequence
  Viral peptide
ISIPSRNWAAFRGWS

SEQ ID NO: 303 - protein, 15 aa, Artificial Sequence
  Viral peptide
SRNWAAFRGWSFTRL

SEQ ID NO: 304 - protein, 15 aa, Artificial Sequence
  Viral peptide
AAFRGWSFTRLKTKE

SEQ ID NO: 305 - protein, 15 aa, Artificial Sequence
  Viral peptide
GWSFTRLKTKETPSL

SEQ ID NO: 306 - protein, 15 aa, Artificial Sequence
  Viral peptide
TRLKTKETPSLGSGF

SEQ ID NO: 307 - protein, 15 aa, Artificial Sequence
  Viral peptide
TKETPSLGSGFDPYF

SEQ ID NO: 308 - protein, 15 aa, Artificial Sequence
  Viral peptide
PSLGSGFDPYFVYSG

SEQ ID NO: 309 - protein, 15 aa, Artificial Sequence
  Viral peptide
SGFDPYFVYSGSIPY

SEQ ID NO: 310 - protein, 15 aa, Artificial Sequence
  Viral peptide
PYFVYSGSIPYLDGT

SEQ ID NO: 311 - protein, 15 aa, Artificial Sequence
  Viral peptide
YSGSIPYLDGTFYLN

SEQ ID NO: 312 - protein, 15 aa, Artificial Sequence
  Viral peptide
IPYLDGTFYLNHTFK

SEQ ID NO: 313 - protein, 15 aa, Artificial Sequence
  Viral peptide
DGTFYLNHTFKKVAI

SEQ ID NO: 314 - protein, 15 aa, Artificial Sequence
  Viral peptide
YLNHTFKKVAIMFDS

SEQ ID NO: 315 - protein, 15 aa, Artificial Sequence
  Viral peptide
TFKKVAIMFDSSVSW

SEQ ID NO: 316 - protein, 15 aa, Artificial Sequence
  Viral peptide
VAIMFDSSVSWPGND

SEQ ID NO: 317 - protein, 15 aa, Artificial Sequence
  Viral peptide
FDSSVSWPGNDRLLS

SEQ ID NO: 318 - protein, 15 aa, Artificial Sequence
  Viral peptide
VSWPGNDRLLSPNEF

SEQ ID NO: 319 - protein, 15 aa, Artificial Sequence
  Viral peptide
GNDRLLSPNEFEIKR

SEQ ID NO: 320 - protein, 15 aa, Artificial Sequence
  Viral peptide
LLSPNEFEIKRTVDG

SEQ ID NO: 321 - protein, 15 aa, Artificial Sequence
  Viral peptide
NEFEIKRTVDGEGYN

SEQ ID NO: 322 - protein, 15 aa, Artificial Sequence
  Viral peptide
IKRTVDGEGYNVAQC

SEQ ID NO: 323 - protein, 15 aa, Artificial Sequence
  Viral peptide
VDGEGYNVAQCNMTK

SEQ ID NO: 324 - protein, 15 aa, Artificial Sequence
  Viral peptide
GYNVAQCNMTKDWFL

SEQ ID NO: 325 - protein, 15 aa, Artificial Sequence
  Viral peptide
AQCNMTKDWFLVQML

SEQ ID NO: 326 - protein, 15 aa, Artificial Sequence
  Viral peptide
MTKDWFLVQMLANYN

SEQ ID NO: 327 - protein, 15 aa, Artificial Sequence
  Viral peptide
WFLVQMLANYNIGYQ

SEQ ID NO: 328 - protein, 15 aa, Artificial Sequence
  Viral peptide
QMLANYNIGYQGFYI

SEQ ID NO: 329 - protein, 15 aa, Artificial Sequence
  Viral peptide
NYNIGYQGFYIPEGY

SEQ ID NO: 330 - protein, 15 aa, Artificial Sequence
  Viral peptide
GYQGFYIPEGYKDRM

SEQ ID NO: 331 - protein, 15 aa, Artificial Sequence
  Viral peptide
FYIPEGYKDRMYSFF

SEQ ID NO: 332 - protein, 15 aa, Artificial Sequence
  Viral peptide
EGYKDRMYSFFRNFQ

SEQ ID NO: 333 - protein, 15 aa, Artificial Sequence
  Viral peptide
DRMYSFFRNFQPMSR

SEQ ID NO: 334 - protein, 15 aa, Artificial Sequence
  Viral peptide
SFFRNFQPMSRQVVD

SEQ ID NO: 335 - protein, 15 aa, Artificial Sequence
  Viral peptide
NFQPMSRQVVDEVNY

SEQ ID NO: 336 - protein, 15 aa, Artificial Sequence
  Viral peptide
MSRQVVDEVNYTDYK

SEQ ID NO: 337 - protein, 15 aa, Artificial Sequence
  Viral peptide
VVDEVNYTDYKAVTL

SEQ ID NO: 338 - protein, 15 aa, Artificial Sequence
  Viral peptide
VNYTDYKAVTLPYQH

SEQ ID NO: 339 - protein, 15 aa, Artificial Sequence
  Viral peptide
DYKAVTLPYQHNNSG

SEQ ID NO: 340 - protein, 15 aa, Artificial Sequence
  Viral peptide
VTLPYQHNNSGFVGY

SEQ ID NO: 341 - protein, 15 aa, Artificial Sequence
  Viral peptide
YQHNNSGFVGYLAPT

SEQ ID NO: 342 - protein, 15 aa, Artificial Sequence
  Viral peptide
NSGFVGYLAPTMRQG

SEQ ID NO: 343 - protein, 15 aa, Artificial Sequence
  Viral peptide
VGYLAPTMRQGEPYP

SEQ ID NO: 344 - protein, 15 aa, Artificial Sequence
  Viral peptide
APTMRQGEPYPANYP

SEQ ID NO: 345 - protein, 15 aa, Artificial Sequence
  Viral peptide
RQGEPYPANYPYPLI

SEQ ID NO: 346 - protein, 15 aa, Artificial Sequence
  Viral peptide
PYPANYPYPLIGTTA

SEQ ID NO: 347 - protein, 15 aa, Artificial Sequence
  Viral peptide
NYPYPLIGTTAVKSV

SEQ ID NO: 348 - protein, 15 aa, Artificial Sequence
  Viral peptide
PLIGTTAVKSVTQKK

SEQ ID NO: 349 - protein, 15 aa, Artificial Sequence
  Viral peptide
TTAVKSVTQKKFLCD

SEQ ID NO: 350 - protein, 15 aa, Artificial Sequence
  Viral peptide
KSVTQKKFLCDRTMW

SEQ ID NO: 351 - protein, 15 aa, Artificial Sequence
  Viral peptide
QKKFLCDRTMWRIPF

SEQ ID NO: 352 - protein, 15 aa, Artificial Sequence
  Viral peptide
LCDRTMWRIPFSSNF

SEQ ID NO: 353 - protein, 15 aa, Artificial Sequence
  Viral peptide
TMWRIPFSSNFMSMG

SEQ ID NO: 354 - protein, 15 aa, Artificial Sequence
  Viral peptide
IPFSSNFMSMGALTD

SEQ ID NO: 355 - protein, 15 aa, Artificial Sequence
  Viral peptide
SNFMSMGALTDLGQN

SEQ ID NO: 356 - protein, 15 aa, Artificial Sequence
  Viral peptide
SMGALTDLGQNMLYA

SEQ ID NO: 357 - protein, 15 aa, Artificial Sequence
  Viral peptide
LTDLGQNMLYANSAH

SEQ ID NO: 358 - protein, 15 aa, Artificial Sequence
  Viral peptide
GQNMLYANSAHALDM

SEQ ID NO: 359 - protein, 15 aa, Artificial Sequence
  Viral peptide
LYANSAHALDMTFEV

SEQ ID NO: 360 - protein, 15 aa, Artificial Sequence
  Viral peptide
SAHALDMTFEVDPMD

SEQ ID NO: 361 - protein, 15 aa, Artificial Sequence
  Viral peptide
LDMTFEVDPMDEPTL

SEQ ID NO: 362 - protein, 15 aa, Artificial Sequence
  Viral peptide
FEVDPMDEPTLLYLL

SEQ ID NO: 363 - protein, 15 aa, Artificial Sequence
  Viral peptide
PMDEPTLLYLLFEVF

SEQ ID NO: 364 - protein, 15 aa, Artificial Sequence
  Viral peptide
PTLLYLLFEVFDVVR

SEQ ID NO: 365 - protein, 15 aa, Artificial Sequence
  Viral peptide
YLLFEVFDVVRVHQP

SEQ ID NO: 366 - protein, 15 aa, Artificial Sequence
  Viral peptide
EVFDVVRVHQPHRGV

SEQ ID NO: 367 - protein, 15 aa, Artificial Sequence
  Viral peptide
VVRVHQPHRGVIEAV

SEQ ID NO: 368 - protein, 15 aa, Artificial Sequence
  Viral peptide
HQPHRGVIEAVYLRT

SEQ ID NO: 369 - protein, 15 aa, Artificial Sequence
  Viral peptide
RGVIEAVYLRTPFSA

SEQ ID NO: 370 - protein, 15 aa, Artificial Sequence
  Viral peptide
EAVYLRTPFSAGNAT

SEQ ID NO: 371 - protein, 12 aa, Artificial Sequence
  Viral peptide
LRTPFSAGNATT

SEQ ID NO: 372 - protein, 15 aa, Artificial Sequence
  Viral peptide
MQPATLQWSSYVLQL

SEQ ID NO: 373 - protein, 15 aa, Artificial Sequence
  Viral peptide
TLQWSSYVLQLRLTT

SEQ ID NO: 374 - protein, 15 aa, Artificial Sequence
  Viral peptide
SSYVLQLRLTTAAIL

SEQ ID NO: 375 - protein, 15 aa, Artificial Sequence
  Viral peptide
LQLRLTTAAILKPGE

SEQ ID NO: 376 - protein, 15 aa, Artificial Sequence
  Viral peptide
LTTAAILKPGELRYF

SEQ ID NO: 377 - protein, 15 aa, Artificial Sequence
  Viral peptide
AILKPGELRYFKCGL

SEQ ID NO: 378 - protein, 15 aa, Artificial Sequence
  Viral peptide
PGELRYFKCGLGICP

SEQ ID NO: 379 - protein, 15 aa, Artificial Sequence
  Viral peptide
RYFKCGLGICPPSSS

SEQ ID NO: 380 - protein, 15 aa, Artificial Sequence
  Viral peptide
CGLGICPPSSSVVCT

SEQ ID NO: 381 - protein, 15 aa, Artificial Sequence
  Viral peptide
ICPPSSSVVCTCRDE

SEQ ID NO: 382 - protein, 15 aa, Artificial Sequence
  Viral peptide
SSSVVCTCRDESSFA

SEQ ID NO: 383 - protein, 15 aa, Artificial Sequence
  Viral peptide
VCTCRDESSFAASPF

SEQ ID NO: 384 - protein, 15 aa, Artificial Sequence
  Viral peptide
RDESSFAASPFTYID

SEQ ID NO: 385 - protein, 15 aa, Artificial Sequence
  Viral peptide
SFAASPFTYIDPKDY

SEQ ID NO: 386 - protein, 15 aa, Artificial Sequence
  Viral peptide
SPFTYIDPKDYGNIP

SEQ ID NO: 387 - protein, 15 aa, Artificial Sequence
  Viral peptide
YIDPKDYGNIPFAVH

SEQ ID NO: 388 - protein, 15 aa, Artificial Sequence
  Viral peptide
KDYGNIPFAVHNISD

SEQ ID NO: 389 - protein, 15 aa, Artificial Sequence
  Viral peptide
NIPFAVHNISDLDLD

SEQ ID NO: 390 - protein, 15 aa, Artificial Sequence
  Viral peptide
AVHNISDLDLDLGRL

SEQ ID NO: 391 - protein, 15 aa, Artificial Sequence
  Viral peptide
ISDLDLDLGRLPIVL

SEQ ID NO: 392 - protein, 15 aa, Artificial Sequence
  Viral peptide
DLDLGRLPIVLNIFA

SEQ ID NO: 393 - protein, 15 aa, Artificial Sequence
  Viral peptide
GRLPIVLNIFALPYA

SEQ ID NO: 394 - protein, 15 aa, Artificial Sequence
  Viral peptide
IVLNIFALPYANVTV

SEQ ID NO: 395 - protein, 15 aa, Artificial Sequence
  Viral peptide
IFALPYANVTVSNLP

SEQ ID NO: 396 - protein, 15 aa, Artificial Sequence
  Viral peptide
PYANVTVSNLPVQRI

SEQ ID NO: 397 - protein, 15 aa, Artificial Sequence
  Viral peptide
VTVSNLPVQRIEAYD

SEQ ID NO: 398 - protein, 15 aa, Artificial Sequence
  Viral peptide
NLPVQRIEAYDRHAI

SEQ ID NO: 399 - protein, 15 aa, Artificial Sequence
  Viral peptide
QRIEAYDRHAIPHGQ

SEQ ID NO: 400 - protein, 15 aa, Artificial Sequence
  Viral peptide
AYDRHAIPHGQCEAQ

SEQ ID NO: 401 - protein, 15 aa, Artificial Sequence
  Viral peptide
HAIPHGQCEAQFILY

SEQ ID NO: 402 - protein, 15 aa, Artificial Sequence
  Viral peptide
HGQCEAQFILYGPLT

SEQ ID NO: 403 - protein, 15 aa, Artificial Sequence
  Viral peptide
EAQFILYGPLTRIKI

SEQ ID NO: 404 - protein, 15 aa, Artificial Sequence
  Viral peptide
ILYGPLTRIKIYAAD

SEQ ID NO: 405 - protein, 15 aa, Artificial Sequence
  Viral peptide
PLTRIKIYAADIRWT

SEQ ID NO: 406 - protein, 15 aa, Artificial Sequence
  Viral peptide
IKIYAADIRWTEKTP

SEQ ID NO: 407 - protein, 15 aa, Artificial Sequence
  Viral peptide
AADIRWTEKTPEEPT

SEQ ID NO: 408 - protein, 15 aa, Artificial Sequence
  Viral peptide
RWTEKTPEEPTRYLF

SEQ ID NO: 409 - protein, 15 aa, Artificial Sequence
  Viral peptide
KTPEEPTRYLFNADI

SEQ ID NO: 410 - protein, 15 aa, Artificial Sequence
  Viral peptide
EPTRYLFNADIWINL

SEQ ID NO: 411 - protein, 15 aa, Artificial Sequence
  Viral peptide
YLFNADIWINLQNTP

SEQ ID NO: 412 - protein, 15 aa, Artificial Sequence
  Viral peptide
ADIWINLQNTPLDQI

SEQ ID NO: 413 - protein, 15 aa, Artificial Sequence
  Viral peptide
INLQNTPLDQIFNSA

SEQ ID NO: 414 - protein, 15 aa, Artificial Sequence
  Viral peptide
NTPLDQIFNSAKIEF

SEQ ID NO: 415 - protein, 15 aa, Artificial Sequence
  Viral peptide
DQIFNSAKIEFISHR

SEQ ID NO: 416 - protein, 15 aa, Artificial Sequence
  Viral peptide
NSAKIEFISHRHVSI

SEQ ID NO: 417 - protein, 15 aa, Artificial Sequence
  Viral peptide
IEFISHRHVSISRIL

SEQ ID NO: 418 - protein, 15 aa, Artificial Sequence
  Viral peptide
SHRHVSISRILLCGN

SEQ ID NO: 419 - protein, 15 aa, Artificial Sequence
  Viral peptide
VSISRILLCGNLLIL

SEQ ID NO: 420 - protein, 15 aa, Artificial Sequence
  Viral peptide
RILLCGNLLILKTVY

SEQ ID NO: 421 - protein, 15 aa, Artificial Sequence
  Viral peptide
CGNLLILKTVYENDY

SEQ ID NO: 422 - protein, 15 aa, Artificial Sequence
  Viral peptide
LILKTVYENDYLLDD

SEQ ID NO: 423 - protein, 15 aa, Artificial Sequence
  Viral peptide
TVYENDYLLDDTFFP

SEQ ID NO: 424 - protein, 15 aa, Artificial Sequence
  Viral peptide
NDYLLDDTFFPEKLN

SEQ ID NO: 425 - protein, 15 aa, Artificial Sequence
  Viral peptide
LDDTFFPEKLNIQLS

SEQ ID NO: 426 - protein, 15 aa, Artificial Sequence
  Viral peptide
FFPEKLNIQLSLTPH

SEQ ID NO: 427 - protein, 15 aa, Artificial Sequence
  Viral peptide
KLNIQLSLTPHTADI

SEQ ID NO: 428 - protein, 15 aa, Artificial Sequence
  Viral peptide
QLSLTPHTADITMSQ

SEQ ID NO: 429 - protein, 15 aa, Artificial Sequence
  Viral peptide
TPHTADITMSQNQET

SEQ ID NO: 430 - protein, 15 aa, Artificial Sequence
  Viral peptide
ADITMSQNQETLLKC

SEQ ID NO: 431 - protein, 15 aa, Artificial Sequence
  Viral peptide
MSQNQETLLKCNVGS

SEQ ID NO: 432 - protein, 15 aa, Artificial Sequence
  Viral peptide
QETLLKCNVGSITVG

SEQ ID NO: 433 - protein, 15 aa, Artificial Sequence
  Viral peptide
LKCNVGSITVGATKN

SEQ ID NO: 434 - protein, 15 aa, Artificial Sequence
  Viral peptide
VGSITVGATKNIVSN

SEQ ID NO: 435 - protein, 15 aa, Artificial Sequence
  Viral peptide
TVGATKNIVSNLITP

SEQ ID NO: 436 - protein, 15 aa, Artificial Sequence
  Viral peptide
TKNIVSNLITPFHCS

SEQ ID NO: 437 - protein, 15 aa, Artificial Sequence
  Viral peptide
VSNLITPFHCSFHTI

SEQ ID NO: 438 - protein, 15 aa, Artificial Sequence
  Viral peptide
ITPFHCSFHTIFESN

SEQ ID NO: 439 - protein, 15 aa, Artificial Sequence
  Viral peptide
HCSFHTIFESNSNFT

SEQ ID NO: 440 - protein, 15 aa, Artificial Sequence
  Viral peptide
HTIFESNSNFTGFFI

SEQ ID NO: 441 - protein, 15 aa, Artificial Sequence
  Viral peptide
ESNSNFTGFFIPKLL

SEQ ID NO: 442 - protein, 15 aa, Artificial Sequence
  Viral peptide
NFTGFFIPKLLHGIS

SEQ ID NO: 443 - protein, 15 aa, Artificial Sequence
  Viral peptide
FFIPKLLHGISITTC

SEQ ID NO: 444 - protein, 15 aa, Artificial Sequence
  Viral peptide
KLLHGISITTCIWDE

SEQ ID NO: 445 - protein, 15 aa, Artificial Sequence
  Viral peptide
GISITTCIWDETRPL

SEQ ID NO: 446 - protein, 15 aa, Artificial Sequence
  Viral peptide
TTCIWDETRPLYITM

SEQ ID NO: 447 - protein, 15 aa, Artificial Sequence
  Viral peptide
WDETRPLYITMKAQK

SEQ ID NO: 448 - protein, 15 aa, Artificial Sequence
  Viral peptide
RPLYITMKAQKKNCR

SEQ ID NO: 449 - protein, 15 aa, Artificial Sequence
  Viral peptide
ITMKAQKKNCRINYS

SEQ ID NO: 450 - protein, 15 aa, Artificial Sequence
  Viral peptide
AQKKNCRINYSQELG

SEQ ID NO: 451 - protein, 15 aa, Artificial Sequence
  Viral peptide
NCRINYSQELGKVFF

SEQ ID NO: 452 - protein, 15 aa, Artificial Sequence
  Viral peptide
NYSQELGKVFFFPKQ

SEQ ID NO: 453 - protein, 15 aa, Artificial Sequence
  Viral peptide
ELGKVFFFPKQILPP

SEQ ID NO: 454 - protein, 15 aa, Artificial Sequence
  Viral peptide
VFFFPKQILPPGNNI

SEQ ID NO: 455 - protein, 15 aa, Artificial Sequence
  Viral peptide
PKQILPPGNNIEFSW

SEQ ID NO: 456 - protein, 15 aa, Artificial Sequence
  Viral peptide
LPPGNNIEFSWTEMS

SEQ ID NO: 457 - protein, 15 aa, Artificial Sequence
  Viral peptide
NNIEFSWTEMSNIYV

SEQ ID NO: 458 - protein, 15 aa, Artificial Sequence
  Viral peptide
FSWTEMSNIYVNIES

SEQ ID NO: 459 - protein, 15 aa, Artificial Sequence
  Viral peptide
EMSNIYVNIESPNGP

SEQ ID NO: 460 - protein, 15 aa, Artificial Sequence
  Viral peptide
IYVNIESPNGPVKDI

SEQ ID NO: 461 - protein, 15 aa, Artificial Sequence
  Viral peptide
IESPNGPVKDIVALA

SEQ ID NO: 462 - protein, 15 aa, Artificial Sequence
  Viral peptide
NGPVKDIVALASQPV

SEQ ID NO: 463 - protein, 15 aa, Artificial Sequence
  Viral peptide
KDIVALASQPVSRAP

SEQ ID NO: 464 - protein, 15 aa, Artificial Sequence
  Viral peptide
ALASQPVSRAPSLTT

SEQ ID NO: 465 - protein, 15 aa, Artificial Sequence
  Viral peptide
QPVSRAPSLTTVAHV

SEQ ID NO: 466 - protein, 15 aa, Artificial Sequence
  Viral peptide
RAPSLTTVAHVTPEI

SEQ ID NO: 467 - protein, 15 aa, Artificial Sequence
  Viral peptide
LTTVAHVTPEISMGI

SEQ ID NO: 468 - protein, 15 aa, Artificial Sequence
  Viral peptide
AHVTPEISMGIFLKS

SEQ ID NO: 469 - protein, 15 aa, Artificial Sequence
  Viral peptide
PEISMGIFLKSLRIA

SEQ ID NO: 470 - protein, 15 aa, Artificial Sequence
  Viral peptide
MGIFLKSLRIAFDKE

SEQ ID NO: 471 - protein, 15 aa, Artificial Sequence
  Viral peptide
LKSLRIAFDKEHLVP

SEQ ID NO: 472 - protein, 15 aa, Artificial Sequence
  Viral peptide
RIAFDKEHLVPLHFF

SEQ ID NO: 473 - protein, 15 aa, Artificial Sequence
  Viral peptide
DKEHLVPLHFFLKPG

SEQ ID NO: 474 - protein, 15 aa, Artificial Sequence
  Viral peptide
LVPLHFFLKPGESTR

SEQ ID NO: 475 - protein, 15 aa, Artificial Sequence
  Viral peptide
HFFLKPGESTRMEFM

SEQ ID NO: 476 - protein, 15 aa, Artificial Sequence
  Viral peptide
KPGESTRMEFMPPET

SEQ ID NO: 477 - protein, 15 aa, Artificial Sequence
  Viral peptide
STRMEFMPPETPQSL

SEQ ID NO: 478 - protein, 15 aa, Artificial Sequence
  Viral peptide
EFMPPETPQSLTILE

SEQ ID NO: 479 - protein, 15 aa, Artificial Sequence
  Viral peptide
PETPQSLTILEGDVG

SEQ ID NO: 480 - protein, 15 aa, Artificial Sequence
  Viral peptide
QSLTILEGDVGIRFI

SEQ ID NO: 481 - protein, 15 aa, Artificial Sequence
  Viral peptide
ILEGDVGIRFIPCHN

SEQ ID NO: 482 - protein, 15 aa, Artificial Sequence
  Viral peptide
DVGIRFIPCHNNYSH

SEQ ID NO: 483 - protein, 15 aa, Artificial Sequence
  Viral peptide
RFIPCHNNYSHRSSP

SEQ ID NO: 484 - protein, 15 aa, Coronavirus
LRGHLRIAGHHLGRC

SEQ ID NO: 485 - protein, 14 aa, Coronavirus
RGHLRMAGHPLGRC

SEQ ID NO: 486 - protein, 12 aa, Coronavirus
RGHLYIQGVKLG

SEQ ID NO: 487 - protein, 13 aa, Coronavirus
GHLKIAGMHFGAC

SEQ ID NO: 488 - protein, 15 aa, Coronavirus
LRIAGHHLGRCDIKD

SEQ ID NO: 489 - protein, 15 aa, Coronavirus
LRMAGHPLGRCDIKD

SEQ ID NO: 490 - protein, 15 aa, Coronavirus
SRTLSYYKLGASQRV

SEQ ID NO: 491 - protein, 15 aa, Coronavirus
SRTLSYYKLGASQRV

SEQ ID NO: 492 - protein, 15 aa, Coronavirus
SYYKLGASQRVAGDS

SEQ ID NO: 493 - protein, 15 aa, Coronavirus
SYYKLGASQRVGTDS

SEQ ID NO: 494 - protein, 15 aa, Coronavirus
LGASQRVAGDSGFAA

SEQ ID NO: 495 - protein, 15 aa, Coronavirus
LGASQRVGTDSGFAA

SEQ ID NO: 496 - protein, 13 aa, Coronavirus
LGASVTEDVKFAA

SEQ ID NO: 497 - protein, 15 aa, Coronavirus
KPRQKRTATKAYNVT

SEQ ID NO: 498 - protein, 15 aa, Coronavirus
KPRQKRTATKQYNVT

SEQ ID NO: 499 - protein, 10 aa, Coronavirus
KPRQKRSPNK

SEQ ID NO: 500 - protein, 14 aa, Coronavirus
KPRWKRVPTREENV

SEQ ID NO: 501 - protein, 12 aa, Coronavirus
RHKRVATKSFNV

SEQ ID NO: 502 - protein, 15 aa, Coronavirus
AFFGMSRIGMEVTPS

SEQ ID NO: 503 - protein, 15 aa, Coronavirus
AFFGMSRIGMEVTPS

SEQ ID NO: 504 - protein, 15 aa, Coronavirus
MSRIGMEVTPSGTWL

SEQ ID NO: 505 - protein, 15 aa, Coronavirus
MSRIGMEVTPSGTWL

SEQ ID NO: 506 - protein, 15 aa, Coronavirus
GMEVTPSGTWLTYTG

SEQ ID NO: 507 - protein, 13 aa, Coronavirus
GMEVTPSGTWLTY

SEQ ID NO: 508 - protein, 15 aa, Coronavirus
SKHTPINLVRDLPQG

SEQ ID NO: 509 - protein, 9 aa, Coronavirus
PANIVLPQG

SEQ ID NO: 510 - protein, 11 aa, Coronavirus
PIDVVRDLPSG

SEQ ID NO: 511 - protein, 15 aa, Coronavirus
PINLVRDLPQGFSAL

SEQ ID NO: 512 - protein, 15 aa, Coronavirus
PIDVVRDLPSGFNTL

SEQ ID NO: 513 - protein, 9 aa, Coronavirus
PANIVLPQG

SEQ ID NO: 514 - protein, 15 aa, Coronavirus
YNYLYRLFRKSNLKP

SEQ ID NO: 515 - protein, 15 aa, Coronavirus
YNYKYRYLRHGKLRP

(19) United States
(12) Patent Application Publication
(10) Pub. No.: US 2022/0160767 A1
(43) Pub. Date: May 26, 2022

(54) CYTOKINE COCKTAILS FOR SELECTIVE EXPANSION OF T CELL SUBSETS

(71) Applicant: CHILDREN'S NATIONAL MEDICAL CENTER, Washington, DC (US)

(72) Inventors: Patrick HANLEY, Washington, DC (US); Christopher LAZARSKI, Germantown, MD (US); Michael KELLER, Laurel, MD (US); Catherine BOLLARD, Bethesda, MD (US)

(73) Assignee: CHILDREN'S NATIONAL MEDICAL CENTER, Washington, DC (US)

(21) Appl. No.: 17/537,816

(22) Filed: Nov. 30, 2021

Related U.S. Application Data
(63) Continuation-in-part of application No. PCT/US2020/035618, filed on Jun. 1, 2020.
Application No. PCT/US2020/035618 is a continuation-in-part of application No. PCT/US2021/049902, filed on Sep. 10, 2021.
(60) Provisional application No. 62/855,889, filed on May 31, 2019.
(60) Provisional application No. 63/076,923, filed on Sep. 10, 2020.

(51) Int. Cl. A61K 35/17; A61K 39/00; A61K 39/12; C07K 14/005; C07K 14/54; A61P 31/14

(52) U.S. Cl. CPC A61K 35/17; A61K 39/00115; A61K 39/001153; A61K 39/001189; A61K 39/12; A61K 2039/5158; C07K 14/5406; C07K 14/5412; C07K 14/5418; C07K 14/5443; A61P 31/14; C07K 14/005

(57) ABSTRACT

The disclosure relates to methods of culturing and expanding CD4+ and/or CD8+ T cells in culture. In some embodiments, the methods include expanding, proliferating and storing lymphocytes in tissue culture by exposing the lymphocytes to a combination of cytokines and/or nucleic acids expressing cytokines or functional fragments or variants thereof. The disclosure further relates to methods of generating and manufacturing CD4+ and/or CD8+ T cells that are specific to one or a plurality of viral antigens.

### CROSS-REFERENCE TO RELATED APPLICATIONS

This application claims priority to PCT/US2021/049902, filed Sep. 10, 2021, which claims priority to U.S. Provisional Application No. 63/076,923, filed Sep. 10, 2020 and to PCT/US2020/035618, filed Jun. 1, 2020 which claims priority to U.S. Provisional 62/855,889, filed May 31, 2019. Each of these four applications is incorporated by reference in its entirety.

### REFERENCE TO A SEQUENCE LISTING

In accordance with 37 CFR § 1.52(e)(5), the present specification makes reference to a Sequence Listing (submitted electronically as a .txt file named “538685US_ST25.txt”. The .txt file was generated on Nov. 18, 2021 and is 158,547 bytes in size. The entire contents of the Sequence Listing are herein incorporated by reference.

### TECHNOLOGY FIELD

The disclosure relates to methods of culturing and expanding CD4+ and/or CD8+ T cells in culture. In some embodiments, the methods include expanding, proliferating and storing lymphocytes in tissue culture by exposing the lymphocytes to a combination of cytokines and/or nucleic acids expressing cytokines (or functional fragments or variants thereof). The disclosure further relates to methods of generating and manufacturing CD4+ and/or CD8+ T cells that are specific to one or a plurality of viral antigens.

### BACKGROUND

Viral infections after transplant are one of the greatest complications for patients. Antiviral T cells have demonstrated promise in early phase clinical studies. Moreover, because T cell immunity contributes to the control of many viral pathogens, adoptive immunotherapy with virus-specific T cells (VSTs) has been a logical and effective way of combating severe viral disease in immunocompromised patients in multiple phase 1 and 2 clinical trials. Adoptive immunotherapy with virus-specific T cells (VSTs) therefore can be an alternative and effective way to treat viral infections that lack any effective treatment options. For example, T cells specific to coronavirus antigens, specifically antigens from SARS-CoV-2 (formerly known as 2019-nCoV or novel coronavirus 2019) can be an alternative and effective way to treat COVID-19.

Manufacture of viral specific T cell products expands a heterogeneous pool of pre-existing memory T cells from donor peripheral blood within in vitro culture vessels, with the final product containing a polyclonal mixture of CD4+ helper and CD8+ cytotoxic T cells. This diversity in their composition increases the complexity of their manufacture, as CD4+ T cells and CD8+ T cells can respond differently to cytokine stimulation. For example, IL-4 has been shown to enhance survival of resting T cells and induce CD4+ Th2 helper differentiation [4-6], while IL-15 promotes survival and diversity of CD8+ memory T cells [7,8].

Other examples include IL-6, which has been proposed to enhance Th17 development and inhibit the differentiation of T regulatory cells (T regs) [9], IL-7, which promotes T cell homeostatic survival [10-12], and IL-21, which also promotes the activity of CD8+ T cells and formation or maintenance of central memory [13-15]. In addition, IL-2 is a canonical T cell growth cytokine, which continues to be used in clinical trials due to its demonstrated effectiveness in expanding T cells derived from tumor infiltrating lymphocytes [16]. However, engraftment and survival of sufficient numbers of highly differentiated memory T cell subsets upon adoptive transfer is difficult to achieve; Bush, D. H., et al., Role of memory T cell subsets for adoptive immunotherapy, Seminars in Immunology, 2016, 28, 28-34. For example, such T cell based therapies are limited by rapid acquisition of tolerant phenotypes of T cells or limited by low percentages of memory T cells produced in vitro.

Our previous manufacturing methods have transitioned from culturing T cells in 24 well plates with IL-2 and APC transduced with viral antigens [17,18] to a simplified culture containing a combination of IL-4 and IL-7 in G-Rex gas permeable vessels with soluble mixes of peptides [3,19,20]. This culture system has proven rapid and efficacious for expanding T cells specific for multiple viruses; however, it is desirable to expand more CD4+ and CD8+ T cells in a reliable method or improved clinical scalability and expanding more consistently simplistic T cell products. While such T cell culture systems have been shown to expand T cells specific for a virus using specific cytokines, there is a need for a safer, more reliable, simpler, clinically scalable method for inducing, expanding, or recovering clinically relevant numbers of virus-specific or tumor-specific T cells, especially for T cells having phenotypes useful for sustained prevention or treatment of immunosuppressed patients at risk of opportunistic viral infections or for cancer relapse.

### SUMMARY OF EMBODIMENTS

The disclosure relates to certain cytokine compositions as well as the same compositions that induce or stimulate growth and proliferation of CD4+ and CD8+ T cell subpopulations after exposure for a time period sufficient to induce the growth or proliferation. The method allows for a scientist to choose how and when to grow more cytotoxic T cells instead of helper CD4+ T cells. Essentially, this allows one of ordinary skill to toggle between CD4 and CD8 dominance by choosing a different cytokine mixture, which ultimately creates T cell products of different compositions. Additional advantages of the disclosed method and compositions will be set forth in part in the description which follows, and in part will be understood from the description, or may be learned by practice of the disclosed methods and compositions.

The disclosure also relates to a tissue culture system comprising a plurality of lymphocytes positioned within at least one vessel, cell culture media and a composition comprising at least two cytokines from Table 1 or functional fragments or variants thereof, wherein the functional fragments thereof or the variants thereof comprises at least about 75% sequence identity to the sequences identified in Table 1.

The disclosure relates to a method of selectively growing memory effector T cells from a cell composition comprising naïve T cells comprising: contacting one or plurality of lymphocytes comprising the naïve T cells with at least two peptides or nucleic acids encoding peptides that comprise at least about 70%, 75%, 80%, 85%, 90%, 91%, 92%, 93%, 94%, 95%, 96%, 97%, 98%, 99%, <100% or 100% sequence identity to IL-18, IL-15, IL-6, IL-7, IL-4 or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4, functional fragments or variants thereof for a time period sufficient to stimulate growth and proliferation of the one or plurality of CD4+ and/or CD8+ T cells within the composition of cultured cells. The disclosure relates to a method of selectively growing memory effector T cells from a cell composition comprising naïve T cells comprising: contacting one or plurality of lymphocytes comprising the naïve T cells with at least two peptides or nucleic acids encoding peptides that comprise at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4 or functional fragments or variants thereof; or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4, functional fragments or variants thereof for a time period sufficient to stimulate growth and proliferation of the one or plurality of CD4+ and/or CD8+ T cells within the composition of cultured cells.

The disclosure also relates to a method of inducing an antigen-specific immune response against a viral antigen, the method comprising: (a) contacting one or plurality of lymphocytes with at least two peptides or nucleic acids encoding peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4 or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4, functional fragments or variants thereof for a time period sufficient to stimulate growth and proliferation of the one or plurality of CD4+ and/or CD8+ T cells within the composition of cultured cells. In some embodiments, the viral antigen is from a virus of the family Coronaviridae. In some embodiments, the viral antigen is from a coronavirus. In some embodiments, the viral antigen comprises at least a portion of a coronavirus membrane protein. In some embodiments, the viral antigen comprises at least a portion of a coronavirus envelope protein. In some embodiments, the viral antigen comprises at least a portion of a coronavirus spike protein. In some embodiments, the viral antigen comprises at least a portion of a coronavirus nucleocapsid protein. In some embodiments, the viral antigen is from SARS-CoV-2. In some embodiments, the viral antigen comprises at least a portion of SARS-CoV-2 membrane protein. In some embodiments, the viral antigen comprises at least a portion of SARS-CoV-2 envelope protein. In some embodiments, the viral antigen comprises at least a portion of SARS-CoV-2 spike protein. In some embodiments, the viral antigen comprises at least a portion of SARS-CoV-2 nucleocapsid protein.

In some alternative embodiments, the methods disclosed herein may be used to produce T cells which recognize other opportunistic pathogens such as bacterial (mycobacterium including Mycobacterium tuberculosis, Mycobacterium avium, Mycobacterium intracellulare, or Mycobacterium kansasii; Salmonella), fungal (Candida, Coccidiodomycosis, Cryptococcus, Histoplasmosis, Pneumocystis pneumonia), tumor (invasive cervical cancer), or parasite (Cryptosporidiosis, Toxoplasmosis) pathogens. T cell immunity is often directed against intracellular pathogens which may be targeted by T cells produced as disclosed herein. Opportunistic infections, which are infections that are generally of lower virulence within a healthy host but cause more severe and frequent disease in immunosuppressed individuals, typically occur in the period 1 month to 1 year after transplantation. Some nonlimited examples of opportunistic viral infections include cytomegalovirus (CMV), herpes simplex virus (HSV, type 1 or type 2, HHV-8, or HIV.

In an alternative embodiment, the antigen-specific T cells are used to treat infections by neonatal, congenital, and/or intrauterine pathogens including rubella, cytomegalovirus (CMV), parvovirus B19, varicella-zoster (VZV), enteroviruses, HIV, HTLV-1, hepatitis A, hepatitis B, hepatitis C, Lassa Fever, and Japanese Encephalitis. Perinatal and neonatal infections agents include Herpes Simplex Virus (including Human Herpes Simplex types 1 and 2), VZV, Enteroviruses, HIV, Hepatitis B, Hepatitis C, and HTLV-1.

Other pathogens include respiratory syncytial virus (RSV), metapneumovirus (hMPV), rhinovirus, parainfluenza (PIV), and human coronavirus, norovirus, Herpes simplex virus (HSV), SARS-1, SARS2, Zika virus, and encephalitis viruses.

The disclosure further relates to a method of generating, culturing and/or manufacturing CD4+ and/or CD8+ effector memory cells comprising: contacting one or plurality of lymphocytes with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4 or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4, functional fragments or variants thereof for a time period sufficient to stimulate growth and proliferation of the one or plurality of CD4+ and/or CD8+ T cells within the composition of cultured cells. The disclosure further relates to a method of generating, culturing and/or manufacturing CD4+ and/or CD8+ effector memory cells comprising: contacting one or plurality of lymphocytes with at least two peptides that comprise at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4 or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4, functional fragments or variants thereof for a time period sufficient to stimulate growth and proliferation of the one or plurality of CD4+ and/or CD8+ T cells within the composition of cultured cells.

The disclosure relates to a method of expanding a population of CD4+ and/or CD8+ memory effector T cells in a composition of cultured cells comprising contacting one or plurality of lymphocytes with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4 or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4, functional fragments or variants thereof for a time period sufficient to stimulate growth and proliferation of the one or plurality of CD4+ and/or CD8+ T cells within the composition of cultured cells. The disclosure relates to a method of expanding a population of CD4+ and/or CD8+ memory effector T cells in a composition of cultured cells comprising contacting one or plurality of lymphocytes with at least two peptides that comprise at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4 or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4, functional fragments or variants thereof for a time period sufficient to stimulate growth and proliferation of the one or plurality of CD4+ and/or CD8+ T cells within the composition of cultured cells.

The disclosure relates to a method of culturing a composition comprising a population of one or a plurality of T cells comprising contacting the one or plurality of T cells with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4 or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4, functional fragments or variants thereof for a time period sufficient to stimulate growth and proliferation of the one or plurality of CD4+ and/or CD8+ T cells. The disclosure relates to a method of culturing a composition comprising a population of one or a plurality of T cells comprising contacting the one or plurality of T cells with at least two peptides that comprise at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4 or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4, functional fragments or variants thereof for a time period sufficient to stimulate growth and proliferation of the one or plurality of CD4+ and/or CD8+ T cells.

The disclosure also relates to a T cell composition comprising from about 2% to about 23% CD4+ and/or CD8+ memory effector cells manufactured or grown from a population of naïve T cells or lymphocytes by any of the disclosed methods provided herein. The disclosure further relates to a tissue culture system comprising: lymphocytes from a subject, cell culture media, and a composition comprising at least two polypeptide or nucleic acid encoding peptides chosen from a polypeptide that comprises at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4 or functional fragments or variants thereof, or one or a plurality of vectors that encode at least two polypeptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, IL-4 or functional fragments or variants thereof. The disclosure also relates to a T cell composition comprising from about 2% to about 23% CD4+ and/or CD8+ memory effector cells manufactured or grown from a population of naïve T cells or lymphocytes by any of the disclosed methods provided herein. The disclosure further relates to a tissue culture system comprising: lymphocytes from a subject, cell culture media, and a composition comprising at least two polypeptide or nucleic acid encoding peptides chosen from a polypeptide that comprises at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4 or functional fragments or variants thereof, or one or a plurality of vectors that encode at least two polypeptides that comprise at least about 70%, 75%, 80%, 85%, 90%, 91%, 92%, 93%, 94%, 95%, 96%, 97%, 98%, 99%, <100%, or 100% sequence identity to human IL-18, IL-15, IL-6, IL-7, IL-4 or functional fragments or variants thereof.

In some embodiments, any of the disclosed methods further comprise wherein the one or plurality of T cells are naïve prior to step of contacting and the one or plurality of T cells are CCR7+ CD45RO+ after performing the step of contacting.

The disclosure relates to methods of treating a Coronaviridae infection comprising administering one or a plurality of compositions comprising a therapeutically effective amount of T cells of the disclosure stimulated with: one or a plurality of cytokine compositions disclosed herein; and/or one or a plurality of viral antigens that comprise from about 70% to about 100% sequence identity to SEQ ID: 11, 12, 13, and/or 14, or antigen fragments thereof. In some embodiments the Coronaviridae infection is a COVID-19 infection. The disclosure also relates to a method of treatment, wherein the step of administering comprises intravenous or parenteral administration injection of cells.

The advantages of the disclosed method and compositions will be realized and attained by means of the elements and combinations particularly pointed out in the appended claims. It is to be understood that both the foregoing general description and the following detailed description are exemplary and explanatory only and are not restrictive of the subject matter as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 depicts the experimental design described in Example 1. Peripheral blood was collected from consenting donors and cells were purified by Ficoll gradient isolation. 1×10⁵ cells were plated in each well on Day 0 with CMV peptide pools (IE1 and pp65) and the indicated cytokine concentration alone or in combination. On Day 7, plates were split equally into fresh media and with fresh cytokines. On Day 10, plates were re-stimulated with or without additional peptides for 6 hours, fixed, and stained for phenotype (CD3, CD56, CD4, CD8), viability, and effector cytokine secretion (IFNγ, TNFα).

FIGS. 2A, 2B, 2C and 2D depict the plate layouts described in Example 1. 1×10⁵ cells were plated in each well on Day 0 with CMV peptide pools encompassing IE1 and pp65 and the indicated cytokine concentration alone or in combination for plate layout 3 (FIG. 2A). On Day 7, plates were split equally into fresh media and with fresh cytokines. On Day 10, plates were re-stimulated with or without peptides for 6 hours, fixed, and stained for phenotype (CD3, CD56, CD4, CD8), viability, and effector cytokine secretion (IFNγ, TNFα).

FIG. 3 depicts the gating strategy described in Example 1. A representative sample was analyzed in Flowjo for staining and gating of effector lymphocyte populations. Cells were separated from debris by gating forward scatter vs. side scatter. Viable cells were identified by low staining of Live Dead viability dye. T cells were identified by CD3+ staining, and effector subsets were subsequently characterized by CD4+ vs CD8+ staining. Cells were then evaluated for cytokine production after culture with peptide pools by comparing staining of IFNγ and TNFα with wells cultured in media alone.

FIG. 4 depicts a summary of representative heat map data obtained from the screening of additional cytokine combinations described in Example 1. 1×10⁵ cells from Sample 3 and Sample 4 were plated in each well on Day 0 with CMV peptide pools encompassing IE1 and pp65 and the indicated cytokine alone or in combination for plate layout 3. Cells were analyzed for the total count of viable CD3+ T cells and the frequency of effector cytokine secretion over background (IFNγ, TNFα).

FIGS. 5A and 5B depict identification of superior cytokine combinations via high throughput flow cytometry analysis. FIG. 5A: Quantifications of phenotype and function of two plates from Sample 4 were analyzed by flow cytometry on Day 10, with entire contents of wells collected and visualized by heat map. The total count of viable CD3+ T cells were quantified from wells re-stimulated with media alone. The frequency of IFNγ+ CD3+ cells was derived from the frequency of IFNγ+ CD3+ cells after re-stimulation with IE1 and pp65 peptide pools and subtracted from media alone control wells. FIG. 5B: Wells containing the highest concentration of cytokines were compared between Samples 1-4 when cultured using plate layouts 1 and 2. The total recovered viable CD3+ count and the frequency of CMV specific CD3+ IFNγ+ cells (n≥8) was compared across each sample.

FIGS. 6A, 6B and 6C show that culture in IL-15 and IL-6 stimulates expansion of CMV specific CD3+ T cells equal or better than IL-4 and IL-7. Cells were re-stimulated with CMV peptide pools for 6 hours and evaluated for phenotype and function by flow cytometry, with entire well contents analyzed for the top dilutions of IL-15 and IL-6, IL-4 and IL-7, IL-15 alone, and no cytokine controls. The median of individual replicates (n≥8) were analyzed across experiments and individual patient samples (n=6), and statistics were analyzed by 2-way ANOVA with Tukey's correction with ****p<0.0001. From wells re-stimulated with media alone, the total count of viable CD3+ T cells, the percentage of viability of all CD3+ cells, and viable CD56+CD3− NK cell count were calculated from wells and shown in FIG. 6A. The median total count of viable CD3+CD4+ cells, CD3+CD8+ cells, and the ratio of CD4+/CD8+ cells were calculated from wells and shown in FIG. 6B. From wells re-stimulated with IE1 and pp65 peptide pools, the median frequency of CD3+ IFNγ+ cells and CD3+ IFNγ+ TNFα+ cells were calculated from wells and shown in FIG. 6C, and the frequency of IFNγ+ cells within CD4+ CD3+ and CD8+ CD3+ subtypes were analyzed.

FIGS. 7A, 7B, 7C and 7D shown that T cell therapy products are effector memory in phenotype (CCR7− CD45RO+). Cells were analyzed for the expression of memory markers CCR7 and CD45RO and divided into four populations both pre and post-culture with cytokines. The pre-culture memory phenotype of viable cells was quantified for all samples (FIG. 7A) according to the layout presented for representative Sample 1 (FIG. 7B). After 10 day culture, samples were analyzed again for memory markers CCR7 and CD45RO and averaged samples cultured in IL-15/IL-6 and IL-4/IL-7 growth conditions (FIG. 7C) and analyzed using 2-way ANOVA with Tukey's correction (* corresponds to p<0.05). One representative sample was examined for the memory phenotype of CD3+ cells which were positive or negative for IFNγ after re-stimulation with CMV specific peptides (FIG. 7D).

FIGS. 8A and 8B shown that cells cultured in Grex-10 vessels with IL-15/IL-6 produce equivalent levels of IFNγ compared with culture in IL-4/IL-7. Cells were grown in Grex-10 culture vessels with IL-15 and IL-6 or IL-4 and IL-7 for 10 days and tested in ELISPOT assays for IFNγ production when re-stimulated with media alone, actin (1 μg/mL), IE1 and pp65 peptide pools (1 μg/mL), or SEB (0.5 μg/mL). A representative sample is given in FIG. 8A and the mean of four different samples was compared using 2-way ANOVA with Tukey's correction (FIG. 8B). ***p<0.001; ****p<0.0001.

FIG. 9, shown on pages 9-1 to 9-10, depicts short amino acid sequences from viruses used in the production of amino acid mixes exposed to the cells of the disclosure.

FIGS. 10A, 10B, 10C, 10D and 10E show T-cell recognition of SARS-CoV-2 viral antigens. FIG. 10A: Specificity of the expanded cells in response to SARS-CoV-2 antigens from convalescent patients (n=23) and unexposed controls (n=10) was assayed by IFN-γ ELISpot assay (bars=median). Unstimulated T-cells (CTL only) and stimulation with actin were used as negative controls. Results are presented as Spot forming units (SFC) per 1×10⁵ cells. The number of spots was compared with those from actin control via two-tailed Student's t-test. *p=0.021, **p<0.004. FIG. 10B: Specificity of CD4, CD8 and g/d T-cell populations for membrane and spike proteins was assessed by intracellular cytokine staining for IFN-γ and TNFα. Summary data of the response of expanded CD4⁺ T-cells (FIG. 10C) and CD8+ T-cells (FIG. 10D) in response to Membrane and Spike proteins by intracellular cytokine staining was analyzed in convalescent donors (n=9), and the percentage of T-cell were compared to actin-stimulated controls via two-tailed Student's t-test. ♦p<0.05, ♦♦ p<0.01. FIG. 10E: Phenotype of the expanded cells was accessed by flow cytometry with markers for T-cells (CD3, CD4, CD8, TCRαβ, TCRγδ), NK cells (CD16/CD56), and B-cells (CD19).

FIGS. 11A, 11B, 11C and 11D show T-cell recognition of epitopes within membrane protein. FIG. 11A: T-cell epitope mapping of membrane protein was performed using 17 mini-pools containing 5-12 peptides each, with responses measured via IFN-γ ELISpot (SFC: spot forming units). FIG. 11AB: Epitope mapping identified responses to four peptides within AA 145-173 and 192-222 of the C-terminal intravirion domain. Intracellular cytokine staining demonstrated a predominant CD4-mediated response to membrane peptides 37-38 (FIG. 11C) as well as peptides 44-45 (FIG. 11D). SEB=staphylococcal enterotoxin beta.

FIGS. 12A and 12B show clinical characteristics of convalescent COVID-19 patients. FIG. 12A: Flow diagram of illness severity (based on WHO classifications), T-cell and antibody immune response to SARS-CoV-2, and basis of COVID-19 diagnosis. FIG. 12B: Ribbon diagram of primary clinical symptoms of the 23 convalescent patients, as well as timing of PCR testing and research evaluation.

FIGS. 13A and 13B show SARS-CoV-2 antibody testing of normal controls and convalescent patients. Testing for antibodies to nucleocapsid (FIG. 13A) and spike proteins (FIG. 13B) was performed via luciferase immunoprecipitation assay. Positivity thresholds (dotted lines) were set based on previous data using unexposed normal control samples.

FIGS. 14A and 14B show T-cell extended phenotyping of coronavirus-specific T-cells. FIG. 14A: T-cell populations following expansion were determined via flow cytometry. T-cells were classified as naïve (CD45RO−/CCR7+/CD95−), central memory (CD45RO+CCR7+/CD95+), effector memory (CD45RO+CCR7−), and stem cell memory (CD45RO−/CCR7+/CD95+), and terminal effector (CD45RO−/CCR7−). FIG. 14B: Gating strategy for T-cell memory/naïve subsets.

FIG. 15 shows detection of T-cell responses to SARS CoV-2 proteins from peripheral blood. Peripheral blood mononuclear cells (PBMC) from convalescent patients (triangles) and unexposed controls (circles) were tested for responses to peptide libraries encompassing SARS-CoV-2 structural proteins by IFN-γ ELISpot. Results are reported as spot forming colonies (SFC) per 1×10⁵ cells per well. PBMC alone and actin stimulation were utilized as negative controls. Peptide libraries from cytomegalovirus pp65 and IE1 as well as adenovirus hexon and penton were utilized as additional viral controls.

FIG. 16 shows T-cell responses to SARS-CoV-2 versus illness severity in convalescent patients. Expanded coronavirus-specific T-cells were tested for specificity to SARS-CoV-2 structural protein libraries on day 10 of culture via IFN-γ ELISpot. Control unexposed donors (circles) and convalescent patients with mild disease (upward triangles) or moderate to severe disease (downward triangles) by WHO criteria were tested. Expanded cells alone (CTL alone) and actin stimulated cells were used as negative controls. Results are reported as spot forming colonies (SFC) per 1×10⁵ cells/well.

FIG. 17 shows that wells containing the highest concentration of cytokines were compared between Samples 1-4 when cultured using plate layouts 1 and 2. The average total viable CD3+ count and the average frequency of CMV specific CD3+ IFNγ+ cells (n≥8) was compared across each sample.

FIGS. 18A, 18B and 18C show that cells were re-stimulated with CMV peptide for 6 hour and evaluated for phenotype and function by flow cytometry, with entire well contents analyzed for the top dilutions of IL-15 and IL-6, IL-4 and IL-7, IL-15 alone, and no cytokine controls. Individual replicates (n≥8) were averaged across experiments and individual patient samples (n=6), and statistics were analyzed by 2-way ANOVA with Tukey's correction with ****p<0.0001. From wells re-stimulated with media alone, the average total count of viable CD3+ T cells, the viability of all CD3+ cells, and average viable CD56+CD3− NK cell count were calculated from wells and shown in FIG. 18A. The average total count of viable CD3+CD4+ cells, CD3+CD8+ cells, and the ratio of CD4+/CD8+ cells were calculated from wells and shown in FIG. 18B. From wells re-stimulated with IE1 and pp65 peptide, the average frequency of CD3+ IFNγ+ cells and CD3+ IFNγ+ TNFα+ cells were calculated from wells and shown in FIG. 18C and analyzed.

FIG. 19 shows 1×10⁵ cells from Sample 3 and Sample 4 were plated in each well on Day 0 with CMV peptides IE1 and pp65 and the indicated cytokine concentration alone or in combination for plate layout 3. Cells were analyzed for the total count of viable CD3+ T cells and the frequency of effector cytokine secretion over background (IFNγ, TNFα).

FIG. 20 shows the T-cell recognition of SARS-CoV-2 viral antigens. Specificity of the expanded cells in response to SARS-CoV-2 antigens from convalescent patients (n=45) and unexposed controls (n=15) was assayed by IFN-γ ELISpot assay (bars=median). Unstimulated T-cells (CTL only) and stimulation with actin were used as negative controls. Results are presented as Spot forming units (SFC) per 1×10⁵ cells. Specificity was defined as ≥20 spots per well with significance above background (actin) via two-tailed Student's t-test. *p=0.0015, **p=0.000013.

FIG. 21A-21D show the specificity of ex vivo expanded CST. Following 10-12 days of culture, specificity of CD4 and CD8 T-cell populations for membrane, spike, and nucleocapsid proteins was assessed by intracellular cytokine staining for IFN-γ and TNFα(FIG. 21A). Subject 2 demonstrates a CD4-predominant response targeting structural proteins. Summary data of the response of expanded CD4+ T-cells (FIG. 21B), CD8+ T-cells (FIG. 21C) and γδ T cells (FIG. 21D) in response to membrane, spike, and nucleocapsid proteins by intracellular cytokine staining was analyzed in convalescent donors (n=11), and the percentage of T-cell were compared to actin-stimulated controls via two-tailed Student's t-test. *p<0.05, **p<0.01.

FIG. 22 shows the T-cell specificity of seropositive versus seronegative patients. Comparison of IFN-γ ELISpot results from post-expansion CSTs from SARS COV-2 seropositive vs seronegative convalescent patients was performed via student's T-test. * p=0.0027, **p=0.0014.

FIG. 23A-23C show SARS-CoV-2 epitope mapping of CSTs. T-cell epitope mapping of structural proteins was performed using mini-pools containing 5-12 peptides each, with responses measured via IFN-γ ELISpot (SFC: spot forming units per 1×10⁵ cells (FIG. 23A). Epitopes within membrane protein were identified within the C-terminus at AA 144-163 and 173-192, which were recognized by 8 and 6 donors respectively (FIG. 23B). Mapping of spike epitopes demonstrated three regions at AA 57-75, 205-224, and 449-463, which were recognized by 3 donors (FIG. 23B). Mapping of nucleocapsid epitopes showed two regions at AA 257-271 and 313-335 were recognized by 3 donors (FIG. 23C).

FIG. 24A-24E show the T-cell restrictions of SARS-CoV-2 epitopes. Identification of the T-cells responding to each identified epitope was performed via intracellular cytokine staining on expanded CSTs, with percentages of TNFα⁺/IFNγ⁺ populations depicted. Intracellular cytokine staining demonstrated a predominant CD4-mediated response to membrane peptides 37-38 (FIG. 24A), membrane peptides 44-45 (FIG. 24B), nucleocapsid peptide 65 (FIG. 24C), and spike proteins 15-16 (FIG. 24D), and a predominant CD8-mediated response to nucleocapsid peptide 81 (FIG. 24E). SEB=staphylococcal enterotoxin beta.

FIG. 25A-25C show the epitope locations within SARS-CoV-2 structural proteins.

FIG. 25A. Epitopes within membrane protein were identified at the C-terminal intravirion domain. TMD=transmembrane domains.

FIG. 25B. Within spike proteins, epitopes were found within the S1 region, including one epitope within the receptor binding domain (RBD).

FIG. 25C. In nucleocapsid protein, epitopes were identified in the region of the dimerization domain (DD).

FIG. 26A-26C show ICS (CD4+ and CD8+) and ELISpot specificities.

FIG. 26A shows significantly increased CD4+ specificity using IL-15/7 compared to IL-4/7 in Grex validation.

FIG. 26B shows increased CD8+ specificity using IL-15/7 compared to IL-4/7 in Grex validation.

FIG. 26C shows increased specificity using IL-15/7 compared to IL-4/7 in Grex-ELISpot. In conclusion, IL-15/7 showed increased CD4+ and CD8+ specificity compared to IL-4/7.

### DETAILED DESCRIPTION

The disclosed methods and compositions may be understood more readily by reference to the following detailed description of particular embodiments and the Examples included therein and to the Figures and their previous and following description. It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a cytokine is disclosed and discussed and a number of modifications that can be made to a number of molecules including the polypeptide or nucleic acid expressing the polypeptides are discussed, each and every combination and permutation of cytokines and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### Definitions/Terms

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

The terminology used herein is for the purpose of describing particular embodiments only and is not intended to be limiting. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

The following terms or definitions are provided solely to aid in the understanding of the disclosure. Unless specifically defined herein, all terms used herein have the same meaning as they would to one skilled in the art of the present disclosure. Practitioners are particularly directed to Sambrook et al., MOLECULAR CLONING: A LABORATORY MANUAL, 2nd ed., Cold Spring Harbor Press, Plainsview, N.Y. (1989); and Ausubel et al., CURRENT PROTOCOLS IN MOLECULAR BIOLOGy (Supplement 47), John Wiley & Sons, New York (1999), for definitions and terms of the art, the volume of which is incorporated by reference in its entirety. The definitions provided herein should not be construed to have a scope less than understood by a person of ordinary skill in the art.

As used in the specification and the appended claims, the singular forms “a,” “an” and “the” include plural referents unless the context clearly dictates otherwise.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods. When “about” is present before a series of numbers or a range, it is understood that “about” can modify each of the numbers in the series or range. For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in some embodiments, to A without B (optionally including elements other than B); in another embodiments, to B without A (optionally including elements other than A); in yet another embodiments, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

The term “antigen” as used herein refers to any substances or molecules, such as polypeptides, peptides, or glyco- or lipo-peptides, that are recognized by the immune system, such as by the cellular or humoral arms of the human immune system, to elicit an immune response. The term “antigen” includes antigenic determinants, such as peptides with lengths of 6, 7, 8, 9, 10, 11, 12, 13, 14, 15, 16, 17, 18, 19, 20, 21, 22 or more amino acid residues that bind to major histocompatibility complex (MHC) molecules, form parts of MHC Class I or II complexes, or that are recognized when complexed with such molecules.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” in context of an immunotherapy refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, indirect or direct ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. As used herein, the terms “activating CD8+ T cells” or “CD8+ T cell activation” refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein.

The term “allogeneic” as used herein refers to medical therapy in which the donor and recipient are different individuals of the same species.

The term “autologous” as used herein refers to medical therapy in which the donor and recipient are the same person.

“Coding sequence” or “encoding nucleic acid” as used herein refers to a nucleic acid (RNA, DNA, or RNA/DNA hybrid molecule) that comprises a nucleotide sequence which encodes a protein. The coding sequence may further include initiation and termination signals operably linked to regulatory elements including a promoter and polyadenylation signal capable of directing expression in the cells of an individual or mammal to whom the nucleic acid is administered.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

The term “cytokine” as used herein has its normal meaning in the art. Non-limiting examples of cytokines used in the disclosure include interleukin-2 (IL-2), IL-4, IL-6, IL-7, IL-12, IL-15, IL-18, IL-21 and IL-27.

The term “cytotoxic T cell” or “cytotoxic T lymphocyte” as used herein is a type of immune cell that bears a CD8+ antigen and that can kill certain cells, including foreign cells, tumor cells, and cells infected with a virus. Cytotoxic T cells can be separated from other blood cells, grown ex vivo, and then given to a patient to kill tumor or viral cells. A cytotoxic T cell is a type of white blood cell and a type of lymphocyte.

The term “dendritic cell” or “DC” as used herein describes a diverse population of morphologically similar cell types found in a variety of lymphoid and non-lymphoid tissues, see Steinman, Ann. Rev. Immunol. 9:271-296 (1991).

The term “effector cell” as used herein describes a cell that can bind to or otherwise recognize an antigen and mediate an immune response. Tumor-, virus-, or other antigen-specific T cells and NKT cells are examples of effector cells.

The term “endogenous” as used herein refers to any material from or produced inside an organism, cell, tissue or system.

The term “epitope” or “antigenic determinant” as used herein refers to the part of an antigen that is recognized by the immune system, specifically by antibodies, B cells, or T cells.

The term “exogenous” as used herein refers to any material introduced from or produced outside an organism, cell, tissue or system.

As used herein, the term “functional fragment” means any portion of a polypeptide that is of a sufficient length to retain at least partial biological function that is similar to or substantially similar to the wild-type polypeptide upon which the fragment is based. In some embodiments, a functional fragment of a polypeptide is a polypeptide that comprises or possesses at least about 70, 75, 80, 85, 90, 95, 96, 97, 98, or 99% sequence identity to any polypeptides disclosed in Table 1 and has sufficient length to retain at least partial binding affinity to one or a plurality of ligands that bind to the polypeptides in Table 1. In some embodiments, a functional fragment of a nucleic acid is a nucleic acid that comprises or possesses at least about 70, 75, 80, 85, 90, 95, 96, 97, 98, or 99% sequence identity to any nucleic acid to which it is being compared and has sufficient length to retain at least partial function related to the nucleic acid to which it is being compared. In some embodiments, the fragment is a fragment of any polypeptide disclosed in Table 1 and has a length of at least about 10, about 20, about 30, about 40, about 50, about 60, about 70, about 80, about 90, or about 100 contiguous amino acids. In some embodiments, the fragment is a fragment of any polypeptide disclosed in Table 1 and has a length of at least about 50 amino acids. In some embodiments, the fragment is a fragment of any polypeptide disclosed in Table 1 and has a length of at least about 100 amino acids. In some embodiments, the fragment is a fragment of any polypeptide disclosed in Table 1 and has a length of at least about 150 amino acids. In some embodiments, the fragment is a fragment of any polypeptide disclosed in Table 1 and has a length of at least about 200 amino acids. In some embodiments, the fragment is a fragment of any polypeptide disclosed in Table 1 and has a length of at least about 250 amino acids. In some embodiments, the fragment is a fragment of any polypeptide disclosed in Table 1 and has a length of at least about 300 amino acids. In some embodiments, the fragment is a fragment of any polypeptide disclosed in Table 1 and has a length of at least about 350 amino acids. In some embodiments, the fragment is a fragment of any polypeptide disclosed in Table 1 and has a length of at least about 400 amino acids. In some embodiments, the fragment is a fragment of any polypeptide disclosed in Table 1 and has a length of at least about 450 amino acids. In some embodiments, the fragment is a fragment of any polypeptide disclosed in Table 1 and has a length of at least about 500 amino acids. In some embodiments, the fragment is a fragment of any polypeptide disclosed in Table 1 and has a length of at least about 550 amino acids. In some embodiments, the fragment is a fragment of any polypeptide disclosed in Table 1 and has a length of at least about 600 amino acids. In some embodiments, the fragment is a fragment of any polypeptide disclosed in Table 1 and has a length of at least about 650 amino acids. In some embodiments, the fragment is a fragment of any polypeptide disclosed in Table 1 and has a length of at least about 700 amino acids. In some embodiments, the fragment is a fragment of any polypeptide disclosed in Table 1 and has a length of at least about 750 amino acids. In some embodiments, the fragment is a fragment of any polypeptide disclosed in Table 1 and has a length of at least about 800 amino acids. In some embodiments, the fragment is a fragment of any polypeptide disclosed in Table 1 and has a length of at least about 850 amino acids. In some embodiments, the fragment is a fragment of any polypeptide disclosed in Table 1 and has a length of at least about 900 amino acids. In some embodiments, the fragment is a fragment of any polypeptide disclosed in Table 1 and has a length of at least about 950 amino acids. In some embodiments, the fragment is a fragment of any polypeptide disclosed in Table 1 and has a length of at least about 1000 amino acids. In some embodiments, the fragment is a fragment of any polypeptide disclosed in Table 1 and has a length of at least about 1050 amino acids. In some embodiments, the fragment is a fragment of any polypeptide disclosed in Table 1 and has a length of at least about 1250 amino acids. In some embodiments, the fragment is a fragment of any polypeptide disclosed in Table 1 and has a length of at least about 1500 amino acids. In some embodiments, the fragment is a fragment of any polypeptide disclosed in Table 1 and has a length of at least about 1750 amino acids. In some embodiments, the fragment is a fragment of any polypeptide disclosed in Table 1 and has a length of at least about 2000 amino acids. In some embodiments, the fragment is a fragment of any polypeptide disclosed in Table 1 and has a length of at least about 2250 amino acids. In some embodiments, the fragment is a fragment of any polypeptide disclosed in Table 1 and has a length of at least about 2500 amino acids. In some embodiments, the fragment is a fragment of any polypeptide disclosed in Table 1 and has a length of at least about 2750 amino acids. In some embodiments, the fragment is a fragment of any polypeptide disclosed in Table 1 and has a length of at least about 3000 amino acids.

In some embodiments, the fragment is a fragment of any polypeptide disclosed in Table 1 and has a length of no more than about 10, about 20, about 30, about 40, about 50, about 60, about 70, about 80, about 90, or about 100 contiguous amino acids. In some embodiments, the fragment is a fragment of any polypeptide disclosed in Table 1 and has a length of no more than about 50 amino acids. In some embodiments, the fragment is a fragment of any polypeptide disclosed in Table 1 and has a length of no more than about 100 amino acids. In some embodiments, the fragment is a fragment of any polypeptide disclosed in Table 1 and has a length of no more than about 150 amino acids. In some embodiments, the fragment is a fragment of any polypeptide disclosed in Table 1 and has a length of no more than about 200 amino acids. In some embodiments, the fragment is a fragment of any polypeptide disclosed in Table 1 and has a length of no more than about 250 amino acids. In some embodiments, the fragment is a fragment of any polypeptide disclosed in Table 1 and has a length of no more than about 300 amino acids. In some embodiments, the fragment is a fragment of any polypeptide disclosed in Table 1 and has a length of no more than about 350 amino acids. In some embodiments, the fragment is a fragment of any polypeptide disclosed in Table 1 and has a length of no more than about 400 amino acids. In some embodiments, the fragment is a fragment of any polypeptide disclosed in Table 1 and has a length of no more than about 450 amino acids. In some embodiments, the fragment is a fragment of any polypeptide disclosed in Table 1 and has a length of no more than about 500 amino acids. In some embodiments, the fragment is a fragment of any polypeptide disclosed in Table 1 and has a length of no more than about 550 amino acids. In some embodiments, the fragment is a fragment of any polypeptide disclosed in Table 1 and has a length of no more than about 600 amino acids. In some embodiments, the fragment is a fragment of any polypeptide disclosed in Table 1 and has a length of no more than about 650 amino acids. In some embodiments, the fragment is a fragment of any polypeptide disclosed in Table 1 and has a length of no more than about 700 amino acids. In some embodiments, the fragment is a fragment of any polypeptide disclosed in Table 1 and has a length of no more than about 750 amino acids. In some embodiments, the fragment is a fragment of any polypeptide disclosed in Table 1 and has a length of no more than about 800 amino acids. In some embodiments, the fragment is a fragment of any polypeptide disclosed in Table 1 and has a length of no more than about 850 amino acids. In some embodiments, the fragment is a fragment of any polypeptide disclosed in Table 1 and has a length of no more than about 900 amino acids. In some embodiments, the fragment is a fragment of any polypeptide disclosed in Table 1 and has a length of no more than about 950 amino acids. In some embodiments, the fragment is a fragment of any polypeptide disclosed in Table 1 and has a length of no more than about 1000 amino acids. In some embodiments, the fragment is a fragment of any polypeptide disclosed in Table 1 and has a length of no more than about 1050 amino acids. In some embodiments, the fragment is a fragment of any polypeptide disclosed in Table 1 and has a length of no more than about 1250 amino acids. In some embodiments, the fragment is a fragment of any polypeptide disclosed in Table 1 and has a length of no more than about 1500 amino acids. In some embodiments, the fragment is a fragment of any polypeptide disclosed in Table 1 and has a length of no more than about 1750 amino acids. In some embodiments, the fragment is a fragment of any polypeptide disclosed in Table 1 and has a length of no more than about 2000 amino acids. In some embodiments, the fragment is a fragment of any polypeptide disclosed in Table 1 and has a length of no more than about 2250 amino acids. In some embodiments, the fragment is a fragment of any polypeptide disclosed in Table 1 and has a length of no more than about 2500 amino acids. In some embodiments, the fragment is a fragment of any polypeptide disclosed in Table 1 and has a length of no more than about 2750 amino acids. In some embodiments, the fragment is a fragment of any polypeptide disclosed in Table 1 and has a length of no more than about 3000 amino acids.

In some embodiments, the fragment is a fragment of any polypeptide disclosed in Table 1 and has a length from about 10 to about 3000 amino acids. In some embodiments, the fragment is a fragment of any polypeptide disclosed in Table 1 and has a length from about 10 to about 3000 amino acids. In some embodiments, the fragment is a fragment of any polypeptide disclosed in Table 1 and has a length from about 10 to about 2750 amino acids. In some embodiments, the fragment is a fragment of any polypeptide disclosed in Table 1 and has a length from about 10 to about 2500 amino acids. In some embodiments, the fragment is a fragment of any polypeptide disclosed in Table 1 and has a length from about 10 to about 2250 amino acids. In some embodiments, the fragment is a fragment of any polypeptide disclosed in Table 1 and has a length from about 10 to about 2000 amino acids. In some embodiments, the fragment is a fragment of any polypeptide disclosed in Table 1 and has a length from about 10 to about 1750 amino acids. In some embodiments, the fragment is a fragment of any polypeptide disclosed in Table 1 and has a length from about 10 to about 1500 amino acids. In some embodiments, the fragment is a fragment of any polypeptide disclosed in Table 1 and has a length from about 10 to about 1250 amino acids. In some embodiments, the fragment is a fragment of any polypeptide disclosed in Table 1 and has a length from about 10 to about 1000 amino acids. In some embodiments, the fragment is a fragment of any polypeptide disclosed in Table 1 and has a length from about 10 to about 950 amino acids. In some embodiments, the fragment is a fragment of any polypeptide disclosed in Table 1 and has a length from about 10 to about 850 amino acids. In some embodiments, the fragment is a fragment of any polypeptide disclosed in Table 1 and has a length from about 10 to about 800 amino acids. In some embodiments, the fragment is a fragment of any polypeptide disclosed in Table 1 and has a length from about 10 to about 750 amino acids. In some embodiments, the fragment is a fragment of any polypeptide disclosed in Table 1 and has a length from about 10 to about 700 amino acids. In some embodiments, the fragment is a fragment of any polypeptide disclosed in Table 1 and has a length from about 10 to about 650 amino acids. In some embodiments, the fragment is a fragment of any polypeptide disclosed in Table 1 and has a length from about 10 to about 600 amino acids. In some embodiments, the fragment is a fragment of any polypeptide disclosed in Table 1 and has a length from about 10 to about 550 amino acids. In some embodiments, the fragment is a fragment of any polypeptide disclosed in Table 1 and has a length from about 10 to about 500 amino acids. In some embodiments, the fragment is a fragment of any polypeptide disclosed in Table 1 and has a length from about 10 to about 450 amino acids. In some embodiments, the fragment is a fragment of any polypeptide disclosed in Table 1 and has a length from about 10 to about 400 amino acids. In some embodiments, the fragment is a fragment of any polypeptide disclosed in Table 1 and has a length from about 10 to about 350 amino acids. In some embodiments, the fragment is a fragment of any polypeptide disclosed in Table 1 and has a length from about 10 to about 300 amino acids. In some embodiments, the fragment is a fragment of any polypeptide disclosed in Table 1 and has a length from about 10 to about 250 amino acids. In some embodiments, the fragment is a fragment of any polypeptide disclosed in Table 1 and has a length from about 10 to about 200 amino acids. In some embodiments, the fragment is a fragment of any polypeptide disclosed in Table 1 and has a length from about 10 to about 150 amino acids. In some embodiments, the fragment is a fragment of any polypeptide disclosed in Table 1 and has a length from about 10 to about 100 amino acids. In some embodiments, the fragment is a fragment of any polypeptide disclosed in Table 1 and has a length from about 10 to about 90 amino acids. In some embodiments, the fragment is a fragment of any polypeptide disclosed in Table 1 and has a length from about 10 to about 80 amino acids. In some embodiments, the fragment is a fragment of any polypeptide disclosed in Table 1 and has a length from about 10 to about 70 amino acids. In some embodiments, the fragment is a fragment of any polypeptide disclosed in Table 1 and has a length from about 10 to about 60 amino acids. In some embodiments, the fragment is a fragment of any polypeptide disclosed in Table 1 and has a length from about 10 to about 50 amino acids. In some embodiments, the fragment is a fragment of any polypeptide disclosed in Table 1 and has a length from about 10 to about 40 amino acids. In some embodiments, the fragment is a fragment of any polypeptide disclosed in Table 1 and has a length from about 10 to about 30 amino acids. In some embodiments, the fragment is a fragment of any polypeptide disclosed in Table 1 and has a length from about 10 to about 20 amino acids.

In some embodiments, the fragment is a fragment of any polypeptide disclosed in Table 1 and has a length from about 10 to about 3000 amino acids. In some embodiments, the fragment is a fragment of any polypeptide disclosed in Table 1 and has a length from about 20 to about 3000 amino acids. In some embodiments, the fragment is a fragment of any polypeptide disclosed in Table 1 and has a length from about 30 to about 3000 amino acids. In some embodiments, the fragment is a fragment of any polypeptide disclosed in Table 1 and has a length from about 40 to about 3000 amino acids. In some embodiments, the fragment is a fragment of any polypeptide disclosed in Table 1 and has a length from about 50 to about 3000 amino acids. In some embodiments, the fragment is a fragment of any polypeptide disclosed in Table 1 and has a length from about 60 to about 3000 amino acids. In some embodiments, the fragment is a fragment of any polypeptide disclosed in Table 1 and has a length from about 70 to about 3000 amino acids. In some embodiments, the fragment is a fragment of any polypeptide disclosed in Table 1 and has a length from about 80 to about 3000 amino acids. In some embodiments, the fragment is a fragment of any polypeptide disclosed in Table 1 and has a length from about 90 to about 3000 amino acids. In some embodiments, the fragment is a fragment of any polypeptide disclosed in Table 1 and has a length from about 100 to about 3000 amino acids. In some embodiments, the fragment is a fragment of any polypeptide disclosed in Table 1 and has a length from about 150 to about 3000 amino acids. In some embodiments, the fragment is a fragment of any polypeptide disclosed in Table 1 and has a length from about 200 to about 3000 amino acids. In some embodiments, the fragment is a fragment of any polypeptide disclosed in Table 1 and has a length from about 250 to about 3000 amino acids. In some embodiments, the fragment is a fragment of any polypeptide disclosed in Table 1 and has a length from about 300 to about 3000 amino acids. In some embodiments, the fragment is a fragment of any polypeptide disclosed in Table 1 and has a length from about 350 to about 3000 amino acids. In some embodiments, the fragment is a fragment of any polypeptide disclosed in Table 1 and has a length from about 400 to about 3000 amino acids. In some embodiments, the fragment is a fragment of any polypeptide disclosed in Table 1 and has a length from about 450 to about 3000 amino acids. In some embodiments, the fragment is a fragment of any polypeptide disclosed in Table 1 and has a length from about 500 to about 3000 amino acids. In some embodiments, the fragment is a fragment of any polypeptide disclosed in Table 1 and has a length from about 550 to about 3000 amino acids. In some embodiments, the fragment is a fragment of any polypeptide disclosed in Table 1 and has a length from about 600 to about 3000 amino acids. In some embodiments, the fragment is a fragment of any polypeptide disclosed in Table 1 and has a length from about 650 to about 3000 amino acids. In some embodiments, the fragment is a fragment of any polypeptide disclosed in Table 1 and has a length from about 700 to about 3000 amino acids. In some embodiments, the fragment is a fragment of any polypeptide disclosed in Table 1 and has a length from about 750 to about 3000 amino acids. In some embodiments, the fragment is a fragment of any polypeptide disclosed in Table 1 and has a length from about 800 to about 3000 amino acids. In some embodiments, the fragment is a fragment of any polypeptide disclosed in Table 1 and has a length from about 850 to about 3000 amino acids. In some embodiments, the fragment is a fragment of any polypeptide disclosed in Table 1 and has a length from about 900 to about 3000 amino acids. In some embodiments, the fragment is a fragment of any polypeptide disclosed in Table 1 and has a length from about 950 to about 3000 amino acids. In some embodiments, the fragment is a fragment of any polypeptide disclosed in Table 1 and has a length from about 1000 to about 3000 amino acids. In some embodiments, the fragment is a fragment of any polypeptide disclosed in Table 1 and has a length from about 1050 to about 3000 amino acids. In some embodiments, the fragment is a fragment of any polypeptide disclosed in Table 1 and has a length from about 1250 to about 3000 amino acids. In some embodiments, the fragment is a fragment of any polypeptide disclosed in Table 1 and has a length from about 1500 to about 3000 amino acids. In some embodiments, the fragment is a fragment of any polypeptide disclosed in Table 1 and has a length from about 1750 to about 3000 amino acids. In some embodiments, the fragment is a fragment of any polypeptide disclosed in Table 1 and has a length from about 2000 to about 3000 amino acids. In some embodiments, the fragment is a fragment of any polypeptide disclosed in Table 1 and has a length from about 2250 to about 3000 amino acids. In some embodiments, the fragment is a fragment of any polypeptide disclosed in Table 1 and has a length from about 2500 to about 3000 amino acids. In some embodiments, the fragment is a fragment of any polypeptide disclosed in Table 1 and has a length from about 2750 to about 3000 amino acids.

“Optional” or “optionally” means that the subsequently described event, circumstance, or material may or may not occur or be present, and that the description includes instances where the event, circumstance, or material occurs or is present and instances where it does not occur or is not present.

As used herein, the term “heterologous” refers to a nucleic acid sequence that is operably linked to another nucleic acid sequence to which it is not operably linked in nature, or to which it is operably linked at a different location in nature. For example, a protein-coding nucleic acid sequence operably linked to a promoter which is not the native promoter of this protein-coding sequence is considered to be heterologous to the promoter. In some embodiments, the heterologous sequence comprises a plasmid or episome.

A “naïve” T cell or other immune effector cell as used herein is one that has not been exposed to or primed by an antigen or to an antigen-presenting cell presenting a peptide antigen capable of activating that cell. A “naïve” donor is an individual whose immune system has not been exposed to, immunized with, or challenged with a particular epitope, antigen, or microorganism and thus substantially lacks immunological memory to an epitope, antigen or microorganism.

The term “non-engineered” when referring to the cells of the compositions means a cell that does not contain or express an exogenous nucleic acid or amino acid sequence. For example, the cells of the compositions do not express, for example, a chimeric antigen receptor.

A “peptide library” or “overlapping peptide library” as used herein within the meaning of the application is a complex mixture of peptides which in the aggregate covers the partial or complete sequence of a protein antigen. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries may be commercially available or may be custom-made for particular antigens.

A “peripheral blood mononuclear cell” or “PBMC” as used herein is any peripheral blood cell having a round nucleus. These cells consist of lymphocytes (T cells, B-cells, NK cells) and monocytes. In humans, lymphocytes make up the majority of the PBMC population, followed by monocytes, and a small percentage of dendritic cells.

The term “precursor cell” as used herein refers to a cell which can differentiate or otherwise be transformed into a particular kind of cell. For example, a “T cell precursor cell” or “pre-T cell” can differentiate into a T cell and a “dendritic precursor cell” can differentiate into a dendritic cell.

A “T cell population” or “T cell subpopulation” can include thymocytes, immature T-lymphocytes, mature T-lymphocytes, resting T-lymphocytes and activated T-lymphocytes. The T cell population or subpopulation can include αβ T cells, including CD4+ T cells, CD8+ T cells, γδ T cells, natural killer T cells, or any other subset of T cells.

Types of T cells (and other mononuclear cell) include helper T cells (CD4), CD4 TREGS cells (e.g. CTLA-4⁺ GITR⁺ PD-1⁺ CCR⁺ CCR4⁺ CXCR4⁺ GITR⁺ LAG3⁺ OX40⁺ ICOS), cytotoxic T cells (CD8), CD8 mucosal associated invariant T cells (Rearranged TCRβ chains with Vβ gene segments), CD8 memory T cells (e.g., CD45RO^{hi} and CD95⁺ CD45RO^{low} subsets), B cells, or gamma/delta T cells markers. Other T cell phenotypes include cells with one or more of the following markers: CD4+, CD8+, CD4+/CD25+, CD45RO+, CD27+, CD28+, and/or PD1. T cell phenotypes include CD4+CD8+; CD27+CD28+ and CD4+, CD45RO+ and CD27+.

Other T cell phenotypes, transcription factors, functions, and other features comprise those described by, and incorporated by reference to, Dong, G. and Martinez, G. J., T cells: the usual subsets, NATURE REVIEWS, 2010, hypertext transfer protocol://www.nature.com/reviews/posters/Tcellsubsets. These include cytotoxic T cells (surface phenotype: αβ TCR, CD3, CD8; effector molecules secreted: perforin, granzyme, IFNγ), exhausted T cells (surface phenotype: CD3, CD8, PD1, TIM3, 1B11, LAG3), anergic T cells (surface phenotype: αβ TCR, CD3, BTLA; effector factors: GRAIL, CBL-B, ITCH, NEDD4), Tr₁ cells (surface phenotype: αβ TCR, CD3, CD4; effector molecules secreted: IL-10), Natural TR_{eg} T cells (surface phenotype: αβ TCR, CD3, CD4, CD25, CTLAA4, GITR; effector molecules secreted: IL-10, TGFβ, IL-35), inducible T_{Reg} cells (surface phenotype: αβ TCR, CD3, CD4, CD25, CTLAA4, GITR; effector molecules secreted: IL-10, TGFβ), NKT cells (surface phenotype NK1.1, SLAMF1, SLAMF6, TGFβR, Vα24, Jα18; Effector molecules secreted: IL-4, IFNγ, IL-17A), and CD8. T cells (surface phenotype: αβ or γδ TCR, CD3, CD8_{αα}, B220; Effector molecules secreted IL-10, TGFβ).

Isolation of T cells or T cell populations as disclosed above such as those produced by the disclosed methods may be performed using cell sorting or by antibody-based removal or ablation of cells bearing particular markers. Naïve human CD4+ T cells express CD45RA, CCR7, CD62L and CD27 and may be enriched or recovered using these markers. Antibodies recognizing T and B cell markers are commercially available and incorporated by reference to MATER METHODS 2016; 6:1502. Kits and materials for isolation of T cells are commercially available, for example, from STEMCELL Technologies, hypertext transfer protocol secure://www.stemcell.com/cell-and-tissue-types/popular-cell-and-tissue-types/t-cells/cell-isolation.html (incorporated by reference). Cell sorting methods including single-cell sorting, fluorescent-activated cell sorting, magnetic-activated cell sorting, and cell sorting by microfluidic devices, are known and may be used to isolated T cells by phenotype; hypertext transfer protocol secure://en.wikipedia.org/wiki/Cell_sorting (incorporated by reference, last accessed Aug. 20, 2021).

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. In some embodiments, the disclosed compositions are administered with at least one pharmaceutically acceptable carrier.

The term “pharmaceutically acceptable carrier” is art recognized and includes a pharmaceutically acceptable material, composition or vehicle, suitable for administering compositions of the present disclosure to subjects. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but not limited to, sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. In some embodiments, the pharmaceutically acceptable carrier is sterile and pyrogen-free water. In some embodiments, the pharmaceutically acceptable carrier is Ringer's Lactate, sometimes known as lactated Ringer's solution. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

As used herein, “sequence identity,” “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The term “subject” is used throughout the specification to describe an animal from which a cell sample is taken or an animal to which a disclosed cell or nucleic acid sequences have been administered. In some embodiment, the animal is a human. For diagnosis of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a human suspected of having or being identified as at risk to develop cancer of the blood. In some embodiments, the subject may be diagnosed as having cancer of the blood or being identified as at risk to develop cancer of the blood. In some embodiments, the subject is suspected of having or has been diagnosed with requiring a bone marrow transplant. In some embodiments, the subject may be a human suspected of having or being identified as at risk to develop bone marrow transplants. In some embodiments, the subject may be a mammal which functions as a source of the endothelial cell sample. In some embodiments, the subject may be a non-human animal from which an endothelial cell sample is isolated or provided. The term “mammal” encompasses both humans and non-humans and includes but is not limited to humans, non-human primates, canines, felines, murines, bovines, equines, caprines, and porcines.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. In some embodiments, the nucleic acid is isolated from an organism.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

The terms “polypeptide,” “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the terms “purified” and “isolated” when used in the context of a compound or agent (including proteinaceous agents such as antibodies and polypeptides) that can be obtained from a natural source, e.g., cells, refers to a compound or agent which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. The phrase “substantially free of natural source materials” refers to preparations of a compound or agent that has been separated from the material (e.g., cellular components of the cells) from which it is isolated. Thus, a compound or agent that is isolated includes preparations of a compound or agent having less than about 30%, 20%, 10%, 5%, 2%, or 1% (by dry weight) of cellular materials and/or contaminating materials.

An isolated T cell, precursor T cell, or PBMC may be separated from its natural environment, for example, by removal of red blood cells or leukocytes other than the T cell, precursor T cell or PBMC or by removal of plasma or serum proteins or other components. In some instances, a T cell may be isolated from T cells expressing different T cell markers or phenotypes. T cells may be separated from other cellular and non-cellular components of blood or other biological fluid, or from other components of a biological sample, culture medium or buffer. For example, they may be isolated from red blood cells on a density gradient and recovered from a buffy coat layer or may be sorted using a cell sorter. T cells may also be separated by filtration or centrifugation from other culture components, such as culture medium containing particular cytokines.

An “isolated” nucleic acid sequence or nucleotide sequence is one which is separated from other nucleic acid molecules which are present in a natural source of the nucleic acid sequence or nucleotide sequence. Moreover, an “isolated”, nucleic acid sequence or nucleotide sequence, such as a cDNA molecule, can be substantially free of other cellular material or culture medium when produced by recombinant techniques, or substantially free of chemical precursors when chemically synthesized. In certain embodiments, an “isolated” nucleic acid sequence or nucleotide sequence is a nucleic acid sequence or nucleotide sequence that is recombinantly expressed in a heterologous cell.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50%> of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50%> formamide, 5×SSC, and 1% SDS, incubating at 42° C., or, 5×SSC, 1% SDS, incubating at 65° C., with wash in 0.2×SSC, and 0.1% SDS at 65° C.

The term “hybridization” or “hybridizes” as used herein refers to the formation of a duplex between nucleotide sequences that are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary.” Usually two sequences are sufficiently complementary when at least about 90% (preferably at least about 95%) of the nucleotides share base pair organization over a defined length of the molecule.

“Substantially complementary” as used herein may mean that a first sequence is at least 60%, 65%, 70%, 75%, 80%, 85%, 90%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that, in respect to a first and a second sequence, a first and second sequence are at least 60%, 65%, 70%, 75%, 80%, 85%, 90%, 95%, 96%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 1000 or more nucleotides or amino acids, or with respect to nucleic acids, if the first sequence is substantially complementary to the complement of the second sequence.

The term “therapeutic effect” as used herein is meant to refer to some extent of relief of one or more of the symptoms of a disorder (e.g., SARS-CoV-2 infection) or its associated pathology. A “therapeutically effective amount” as used herein is meant to refer to an amount of an agent which is effective, upon single or multiple dose administration (such as a first, second and/or third booster) to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the present disclosure employed in a pharmaceutical composition at levels lower than that required in order to achieve the desired therapeutic effect and gradually increase the dosage until the desired effect is achieved.

For any therapeutic agent described herein, the therapeutically effective amount may be initially determined from preliminary in vitro studies and/or animal models. A therapeutically effective dose may also be determined from human data. The applied dose can be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below. Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained. Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “treat,” “treated,” “treating,” “treatment,” and the like as used herein are meant to refer to reducing or ameliorating a disorder and/or symptoms associated therewith (e.g., a viral infection). In the context of the administration of a therapy to a subject, the terms “treat,” “treated,” “treating,” “treatment,” and the like refer to the beneficial effects that a subject derives from a therapy, such as, but not limited to, the reduction or inhibition of the progression, spread and/or duration of a disease or disorder, the reduction or amelioration of the severity of a disease or disorder, amelioration of one or more symptoms of a disease or disorder, and/or the reduction in the duration of one or more symptom of a disease or disorder resulting from the administration of one or more therapies. In specific embodiments, such terms in the context of viral infection include, but are not limited to, one, two, or three or more results following the administration of a therapy to a subject: (1) a reduction in the growth of virus in the body by measuring serum levels of virus; (2) a reduction in the number of virus-bearing cells; (3) an eradication, removal, or control of cell number expressing virus; (4) a reduction in mortality; (5) an increase in survival rate; (6) an increase in length of survival; (7) an increase in the number of patients with latent viral infection; (8) a decrease in hospitalization rate; (9) a decrease in hospitalization lengths; and (10) the maintenance in the numbers of virus in serum so that it does not increase by more than 10%, or by more than 8%, or by more than 6%, or by more than 4%; preferably the size of the tumor does not increase by more than 2% from a sample of a subject.

As used herein, the terms “prevent,” “preventing” and “prevention” in the context of the administration of a therapy to a subject refer to the inhibition of the onset or recurrence of a disease or disorder in a subject.

As used herein, the terms “manage,” “managing,” and “management,” in the context of the administration of a therapy to a subject, refer to the beneficial effects that a subject derives from a therapy, which does not result in a cure of a disease or disorder. In certain embodiments, a subject is administered one or more therapies to “manage” a disease or disorder so as to prevent the progression or worsening of symptoms associated with a disease or disorder.

In some embodiments, any of the nucleic acids disclosed herein can encode variants of any of the polypeptides disclosed herein. “Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof, or (iv) a nucleic acid that hybridizes under stringent conditions to the referenced nucleic acid, complement thereof, or a sequences substantially identical thereto. “Variant” with respect to a peptide or polypeptide that differs in amino acid sequence by the insertion, deletion, or conservative substitution of amino acids, but retain at least one biological activity, such as the biological activity of the one or combination of cytokines presented herein. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Nucleic acid molecules or nucleic acid sequences of the disclosure include those coding sequences comprising one or more of: nucleic acid sequences encoding any of the amino acid sequences disclosed herein, such as those identified in Table 1, and functional fragments thereof that possess no less than about 65, 70, 75, 80, 85, 90, 95, 96, 97, 98, or 99% sequence identity with the coding sequences of the amino acid sequences disclosed herein.

“Vector” used herein means, in respect to a nucleic acid sequence, a nucleic acid sequence comprising a regulatory nucleic acid sequence that controls the replication or expression of an expressible gene. A vector may be either a self-replicating, extrachromosomal vector or a vector which integrates into a host genome. Alternatively, a vector may also be a vehicle comprising the aforementioned nucleic acid sequence. A vector may be a plasmid, bacteriophage, viral particle (isolated, attenuated, recombinant, etc.). A vector may comprise a double-stranded or single-stranded DNA, RNA, or hybrid DNA/RNA sequence comprising double-stranded and/or single-stranded nucleotides. In some embodiments, the vector is a viral vector that comprises a nucleic acid sequence that is a viral packaging sequence responsible for packaging one or plurality of nucleic acid sequence that encode one or a plurality of polypeptides. In some embodiments, the vector comprises a viral particle comprising a nucleic acid sequence operably linked to a regulatory sequence, wherein the nucleic acid sequence encodes a fusion protein comprising one or a plurality of structural viral polypeptides or fragments thereof. The disclosure relates to any vector comprising one or a plurality of nucleic acid sequences encoding any two of the disclosed cytokines of Table 1, and/or any functional fragment or variant thereof comprising at least about 75%, 80%, 85%, 90%, 91%, 92%, 93%, 94%, 95%, 96%, 97%, 98%, or 99% sequence identity to SEQ ID NO: 1, SEQ ID NO: 3, SEQ ID NO: 5, SEQ ID NO: 7 or SEQ ID NO: 9. In some embodiments, the disclosure relates to the vectors comprising, consisting of, or consisting essentially of SEQ ID NO: 2, SEQ ID NO: 4, SEQ ID NO: 6, SEQ ID NO: 8 or SEQ ID NO: 10. In some embodiments, the disclosure relates to the vectors comprising a nucleic acid that encodes SEQ ID NO: 1, SEQ ID NO: 3, SEQ ID NO: 5, SEQ ID NO: 7 or SEQ ID NO: 9, or a functional fragment thereof. In some embodiments, the disclosure relates to the vectors comprising variants or functional fragments of SEQ ID NO: 2, SEQ ID NO: 4, SEQ ID NO: 6, SEQ ID NO: 8 or SEQ ID NO: 10. In some embodiments, the disclosure relates to the vectors comprising variants or functional fragments of SEQ ID NO: 2, SEQ ID NO: 4, SEQ ID NO: 6, SEQ ID NO: 8 or SEQ ID NO: 10 that comprises at least about 75%, 80%, 85%, 90%, 91%, 92%, 93%, 94%, 95%, 96%, 97%, 98%, 99%, <100%, or 100% sequence identity to SEQ ID NO: 2, SEQ ID NO: 4, SEQ ID NO: 6, SEQ ID NO: 8 or SEQ ID NO: 10. In some embodiments, the functional fragment is a subunit of the cytokine known to have biological effect without association with another subunit.

“Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as a endothelial cell or hematopoietic cell in culture.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Methods and Compositions

The disclosure relates to T cell compositions for the treatment of disorders, such as blood disorders including myeloid cell-based cancers, and viral infections, such as coronaviral infections including COVID-19, can be administered as a single composition comprising T cell subpopulations stimulated by the methods disclosed herein. In some embodiments, the T cell compositions are stimulated with a combination of cytokines disclosed herein for a time period sufficient to stimulate growth and proliferation of the one or plurality of CD4+ and/or CD8+ T cells. In some embodiments, the T cell compositions are stimulated with a combination of cytokines disclosed herein and are exposed to one or more tumor-associated antigens or viral antigens for a time period sufficient to stimulate growth and proliferation of the one or plurality of CD4+ and/or CD8+ T cells that are specific to the tumor-associated antigens or viral antigens used for stimulation. In some embodiments, the time period sufficient to stimulate growth or proliferation of the one or plurality of CD4+ and/or CD8+ T cells is from about 1 day to about 12 days. In some embodiments, the time period sufficient to stimulate growth or proliferation of the one or plurality of CD4+ and/or CD8+ T cells is from about 2 to about 10 days. In some embodiments, the time period sufficient to stimulate growth or proliferation of the one or plurality of CD4+ and/or CD8+ T cells is from about 3 to about 7 days. In some embodiments, the time period sufficient to stimulate growth or proliferation of the one or plurality of CD4+ and/or CD8+ T cells is from about 4 to about 8 days. In some embodiments, the time period sufficient to stimulate growth or proliferation of the one or plurality of CD4+ and/or CD8+ T cells is from about 5 to about 7 days. In some embodiments, the time period sufficient to stimulate growth or proliferation of the one or plurality of CD4+ and/or CD8+ T cells is about 1 day. In some embodiments, the time period sufficient to stimulate growth or proliferation of the one or plurality of CD4+ and/or CD8+ T cells is about 2 days. In some embodiments, the time period sufficient to stimulate growth or proliferation of the one or plurality of CD4+ and/or CD8+ T cells is about 3 days. In some embodiments, the time period sufficient to stimulate growth or proliferation of the one or plurality of CD4+ and/or CD8+ T cells is about 4 days. In some embodiments, the time period sufficient to stimulate growth or proliferation of the one or plurality of CD4+ and/or CD8+ T cells is about 5 days. In some embodiments, the time period sufficient to stimulate growth or proliferation of the one or plurality of CD4+ and/or CD8+ T cells is about 6 days. In some embodiments, the time period sufficient to stimulate growth or proliferation of the one or plurality of CD4+ and/or CD8+ T cells is about 7 days. In some embodiments, the time period sufficient to stimulate growth or proliferation of the one or plurality of CD4+ and/or CD8+ T cells is about 8 days. In some embodiments, the time period sufficient to stimulate growth or proliferation of the one or plurality of CD4+ and/or CD8+ T cells is about 9 days. In some embodiments, the time period sufficient to stimulate growth or proliferation of the one or plurality of CD4+ and/or CD8+ T cells is about 10 days. In some embodiments, the time period sufficient to stimulate growth or proliferation of the one or plurality of CD4+ and/or CD8+ T cells is about 11 days. In some embodiments, the time period sufficient to stimulate growth or proliferation of the one or plurality of CD4+ and/or CD8+ T cells is about 12 days.

In some embodiments, the time period sufficient to stimulate growth or proliferation of the one or plurality of CD4+ and/or CD8+ T cells is a time period in which no less than about 5% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 6% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 7% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 8% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 9% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 10% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 11% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 12% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 13% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 14% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 15% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 16% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 17% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 18% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 19% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 20% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 21% of CD8+ or CD4+ T cells produce either IFNγ or TNFα. In some embodiments, the time period sufficient to stimulate growth or proliferation of the one or plurality of CD4+ and/or CD8+ T cells is a time period in which no less than about 22% of CD8+ or CD4+ T cells produce either IFNγ or TNFα.

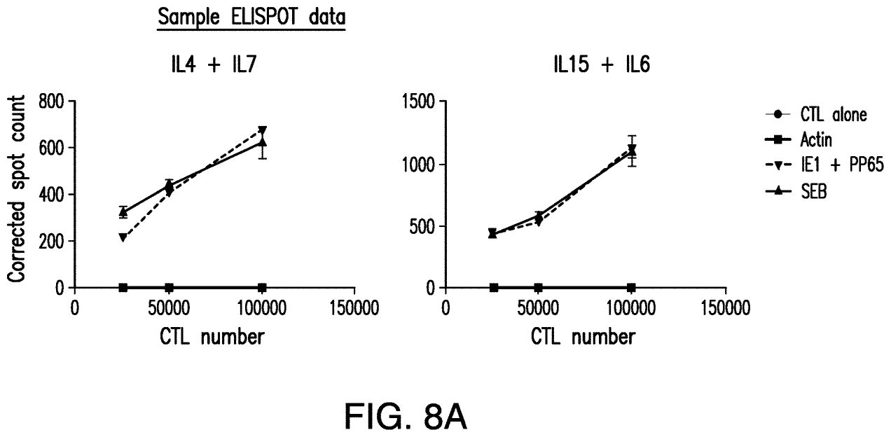

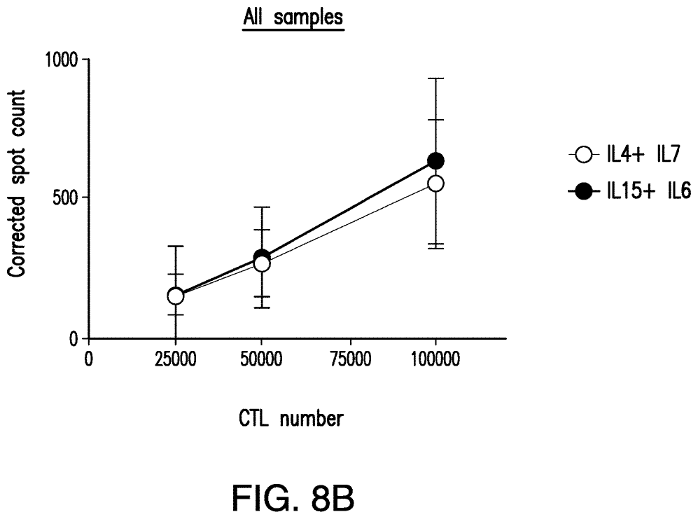

In some embodiments, the subpopulations of T cells are derived through the ex vivo expansion of a single population of T cells, wherein the single population of T cells are exposed to a pool of one or more antigenic peptides (epitopes) of each of the viral antigens in the presence of a combination of two or more cytokines chosen from: IL-18, IL-15, IL-6, IL-7 and IL-4. In some embodiments, the combination of cytokines is IL-15 and IL-6. In some embodiments, the combination of cytokines is IL-15 and IL-7. In some embodiments, the combination of cytokines is IL-7 and IL-4. The disclosure relates to T cell compositions comprising memory effector T cells that are antigen-specific for one or more viral antigens. In some embodiments, the T cell compositions are activated to recognize one or a plurality of viral antigen peptides provided in FIG. 8 or peptides that comprise at least about 70%, 75%, 80%, 85%, 90%, 91%, 92%, 93%, 94%, 95%, 96%, 97%, 98% or 99% sequence identity to the amino acid sequences depicted in FIG. 8. In some embodiments, the T cell compositions are activated to recognize one or more viral antigens of a virus of the family Coronaviridae. In some embodiments, the T cell compositions are activated to recognize one or more viral antigens of a coronavirus. In some embodiments, the T cell compositions are activated to recognize one or more viral antigens of SARS-CoV-2.

Coronaviruses are a group of viruses that cause diseases in mammals and birds. Coronaviruses were first discovered in the 1960s. The earliest virus from the family of Coronaviridae discovered were infectious bronchitis virus in chickens and two viruses from the nasal cavities of human patients with the common cold that were subsequently named human coronavirus 229E (HCoV-229E) and human coronavirus OC43 (HCoV-OC43). Other members of this family have since been identified, including SARS-CoV in 2003, HCoV NL63 in 2004, HKU1 in 2005, MERS-CoV in 2012, and SARS-CoV-2 (formerly known as 2019-nCoV or novel coronavirus 2019, which caused the global COVID-19 pandemic). Most of these have involved serious respiratory tract infections.

SARS-CoV-2 is the virus strain that causes the coronavirus disease 2019 (COVID-19) pandemic, which infected millions of people and caused hundreds of thousand of death worldwide. Like other coronaviruses, SARS-CoV-2 has four structural proteins, known as the S (spike), E (envelope), M (membrane), and N (nucleocapsid) proteins. The N protein holds the viral RNA genome, and the S, E, and M proteins together create the viral envelope.

The complete genome of SARS-CoV-2 has been sequenced and the sequence is publically available in the GenBank database under the accession No. NC_045512. The spike (S) protein is the protein responsible for allowing the virus to attach to and fuse with the membrane of a host cell and has the amino acid sequence of SEQ ID NO: 11 (GenBank Accession No. QHD43416).

(SEQ ID NO: 11)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

The envelope (E) protein of SARS-CoV-2 has the amino acid sequence of SEQ ID NO: 12 (GenBank Accession No. QHD43418).

(SEQ ID NO: 12)
MYSFVSEETGTLIVNSVLLFLAFVVFLLVTLAILTALRLCA
YCCNIVNVSLVKPSFYVYSRVKNLNSSRVPDLLV

The membrane (M) protein of SARS-CoV-2 has the amino acid sequence of SEQ ID NO: 13 (GenBank Accession No. QHD43419).

(SEQ ID NO: 13)
MADSNGTITVEELKKLLEQWNLVIGFLFLTWICLLQFAYANR
NRFLYIIKLIFLWLLWPVTLACFVLAAVYRINWITGGIAIAM
ACLVGLMWLSYFIASFRLFARTRSMWSFNPETNILLNVPLHG
TILTRPLLESELVIGAVILRGHLRIAGHHLGRCDIKDLPKEI
TVATSRTLSYYKLGASQRVAGDSGFAAYSRYRIGNYKLNTDH
SSSSDNIALLVQ

The nucleocapsid (N) protein of SARS-CoV-2 has the amino acid sequence of SEQ ID NO: 14 (GenBank Accession No. QHD43423).

(SEQ ID NO: 14)
MSDNGPQNQRNAPRITFGGPSDSTGSNQNGERSGAR
SKQRRPQGLPNNTASWFTALTQHGKEDLKFPRGQG
VPINTNSSPDDQIGYYRRATRRIRGGDGKMKDLSP
RWYFYYLGTGPEAGLPYGANKDGIIWVATEGALNT
PKDHIGTRNPANNAAIVLQLPQGTTLPKGFYAEGS
RGGSQASSRSSSRSRNSSRNSTPGSSRGTSPARMA
GNGGDAALALLLLDRLNQLESKMSGKGQQQQGQTV
TKKSAAEASKKPRQKRTATKAYNVTQAFGRRGPEQ
TQGNFGDQELIRQGTDYKHWPQIAQFAPSASAFFG
MSRIGMEVTPSGTWLTYTGAIKLDDKDPNFKDQVI
LLNKHIDAYKTFPPTEPKKDKKKKADETQALPQRQ
KKQQTVTLLPAADLDDFSKQLQQSMSSADSTQA

T cell compositions activated to recognize one or more viral antigens of SARS-CoV-2 can be prepared by exposing or pulsing antigen presenting cells or artificial antigen presenting cells with one or more peptides or epitopes from SARS-CoV-2. In some embodiments, if more than one peptide from a single SARS-CoV-2 antigen is used, the peptide segments can be generated by making overlapping peptide fragments of the SARS-CoV-2 antigen, as provided for example in commercially available overlapping peptide libraries or “PepMix™”. In some embodiments, the overlapping peptide libraries include peptides that are about 7, 8, 9, 10, 11, 12, 13, 14, 15, 16 or more amino acids long and overlapping one another by about 5, 6, 7, 8, 9, 10, 11 or more amino acids. In some embodiments, the peptides are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length.

In some embodiments, the SARS-CoV-2 specific T cell compositions are generated using one or more antigenic peptides to SARS-CoV-2, or a modified or heteroclitic peptide derived from a SARS-CoV-2 antigenic peptide. In some embodiments, SARS-CoV-2 specific T cell compositions are generated using a SARS-CoV-2 antigen library comprising a pool of peptides (for example, peptides that are from about 10 to about 20 amino acids in length) from one or a combination of the structural proteins of SARS-CoV-2 disclosed herein, such as 15-mers peptides containing amino acids overlap (for example 11 amino acids of overlap) between each peptide formed by scanning the proteins having the amino acid sequences of SEQ ID NO: 11, SEQ ID NO: 12, SEQ ID NO: 13 and/or SEQ ID NO: 14, or functional fragments or variants thereof. In some embodiments, the SARS-CoV-2 specific T cell compositions are generated using a SARS-CoV-2 antigen library comprising a pool of peptides formed by scanning the protein having the amino acid sequence of SEQ ID NO: 11, or functional fragments or variants thereof. In some embodiments, the SARS-CoV-2 specific T cell compositions are generated using a SARS-CoV-2 antigen library comprising a pool of peptides formed by scanning the protein having the amino acid sequence of SEQ ID NO: 12, or functional fragments or variants thereof. In some embodiments, the SARS-CoV-2 specific T cell compositions are generated using a SARS-CoV-2 antigen library comprising a pool of peptides formed by scanning the protein having the amino acid sequence of SEQ ID NO: 13, or functional fragments or variants thereof. In some embodiments, the SARS-CoV-2 specific T cell compositions are generated using a SARS-CoV-2 antigen library comprising a pool of peptides formed by scanning the protein having the amino acid sequence of SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11 and SEQ ID NO: 12, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11 and SEQ ID NO: 13, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 12 and SEQ ID NO: 13, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 12 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 13 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11, SEQ ID NO: 12 and SEQ ID NO: 13, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11, SEQ ID NO: 12 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11, SEQ ID NO: 13 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 12, SEQ ID NO: 13 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11, SEQ ID NO: 12, SEQ ID NO: 13 and SEQ ID NO: 14, or functional fragments or variants thereof.

In some embodiments, the SARS-CoV-2 specific T cell compositions are generated using one or a plurality of epitopes located in the protein of SEQ ID NO: 11. In some embodiments, the SARS-CoV-2 specific T cell compositions are generated using one or a plurality of epitopes located in amino acid residue from about 12 to about 524 of the protein of SEQ ID NO: 11. In some embodiments, the SARS-CoV-2 specific T cell compositions are generated using one or a plurality of epitopes located in amino acid residue from about 12 to about 331 of the protein of SEQ ID NO: 11. In some embodiments, the SARS-CoV-2 specific T cell compositions are generated using one or a plurality of epitopes located in amino acid residue from about 57 to about 75 of the protein of SEQ ID NO: 11. In some embodiments, the SARS-CoV-2 specific T cell compositions are generated using one or a plurality of epitopes located in amino acid residue from about 205 to about 224 of the protein of SEQ ID NO: 11. In some embodiments, the SARS-CoV-2 specific T cell compositions are generated using one or a plurality of epitopes located in amino acid residue from about 331 to about 524 of the protein of SEQ ID NO: 11. In some embodiments, the SARS-CoV-2 specific T cell compositions are generated using one or a plurality of epitopes located in amino acid residue from about 449 to about 463 of the protein of SEQ ID NO: 11. In some embodiments, the SARS-CoV-2 specific T cell compositions are generated using one or a plurality of epitopes selected from Table A.

TABLE A
Epitopes of the spike protein of
SEQ ID NO: 11.

| SEQ ID NO. | Sequence |
|---|---|
| 11 (residues 57-71) | PFFSNVTWFHAIHVS |
| 11 (residues 61-75) | NVTWFHAIHVSGTNG |
| 11 (residues 205-219) | SKHTPINLVRDLPQG |
| 11 (residues 209-223) | PINLVRDLPQGFSAL |
| 11 (residues 449-463) | YNYLYRLFRKSNLKP |
| 11 (residues 891-906) | GAALQIPFAMQMAYRF |
| 11 (residues 902-917) | MAYRFNGIGVTQNVLY |
| 11 (residues 1011-1028) | QLIRAAEIRASANLAATK |
| 11 (residues 1220-1228) | FIAGLIAIV |
| 11 (residues 958-966) | ALNTLVKQL |
| 11 (residues 996-1004) | LITGRLQSL |
| 11 (residues 1192-1200) | NLNESLIDL |
| 11 (residues 957-973) | QALNTLVKQLSSNFGAI |
| 11 (residues 1185-1193 | RLNEVAKNL |
| 11 (residues 976-984) | VLNDILSRL |
| 11 (residues 1060-1068) | VVFLHVTYV |

In some embodiments, the SARS-CoV-2 specific T cell compositions are generated using one or a plurality of epitopes located in the protein of SEQ ID NO: 12.

In some embodiments, the SARS-CoV-2 specific T cell compositions are generated using one or a plurality of epitopes located in the protein of SEQ ID NO: 13. In some embodiments, the SARS-CoV-2 specific T cell compositions are generated using one or a plurality of epitopes located in amino acid residue from about 100 to about 222 of the protein of SEQ ID NO: 13. In some embodiments, the SARS-CoV-2 specific T cell compositions are generated using one or a plurality of epitopes located in amino acid residue from about 144 to about 192 of the protein of SEQ ID NO: 13. In some embodiments, the SARS-CoV-2 specific T cell compositions are generated using one or a plurality of epitopes located in amino acid residue from about 144 to about 163 of the protein of SEQ ID NO: 13. In some embodiments, the SARS-CoV-2 specific T cell compositions are generated using one or a plurality of epitopes located in amino acid residue from about 163 to about 192 of the protein of SEQ ID NO: 13. In some embodiments, the SARS-CoV-2 specific T cell compositions are generated using one or a plurality of epitopes located in amino acid residue from about 173 to about 192 of the protein of SEQ ID NO: 13. In some embodiments, the SARS-CoV-2 specific T cell compositions are generated using one or a plurality of epitopes selected from Table B.

TABLE B
Epitopes of the M protein of
SEQ ID NO: 13.

| SEQ ID NO. | Sequence |
|---|---|
| 13 (residues 145-159) | LRGHLRIAGHHLGRC |
| 13 (residues 149-163) | LRIAGHHLGRCDIKD |
| 13 (residues 173-187) | SRTLSYYKLGASQRV |
| 13 (residues 177-191) | SYYKLGASQRVAGDS |
| 13 (residues 181-195) | LGASQRVAGDSGFAA |

In some embodiments, the SARS-CoV-2 specific T cell compositions are generated using one or a plurality of epitopes located in the protein of SEQ ID NO: 14. In some embodiments, the SARS-CoV-2 specific T cell compositions are generated using one or a plurality of epitopes located in amino acid residue from about 257 to about 361 of the protein of SEQ ID NO: 14. In some embodiments, the SARS-CoV-2 specific T cell compositions are generated using one or a plurality of epitopes located in amino acid residue from about 257 to about 271 of the protein of SEQ ID NO: 14. In some embodiments, the SARS-CoV-2 specific T cell compositions are generated using one or a plurality of epitopes selected from Table C.

TABLE C
Epitopes of the N protein of
SEQ ID NO: 14.

| SEQ ID NO. | Sequence |
|---|---|
| 14 (residues 257-271) | KPRQKRTATKAYNVT |
| 14 (residues 313-327) | AFFGMSRIGMEVTPS |
| 14 (residues 351-359) | ILLNKHIDH |
| where the last | |
| residue is H. | |
| 14 (residues 321-331) | MEVTPSGTWL |
| 14 (residues 316-324) | GMSRIGMEV |
| 14 (residues 351-359) | ILLNKHIDA |
| where the last | |
| residue is A. | |
| 14 (residues 138-146) | ALNTPKDHI |
| 14 (residues 219-227) | LALLLLDRL |
| 14 (residues 222-230) | LLLDRLNQL |
| 14 (residues 159-167) | LQLPQGTTL |

Alternatively, the T cell compositions can be generated through the ex vivo expansion of a first T cell population exposed to one or more antigenic peptides of each of the selected viral antigens separately, wherein following activation and expansion of lymphocytes, the first T cell population is combined with a second T cell population stimulated by a cytokine composition disclosed herein into a single composition for administration. In another alternative, the T cell compositions can be derived through the ex vivo expansion of a first and second T cell populations exposed to one or more antigenic peptides of each of the selected viral antigens separately, wherein following activation and expansion, the separate T cell populations are each individually administered simultaneously or sequentially to the subject. In some embodiments, the first and second T cell populations are derived from the same donor source. In some embodiments, the first or second T cell populations are derived from one or more different donor sources. In some embodiments, the cells are contacted sequentially or simultaneously with any of the cytokine combinations disclosed herein and one or more polypeptides or nucleic acid sequences encoding polypeptides chosen from one or a combination of peptides that comprise at least about 70/a, 75%, 80%, 85%, 90%, 91%, 92%, 93%, 94%, 95%, 96%, 97%, 98%, 99%, <100%, or 100% sequence identity to the amino acid sequences depicted in FIG. 8 or variants or functional fragments thereof. In some embodiments, the cells are contacted sequentially or simultaneously with any of the cytokine combinations disclosed herein and one or more peptides (for example, peptides that are from about 10 to about 20 amino acids in length) from one or a combination of SARS-CoV-2 antigens having the amino acid sequences of SEQ ID NO: 11, SEQ ID NO: 12, SEQ ID NO: 13 and/or SEQ ID NO: 14, or functional fragments or variants thereof.

The T cell compositions described herein may be derived from a population of cells from an autologous source, an allogeneic source, for example a healthy donor not suffering from a disorder, or cord blood.

In some embodiments, the methods disclosed herein use PBMCs, stem cells, pre-T cells, or cord blood, from a partially histocompatible sibling, parent, son or daughter, grandparent, grandson or grand daughter, first or second cousin, or other blood relative. In other embodiments, T cells may be obtained from autologous cells. Those skilled in the art may select an appropriate match by minimizing mismatches of HLA type-I genes (e.g. HLA-A, HLA-B, or HLA-C) which increase the risk of graft rejection, and/or by minimizing the mismatches of an HLA type II gene (e.g. HLA-DR or HLA-DQB1) which increase the risk of graft-versus-host disease. Typically, antigen-specific T cells are produced from non-naïve or naïve cells that share at least 1, 2, 3, 4, 5, 6, 7, 8, 9, 10, 11 or 12 HLA alleles (e.g., HLA-A, HLA-B, HLA-C, HLA-DPA1, HLA-DPB1, HLA-DQA1, HLA-DQB1, HLA-DRA, and HLA-DRB1) with a prospective donor.

In some embodiments, the T cells compositions described herein may be derived from a population of cells from a subject diagnosed with or suspected of having a viral infection. In some embodiments, the T cells compositions described herein may be derived from a population of cells from a subject diagnosed with or suspected of having coronaviral infection. In some embodiments, the T cells compositions described herein may be derived from a population of cells from a subject diagnosed with or suspected of having infection of SARS-CoV-2. In some embodiments, the T cells compositions described herein may be derived from a population of cells from a subject diagnosed with or suspected of having COVID-19. Non-limiting exemplary methods of generating ex vivo primed and expanded T cells capable of recognizing at least one antigenic peptide of a tumor antigen can be found in Shafer et al., Leuk Lymphoma (2010) 51(5):870-880; Cruz et al., Clin Cancer Res., (2011) 17(22): 7058-7066; Quintarelli et al., Blood (2011) 117(12): 3353-3362; Chapuis et al., Sci Transl Med (2013) 5(174):174ra27; and US 2017/0037369, all incorporated herein by reference in their entireties.

In order to prime and activate the particular T cell subpopulations of the described T cell compositions, one or more antigenic peptides (epitopes) from the target viral antigen is used in addition to the cytokine compositions disclosed herein. For example, a single antigenic peptide, multiple antigenic peptides, or a library of antigenic peptides can be used to prime and activate a T cell subpopulation targeting each of the specific viral antigens. In some embodiments, if more than one peptide from the viral antigen is used, the peptide segments can be generated by making overlapping peptide fragments of viral antigen that are from about 5 to about 15 amino acids in length as discussed above. Alternatively, generation of the T cell composition can be accomplished through the ex vivo priming and activation of the T cell subpopulations with selected antigenic epitopes of the targeted viral antigen, for example, a single epitope or multiple specific epitopes of the viral antigen. In some embodiments, the T cell subpopulation is activated and primed with pooled peptides to a viral antigen, wherein the pooled peptides include a library of overlapping peptides from the viral antigen (peptide mix) which has been enriched by additionally including one or more specific known, identified, or heteroclitic epitopes of the viral antigen. In some embodiments, the peptides used to prime the T cells are the same length. In some embodiments, the peptides are of varying lengths. In other embodiments, the peptides included in the pool for priming the T cells substantially only include known viral antigenic epitopes. In some embodiments, the T cell subpopulation is primed with one or more antigenic peptides expressed by a patient's tumor. In some embodiments, the T cell subpopulation is primed with one or more neoantigens. In some embodiments, the neoantigen is a mutated form of an endogenous protein derived through a single point mutation, a deletion, an insertion, a frameshift mutation, a fusion, misspliced peptide, or intron translation of the targeted tumor cells comprising the viral antigens.

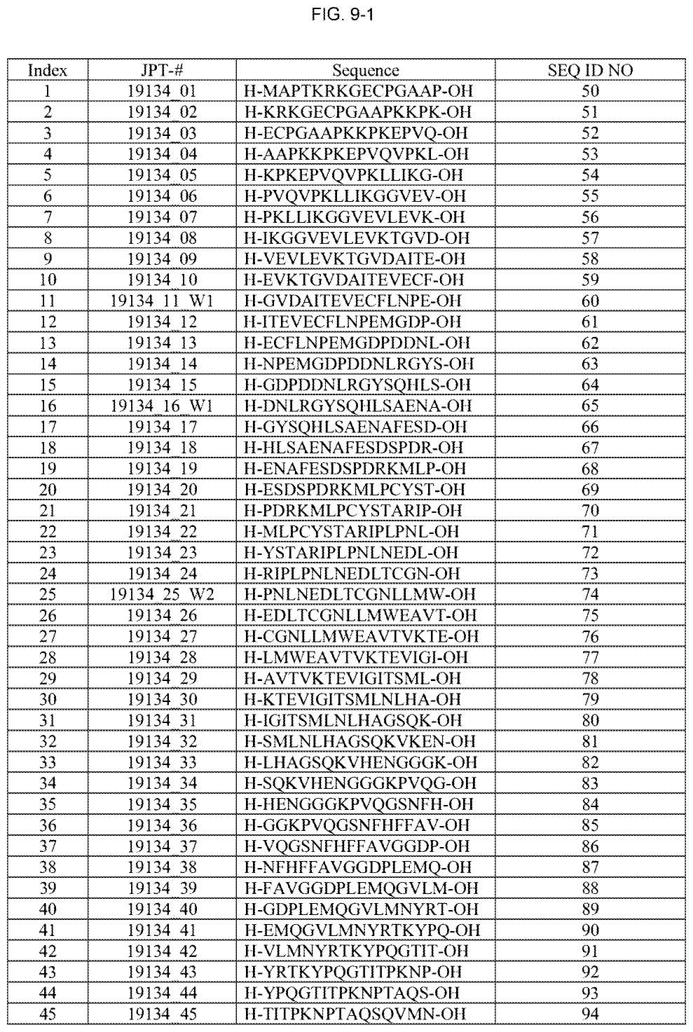

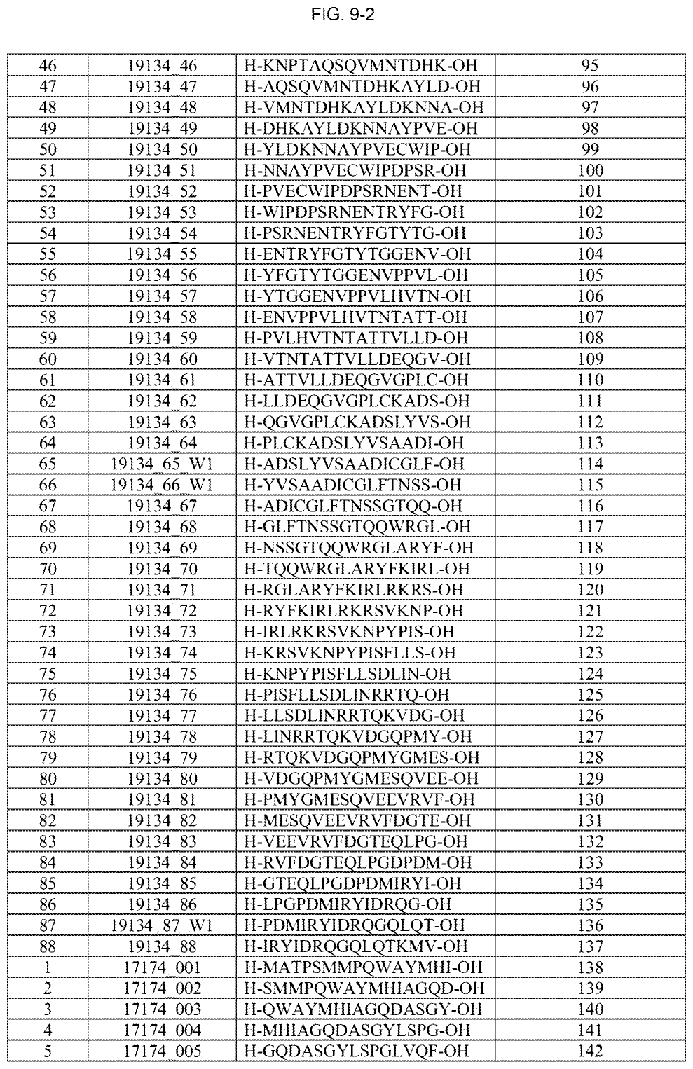

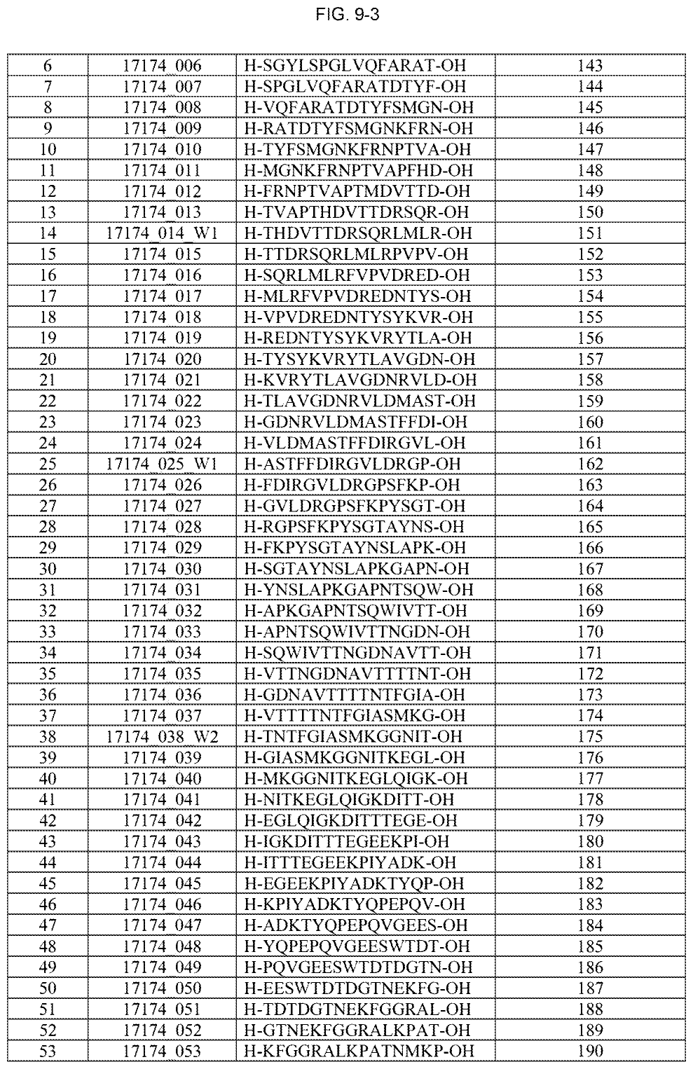

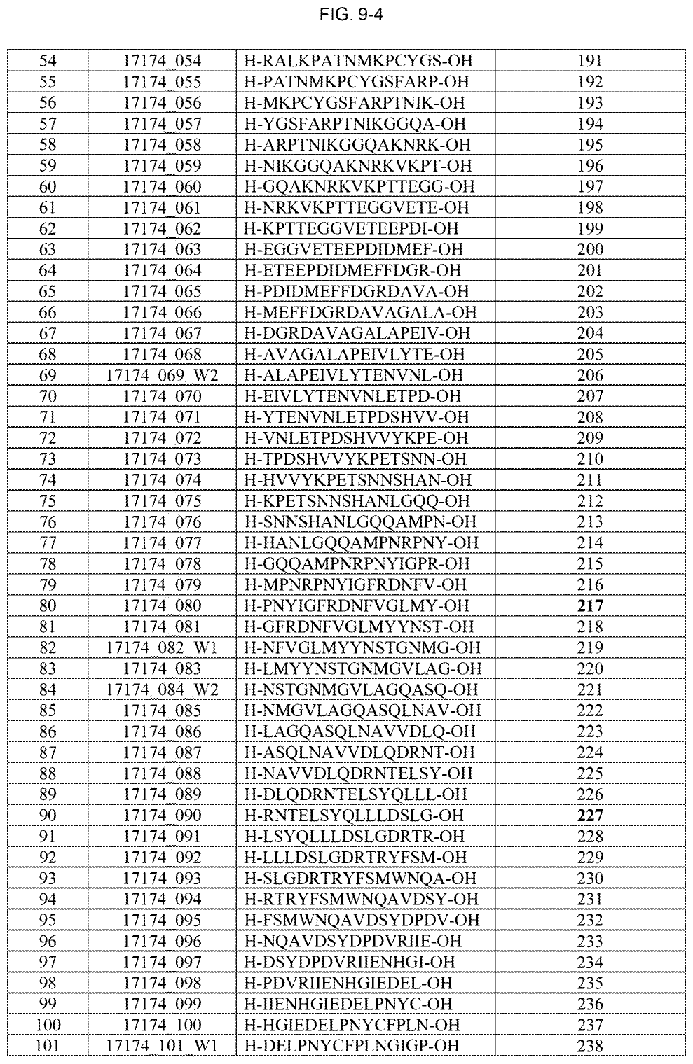

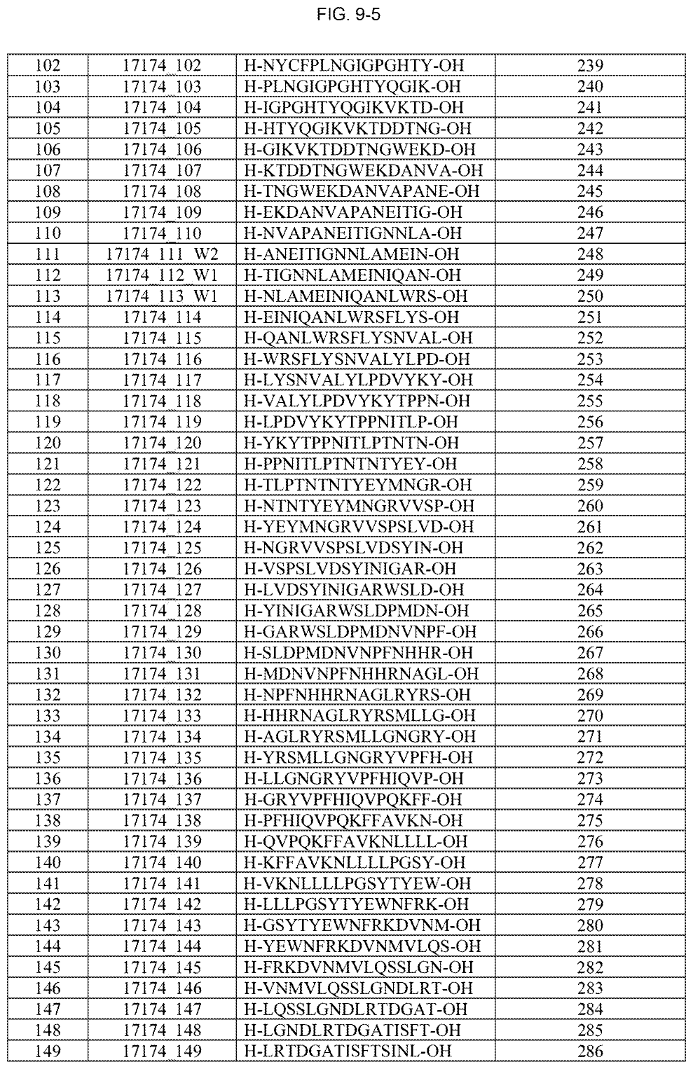

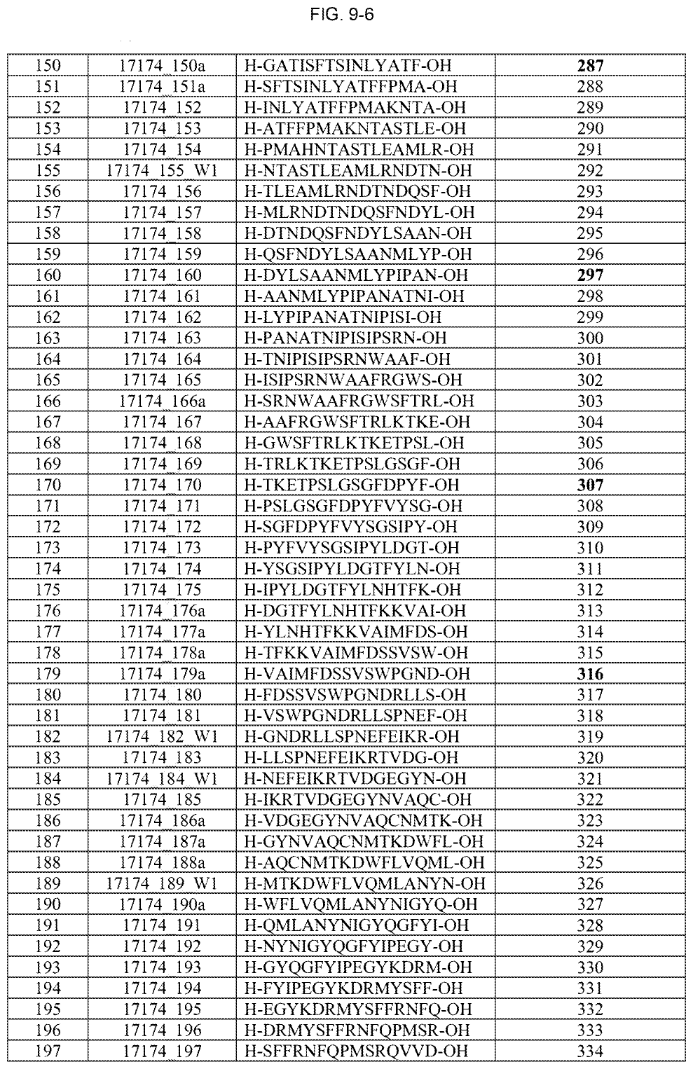

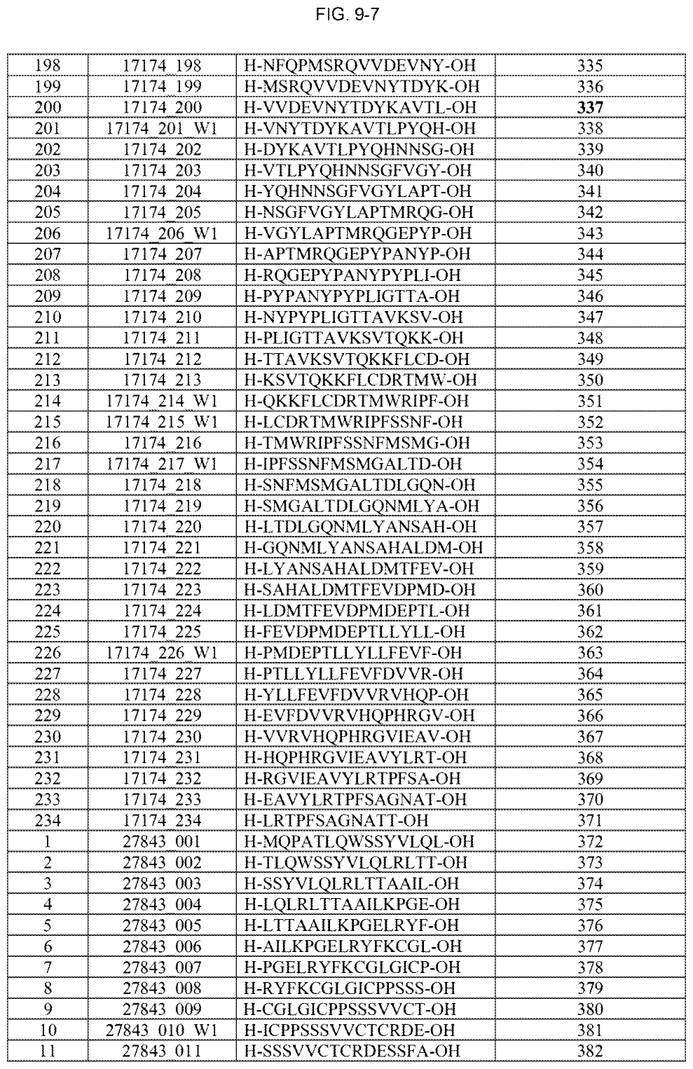

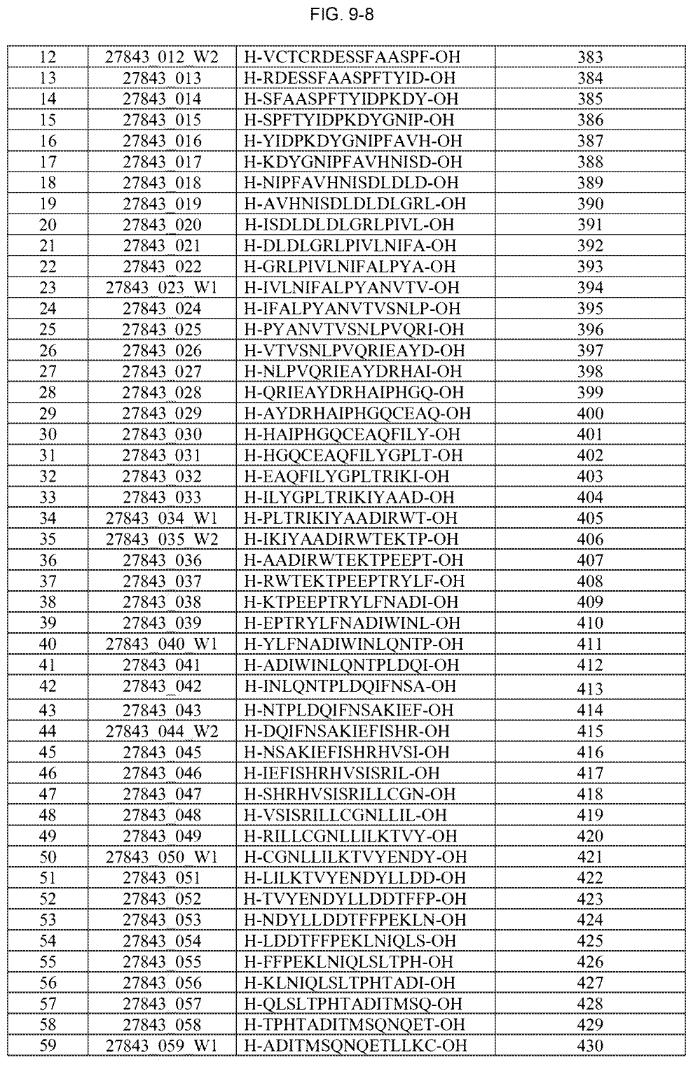

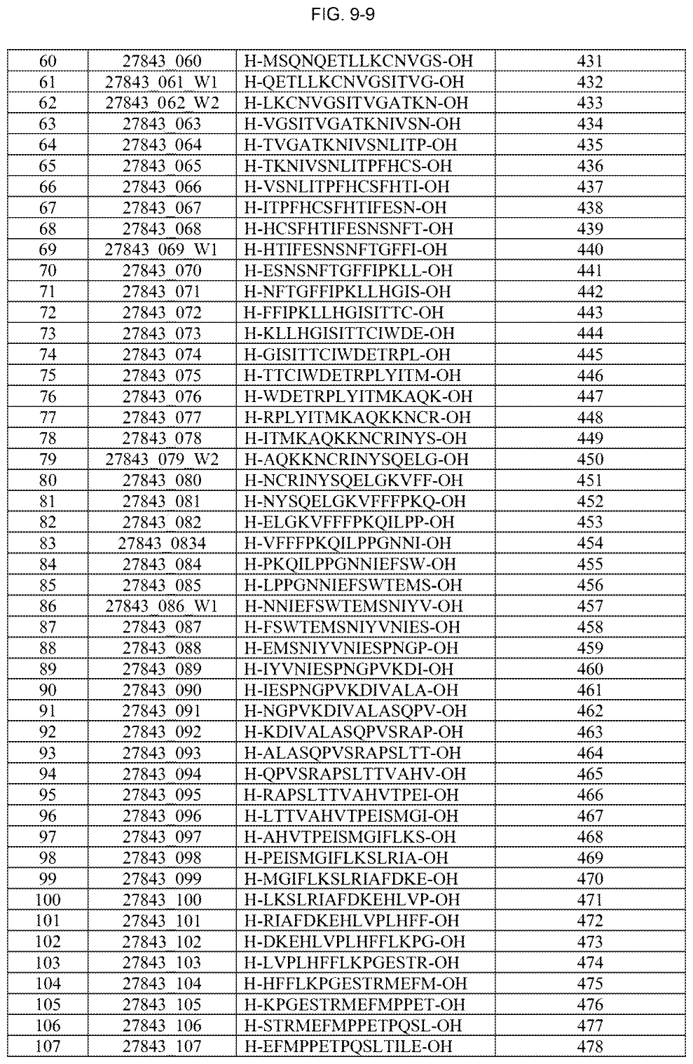

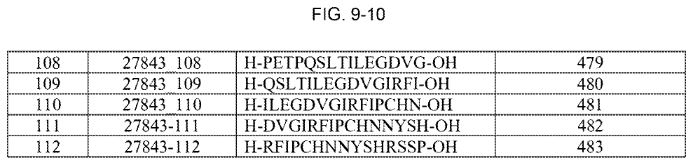

In some embodiments, the T cell composition is derived through the ex vivo expansion of separate T cell populations (a first T cell population and a second T cell population), wherein the T cell composition includes T cell subpopulations primed separately to a pool of viral peptides, wherein each T cell subpopulation is specific for a single viral antigenic peptide. In some embodiments, the pooled viral peptides are comprised of overlapping peptides derived from viral peptides selected from those provided in FIG. 9. Alternatively, individual or mixed peptides (pepmix) may be obtained commercially, for example, from JPT (hypertext transfer protocol secure//shop.jpt.com). These include PepMix™ SARS-CoV-2 (Spike B.1.1.7), PepMix™ SARS-CoV (Spike Glycoprotein); PepMix™ SARS-CoV-2 (AP3A); PepMix™ SARS-CoV-2 (C+NSP 11); PepMix™ SARS-CoV-2 (NCAP); PepMix™ SARS-CoV-2 (NCPMUT); PepMix™ SARS-CoV-2 (NS6); PepMix™ SARS-CoV-2 (NS7a); PepMix™ SARS-CoV-2 (NS7B); PepMix™ SARS-CoV-2 (NS8); PepMix™ SARS-CoV-2 (Nsp1); PepMix™ SARS-CoV-2 (Nsp2-Nsp16) as well as the other SARS-CoV-2, coronavirus, and viral peptides and pepmixes described in the JPT catalog (last accessed Sep. 8, 2021, incorporated by reference).

In some embodiments, the pooled viral peptides are comprised of overlapping peptides derived from viral peptides chosen from SEQ ID NO: 11, SEQ ID NO: 12, SEQ ID NO: 13 and SEQ ID NO: 14, or functional fragments or variants thereof having at least about 70%, 75%, 80%, 85%, 90%, 91%, 92%, 93%, 94%, 95%, 96%, 97%, 98% or 99% sequence identity to SEQ ID NO: 11, SEQ ID NO: 12, SEQ ID NO: 13 or SEQ ID NO: 14. In some embodiments, the pooled viral peptides are comprised of overlapping peptides derived from the viral antigen of SEQ ID NO: 11, or functional fragments or variants thereof having at least about 70%, 75%, 80%, 85%, 90%, 91%, 92%, 93%, 94%, 95%, 96%, 97%, 98% or 99% sequence identity to SEQ ID NO: 11. In some embodiments, the pooled viral peptides are comprised of overlapping peptides derived from the viral antigen of SEQ ID NO: 12, or functional fragments or variants thereof having at least about 70%, 75%, 80%, 85%, 90%, 91%, 92%, 93%, 94%, 95%, 96%, 97%, 98% or 99% sequence identity to SEQ ID NO: 12. In some embodiments, the pooled viral peptides are comprised of overlapping peptides derived from the viral antigen of SEQ ID NO: 13, or functional fragments or variants thereof having at least about 70%, 75%, 80%, 85%, 90%, 91%, 92%, 93%, 94%, 95%, 96%, 97%, 98% or 99% sequence identity to SEQ ID NO: 13. In some embodiments, the pooled viral peptides are comprised of overlapping peptides derived from the viral antigen of SEQ ID NO: 14, or functional fragments or variants thereof having at least about 70%, 75%, 80%, 85%, 90%, 91%, 92%, 93%, 94%, 95%, 96%, 97%, 98% or 99% sequence identity to SEQ ID NO: 14. In some embodiments, the pooled viral peptides derived from the viral antigen of SEQ ID NO: 11, or functional fragments or variants thereof, are further enriched with one or more additional peptides derived from SEQ ID NO: 12, SEQ ID NO: 13 and/or SEQ ID NO: 14, or functional fragments or variants thereof. In some embodiments, the pooled viral peptides derived from the viral antigen of SEQ ID NO: 12, or functional fragments or variants thereof, are further enriched with one or more additional peptides derived from SEQ ID NO: 11, SEQ ID NO: 13 and/or SEQ ID NO: 14, or functional fragments or variants thereof. In some embodiments, the pooled viral peptides derived from the viral antigen of SEQ ID NO: 13, or functional fragments or variants thereof, are further enriched with one or more additional peptides derived from SEQ ID NO: 11, SEQ ID NO: 12 and/or SEQ ID NO: 14, or functional fragments or variants thereof. In some embodiments, the pooled viral peptides derived from the viral antigen of SEQ ID NO: 14, or functional fragments or variants thereof, are further enriched with one or more additional peptides derived from SEQ ID NO: 11, SEQ ID NO: 12 and/or SEQ ID NO: 13, or functional fragments or variants thereof. In some embodiments, the pooled viral peptides are comprised of epitopes derived from viral peptides selected from those provided in Table A, Table B and/or Table C.

In some embodiments, the T cell composition is derived through the ex vivo expansion of separate T cell populations, wherein the T cell composition includes separate T cell subpopulations primed to a pool of peptides comprising one or more antigenic peptides or epitopes thereof selected from PRAME, Survivin, and WT1.

In other embodiments, 1, 2, 3, 4, 5 or more peptides from PRAME, Survivin, and/or WT1 may be used to induce or expand T cells (or in some cases excluded). These peptides may comprise, consist essentially of, or consist of the immunologically active peptides used to induce or expand the T cells to specific epitopes. In other embodiments, peptides specific to other tumor antigens, such as NYESO, MAGE A4, MAGE A3, MAGE A1, neuroelastase, proteinase 3, p53, CEA, claudin6, Histone H1, Histone H2, Histone H3, Histone H4, MART1, gp100, PSA, SOX2, SSX2, Nanog, Oct4, Myc, and Ras, may be include or excluded from peptide(s) used to induce or expand T cells. In some embodiments, peptides from the following types of classes of tumor antigen may be used (or excluded): Alphafetoprotein (AFP), carcinoembryonic antigen (CEA), CA-125, MUC-1, Epithelial tumor antigen (ETA), tyrosinase, melanoma-associated antigen (MAGE), or abnormal products of ras or p53.

In some embodiments, the pooled peptides are comprised of overlapping peptides derived from antigens selected from PRAME, Survivin, and WT1, or combinations thereof. In some embodiments, the pooled viral antigens are further enriched with one or more additional peptides selected from PRAME, Survivin, and WT1 having the following sequences, or functional fragments or variants thereof having at least about 70%, 75%, 80%, 85%, 90%, 91%, 92%, 93%, 94%, 95%, 96%, 97%, 98%, 99%, <100%, or 100% sequence identity thereto.

PRAME
(SEQ ID NO: 15)
MERRRLWGSIQSRYISMSVWTSPRRLVELAGQSLL
KDEALAIAALELLPRELFPPLFMAAFDGRHSQTLK
AMVQAWPFTCLPLGVLMKGQHLHLETFKAVLDGLD
VLLAQEVRPRRWKLQVLDLRKNSHQDFWTVWSGNR
ASLYSFPEPEAAQPMTKKRKVDGLSTEAEQPFIPV
EVLVDLFLKEGACDELFSYLIEKVKRKKNVLRLCC
KKLKIFAMPMQDIKMILKMVQLDSIEDLEVTCTWK
LPTLAKFSPYLGQMINLRRLLLSHIHASSYISPEK
EEQYIAQFTSQFLSLQCLQALYVDSLFFLRGRLDQ
LLRHVMNPLETLSITNCRLSEGDVMHLSQSPSVSQ
LSVLSLSGVMLTDVSPEPLQALLERASATLQDLVF
DECGITDDQLLALLPSLSHCSQLTTLSFYGNSISI
SALQSLLQHLIGLSNLTHVLYPVPLESYEDIHGTL
HLERLAYLHARLRELLCELGRPSMVWLSANPCPHC
GDRTFYDPEPELCPCFMPN
Survivin
(SEQ ID NO: 16)
MGAPTLPPAWQPFLKDHRISTFKNWPFLEGCACTP
ERMAEAGFIHCPTENEPDLQCFFCFKELEGWEPDD
DPIEEHKKHSSGCAFLSVKKQFEELTLGEFLKLDR
ERAKNKIAKETNNKKKEFEETAKKVRRALEQLAAM
D
WT1
(SEQ ID NO: 17)
MGSDVRDLNALLPAVPSLGGGGGCALPVSGAAQWA
PVLDFAPPGASAYGSLGGPAPPPAPPPPPPPPPHS
FIKQEPSWGGAEPHEEQCLSAFTVHFSGQFTGTAG
ACRYGPFGPPPPSQASSGQARMFPNAPYLPSCLES
QPAIRNQGYSTVTFDGTPSYGHTPSHHAAQFPNHS
FKHEDPMGQQGSLGEQQYSVPPPVYGCHTPTDSCT
GSQALLLRTPYSSDNLYQMTSQLECMTWNQMNLGA
TLKGVAAGSSSSVKWTEGQSNHSTGYESDNHTTPI
LCGAQYRIHTHGVFRGIQDVRRVPGVAPTLVRSAS
ETSEKRPFMCAYPGCNKRYFKLSHLQMHSRKHTGE
KPYQCDFKDCERRFSRSDQLKRHQRRHTGVKPFQC
KTCQRKFSRSDHLKTHTRTHTGKTSEKPFSCRWPS
CQKKFARSDELVRHHNMHQRNMTKLQLAL

TABLE 2
(WT1)

| SEQ ID NO. | Sequence |
|---|---|
| 18 | TSEKRPFMCAY |
| 19 | STVTFDGTPSY |
| 20 | HTTPILCGAQY |
| 21 | ESQPAIRNQGY |
| 22 | GSQALLLRTPY |
| 23 | HSRKHTGEKPY |
| 24 | FTGTAGACRY |
| 25 | RTPYSSDNLY |
| 26 | TTPILCGAQY |
| 27 | VTFDGTPSY |

In some embodiments, the epitopes are one or a combination from Table 3:

TABLE 3
(PRAME)

| SEQ ID NO. | Sequence |
|---|---|
| 28 | LTDVSPEPLQA |
| 29 | ITDDQLLALLP |
| 30 | HGTLHLERLAY |
| 31 | GTLHLERLAY |
| 32 | CSQLTTLSFY |
| 33 | LSLQCLQALY |
| 34 | PTLAKFSPY |
| 35 | LSNLTHVLY |
| 36 | WSGNRASLY |
| 37 | LSHIHASSY |

In some embodiments, the epitopes are one or a combination from Table 4:

TABLE 4
(Survivin)

| SEQ ID NO. | Sequence |
|---|---|
| 38 | PIENEPDLAQC |
| 39 | KLDRERAKNKI |
| 40 | LKDHRISTFKN |
| 41 | STFKNWPFLEG |
| 42 | DDDPIEEHKKH |
| 43 | PIENEPDLAQ |
| 44 | PIENEPDLA |
| 45 | LTLGEFLKL |
| 46 | LGEFLKLDR |
| 47 | KLDRERAKN |

In some embodiments, the pooled peptides include one or more peptides selected from SEQ ID NO: 15 (PRAME), one or more peptides selected from SEQ ID NO: 16 (Survivin), and one or more peptides selected from SEQ ID NO: 17 (WT1), or combinations thereof. In some aspects, the ratio of the T cell subpopulations that comprise the T cell composition is correlated with the tumor expression profile of the subject.

The disclosure also elated to methods of generating increased numbers and ratios of CD8+ effector memory cells and CD4+ effector memory cells by exposing the cells to one or more compositions comprising one or more cytokines of Table 1. In some embodiments, the compositions of cytokines comprise functional fragments thereof or variants of the disclosed cytokines of Table 1 that comprise at least about 70%, 75%, 80%, 85%, 90%, 91%, 92%, 93%, 94%, 95%, 96%, 97%, 98%, 99%, <100%, or 100% sequence identity to the amino acids disclosed in Table 1.

TABLE 1

List of cytokines and their sequences.
IL-15 (NCBI Reference Sequence: NP 000576.1)
The amino acid sequence of the immature/
precursor form of native human IL-15,
which comprises the long signal peptide
(residues 1-29, underlined) and the mature
human native IL-15 (residues 30-162,
italicized), are provided:
(SEQ ID NO: 1)
MRISKPHLRSISIQCYLCLLLNSHFLTEAGIHVFIL
GCFSAGLPKTEANWVNVISDLKKIEDLIQSMHIDAT
LYTESDVHPSCKVTAMKCFLLELQVISLESGDASIH
DWENLIILANNSLSSNGNVTESGCKECEELEEKNIK
EFLQSFVHIVQMFINTS.
The nucleotide sequence encoding the immature/
precursor form of native human IL-15, which
comprises the nucleotide sequence encoding
the long signal peptide (residues 1-29,
underlined) and the nucleotide sequence
encoding the mature human native IL-15
(residues 30-162, italicized), is provided:
(SEQ ID NO: 2)
Atgagaatttcgaaaccacatttgagaagtatttc
catccagtgctacttgtgtttacttctaaacagtc
attttctaactgaagct ggcattcatgtcttca
ttttgggctgtttcagtgcagggcttcctaaaaca
gaagccaactgggtgaatgtaataagtgatttga
aaaaaattgaagatcttattcaatctatgcatat
tgatgctactttatatacggaaagtgatgttcacc
ccagttgcaaagtaaca gcaatgaagtgctttc
tcttggagttacaagttatttcacttgagtccgga
gatgcaagtattcatgatacagtagaaaatctgat
catcctagcaaacaacagtttgtcttctaatgg
gaatgtaacagaatctggatgcaaagaatgtgagg
aactggaggaaaaa aatattaaagaatttttgc
agagttttgtacatattgtccaaatgttcatcaac
acttcttga
IL-7 (NCBI Reference Sequence: NP_000871.1).
Signal peptide (underlined) residues 1-25,
mature peptide (italics), residues 26-177.
(SEQ ID NO: 3)
MFHVSFRYIFGLPPLILVLLPVASSDCDIEGKDGKQY
ESVLMVSIDQLLDSMKEIGNCLNNEFNFFKRHICDAN
KEGMFLFRAARKLRQFLKMNSTGDFDLHLLKVSEGTT
ILLNCTGQVKGRKPAALGEAQPTKSLEENKSLKEQKK
LNDLCFLKRLLQEIKTCWNKILMGTKEH
Nucleic acid for IL-7
(SEQ ID NO: 4)
Atgttccatgtttcttttaggtatatctttggact
tcctcccctgatccttgttctgttgccagtagcat
catctgattgtgatattgaaggtaaagatggcaaa
caatatgagagtgttctaatggtcagcatcgatca
attattggacagcatgaaagaaattggtagcaatt
gcctgaataatgaatttaacttttttaaaagacat
atctgtgatgctaataaggaaggtatgtttttatt
ccgtgctgctcgcaagttgaggcaatttcttaaaa
tgaatagcactggtgattttgatctccacttatta
aaagtttcagaaggcacaacaatactgttgaactg
cactggccaggttaaaggaagaaaaccagctgccc
tgggtgaagcccaaccaacaaagagtttggaagaa
aataaatctttaaaggaacagaaaaaactgaatga
cttgtgtttcctaaagagactattacaagagataa
aaacttgttggaataaaattttgatgggcactaaa
gaacactga
IL-6 (NCBI Reference Sequence: NP_000591.1)
Signal peptide (underlined) residues 1-29,
mature peptide (italics), residues 30-212.
(SEQ ID NO: 5)
MNSFSTSAFGPVAFSLGLLLVLPAAFPAPVPPGEDS
KDVAAPHRQPLTSSERIDKQIRYILDGISALRKETC
NKSNMCESSKEALAENNLNLPKAIAEKDGCFQSGEN
EETCLVKIITGLLEFEVYLEYLQNRFESSEEQARAV
QMSTKVLIQFLQKKAKNLDAITTPDPTTNASLLTKL
QAQNQWLQDMTTHLILRSFKEFLQSSLRALRQM
Nucleic Acid sequence for IL-6
(SEQ ID NO: 6)
attctgccctcgagcccaccgggaacgaaagagaa
gctctatctcccctccaggagcccagctatgaact
ccttctccacaagcgcctteggtccagttgccttc
tccctggggctgctectggtgttgcctgctgcctt
ccctgccccagtacccccaggagaagattccaaag
atgtagccgccccacacagacagccactcacctct
tcagaacgaattgacaaacaaattcggtacatcct
cgacggcatctcagccctgagaaaggagacatgta
acaagagtaacatgtgtgaaagcagcaaagaggca
ctggcagaaaacaacctgaaccttccaaagatggc
tgaaaaagatggatgcttccaatctggattcaatg
aggagacttgcctggtgaaaatcatcactggtctf
figgagtttgaggtatacctagagtacctccagaa
cagatttgagagtagtgaggaacaagccagagctg
tgcagatgagtacaaaagtectgatccagttectg
cagaaaaaggcaaagaatctagatgcaataaccac
cectgacccaaccacaaatgccagcctgctgacga
agctgcaggcacagaaccagtggctgcaggacatg
acaactcatctcattctgcgcagctttaaggagtt
cctgcagtccagcctgagggctcttcggcaaatgt
agcatgggcacctcagattgttgttgttaatgggc
attecttatctggtcagaaacctgtccactgggca
cagaacttatgttgttctctatggagaactaaaag
tatgagcgttaggacactattttaattatttttaa
tttattaatatttaaatatgtgaagctgagttaat
ttatgtaagtcatatttatatttttaagaagtacc
acttgaaacattttatgtattagttttgaaataat
aatggaaagtggctatgcagtttgaatatcctttg
tttcagagccagatcatttcttggaaagtgtagge
ttacctcaaataaatggctaacttatacatatttt
taaagaaatatttatattgtatttatataatgtat
aaatggtttttataccaataaatggcattttaaaa
aattca
IL-4 (NCBI Reference Sequence:
NP_001341919.1) Signal peptide (underlined)
residues 1-24, mature peptide (italics),
residues 25-136.
(SEQ ID NO: 7)
MGLTSQLLPPLFFLLACAGNFVHGHKCDITLQEI
IKTLNSLTEQKTLCTELTVTDIFAASKQHACLPTS
RHTGLICRPPSARVPSTSTRLQSPERTQLRRKPSA
GLRLCSGSSTATMRRTLAAWVRLHSSSTGTSS
Nucleic acid sequence of IL-4
(SEQ ID NO: 8)
atgggtctcacctcccaactgcttccccctctgtt
cttcctgctagcatgtgccggcaactttgtccacg
gacacaagtgcgatatcaccttacaggagatcatc
aaaactttgaacagcctcacagagcagaagactct
gtgcaccgagttgaccgtaacagacatctttgctg
cctccaagcagcatgcatgtctgcccacctccaga
cacacaggcaccatctgccgccccccatcagcccg
tgtcccttccacctcgactcgcctacaaagcccag
agagaacacaactgagaaggaaaccttctgcaggg
ctgcgactgtgctccggcagttctacagccaccat
gagaaggacactcgctgcctgggtgcgactgcaca
gcagttccacaggcacaagcagctga
IL-18
(NCBI Reference Sequence: NP_001373349.1)
IL-18 lacks a signal peptide. The IL-18
gene encodes a 193 amino acid precursor
which accumulates in cell cytoplasm and
which is processed intracellularly by
caspace 1 or other caspace into its
mature 18 kDA biologically active form.
Region 74-180 shown in italics.
(SEQ ID NO: 9)
MAAEPVEDNCINFVAMKFIDNTLYFIAEDDENLES
DYFGKLESKLSVIRNLNDQVLFIDQGNRPLFEDMT
DSDCRDNAPRTIFIISMYKDSQPRGMAVTISVKCE
KISTLSCENKIISFKEAINPPDNIKDTKSDIIFFQ
RSVPGHDNKAIQFESSSYEGYFLACEKERDLFKLI
LKKEDELGDRSIMFTVQNED
Nucleic Acid sequence of IL-18
(SEQ ID NO: 10)
ctgaacgatcaggtgctgtttattgatcagggcaa
ccgcccgctgtttgaagatatgaccgatagcgatt
gccgcgataacgcgccgcgcaccatttttattatt
agcatgtataaagatagccagccgcgcggcatgge
ggtgaccattagcgtgaaatgcgaaaaaattagca
ccctgagctgcgaaaacaaaattattagctttaaa
gaaatgaacccgccggataacattaaagataccaa
aagcgatattattttttttcagcgcagcgtgccgg
gccatgataacaaaatgcagtttgaaagcagcagc
tatgaaggctattttctggcgtgcgaaaaagaacg
cgatctgtttaaactgattctgaaaaaagaagatg
aactgggcgatcgcagcattatgtttaccgtgcag
aacgaagat

In some embodiments, the nucleic acid is an isolated or purified nucleic acid. In some embodiments, the nucleic acids encode the immature or precursor form of a naturally occurring mammalian IL-15, IL-7, IL-6, IL-4 or IL-18. In other embodiments, the nucleic acids encode the mature form of a naturally occurring mammalian IL-15, IL-7, IL-6, IL-4 or IL-18 free of signal peptide. In some embodiments, the nucleic acids encoding native IL-15, IL-7, IL-6, IL-4 or IL-18 encode the precursor form of naturally occurring human IL-15, IL-7, IL-6, IL-4 or IL-18. In other embodiments, the nucleic acids encoding native IL-15, IL-7, IL-6, IL-4 or IL-18 encode the mature form of naturally occurring human IL-15, IL-7, IL-6, 11-4 or IL-18. In some embodiments, the nucleic acids encoding IL-15, IL-7, IL-6, IL-4 or IL-18 comprise at least about 70%, 75%, 80%, 85%, 90%, 91%, 92%, 93%, 94%, 95%, 96%, 97%, 98% or 99% sequence identity to the nucleotide sequence of SEQ ID NO: 2, SEQ ID NO: 4, SEQ ID NO: 6, SEQ ID NO: 8, or SEQ ID NO: 10, or functional fragments or variants thereof. In some embodiments, the nucleic acids encoding IL-15, IL-7, IL-6, IL-4 or IL-18 comprise the nucleotide sequence of SEQ ID NO: 2, SEQ ID NO: 4, SEQ ID NO: 6, SEQ ID NO: 8, or SEQ ID NO: 10, or functional fragments or variants thereof.

In some embodiments, the methods of the disclosure comprise exposing cells to a composition of cytokines comprising the immature or precursor form of a naturally occurring mammalian IL-15, or functional fragments or variants thereof. In some embodiments, the methods of the disclosure comprise exposing cells to a composition of cytokines comprising the mature form of a naturally occurring mammalian IL-15, or functional fragments or variants thereof. In some embodiments, the naturally occurring mammalian IL-15 is a human IL-15. In some embodiments, the methods of the disclosure comprise exposing cells to a composition of cytokines comprising an IL-15 comprising at least about 70%, 75%, 80%, 85%, 90%, 91%, 92%, 93%, 94%, 95%, 96%, 97%, 98% or 99% sequence identity to the amino acid sequence of SEQ ID NO: 1, or functional fragments or variants thereof. In some embodiments, the methods of the disclosure comprise exposing cells to a composition of cytokines comprising an IL-15 comprising the amino acid sequence of SEQ ID NO: 1, or functional fragments or variants thereof.

As used herein, the terms “IL-15 variant,” “interleukin-15 variant,” or “functional fragment” or “variant” of IL-15, in the context of proteins or polypeptides, refer to: (a) a polypeptide that has at least about 40%, 45%, 50%, 55%, 60%, 65%, 70%, 75%, 80%, 85%, 90%, 95%, 98% or 99% sequence identity to a native mammalian IL-15 polypeptide, such as the IL-15 polypeptide of SEQ ID NO: 1; (b) a polypeptide encoded by a nucleic acid sequence that has at least about 40%, 45%, 50%, 55%, 60%, 65%, 70%, 75%, 80%, 85%, 90%, 95%, 98% or 99% sequence identity to a nucleic acid sequence encoding a native mammalian IL-15 polypeptide, such as the IL-15 coding nucleic acid of SEQ ID NO: 2; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-15 polypeptide, such as the IL-15 polypeptide of SEQ ID NO: 1; (d) a polypeptide encoded by nucleic acids that can hybridize under high, moderate or typical stringency hybridization conditions to nucleic acids encoding a native mammalian IL-15 polypeptide, such as the IL-15 coding nucleic acid of SEQ ID NO: 2; (e) a polypeptide encoded by a nucleic acid sequence that can hybridize under high, moderate or typical stringency hybridization conditions to a nucleic acid sequence encoding a fragment of a native mammalian IL-15 polypeptide, such as the IL-15 polypeptide of SEQ ID NO: 1, of at least 20 contiguous amino acids, at least 30 contiguous amino acids, at least 40 contiguous amino acids, at least 50 contiguous amino acids, at least 100 contiguous amino acids, or at least 150 contiguous amino acids; or (f) a fragment of a native mammalian IL-15 polypeptide, such as the IL-15 polypeptide of SEQ ID NO: 1. IL-15 variants also include a polypeptide that comprises the amino acid sequence of a naturally occurring mature form of a mammalian IL-15 polypeptide and a heterologous signal peptide amino acid sequence. In some embodiments, an IL-15 variant is a derivative of a native human IL-15 polypeptide, such as the IL-15 polypeptide of SEQ ID NO: 1. In another embodiment, an IL-15 variant is a derivative of an immature or precursor form of naturally occurring human IL-15 polypeptide, such as the IL-15 polypeptide of SEQ ID NO: 1. In another embodiment, an IL-15 variant is a derivative of a mature form of naturally occurring human IL-15 polypeptide, such as the IL-15 polypeptide of SEQ ID NO: 1 without the signal peptide. In one embodiment, an IL-15 variant is isolated or purified.

In some embodiments, IL-15 variants retain at least about 50%, 55%, 60%, 65%, 70%, 75%, 80%, 85%, 90%, 95%, 98% or 99% of the function of a native mammalian IL-15 polypeptide to bind IL-15Ra polypeptide, as measured by assays well known in the art, e.g., ELISA, Biacore, co-immunoprecipitation. In other embodiments, IL-15 variants retain at least about 50%, 55%, 60%, 65%, 70%, 75%, 80%, 85%, 90%, 95%, 98% or 99% of the function of a native mammalian IL-15 polypeptide to induce IL-15-mediated signal transduction, as measured by assays well-known in the art, e.g., electromobility shift assays, ELISAs and other immunoassays.

In some embodiments, the methods of the disclosure comprise exposing cells to a composition of cytokines comprising the immature or precursor form of a naturally occurring mammalian IL-7, or functional fragments or variants thereof. In some embodiments, the methods of the disclosure comprise exposing cells to a composition of cytokines comprising the mature form of a naturally occurring mammalian IL-7, or functional fragments or variants thereof. In some embodiments, the naturally occurring mammalian IL-7 is a human IL-7. In some embodiments, the methods of the disclosure comprise exposing cells to a composition of cytokines comprising an IL-7 comprising at least about 70%, 75%, 80%, 85%, 90%, 91%, 92%, 93%, 94%, 95%, 96%, 97%, 98% or 99% sequence identity to the amino acid sequence of SEQ ID NO: 3, or functional fragments or variants thereof. In some embodiments, the methods of the disclosure comprise exposing cells to a composition of cytokines comprising an IL-7 comprising the amino acid sequence of SEQ ID NO: 3, or functional fragments or variants thereof.

As used herein, the terms “IL-7 variant,” “interleukin-7 variant,” or “functional fragment” or “variant” of IL-7, in the context of proteins or polypeptides, refer to: (a) a polypeptide that has at least about 40%, 45%, 50%, 55%, 60%, 65%, 70%, 75%, 80%, 85%, 90%, 95%, 98% or 99% sequence identity to a native mammalian IL-7 polypeptide, such as the IL-7 polypeptide of SEQ ID NO: 3; (b) a polypeptide encoded by a nucleic acid sequence that has at least about 40%, 45%, 50%, 55%, 60%, 65%, 70%, 75%, 80%, 85%, 90%, 95%, 98% or 99% sequence identity to a nucleic acid sequence encoding a native mammalian IL-7 polypeptide, such as the IL-7 coding nucleic acid of SEQ ID NO: 4; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-7 polypeptide, such as the IL-7 polypeptide of SEQ ID NO: 3; (d) a polypeptide encoded by nucleic acids that can hybridize under high, moderate or typical stringency hybridization conditions to nucleic acids encoding a native mammalian IL-7 polypeptide, such as the IL-7 coding nucleic acid of SEQ ID NO: 4; (e) a polypeptide encoded by a nucleic acid sequence that can hybridize under high, moderate or typical stringency hybridization conditions to a nucleic acid sequence encoding a fragment of a native mammalian IL-7 polypeptide, such as the IL-7 polypeptide of SEQ ID NO: 3, of at least 20 contiguous amino acids, at least 30 contiguous amino acids, at least 40 contiguous amino acids, at least 50 contiguous amino acids, at least 100 contiguous amino acids, or at least 150 contiguous amino acids; or (f) a fragment of a native mammalian IL-7 polypeptide, such as the IL-7 polypeptide of SEQ ID NO: 1. IL-7 variants also include a polypeptide that comprises the amino acid sequence of a naturally occurring mature form of a mammalian IL-7 polypeptide and a heterologous signal peptide amino acid sequence. In some embodiments, an IL-7 variant is a derivative of a native human IL-7 polypeptide, such as the IL-7 polypeptide of SEQ ID NO: 3. In another embodiment, an IL-7 variant is a derivative of an immature or precursor form of naturally occurring human IL-7 polypeptide, such as the IL-7 polypeptide of SEQ ID NO: 3. In another embodiment, an IL-7 variant is a derivative of a mature form of naturally occurring human IL-7 polypeptide, such as the IL-7 polypeptide of SEQ ID NO: 3 without the signal peptide. In one embodiment, an IL-7 variant is isolated or purified.

In some embodiments, IL-7 variants retain at least about 50%, 55%, 60%, 65%, 70%, 75%, 80%, 85%, 90%, 95%, 98% or 99% of the function of a native mammalian IL-7 polypeptide to bind IL-7R polypeptide, as measured by assays well known in the art, e.g., ELISA, Biacore, co-immunoprecipitation. In other embodiments, IL-7 variants retain at least about 50%, 55%, 60%, 65%, 70%, 75%, 80%, 85%, 90%, 95%, 98% or 99% of the function of a native mammalian IL-7 polypeptide to induce IL-7-mediated signal transduction, as measured by assays well-known in the art, e.g., electromobility shift assays, ELISAs and other immunoassays

In some embodiments, the methods of the disclosure comprise exposing cells to a composition of cytokines comprising the immature or precursor form of a naturally occurring mammalian IL-6, or functional fragments or variants thereof. In some embodiments, the methods of the disclosure comprise exposing cells to a composition of cytokines comprising the mature form of a naturally occurring mammalian IL-6, or functional fragments or variants thereof. In some embodiments, the naturally occurring mammalian IL-6 is a human IL-6. In some embodiments, the methods of the disclosure comprise exposing cells to a composition of cytokines comprising an IL-6 comprising at least about 70%, 75%, 80%, 85%, 90%, 91%, 92%, 93%, 94%, 95%, 96%, 97%, 98% or 99% sequence identity to the amino acid sequence of SEQ ID NO: 5, or functional fragments or variants thereof. In some embodiments, the methods of the disclosure comprise exposing cells to a composition of cytokines comprising an IL-6 comprising the amino acid sequence of SEQ ID NO: 5, or functional fragments or variants thereof.

As used herein, the terms “IL-6 variant,” “interleukin-6 variant,” or “functional fragment” or “variant” of IL-6, in the context of proteins or polypeptides, refer to: (a) a polypeptide that has at least about 40%, 45%, 50%, 55%, 60%, 65%, 70%, 75%, 80%, 85%, 90%, 95%, 98% or 99% sequence identity to a native mammalian IL-6 polypeptide, such as the IL-6 polypeptide of SEQ ID NO: 5; (b) a polypeptide encoded by a nucleic acid sequence that has at least about 40%, 45%, 50%, 55%, 60%, 65%, 70%, 75%, 80%, 85%, 90%, 95%, 98% or 99% sequence identity to a nucleic acid sequence encoding a native mammalian IL-6 polypeptide, such as the IL-6 coding nucleic acid of SEQ ID NO: 6; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-6 polypeptide, such as the IL-6 polypeptide of SEQ ID NO: 5; (d) a polypeptide encoded by nucleic acids that can hybridize under high, moderate or typical stringency hybridization conditions to nucleic acids encoding a native mammalian IL-6 polypeptide, such as the IL-6 coding nucleic acid of SEQ ID NO: 6; (e) a polypeptide encoded by a nucleic acid sequence that can hybridize under high, moderate or typical stringency hybridization conditions to a nucleic acid sequence encoding a fragment of a native mammalian IL-6 polypeptide, such as the IL-6 polypeptide of SEQ ID NO: 5, of at least 20 contiguous amino acids, at least 30 contiguous amino acids, at least 40 contiguous amino acids, at least 50 contiguous amino acids, at least 100 contiguous amino acids, or at least 150 contiguous amino acids; or (f) a fragment of a native mammalian IL-6 polypeptide, such as the IL-6 polypeptide of SEQ ID NO: 5. IL-6 variants also include a polypeptide that comprises the amino acid sequence of a naturally occurring mature form of a mammalian IL-6 polypeptide and a heterologous signal peptide amino acid sequence. In some embodiments, an IL-6 variant is a derivative of a native human IL-6 polypeptide, such as the IL-6 polypeptide of SEQ ID NO: 5. In another embodiment, an IL-6 variant is a derivative of an immature or precursor form of naturally occurring human IL-6 polypeptide, such as the IL-6 polypeptide of SEQ ID NO: 5. In another embodiment, an IL-6 variant is a derivative of a mature form of naturally occurring human IL-6 polypeptide, such as the IL-6 polypeptide of SEQ ID NO: 5 without the signal peptide. In one embodiment, an IL-6 variant is isolated or purified.

In some embodiments, IL-6 variants retain at least about 50%, 55%, 60%, 65%, 70%, 75%, 80%, 85%, 90%, 95%, 98% or 99% of the function of a native mammalian IL-6 polypeptide to bind IL-6R polypeptide, as measured by assays well known in the art, e.g., ELISA, Biacore, co-immunoprecipitation. In other embodiments, IL-15 variants retain at least about 50%, 55%, 60%, 65%, 70%, 75%, 80%, 85%, 90%, 95%, 98% or 99% of the function of a native mammalian IL-6 polypeptide to induce IL-6-mediated signal transduction, as measured by assays well-known in the art, e.g., electromobility shift assays, ELISAs and other immunoassays.

In some embodiments, the methods of the disclosure comprise exposing cells to a composition of cytokines comprising the immature or precursor form of a naturally occurring mammalian IL-4, or functional fragments or variants thereof. In some embodiments, the methods of the disclosure comprise exposing cells to a composition of cytokines comprising the mature form of a naturally occurring mammalian IL-4, or functional fragments or variants thereof. In some embodiments, the naturally occurring mammalian IL-4 is a human IL-4. In some embodiments, the methods of the disclosure comprise exposing cells to a composition of cytokines comprising an IL-4 comprising at least about 70%, 75%, 80%, 85%, 90%, 91%, 92%, 93%, 94%, 95%, 96%, 97%, 98% or 99% sequence identity to the amino acid sequence of SEQ ID NO: 7, or functional fragments or variants thereof. In some embodiments, the methods of the disclosure comprise exposing cells to a composition of cytokines comprising an IL-4 comprising the amino acid sequence of SEQ ID NO: 7, or functional fragments or variants thereof.

As used herein, the terms “IL-4 variant,” “interleukin-4 variant,” or “functional fragment” or “variant” of IL-4, in the context of proteins or polypeptides, refer to: (a) a polypeptide that has at least about 40%, 45%, 50%, 55%, 60%, 65%, 70%, 75%, 80%, 85%, 90%, 95%, 98% or 99% sequence identity to a native mammalian IL-4 polypeptide, such as the IL-4 polypeptide of SEQ ID NO: 7; (b) a polypeptide encoded by a nucleic acid sequence that has at least about 40%, 45%, 50%, 55%, 60%, 65%, 70%, 75%, 80%, 85%, 90%, 95%, 98% or 99% sequence identity to a nucleic acid sequence encoding a native mammalian IL-4 polypeptide, such as the IL-4 coding nucleic acid of SEQ ID NO: 8; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-4 polypeptide, such as the IL-4 polypeptide of SEQ ID NO: 7; (d) a polypeptide encoded by nucleic acids that can hybridize under high, moderate or typical stringency hybridization conditions to nucleic acids encoding a native mammalian IL-4 polypeptide, such as the IL-4 coding nucleic acid of SEQ ID NO: 8; (e) a polypeptide encoded by a nucleic acid sequence that can hybridize under high, moderate or typical stringency hybridization conditions to a nucleic acid sequence encoding a fragment of a native mammalian IL-4 polypeptide, such as the IL-4 polypeptide of SEQ ID NO: 7, of at least 20 contiguous amino acids, at least 30 contiguous amino acids, at least 40 contiguous amino acids, at least 50 contiguous amino acids, at least 100 contiguous amino acids, or at least 150 contiguous amino acids; or (f) a fragment of a native mammalian IL-4 polypeptide, such as the IL-4 polypeptide of SEQ ID NO: 7. IL-4 variants also include a polypeptide that comprises the amino acid sequence of a naturally occurring mature form of a mammalian IL-4 polypeptide and a heterologous signal peptide amino acid sequence. In some embodiments, an IL-4 variant is a derivative of a native human IL-4 polypeptide, such as the IL-4 polypeptide of SEQ ID NO: 7. In another embodiment, an IL-4 variant is a derivative of an immature or precursor form of naturally occurring human IL-4 polypeptide, such as the IL-4 polypeptide of SEQ ID NO: 7. In another embodiment, an IL-4 variant is a derivative of a mature form of naturally occurring human IL-4 polypeptide, such as the IL-4 polypeptide of SEQ ID NO: 7 without the signal peptide. In one embodiment, an IL-4 variant is isolated or purified.

In some embodiments, IL-4 variants retain at least about 50%, 55%, 60%, 65%, 70%, 75%, 80%, 85%, 90%, 95%, 98% or 99% of the function of a native mammalian IL-4 polypeptide to bind IL-4R polypeptide, as measured by assays well known in the art, e.g., ELISA, Biacore, co-immunoprecipitation. In other embodiments, IL-4 variants retain at least about 50%, 55%, 60%, 65%, 70%, 75%, 80%, 85%, 90%, 95%, 98% or 99% of the function of a native mammalian IL-4 polypeptide to induce IL-4-mediated signal transduction, as measured by assays well-known in the art, e.g., electromobility shift assays, ELISAs and other immunoassays.

In some embodiments, the methods of the disclosure comprise exposing cells to a composition of cytokines comprising the immature or precursor form of a naturally occurring mammalian IL-18, or functional fragments or variants thereof. In some embodiments, the methods of the disclosure comprise exposing cells to a composition of cytokines comprising the mature form of a naturally occurring mammalian IL-18, or functional fragments or variants thereof. In some embodiments, the naturally occurring mammalian IL-18 is a human IL-18. In some embodiments, the methods of the disclosure comprise exposing cells to a composition of cytokines comprising an IL-18 comprising at least about 70%, 75%, 80%, 85%, 90%, 91%, 92%, 93%, 94%, 95%, 96%, 97%, 98% or 99% sequence identity to the amino acid sequence of SEQ ID NO: 9, or functional fragments or variants thereof. In some embodiments, the methods of the disclosure comprise exposing cells to a composition of cytokines comprising an IL-18 comprising the amino acid sequence of SEQ ID NO: 9, or functional fragments or variants thereof.

As used herein, the terms “IL-18 variant,” “interleukin-18 variant,” or “functional fragment” or “variant” of IL-18, in the context of proteins or polypeptides, refer to: (a) a polypeptide that has at least about 40%, 45%, 50%, 55%, 60%, 65%, 70%, 75%, 80%, 85%, 90%, 95%, 98% or 99% sequence identity to a native mammalian IL-18 polypeptide, such as the IL-18 polypeptide of SEQ ID NO: 9; (b) a polypeptide encoded by a nucleic acid sequence that has at least about 40%, 45%, 50%, 55%, 60%, 65%, 70%, 75%, 80%, 85%, 90%, 95%, 98% or 99% sequence identity to a nucleic acid sequence encoding a native mammalian IL-18 polypeptide, such as the IL-18 coding nucleic acid of SEQ ID NO: 10; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-18 polypeptide, such as the IL-18 polypeptide of SEQ ID NO: 9; (d) a polypeptide encoded by nucleic acids that can hybridize under high, moderate or typical stringency hybridization conditions to nucleic acids encoding a native mammalian IL-18 polypeptide, such as the IL-18 coding nucleic acid of SEQ ID NO: 10; (e) a polypeptide encoded by a nucleic acid sequence that can hybridize under high, moderate or typical stringency hybridization conditions to a nucleic acid sequence encoding a fragment of a native mammalian IL-18 polypeptide, such as the IL-18 polypeptide of SEQ ID NO: 9, of at least 20 contiguous amino acids, at least 30 contiguous amino acids, at least 40 contiguous amino acids, at least 50 contiguous amino acids, at least 100 contiguous amino acids, or at least 150 contiguous amino acids; or (f) a fragment of a native mammalian IL-18 polypeptide, such as the IL-18 polypeptide of SEQ ID NO: 9. IL-18 variants also include a polypeptide that comprises the amino acid sequence of a naturally occurring mature form of a mammalian IL-18 polypeptide and a heterologous signal peptide amino acid sequence. In some embodiments, an IL-18 variant is a derivative of a native human IL-18 polypeptide, such as the IL-18 polypeptide of SEQ ID NO: 9. In another embodiment, an IL-18 variant is a derivative of an immature or precursor form of naturally occurring human IL-18 polypeptide, such as the IL-18 polypeptide of SEQ ID NO: 9. In another embodiment, an IL-18 variant is a derivative of a mature form of naturally occurring human IL-18 polypeptide, such as the IL-18 polypeptide of SEQ ID NO: 9 without the signal peptide. In one embodiment, an IL-18 variant is isolated or purified.

In some embodiments, IL-18 variants retain at least about 50%, 55%, 60%, 65%, 70%, 75%, 80%, 85%, 90%, 95%, 98% or 99% of the function of a native mammalian IL-18 polypeptide to bind IL-18R polypeptide, as measured by assays well known in the art, e.g., ELISA, Biacore, co-immunoprecipitation. In other embodiments, IL-18 variants retain at least about 50%, 55%, 60%, 65%, 70%, 75%, 80%, 85%, 90%, 95%, 98% or 99% of the function of a native mammalian IL-18 polypeptide to induce IL-18-mediated signal transduction, as measured by assays well-known in the art, e.g., electromobility shift assays, ELISAs and other immunoassays.

The disclosure relates to a method of expanding CD8+ and/or CD4+ lymphocytes in an vitro culture comprising contacting the lymphocytes with at least two polypeptides, one or a plurality of nucleic acids encoding at least two polypeptides; or a combination of a nucleic acid and a polypeptide; wherein the at least two polypeptides are cytokines chosen from: IL-15, IL-6, IL-7, IL-4, IL-18 and/or functional fragments or variants thereof. In some embodiments, the method relates to contacting one or a plurality of lymphocytes in an in vitro culture with at least one combination of cytokines chosen from a combination of: (i) IL-15 and IL-6, or functional fragments or variants thereof; (ii) IL-15 and IL-7, or functional fragments or variants thereof, (iii) IL-7 and IL-4, or functional fragments or variants thereof, and/or (iv) IL-15 and IL-18, or functional fragments or variants thereof. Any combination of cytokines in nucleic acid form or protein form may be exposed to one or more cells. In some embodiments, where exposure of the cytokines is by nucleic acid, plasmids that stably or transiently express the cytokine or functional fragment or variant thereof may be used. In some embodiments, the cell is a hematopoietic stem cell or a hematopoietic progenitor cell. In some embodiments, the methods disclosed herein comprise a multistep process of differentiating a naïve T cell into a memory effector T cell that is either CD4+ or CD8+ and then, subsequently or simultaneously or prior to differentiating the naïve T cells, stimulating the naïve T cells with one or a plurality of antigens, such as viral antigens. In some embodiments, the method of differentiating the endothelial cells disclosed herein comprises exposing the naïve cells to at least one cytokine composition, such as but not limited to one comprising IL-15, at a concentration and for a time period sufficient to cause growth and proliferation of CD8 and a change in character from a naïve cell to a memory effector cell. In some embodiments, the method of differentiating the endothelial cells disclosed herein comprises exposing the naïve cells to at least one cytokine composition, such as but not limited to one comprising IL-6, at a concentration and for a time period sufficient to cause growth and proliferation of CD8 and a change in character from a naïve cell to a memory effector cell. In some embodiments, the method of differentiating the endothelial cells disclosed herein comprises exposing the naïve cells to at least one cytokine composition, such as but not limited to one comprising IL-18, at a concentration and for a time period sufficient to cause growth and proliferation of CD8 and a change in character from a naïve cell to a memory effector cell. In some embodiments, the method of differentiating the endothelial cells disclosed herein comprises exposing the naïve cells to at least one cytokine composition, such as but not limited to one comprising IL-4, at a concentration and for a time period sufficient to cause growth and proliferation of CD4 and a change in character from a naïve cell to a memory effector cell. In some embodiments, the method of differentiating the endothelial cells disclosed herein comprises exposing the naïve cells to at least one cytokine composition, such as but not limited to one comprising IL-7, at a concentration and for a time period sufficient to cause growth and proliferation of CD4 and a change in character from a naïve cell to a memory effector cell.

In some embodiments, the naïve T cell is exposed to one or a combination of any of the cytokines listed in Table 1, at a concentration and for a time period sufficient to induce expression of CCR7 and CD45RO. In some embodiments, the composition comprising lymphocytes is exposed to one or a combination of any of the cytokines listed in Table 1, optionally after exposure to the one or combination of tumor or viral antigens, at a concentration and for a time period sufficient to alter the change a population of lymphocytes cell to T cell effector memory cells.

The disclosure relates to methods by which T cells can be differentiated into memory T cells. Reprogramming of the T cells may be accomplished by exposing the isolated naïve T cells to a composition comprising one or a plurality of cytokines disclosed herein for a time sufficient to sequentially activate or induce expression of CCR7 and CD45RO. Furthermore, these cells can also be exposed to tumor antigens for priming and eventual introduction or administration into mammals having cancer comprising the tumor antigens. These cells can also be exposed to viral antigens for priming and eventually introduction or administration into mammals having viral infection by the virus from which the viral antigens are used for priming. These findings demonstrate that memory effector T cells can be generated from naïve T cells by simple exposure to cytokine compositions comprising at least two cytokines or functional fragments thereof.

CCR7 (C-C motif chemokine receptor 7) is a member of the G protein-coupled receptor family. This receptor was identified as a gene induced by the Epstein-Barr virus (EBV), and is thought to be a mediator of EBV effects on B lymphocytes. This receptor is expressed in various lymphoid tissues and activates B and T lymphocytes. It has been shown to control the migration of memory T cells to inflamed tissues, as well as stimulate dendritic cell maturation. The chemokine (C-C motif) ligand 19 (CCL19/ECL) has been reported to be a specific ligand of this receptor. Signals mediated by this receptor regulate T cell homeostasis in lymph nodes, and may also function in the activation and polarization of T cells, and in chronic inflammation pathogenesis. CCR7 from Homo sapiens has the following sequence (GenBank Accession No. AAT52232):

(SEQ ID NO: 48)
MDLGKPMKSVLVVALLVIFQVCLCQDEVTDDYIGDN
TTVDYTLFESLCSKKDVRNFKAWFLPIMYSIICFV
GLLGNGLVVLTYIYFKRLKTMTDTYLLNLAVADIL
FLLTLPFWAYSAAKSWVFGVHFCKLIFAIYKMSFF
SGMLLLLCISIDRYVAIVQAVSAHRHRARVLLISK
LSCVGIWILATVLSIPELLYSDLQRSSSEQAMRCS
LITEHVEAFITIQVAQMVIGFLVPLLAMSFCYLVI
IRTLLQARNFERNKAIKVIIAVVVVFIVFQLPYNG
VVLAQTVANFNITSSTCELSKQLNIAYDVTYSLAC
VRCCVNPFLYAFIGVKFRNDLFKLFKDLGCLSQEQ
LRQWSSCRHIRRSSMSVEAETTTTFSP

Protein tyrosine phosphatase, receptor type, C also known as PTPRC is an enzyme that, in humans, is encoded by the PTPRC gene. PTPRC is also known as CD45 antigen (CD stands for cluster of differentiation), which was originally called leukocyte common antigen (LCA). The CD45 protein family consists of multiple members that are all products of a single complex gene. CD45RO, the shortest CD45 isoform, which lacks all three of the A, B, and C regions. This shortest isoform facilitates T cell activation.

Receptor-type tyrosine-protein phosphatase C isoform 5 precursor from Homo sapiens has the following sequence (GenBank Accession No. NP 001254727):

(SEQ ID NO: 49)
MTMYLWLKLLAFGFAFLDTEVFVTGQSPTPSPTGH
LQAEEQGSQSKSPNLKSREADSSAFSWWPKAREPL
TNHWSKSKSPKAEELGV

In some embodiments, reprogramming of the T cells according to the present disclosure may be accomplished by exposing the isolated naïve T cells to a composition comprising one or a plurality of cytokines disclosed herein for a time sufficient to sequentially activate or induce expression of CCR7 comprising at least about 70%, 75%, 80%, 85%, 90%, 91%, 92%, 93%, 94%, 95%, 96%, 97%, 98%, 99%, <100%, or 100% sequence identity to SEQ ID NO: 48 and CD45RO comprising at least about 70%, 75%, 80%, 85%, 90%, 91%, 92%, 93%, 94%, 95%, 96%, 97%, 98%, 99%, <100%, or 100% sequence identity to SEQ ID NO: 49.

In some embodiments, the disclosure relates to a method of expanding viral antigen specific CD8+ and/or CD4+ T cells in an in vitro culture comprising contacting the antigen presenting cells, such as lymphocytes, with one or a plurality of viral antigens in the presence of one or a plurality of cytokines chosen from: IL-15, IL-6, IL-7, IL-4, IL-18 and/or functional fragments or variants thereof. In some embodiments, at least two cytokines are present in the in vitro culture chosen from a combination of: (i) IL-15 and IL-6, or functional fragments or variants thereof, (ii) IL-15 and IL-7, or functional fragments or variants thereof, (iii) IL-7 and IL-4, or functional fragments or variants thereof, and/or (iv) IL-15 and IL-18, or functional fragments or variants thereof. Any combination of cytokines in nucleic acid form or protein form may be exposed to one or more cells. In some embodiments, the one or plurality of viral antigens used in the methods of the disclosure for priming the cells comprise at least about 70%, 75%, 80%, 85%, 90%, 91%, 92%, 93%, 94%, 95%, 96%, 97%, 98% or 99% sequence identity to the amino acid sequences depicted in FIG. 8. In some embodiments, the one or plurality of viral antigens used in the methods of the disclosure for priming the cells are chosen from the viral antigen peptides provided in FIG. 8.

In some embodiments, the disclosure relates to a method of expanding viral antigen specific CD8+ and/or CD4+ T cells in an in vitro culture comprising contacting the antigen presenting cells, such as lymphocytes, with one or a plurality of viral antigens of a virus of the family Coronaviridae in the presence of one or a plurality of cytokines chosen from: IL-15, IL-6, IL-7, IL-4, IL-18 and/or functional fragments or variants thereof. In some embodiments, at least two cytokines are present in the in vitro culture chosen from a combination of: (i) IL-15 and IL-6, or functional fragments or variants thereof, (ii) IL-15 and IL-7, or functional fragments or variants thereof; (iii) IL-7 and IL-4, or functional fragments or variants thereof, and/or (iv) IL-15 and IL-18, or functional fragments or variants thereof.

Any combination of cytokines in nucleic acid form or protein form may be exposed to one or more cells. In some embodiments, the one or plurality of viral antigens used in the methods of the disclosure for priming the cells are from a coronavirus. In some embodiments, the one or plurality of viral antigens used in the methods of the disclosure for priming the cells are from SARS-CoV-2 including SARS-CoV-2 delta variant”. In some embodiments, the one or plurality of viral antigens used in the methods of the disclosure for priming the cells are peptide fragments of the SARS-CoV-2 antigens comprising the amino acid sequences of SEQ ID NO: 11, SEQ ID NO: 12, SEQ ID NO: 13 and/or SEQ ID NO: 14, or functional fragments or variants thereof having at least about 70%, 75%, 80%, 85%, 90%, 91%, 92%, 93%, 94%, 95%, 96%, 97%, 98% or 99% sequence identity SEQ ID NO: 11, SEQ ID NO: 12, SEQ ID NO: 13 and/or SEQ ID NO: 14. In some embodiments, the one or plurality of viral antigens used in the methods of the disclosure for priming the cells are in form of a SARS-CoV-2 antigen library comprising a pool of peptides (for example, peptides that are from about 10 to about 20 amino acids in length) from one or a combination of the structural proteins of SARS-CoV-2 disclosed herein, such as 15-mers peptides containing amino acids overlap (for example 11 amino acids of overlap) between each peptide formed by scanning the proteins having the amino acid sequences of SEQ ID NO: 11, SEQ ID NO: 12, SEQ ID NO: 13 and/or SEQ ID NO: 14, or functional fragments or variants thereof having at least about 70%, 75%, 80%, 85%, 90%, 91%, 92%, 93%, 94%, 95%, 96%, 97%, 98% or 99% sequence identity SEQ ID NO: 11, SEQ ID NO: 12, SEQ ID NO: 13 and/or SEQ ID NO: 14.

In some embodiments, the SARS-CoV-2 specific T cell compositions are generated using a SARS-CoV-2 antigen library comprising a pool of peptides formed by scanning the protein having the amino acid sequence of SEQ ID NO: 11, or functional fragments or variants thereof having at least about 70%, 75%, 80%, 85%, 90%, 91%, 92%, 93%, 94%, 95%, 96%, 97%, 98% or 99% sequence identity SEQ ID NO: 11. In some embodiments, the SARS-CoV-2 specific T cell compositions are generated using a SARS-CoV-2 antigen library comprising a pool of peptides formed by scanning the protein having the amino acid sequence of SEQ ID NO: 12, or functional fragments or variants thereof having at least about 70%, 75%, 80%, 85%, 90%, 91%, 92%, 93%, 94%, 95%, 96%, 97%, 98% or 99% sequence identity SEQ ID NO: 12. In some embodiments, the SARS-CoV-2 specific T cell compositions are generated using a SARS-CoV-2 antigen library comprising a pool of peptides formed by scanning the protein having the amino acid sequence of SEQ ID NO: 13, or functional fragments or variants thereof having at least about 70%, 75%, 80%, 85%, 90%, 91%, 92%, 93%, 94%, 95%, 96%, 97%, 98% or 99% sequence identity SEQ ID NO: 13. In some embodiments, the SARS-CoV-2 specific T cell compositions are generated using a SARS-CoV-2 antigen library comprising a pool of peptides formed by scanning the protein having the amino acid sequence of SEQ ID NO: 14, or functional fragments or variants thereof having at least about 70%, 75%, 80%, 85%, 90%, 91%, 92%, 93%, 94%, 95%, 96%, 97%, 98% or 99% sequence identity SEQ ID NO: 14.

In some embodiments, the SARS-CoV-2 specific T cell compositions are generated using a SARS-CoV-2 antigen library comprising a pool of peptides formed by scanning the proteins having the amino acid sequences of SEQ ID NO: 11 and SEQ ID NO: 12, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11 and SEQ ID NO: 13, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 12 and SEQ ID NO: 13, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 12 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 13 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11, SEQ ID NO: 12 and SEQ ID NO: 13, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11, SEQ ID NO: 12 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11, SEQ ID NO: 13 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 12, SEQ ID NO: 13 and SEQ ID NO: 14, or functional fragments or variants thereof. In some embodiments, the SARS-CoV-2 specific T cell compositions are generated using a SARS-CoV-2 antigen library comprising a pool of peptides formed by scanning the proteins having the amino acid sequences of SEQ ID NO: 11, SEQ ID NO: 12, SEQ ID NO: 13 and SEQ ID NO: 14, or functional fragments or variants thereof. In some embodiments, the SARS-CoV-2 specific T cell compositions are generated using one or plurality of epitopes chosen from those disclosed in Table A, Table B, and/or Table C or from identical or corresponding epitopes of SARS-COV-2 delta, mu variants or other variants, such as a variant containing 1, 2 or 3 insertions, substitutions, or deletions of an amino acid in a sequence disclosed by Tables A, B or C. For example, a corresponding epitope may contain one or more substitutions or deletions to the amino acid sequence of SEQ ID NO: 11 or to a peptide epitope thereof: D614G, T478K, L452R, P681R, T19R, Δ157-158, D950N and K417N; N439K, Y453F, Δ69-70; E484K, E484Q, E484P, E484A, E484D, E484G or E484K; K444E, G446V, L452R or F490S; S477G, S477N or S477R; N148S, K150R, K150E, K150T, K150Q or S151P; Δ69-70 (RDR1), Δ141-144 and Δ146 (RDR2), Δ210 (RDR3) or Δ243-244 (RDR4); ΔF140, N148S, K150R, K150E, K150T, K150Q and S151P) and the RBD (K444R, K444N, K444Q, V445E and E484K; D769H, N501Y, S477N, Δ570D, P681H, T716L, S982A, D1118H, ΔH69-V70 or ΔY144.

The disclosure relates to a T cell composition comprising T cells that specifically bind to a Coronaviridae peptide having at least about a 80%, 85%, 90%, 95% sequence identity to SEQ ID NO:11, SEQ ID NO: 12, SEQ ID NO:13 and/or SEQ ID NO:14. In some embodiments, the T cells of the disclosed composition specifically bind to one or a plurality of epitopes located in the protein of SEQ ID NO: 11, or functional fragments thereof. In some embodiments, the T cells of the disclosed composition specifically bind to one or a plurality of epitopes located at amino acid residue from about 12 to about 524 of the protein of SEQ ID NO: 11, or functional fragments thereof. In some embodiments, the T cells of the disclosed composition specifically bind to one or a plurality of epitopes located at amino acid residue from about 12 to about 331 of the protein of SEQ ID NO: 11, or functional fragments thereof. In some embodiments, the T cells of the disclosed composition specifically bind to one or a plurality of epitopes located at amino acid residue from about 57 to about 75 of the protein of SEQ ID NO: 11, or functional fragments thereof. In some embodiments, the T cells of the disclosed composition specifically bind to one or a plurality of epitopes located at amino acid residue from about 205 to about 224 of the protein of SEQ ID NO: 11, or functional fragments thereof. In some embodiments, the T cells of the disclosed composition specifically bind to one or a plurality of epitopes located at amino acid residue from about 331 to about 524 or functional fragments thereof. In some embodiments, the T cells of the disclosed composition specifically bind to one or a plurality of epitopes located at amino acid residue from about 449 to about 463 of the protein of SEQ ID NO: 11, or functional fragments thereof. In some embodiments, the T cells of the disclosed composition specifically bind to one or a plurality of epitopes selected from Table A. In some embodiments, the T cells of the disclosed composition specifically bind to one or a plurality of epitopes located in the protein of SEQ ID NO: 12, or functional fragments thereof. In some embodiments, the T cells of the disclosed composition specifically bind to one or a plurality of epitopes located in the protein of SEQ ID NO: 13, or functional fragments thereof. In some embodiments, the T cells of the disclosed composition specifically bind to one or a plurality of epitopes located at amino acid residue from about 100 to about 222 of the protein of SEQ ID NO: 13, or functional fragments thereof. In some embodiments, the T cells of the disclosed composition specifically bind to one or a plurality of epitopes located at amino acid residue from about 144 to about 192 of the protein of SEQ ID NO: 13, or functional fragments thereof. In some embodiments, the T cells of the disclosed composition specifically bind to one or a plurality of epitopes located at amino acid residue from about 144 to about 163 of the protein of SEQ ID NO: 13, or functional fragments thereof. In some embodiments, the T cells of the disclosed composition specifically bind to one or a plurality of epitopes located at amino acid residue from about 163 to about 192 of the protein of SEQ ID NO: 13, or functional fragments thereof. In some embodiments, the T cells of the disclosed composition specifically bind to one or a plurality of epitopes located at amino acid residue from about 173 to about 192 of the protein of SEQ ID NO: 13, or functional fragments thereof. In some embodiments, the T cells of the disclosed composition specifically bind to one or a plurality of epitopes selected from Table B. In some embodiments, the T cells of the disclosed composition specifically bind to one or a plurality of epitopes located in the protein of SEQ ID NO: 14, or functional fragments thereof. In some embodiments, the T cells of the disclosed composition specifically bind to one or a plurality of epitopes located at amino acid residue from about 257 to about 361 of the protein of SEQ ID NO: 14, or functional fragments thereof. In some embodiments, the T cells of the disclosed composition specifically bind to one or a plurality of epitopes located at amino acid residue from about 257 to about 271 of the protein of SEQ ID NO: 14, or functional fragments thereof. In some embodiments, the T cells of the disclosed composition specifically bind to one or a plurality of epitopes selected from Table C.

In some embodiments, the T cells of the disclosed composition bind to one or plurality of the aforementioned proteins, fragments thereof, or epitopes thereof, with an affinity of EC₅₀, the concentration required to produce the half-maximal response, in the range of about 0.01 to about 20 ng/mL. In some embodiments, the T cells of the disclosed composition bind to one or plurality of the aforementioned proteins, fragments thereof, or epitopes thereof, with an affinity of EC₅₀ in the range of about 0.05 to about 18 ng/mL. In some embodiments, the T cells of the disclosed composition bind to one or plurality of the aforementioned proteins, fragments thereof, or epitopes thereof, with an affinity of EC₅₀ in the range of about 0.1 to about 15 ng/mL. In some embodiments, the T cells of the disclosed composition bind to one or plurality of the aforementioned proteins, fragments thereof, or epitopes thereof, with an affinity of EC₅₀ in the range of about 0.2 to about 10 ng/mL. In some embodiments, the T cells of the disclosed composition bind to one or plurality of the aforementioned proteins, fragments thereof, or epitopes thereof, with an affinity of EC₅₀ in the range of about 0.3 to about 8 ng/mL. In some embodiments, the T cells of the disclosed composition bind to one or plurality of the aforementioned proteins, fragments thereof, or epitopes thereof, with an affinity of EC₅₀ in the range of about 0.4 to about 6 ng/mL. In some embodiments, the T cells of the disclosed composition bind to one or plurality of the aforementioned proteins, fragments thereof, or epitopes thereof, with an affinity of EC₅₀ in the range of about 0.5 to about 5 ng/mL. In some embodiments, the T cells of the disclosed composition bind to one or plurality of the aforementioned proteins, fragments thereof, or epitopes thereof, with an affinity of EC₅₀ in the range of about 0.6 to about 4 ng/mL.

In some embodiments, the T cells of the disclosed composition bind to one or plurality of the aforementioned proteins, fragments thereof, or epitopes thereof, with an affinity of EC₅₀ of about or at least 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. The above range includes all intermediate subranges and values.

In some embodiments, the T cells of the disclosed composition bind to one or plurality of the aforementioned proteins, fragments thereof, or epitopes thereof, with an affinity of EC₅₀ from about 0.1 nM to about 100 nM. In some embodiments, the T cells of the disclosed composition bind to one or plurality of the aforementioned proteins, fragments thereof, or epitopes thereof, with an affinity of EC₅₀ from about 0.5 nM to about 80 nM. In some embodiments, the T cells of the disclosed composition bind to one or plurality of the aforementioned proteins, fragments thereof, or epitopes thereof, with an affinity of EC₅₀ from about 0.8 nM to about 60 nM. In some embodiments, the T cells of the disclosed composition bind to one or plurality of the aforementioned proteins, fragments thereof, or epitopes thereof, with an affinity of EC₅₀ from about 1 nM to about 50 nM. In some embodiments, the T cells of the disclosed composition bind to one or plurality of the aforementioned proteins, fragments thereof, or epitopes thereof, with an affinity of EC₅₀ from about 5 nM to about 40 nM. In some embodiments, the T cells of the disclosed composition bind to one or plurality of the aforementioned proteins, fragments thereof, or epitopes thereof, with an affinity of EC₅₀ from about 7.5 nM to about 35 nM. In some embodiments, the T cells of the disclosed composition bind to one or plurality of the aforementioned proteins, fragments thereof, or epitopes thereof, with an affinity of EC₅₀ from about 10 nM to about 30 nM. In some embodiments, the T cells of the disclosed composition bind to one or plurality of the aforementioned proteins, fragments thereof, or epitopes thereof, with an affinity of EC₅₀ from about 15 nM to about 35 nM. In some embodiments, the T cells of the disclosed composition bind to one or plurality of the aforementioned proteins, fragments thereof, or epitopes thereof, with an affinity of EC₅₀ from about 17 nM to about 30 nM. In some embodiments, the T cells of the disclosed composition bind to one or plurality of the aforementioned proteins, fragments thereof, or epitopes thereof, with an affinity of EC₅₀ from about 15 nM to about 25 nM.

In some embodiments, the T cells of the disclosed composition bind to one or plurality of the aforementioned proteins, fragments thereof, or epitopes thereof, with an affinity of EC₅₀ of about or of at least of 0.1 nM, 0.5 nM, 1 nM, 5 nM, 7.5 nM, 10 nM, 20 nM, 30 nM, 40 nM, 50 nM, 60 nM, 70 nM, 80 nM, 90 nM, <100 nM or 100 nM.

The disclosure relates to a pharmaceutical composition comprising a therapeutically effective amount of T cells that specifically bind to a Coronaviridae peptide having at least about a 80%, 85%, 90%, 95%, 99%, <100%, or 100% sequence identity to SEQ ID NO:11, SEQ ID NO: 12, SEQ ID NO:13 and/or SEQ ID NO:14.

The naïve T cells in the pharmaceutical compositions may be derived by a biopsy of a donor (optionally frozen after differentiation and harvesting) followed by expansion in culture using the steps disclosed herein. Blood, serum or lymph tissue may be biopsied from a subject. The starting material is composed of three μm punch biopsies collected using standard aseptic practices. Blood may be collected by the treating physician, placed into a vial. The biopsies are shipped at a temperature of about 2, 3, 4, 5, 6, 7 to about 8° C. refrigerated shipper back to the manufacturing facility. After arrival at the manufacturing facility, the biopsy is inspected and, upon acceptance, transferred directly to the manufacturing area.

In some embodiments, preparation of the engineered cells includes one or more culture and/or preparation steps. The cells for introduction of the composition comprising the one or more cytokines, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig. In some embodiments, isolation of the cells includes one or more preparation and/or non-affinity-based cell separation steps. In some examples, cells are washed, centrifuged, and/or incubated in the presence of one or more reagents, for example, to remove unwanted components, enrich for desired components, lyse or remove cells sensitive to particular reagents.

In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. For example, in some aspects, specific subpopulations of T cells, such as cells positive or expressing one or more markers, e.g., CD4+, CD25+, CD27+, CD4+, CD8+ or other markers disclosed herein.

In one example, a sample of PBMCs or other white blood cell sample is subjected to selection of CD4+ cells, where both the negative and positive fractions are retained. The negative fraction then is subjected to negative selection based on expression o, for example, CD14 and CD45RA, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naïve CD4+ T lymphocytes are CD45RO+, CD45RA+, CD62L+, CD4+ T cells. In some embodiments, central memory CD4+ cells are CD62L+ and CD45RO+. In some embodiments, to enrich for CD4+ cells by negative selection, a monoclonal antibody cocktail typically includes antibodies to CD14, CD20, CD11b, CD16, HLA-DR, and CD8.

In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in METHODS IN MOLECULAR MEDICINE, vol. 58: Metastasis Research Protocols, Vol. 2: Cell Behavior In vitro and In vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference in their entireties.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex.

In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml).

In some embodiments, the isolated cells are part of one or more disclosed compositions of T cells and those T cells are stimulated with a composition of cytokines agents including IL-15 and/or IL-6. In some embodiments, the composition of cytokines is free of one or more of: IL-1, IL-12, IL-4, or IL-7. In some embodiments, methods of the disclosure relate to stimulating isolated naïve T cell compositions with a composition of at least two cytokines (such as IL-6 and IL-15) and are free of one or more of: IL-1, IL-12, IL-4, or IL-7. In some embodiments, the methods of expanding, proliferating or stimulating the T cell populations are free of a step of exposing the T cell populations to cytokines one or more of: IL-2, IL-4, IL-4, IL-7, IL-12, IL-18 and IL-27. In some embodiments, the methods disclosed herein are free of a step of using a composition of feeder cells.

The precise amount of T cells to be administered can be determined by a physician with consideration of individual differences in age, weight, extent of disease and condition of the subject.

Typically, administration of T cell therapies is defined by number of cells per kilogram of body weight. However, because T cells will replicate and expand after transfer, the administered cell dose will not resemble the final steady-state number of cells.

In some embodiments, a pharmaceutical composition comprising the T cells of the present invention may be administered at a dosage of 10⁴ to 10⁹ cells/kg body weight. In another embodiment, a pharmaceutical composition comprising the T cells of the present invention may be administered at a dosage of 10⁵ to 10⁶ cells/kg body weight, including all integer values within those ranges.

Compositions comprising the T cells of the present invention may also be administered multiple times at these dosages. The cells can be administered by using infusion techniques that are known in the art (see, for example, Rosenberg et al., 1988, New England Journal of Medicine, 319: 1676). The optimal dosage and treatment regimen for a particular subject can be readily determined by one skilled in the art by monitoring the patient for signs of disease and adjusting the treatment accordingly.

In certain embodiments, administration of any of the compositions embodied herein, e.g. a T cell, for the treatment of a viral infection, can be combined with other cell-based therapies, for example, stem cells, antigen presenting cells, etc.

The composition of the present invention may be prepared in a manner known in the art and are those suitable for parenteral administration to mammals, particularly humans, comprising a therapeutically effective amount of the composition alone, with one or more pharmaceutically acceptable carriers or diluents. The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the invention may also include other supplementary physiologically active agents.

The carrier must be “pharmaceutically acceptable” in the sense of being compatible with the other ingredients of the composition and not injurious to the subject. Compositions include those suitable for parenteral administration, including subcutaneous, intramuscular, intraarticular, intravenous and intradermal administration. The compositions may conveniently be presented in unit dosage form and may be prepared by any method well known in the art of pharmacy. Such methods include preparing the carrier for association with the compositions comprising a therapeutically effective amount of stimulated T cells or T cells produced by the methods disclosed herein. In general, the compositions are prepared by uniformly and intimately bringing into association any active ingredients with liquid carriers.

In an embodiment, the composition is suitable for parenteral administration. In another embodiment, the composition is suitable for intravenous administration. In another embodiment, the composition is suitable for intraarticular administration. In one embodiment, SARS-CoV-2 or other pathogen-specific T cells are administered parenterally, for example, by intravenous infusion, intraperitoneal infusion, or other parenteral mode. T cells may also be infused to a site of SARS-CoV-2 or microbial infection such as into the lungs or upper or lower respiratory system or into or around another infected tissue or organ.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

The invention also contemplates the combination of the T cell composition of the present disclosure with other drugs and/or in addition to other treatment regimens or modalities such as surgery. When the composition of the present invention is used in combination with known therapeutic agents the combination may be administered either in sequence (either continuously or broken up by periods of no treatment) or concurrently or as an admixture.

Systems

In some embodiments, the disclosure relates to a system comprising a cell culture unit that is utilized to culture and expand a T cell population described herein. In some embodiments, the cell culture unit comprises one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet. In some embodiments, the one or plurality of cell reactor surfaces are configured in a cylindrical form with a hollow volume fixed within a cylindrical first compartment; wherein the first media line and the second media line are positioned on opposite faces of the cylindrical first compartment. The first media line can be attached to a first sealable aperture configured for sterile attachment of a cell culture media source. In some embodiments, the system further comprises a pump and a fluid regulator in operable contact with the first media line, wherein the pump is capable of generating pressure in the first media line and wherein the fluid regulator is capable of regulating the speed of fluid from the pump through the first compartment and into the second media line.

The one or plurality of cell reactor surfaces can have a surface area from about 0.5 m² to about 100.0 m², including any value therein, such as about 3 m², about 4 m², about 5 m², about 6 m², about 7 m², about 8 m², about 9 m², about 10 m², about 11 m², about 12 m², about 13 m², about 14 m², about 15 m², about 16 m², about 17 m², about 18 m², about 19 m², about 20 m², about 21 m², about 22 m², about 23 m², about 24 m², about 25 m², about 26 m², about 27 m², about 28 m², about 29 m², about 30 m², about 31 m², about 32 m², about 33 m², about 34 m², about 35 m², about 36 m², about 37 m², about 38 m², about 39 m², about 40 m², about 41 m², about 42 m², about 43 m², about 44 m², about 45 m², about 46 m², about 47 m², about 48 m², about 49 m², about 50 m², about 51 m², about 52 m², about 53 m², about 54 m², about 55 m², about 56 m², about 57 m², about 58 m², about 59 m², about 60 m², about 61 m², about 62 m², about 63 m², about 64 m², about 65 m², about 66 m², about 67 m², about 68 m², about 69 m², about 70 m², about 71 m², about 72 m², about 73 m², about 74 m², about 75 m², about 76 m², about 77 m², about 78 m², about 79 m², about 80 m², about 81 m², about 82 m², about 83 m², about 84 m², about 85 m², about 86 m², about 87 m², about 88 m², about 89 m², about 90 m², about 91 m², about 92 m², about 93 m², about 94 m², about 95 m², about 96 m², about 97 m², about 98 m², or about 99 m², or about 100 m², or about 105 m².

The system further comprises a gas transfer module in operable connection to the one or plurality of cell reactor surfaces. In some embodiments, the gas module comprises a gas pump and a gas regulator connected to the first compartment by a first gas line. In such embodiments, the first compartment comprises at least one gas outlet. The gas pump is capable of generating air pressure from the pump to the first compartment through the first gas line. The gas outlet can be one or more vents or the gas outlet can be configured for sterile connection to one or more vents. The gas regulator is capable of regulating the speed of gas from the pump through the first compartment.

Some embodiments further comprise a first gas inlet in operable connection to the gas transfer module. In some embodiments, the first gas inlet is attached to a second sealable aperture configured for sterile attachment of a gas source. The gas source can be any known gas storage and/or delivery system, such as for example a container or a tank.

The system can further comprise an apheresis unit in fluid communication with the cell culture unit. Suitable apheresis units include the Spectra Optia Apheresis System (TerumoBCT).

Additionally, in some embodiments, the system further comprises a harvesting compartment in fluid communication with the cell culture unit. Suitable harvesting compartments are discussed elsewhere herein.

A cell culture system as described herein can be used to expand memory effector T cells from a subject through culturing one or a plurality of T cells in the system and allowing the T cells to grown in the first compartment for a time period sufficient to proliferate. The T cells can be introduced into the system through the system's first compartment. In some embodiments, the T cells are CD4+ T cells. In some embodiments, the T cells are CD8+ T cells. In some embodiments, the T cells are a mixture of CD4+ and CD8+ T cells. In some embodiments, the T cells are CD45A+ T cells.

The disclosure also relates to a system comprising a cell culture unit comprising one or a plurality of cell reactor surfaces housed in a plurality of compartments, each compartment separated by a removable partition first compartment comprising at least one cell reactor surface, at least one cell reactor surface in fluid connection with a first and second media line, the first media line in fluid communication with a first media inlet, the second media line in fluid communication to a first media outlet. In some embodiments, the cell culture unit comprises a single cell culture chamber comprising multiple partitions, each partition independently removable and independently in fluid connection with the first and the second media line and each partition or set of partitions defining a distinct compartment. In some embodiments, the cell culture unity comprises 2, 3, 4, 5, 6, 7, 8, 9, 10, 11, 12 or more compartments, each compartment separated by and/or defined by one or more partitions. In some embodiments, the compartments are configured in a grid or linear pattern. In some embodiments, each partition separating one compartment from another compartment may be removed such that the cell reactor surface of a first compartment is or becomes contiguous with a cell reactor surface of a second compartment. The removal of one or more partitions allows for an increased surface area onto which cells from one compartment (such as the first compartment) may proliferate and/or grow into another compartment (such as the second compartment) during a method of culturing. In some embodiments, the cell culture unit comprises a set of side walls defining a single surface area divided among 2, 3, 4, 5, 6, 7, 8, 9, 10, 11, 12 or more compartments each compartment with at least one or a plurality of cell reactor surfaces. In some embodiments, each compartment has at least a first cell reactor surface. The disclosure relates to a method of growing T cell populations on a tissue culture system disclosed herein, wherein primary sets of lymphocytes are plated at about a concentration of from about 0.001 to about 10 million cells per milliliter into one or more compartments of the cell culture unit and then allowed to grow to a confluent layer on surface area of from about 1 to about 200 squared centimeters. In some embodiments, the method further comprises removing one or more partitions to allow the cells to grow in a second compartment until confluence, when again, optionally, another partition may successively be removed to allow for more surface for expanded culture. In some embodiments, the method of culturing further comprises repeating the step of removing a partition for each of the compartments into which cells should grow. In some embodiments, the cell culture unit comprises 3, 4, 5, 6, 7, 8, 9, 10, 11, 12 or more partitions each of which corresponding to the physical barrier between a second and third compartment, between a third and fourth compartment, between a fourth and fifth compartment, between a fifth and sixth compartment, between a sixth and seventh compartment, between a seventh and eighth compartment, between an eighth and ninth compartment, between a ninth and tenth compartment, between a tenth and eleventh compartment, and/or between an eleventh and twelfth compartment, respectively.

In some embodiments, one or more of the partitions comprise an interior portion, a frame portion and an exterior portion. The interior portion of the partition is positioned in the closed portion of the system; the frame portion spans a wall of the culture system separating the interior of the culture system to the exterior of the system; and the exterior portion is positioned outside of the system. In some embodiments, a seal operably fits around the frame portion of one or more of the partitions such that removal of the partition does not introduce pathogens to and/or does not expose the environment outside of the tissue culture system to the interior of the tissue culture system.

In some embodiments, the cell density of each compartment is from about 0.1 to about 10 million cells per mL of cell culture media. In some embodiments, the cell density of each compartment is from about 0.1 to about 10 million cells per mL of cell culture media. In some embodiments, the cell density of each compartment is from about 0.5 to about 10 million cells per mL of cell culture media. In some embodiments, the cell density of each compartment is from about 1.0 to about 10 million cells per mL of cell culture media. In some embodiments, the cell density of each compartment is from about 2 to about 10 million cells per mL of cell culture media. In some embodiments, the cell density of each compartment is from about 3 to about 10 million cells per mL of cell culture media. In some embodiments, the cell density of each compartment is from about 4 to about 10 million cells per mL of cell culture media. In some embodiments, the cell density of each compartment is from about 5 to about 10 million cells per mL of cell culture media. In some embodiments, the cell density of each compartment is from about 6 to about 10 million cells per mL of cell culture media. In some embodiments, the cell density of each compartment is from about 7 to about 10 million cells per mL of cell culture media. In some embodiments, the cell density of each compartment is from about 8 to about 10 million cells per mL of cell culture media. In some embodiments, the cell density of each compartment is from about 9 to about 10 million cells per mL of cell culture media. In some embodiments, the cell density of each compartment is from about 0.1 to about 20 million cells per mL of cell culture media. In some embodiments, the cell density of each compartment is from about 0.1 to about 50 million cells per mL of cell culture media.

In some embodiments, the systems disclosed herein comprise a cell density of from about 0.01 million to about 10 million cells per square centimeter. In some embodiments, the systems disclosed herein comprise a cell density of from about 0.03 million to about 5 million cells per square centimeter. In some embodiments, the systems disclosed herein comprise a cell density of from about 0.07 million to about 5 million cells per square centimeter. In some embodiments, the systems disclosed herein comprise a cell density of from about 0.03 million to about 5 million cells per square centimeter. In some embodiments, the systems disclosed herein comprise a cell density of from about 0.001 million to about 5 million cells per square centimeter. In some embodiments, the systems disclosed herein comprise a cell density of from about 0.002 million to about 4 million cells per square centimeter. In some embodiments, the systems disclosed herein comprise a cell density of from about 0.003 million to about 5 million cells per square centimeter of surface area of cell reactor surface. In some embodiments, the systems disclosed herein comprise a cell density of from about 0.004 million to about 5 million cells per square centimeter of surface area of cell reactor surface. In some embodiments, the systems disclosed herein comprise a cell density of from about 0.005 million to about 5 million cells per square centimeter of surface area of cell reactor surface. In some embodiments, the systems disclosed herein comprise a cell density of from about 0.006 million to about 5 million cells per square centimeter of surface area of cell reactor surface. In some embodiments, the systems disclosed herein comprise a cell density of from about 0.007 million to about 5 million cells per square centimeter of surface area of cell reactor surface. In some embodiments, the systems disclosed herein comprise a cell density of from about 0.001 million to about 4 million cells per square centimeter of surface area of cell reactor surface. In some embodiments, the systems disclosed herein comprise a cell density of from about 0.001 million to about 3 million cells per square centimeter of surface area of cell reactor surface. In some embodiments, the systems disclosed herein comprise a cell density of from about 0.003 million to about 3 million cells per square centimeter of surface area of cell reactor surface.

Any and all of the publications cited in this application are incorporated by reference in their entireties.

### EXAMPLES

### Example 1

### Identification of New Cytokine Combinations for Antigen-Specific T Cell Therapy Products Via a High Throughput Multi-Parameter Assay

Infusion of viral-specific T cells (VSTs) is an effective treatment for viral infection after stem cell transplant. Current manufacturing approaches are rapid but growth conditions can still be further improved. To optimize VST cell products, a high-throughput flow cytometry-based assay using 40 cytokine combinations in a 96 well plate was designed to fully characterize T cell viability, function, growth, and differentiation. Peripheral blood mononuclear cells (PBMC) from six consenting donors were seeded at 100,000 cells/well with pools of CMV peptides from IE-1 and pp65, and combinations of IL-15, IL-6, IL-21, IFNα, IL12, IL-18, IL-4, and IL-7. Ten day cultures were tested by 13 color flow cytometry to evaluate viable cell count, lymphocyte phenotype and memory markers as well as IFNγ and TNFα expression. Combinations of IL-15/IL-6 and IL-4/IL-7 were optimal for expansion of viral specific CD3+ T cells, (18-fold and 14-fold respectively compared with unstimulated controls). CD8+ T cells expanded 24-fold in IL-15/IL-6, and 9-fold in IL-4/IL-7 cultures (p<0.0001). CD4+ T cells expanded 27-fold in IL-4/IL-7 and 15-fold in IL-15/IL-6 (p<0.0001). CD45RO+ CCR7− effector memory T cells were the preponderant cells (76.8% and 72.3% in IL-15/IL-6 and IL-15/IL-7 cultures respectively). Cells cultured in both cytokine conditions were potent, with 19.4% of CD3+ cells cultured in IL-15/IL-6 producing IFNγ (7.6% producing both TNFα and IFNγ), and 18.5% of CD3+ cells grown in IL-4/IL-7 (9% producing both TNFα and IFNγ). This study shows the utility of this single plate assay to rapidly identify optimal growth conditions for VST manufacture using only 10⁷ PBMC.

Introduction. Adoptive T cell immunotherapies are increasingly used to treat infection and malignant disease. A common technique involves culturing T cells with antigen presenting cells (APC) exposed to peptide antigens in the presence of a cytokine cocktail. Several immunomodulatory cytokines are currently used to promote T cell division and differentiation, but optimal conditions for the growth and function of peptide stimulated T cell products have yet to be fully defined. Cell products used in clinical trials have typically supplemented T cell cultures with the growth promoting cytokines IL2, IL-15, IL-4 and IL-7 [1-3]. The search to optimize culture conditions is, however, limited by the time and labor needed to screen multiple cytokine combinations. A flow cytometry-based approach was therefore established to rapidly evaluate many cytokine combinations in a single 96 well plate to measure T cell phenotype and potency on a limited numbers of cells.

Manufacture of viral specific T cell products expands a heterogeneous pool of pre-existing memory T cells from donor peripheral blood, with the final product containing a polyclonal mixture of CD4+ helper and CD8+ cytotoxic T cells [4]. This diversity increases the complexity of manufacture, as CD4+ T cells and CD8+ T cells respond differently to cytokine stimulation. For example, IL-4 enhances survival of resting T cells and induces CD4+ Th2 helper differentiation [5-7], while IL-15 promotes survival and diversity of CD8+ memory T cells [8, 9]. IL2 is a canonical T cell growth cytokine which continues to be used in clinical trials due to its effectiveness in expanding T cells derived from tumor infiltrating lymphocytes [10]. However other cytokines also have important functions: IL-6 may enhance Th17 development [11], IL-7 promotes T cell homeostatic survival [12-14], and IL21 promotes the activity of CD8+ T cells [15-17].

The manufacturing methods have transitioned from culturing T cells in 24 well plates with IL2 and APC transduced with viral antigens [18-20] to a simplified culture containing a combination of IL-4 and IL-7 in G-Rex gas permeable devices with soluble mixes of peptides [3, 21, 22]. This system rapidly expands functionally competent T cells specific for multiple viruses. Ten-fold expansions of 1.5×10⁷ donor PBMCs cultured in a G-Rex can provide sufficient VST CD3+ T cells to treat multiple patients in the third party “off the shelf” setting [3, 23-26]. Manufactured T cells are currently characterized for safety, phenotype, and potency in three separate assays: ELISPOT for IFN-γ release, ⁵¹Cr release to measure cytotoxicity, and flow cytometry to identify cellular phenotype. However, routinely available 13 color flow cytometry panels make it possible to measure intracellular cytokines, surface marker phenotype, and correlates of cytotoxicity and alloreactivity, in a single assay to define product quality. Flow cytometric assays minimize culture volume reducing the number of cells needed for validation, while increasing the number of testable conditions that can be applied to donor PBMC. Using this approach, new cytokine combinations controlling phenotypic diversity, growth and function of viral specific T cells products were identified. It was found that high throughput screening by multicolor flow cytometry is affordable and practical for product development.

Materials and Methods

Blood collection. Peripheral blood was collected from de-identified platelet transfusion filters from donors according to IRB-approved protocol.

Cell culture. PBMCs were separated by Ficoll and spun at 800×g for 25 minutes to purify lymphocytes. Cells were washed twice with complete RPMI, and 2×10⁷ cells were re-suspended in 5 mL with 10 μL of 200 μg/mL of peptide libraries encompassing IE1 and PP65. Cells were incubated at 37° C. for 1 hour. Five mL of complete media was added and 100,000 cells/well were plated in 96-well round-bottom plates. Cytokines were added at the indicated concentration in a final volume of 200 μL. Cells were cultured for seven days. Plates were then spun down at 400×g and the cells re-suspended in 200 μL of complete media. Samples were split into two plates of 100 μL each, and fresh media and cytokines were added at the indicated concentration to a final volume of 200 μL. Cells were cultured for three additional days before antigen re-stimulation and antibody staining. For culture within Grex-10 gas permeable chambers (Wilson Wolf; St. Paul, Minn., 1-1.5×10⁷ cells were isolated from PBMCs and re-suspended in 1 mL of complete media with 2 μL of 200 μg/mL of peptide libraries encompassing IE1 and PP65 and cultured for 1 hour at 37° C. 29 mL of complete media was added, along with either 400 U/mL IL-4+10 ng/mL IL-7, or 10 ng/mL of IL-15+100 ng/mL IL-6. Cells were cultured for seven days, after which 15 mL of media was removed and replaced with fresh complete media and cytokines. Cells were cultured for three additional days before antigen re-stimulation in ELISPOT assays.

Intracellular cytokine staining. Plates were spun down at 400×g for 5 minutes and re-suspended in 100 μL of complete media containing the mix of IE1 and PP65 peptide libraries at 1.0 μg/mL final concentration with no peptide controls. Cells were incubated with peptides at 37° C. for 1 hour. Then, 100 μL of complete media containing Brefeldin A or Monensin was added. Cells were cultured for an additional 5 hours, after which cells were stained with antibodies for phenotyping.

ELISPOT assay. ELISPOT plates were coated with 100 μL of 1 μg/mL final concentration anti-IFNγ mAb (Clone 1-D1K; Mabtech, Cincinnati, Ohio) in sterile ELISPOT carbonate coating buffer (1.59 g Na₂CO₃, 2.93 g NaHCO₃ per liter of sterile water) overnight at 4° C. Plates were washed twice with 150 μL of coating buffer, and 100 μL of complete media was added to wells and incubated for 1 hour at 37° C. Cells from G-rex10 culture vessels were plated at the indicated concentrations in 200 μL total volume with actin (1 μg/mL), IE1 and pp65 peptide pools (1 μg/mL), SEB (0.5 μg/mL), or media alone. Plates were incubated for 16 hours at 37° C., after which cells were decanted and plates were washed six times with 1×PBS/0.05% Tween 20. 100 μL of biotinylated anti-IFNγ mAb at 1 μg/mL (Clone 7-B6-1; Mabtech) in biotin buffer (2.5 g biotin in 500 mL 1×PBS) was added to each well and incubated at 37° C. for 1 hour. Plates were washed six times with 1×PBS/0.05% Tween 20. 100 μL of avidin-peroxidase solution in 1×PBS/Tween 20 (APC; Millipore Sigma, Darmstadt, Germany) was added per well and incubated for 1 hour at room temperature. Plates were washed three times with 1×PBS/0.05% Tween 20 and three times with 1×PBS. Spots were developed using 100 μL of AEC substrate (3-amino-9-ethylcarbazole; Vector Labs, Burlingame, Calif.) for 4 minutes at room temperature and rising with tap water. Plates were dried overnight, individual wells were punched out onto adherent film for enumeration by an independent 3^{rd} party. Corrected spot counts were derived according to the formula (Raw spot value+2×[(Raw spot value×% confluence)/(100%−% confluence)]).

Antibody staining. Cells were spun down at 400×g for 5 minutes and washed once in 100 μL 1×PBS. Cells were spun down again and re-suspended in 50 μL of 1×PBS containing Live Dead Aqua at a dilution of 1:500 and then stained for 20 minutes at 4° C., and washed with 100 μL of 1×PBS containing 2% FCS. Cells were fixed in 50 μL of 1× Cytofix/Cytoperm (BD Biosciences, San Jose, Calif.) for 30 minutes at 4° C. and washed twice in 1× Permwash (BD Biosciences, San Jose, Calif.), then re-suspended in 25 μL of staining solution containing 12.5 μL of 1× Permwash and 12.5 μL of Brilliant Violet staining solution (Biolegend, San Diego, Calif.). Markers for staining included CD62L V450 (Biolegend; clone DREG-56), CD4 BV570 (Biolegend; clone RPA-T4), CD45RO BV605 (Biolegend; clone UCHL1), CD8 BV711 (Biolegend; clone SK1), CD56 BV785 (Biolegend; clone 5.1H11), CCR7 FITC (Biolegend; G043H7), CD28 PE (Miltenyi Biotec, San Diego, Calif.; clone REA612), CD95 PE Dazzle CF594 (Biolegend; clone DX2), CD3 PerCP Cy5.5 (Biolegend; clone OKT3), TNFαPE Vio770 (Miltenyi Biotec; clone cA2), IFNγ APC (Biolegend; clone RS.B3), CD107a APC H7 (Miltenyi Biotec; clone REA 792), CD45RA APC H7 (Biolegend; clone HI100). Cells were stained for at least 30 minutes at 4° C. then washed twice with 100 μL of 1× Permwash and re-suspended in 55 μL of 1× Permwash. Cells were loaded onto a Sartorius IQue Screener Plus (Sartorius, Göttingen, Germany) for collection of all samples. Data was analyzed in ForeCyt and Flowjo software (FlowJo, Ashland, Oreg.), and statistics were analyzed in Graphpad Prism software (GraphPad, San Diego, Calif.).

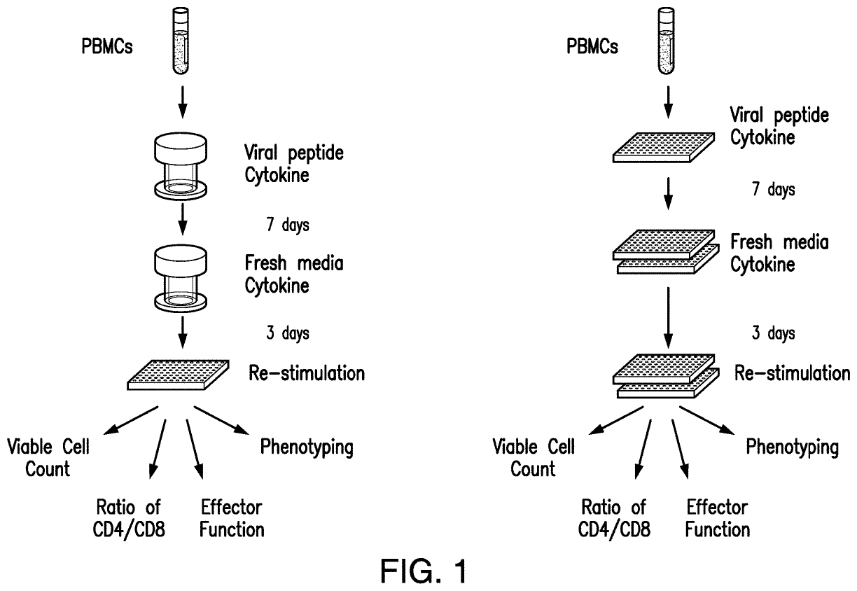

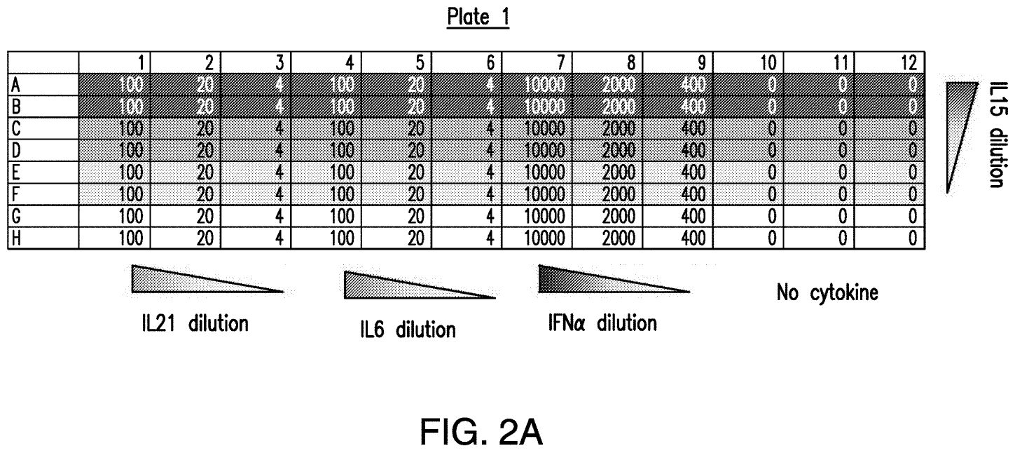

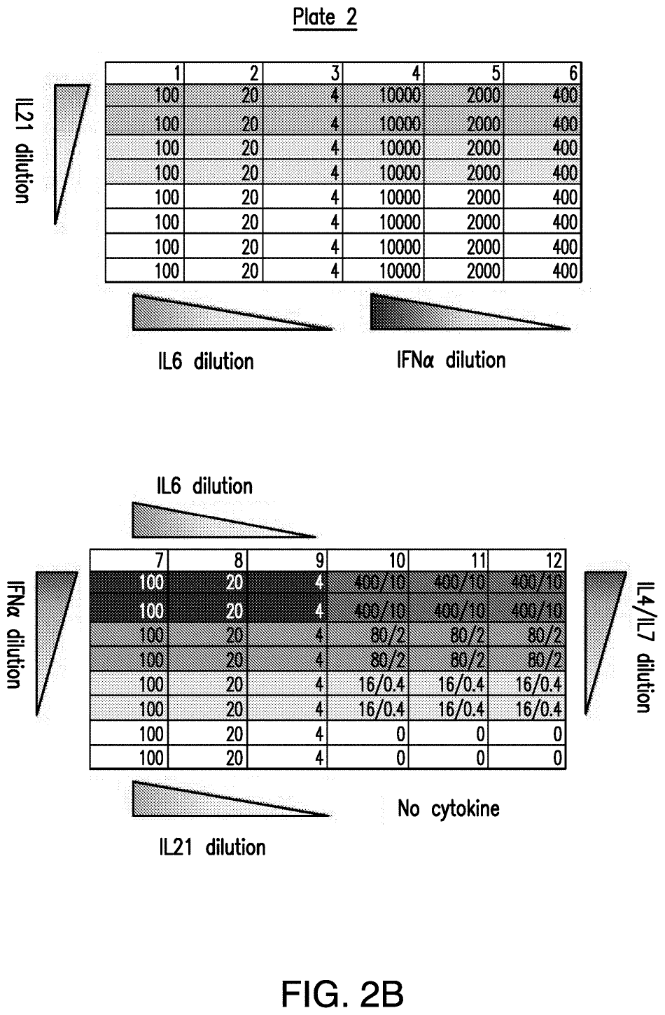

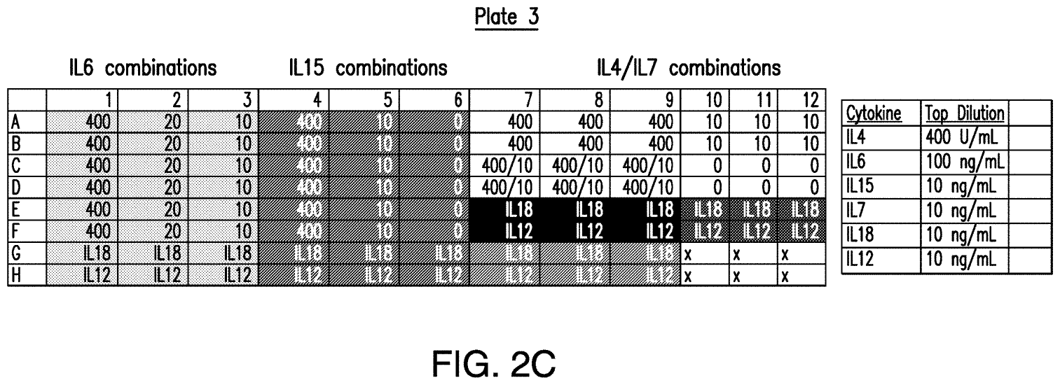

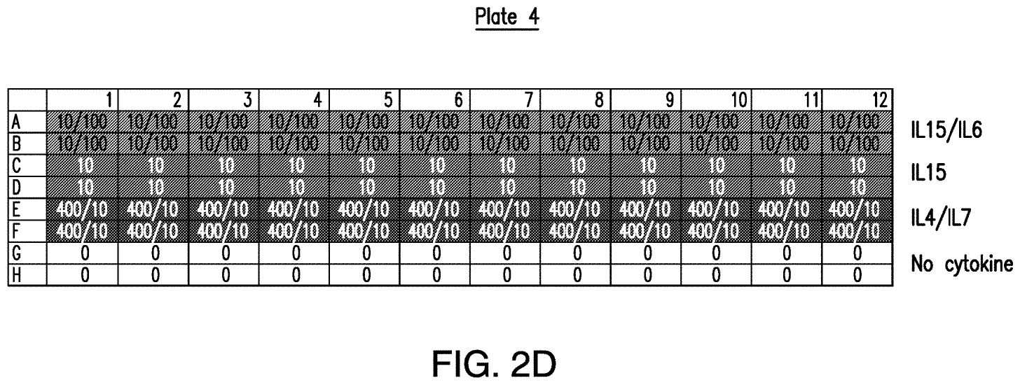

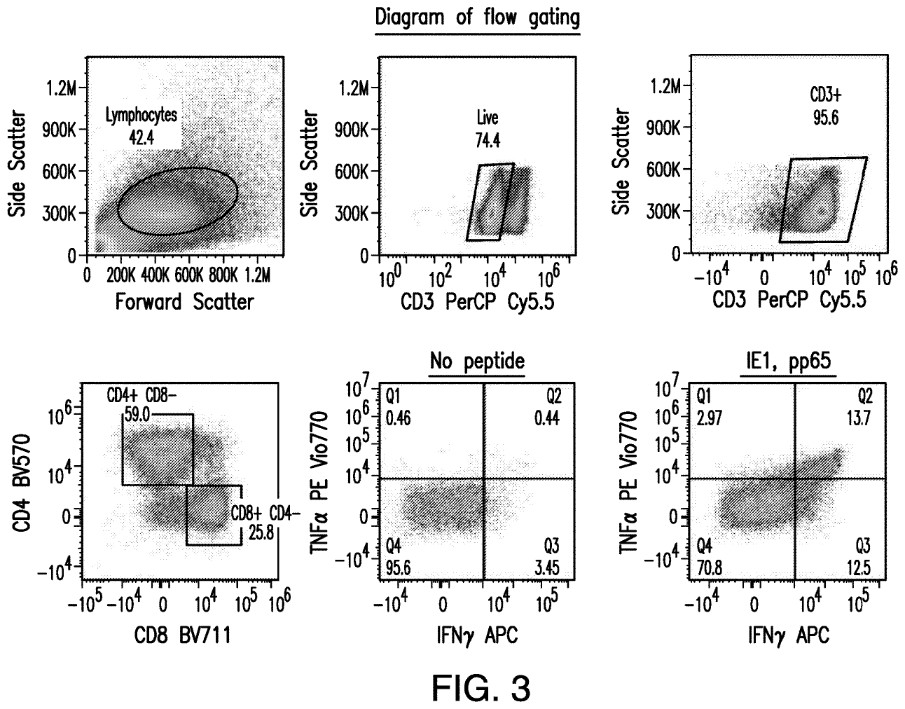

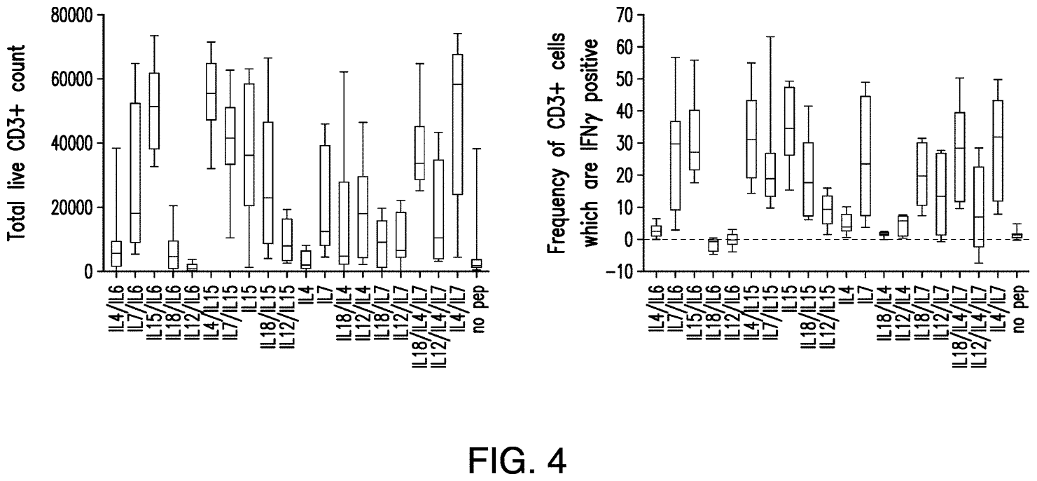

High throughput screening of alternative cytokine combinations. The method allowed for up to 40 cytokine culture conditions to be tested on 1×10⁷ PBMCs in a 96 well plate using the IQue Screener Plus high throughput screener for all flow cytometry. This method combined initial culturing and antigen specific expansion with staining and analysis in a single culture plate (FIG. 1). Flow cytometry was selected as the method for analysis to combine measurements of cellular phenotype, viability, expansion, and effector function in a single 13-color panel. To test for antigen specificity, cells were split on day 7 into two identical plates with fresh media and cytokines, and plates were subsequently challenged with or without peptide pools on day 10. Combinations of IL-15, IL-6, IL21, and IFNα were initially selected as compared against IL-4 and IL-7 as a reference standard for four samples (plate layouts 1, 2; FIGS. 2A and 2B). Modified layouts which added IL12 and IL18, as well as combinations intermixing IL-15, IL-6, IL-4 and IL-7, were also tested. Furthermore, layouts with additional replicates of IL-15+IL-6 and IL-4+IL-7, and replicates challenged with irrelevant peptide pools as an additional measure of antigen specificity were tested (plate layouts 3, 4; FIGS. 2C and 2D). Overall, the use of 96 well plates as both culture and staining vessels for flow cytometric analysis allowed testing of 92 different combinations of cytokines using four different plate layouts. The workflow for analysis of samples utilized a hierarchical gating strategy which categorized positive and negative gates based on initial FMO staining controls as analyzed within Flowjo (FIG. 3). For phenotyping, the frequency of living CD3+, CD3+CD4+, and CD3+CD8+ T cells present within the culture was measured, along with CD3− CD56+NK cells. Viability was measured by vital dye staining using Live Dead Aqua, while cytotoxic function was compared by measuring IFNγ and TNFα intracellular cytokine production in viral wells pulsed with peptide pools over antigen non-specific background wells. Finally, surface expression of T cell memory markers were used to judge the differentiation status of cells, including CD45RA, CD45RO, CCR7, CD28, CD95, and CD62L.

Results

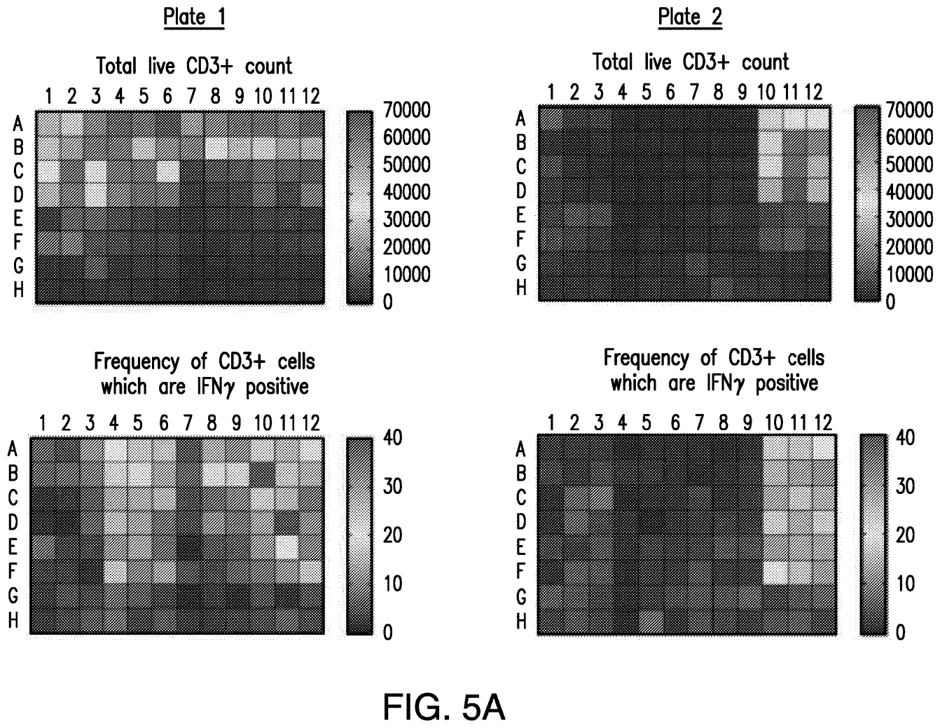

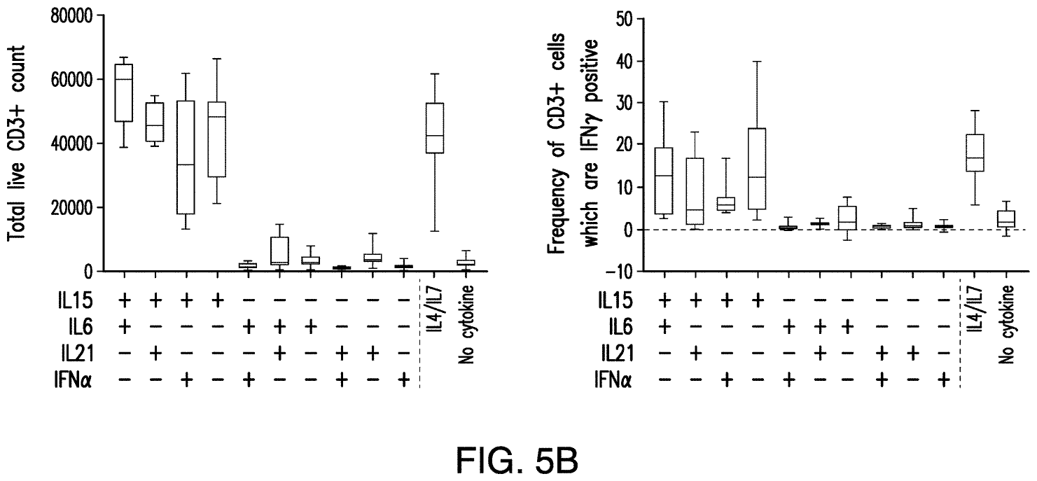

High throughput screening of cytokine combinations. The growth and function of cells in all cytokine conditions was first screened. Representative heat map data are shown FIG. 5A and summarized in FIG. 4. Control culture of PBMCs without added cytokine did not support T cell growth. CD3+ T cell expansion in the standard cytokine mix of IL-4 and IL-7 (optimum results with 400 U/mL and 10 ng/mL respectively) achieved a 12.3-fold increase over control. IL-15 alone or in combination with other cytokines achieved the best expansions at the highest dose of 10 ng/mL representing a 22.5-fold increase over control. Mixtures containing IL-6 and IL21, produced only a modest expansion of 2.9-fold.

The combination of IL-15 (10 ng/mL) and IL-6 (100 ng/mL) was then selected for further investigation based on the favorable expansion of CD3+ T cells and their cytokine production in four experiments (FIG. 5B). It was confirmed that addition of IL-15 or IL-7 was sufficient for expansion of CD3+ T cells, and that the original selection of IL-15/IL-6 was superior to all other combinations. Overall, culture in a combination of IL-15/IL-6 consistently promoted CD3+ T cell expansion and IFNγ production to levels similar to culture in IL-4/IL-7.

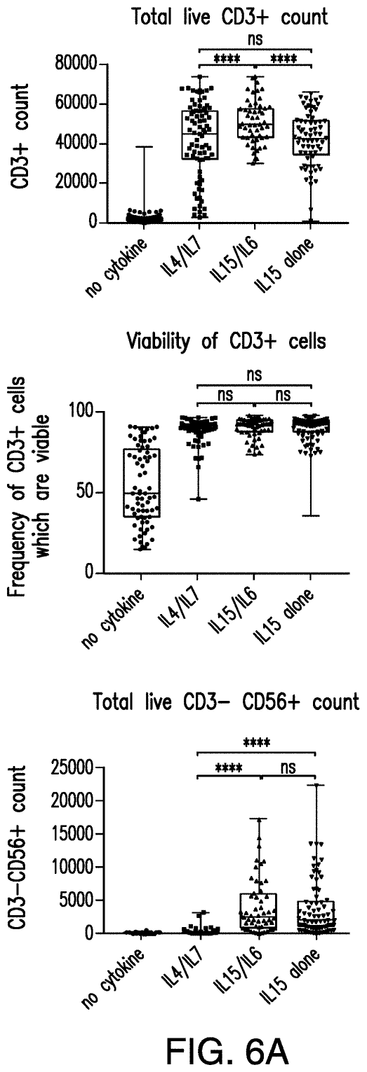

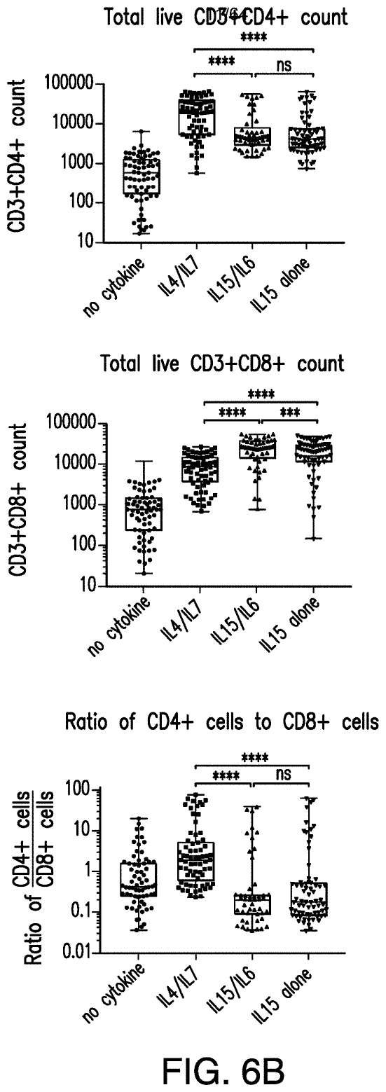

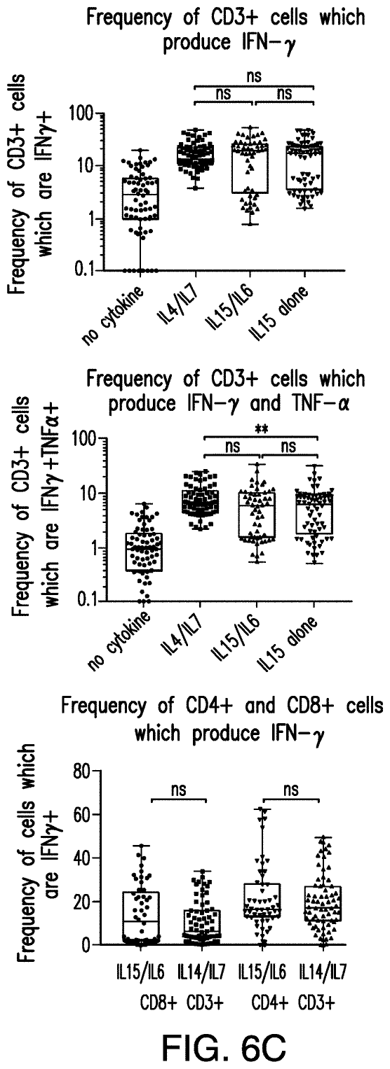

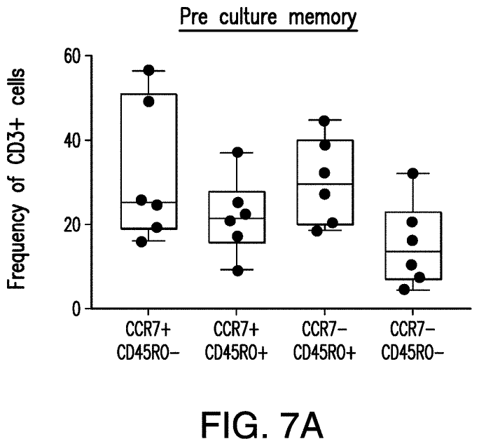

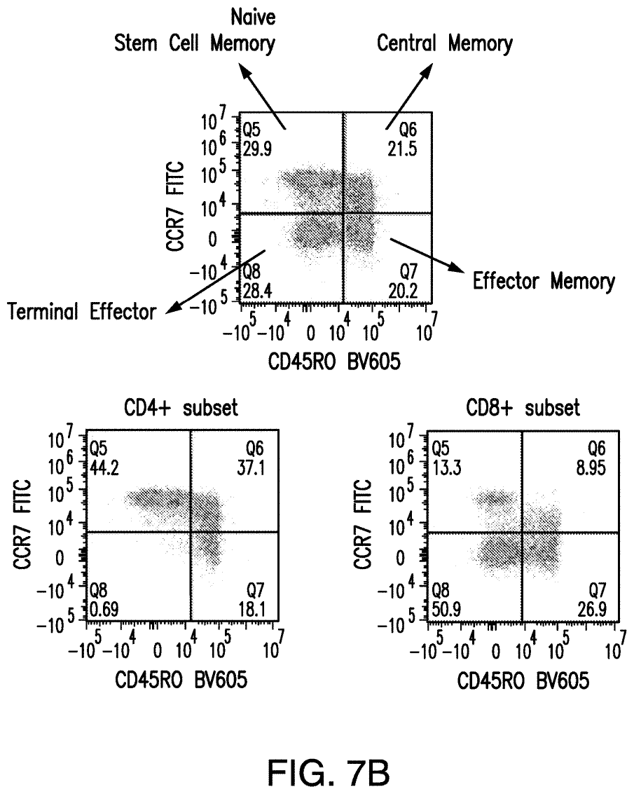

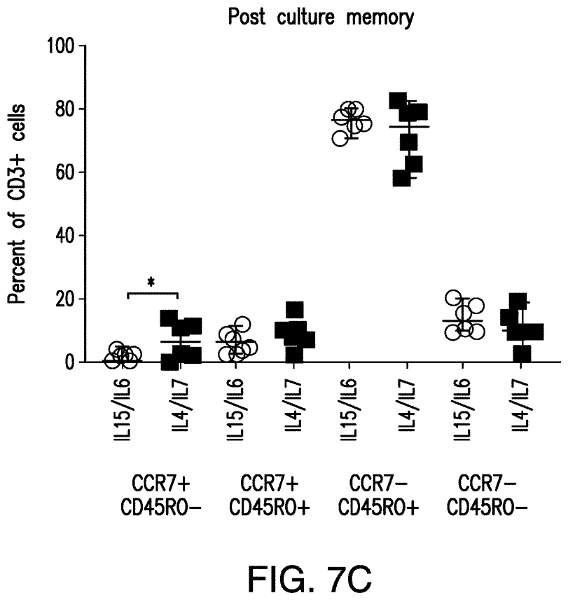

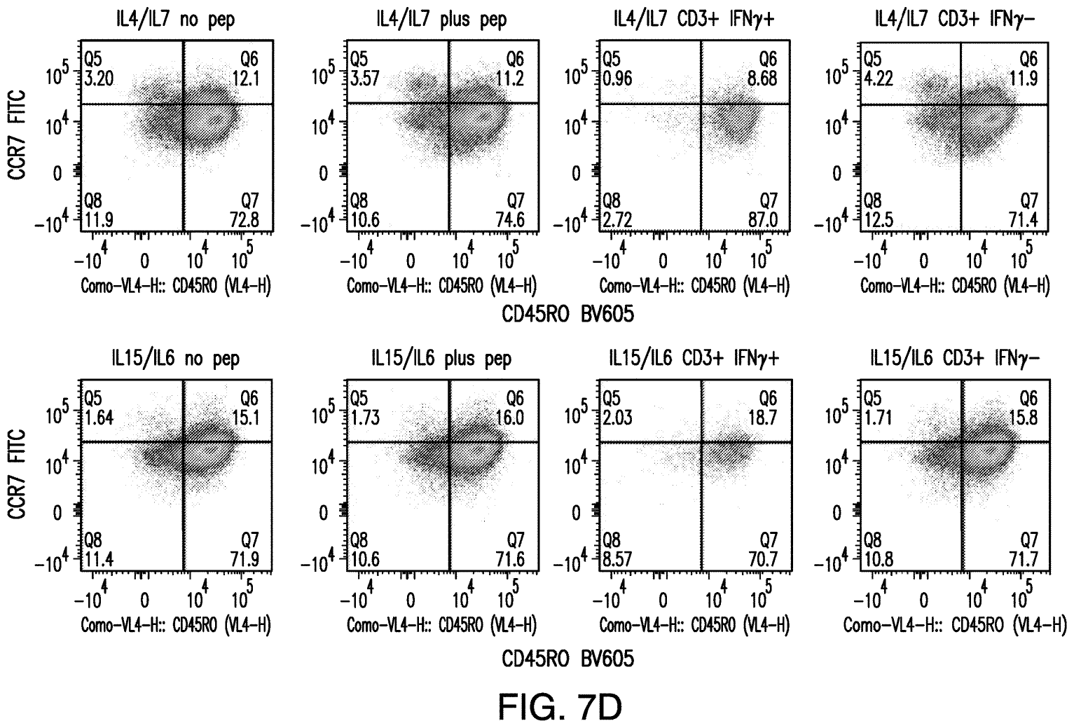

Selective cytokine culture imparts bias on the ratio of CD4 vs CD8 cells in viral specific T cell products. Six CMV reactive patient samples in IL-15 alone, IL-15 and IL-6 and IL-4 and IL-7 were compared in replicate testing (FIG. 6). Culture in IL-15/IL-6 expanded a median of 17.1-fold more CD3+ cells compared with no cytokine controls (p<0.0001). Culture in IL-4/IL-7 expanded a median of 13.8-fold more compared with no cytokine control (p<0.0001; FIG. 7A). Viability of CD3+ cells on average was not significantly different between wells containing IL-15/IL-6 or IL-4/IL-7, with a median of 90% and 89%, respectively (p=0.966). CD3− CD56+ NK cells were present in cultures expanded with IL-15/IL-6, with a median of 6.6% of total cells recovered (4106 cells; FIG. 6A). Less than 300 NK cells were recovered on average from wells containing IL-4/IL-7, representing 0.6% of total cells recovered.

Interestingly, a strong bias in the ratio of CD4+ to CD8+ cells was identified in the final product depending on the cytokines used for initial culture. Culture in IL-15/IL-6 conditions favored outgrowth of CD8+ T cells compared with culture in IL-4/IL-7, which favored outgrowth of CD4+ T cells. Culturing cells in IL-4/IL-7 expanded 2.1-fold more CD4+ T cells than cells cultured in IL-15/IL-6 (FIG. 6B; p<0.0001), while culturing cells in IL-15/IL-6 more than doubled the expansion of CD8+ cells compared with IL-4/IL-7, with 2.8-fold more CD8+ cells on average in IL-15/IL-6 vs IL-4/IL-7 (FIG. 6B; p<0.0001). The viability of CD4+ and CD8+ cells was not significantly different comparing culture in IL-15/IL-6 and IL-4/IL-7 (data not shown; CD4 viability p=0.4381; CD8 viability p=0.1033), suggesting the different cytokine combinations were stimulating outgrowth of either CD4+ or CD8+ cells, rather than preserving the selective survival of individual subsets.

Both culture conditions expanded CD3+ T cells producing IFNγ in response to CMV peptide pool re-stimulation. The highest concentration of IL-15+IL-6 induced a median of 19.3% IFNγ producing CD3+ T cells (range 0.8%-55.3%) while IL-4+IL-7 induced a median of 16.3% of CD3+ T cells recognizing CMV peptides (range 3.8%-49.6%; FIG. 6C; p=0.99). The proportion of multi-cytokine producing cells was also investigated, as evidence suggests these cells offer superior protection against viral infection when compared with cells producing a single cytokine. A median of 6.1% (range 0.6%-35.4%) of cells cultured in IL-15/IL-6 and 6.9% (range 2.3%-25.0%) of cells cultured in IL-4/IL-7 produced both IFNγ and TNFα in response to CMV peptides (FIG. 6C; p=0.22).

Limited production of cytokines by IL-15 expanded CD4+ T cells has been previously reported. Therefore, the production of IFNγ by CD4+ and CD8+ subsets within the CD3+ T cell population was compared and it was found that culture in IL-15/IL-6 was sufficient to expand CMV specific CD8+ and CD4+ cells equivalent to culture in IL-4/IL-7. 6.6% (range 0%/6-34.2%) of CD8+ cells cultured in IL-4/IL-7 and 11.1% (range 0%/6-45.7%) of CD8+ cells cultured in IL-15/IL-6 produced IFNγ in response to CMV (p=0.46). Similarly, a median of 17.3% (range 0%-49.4%) and 16.6% (range 0%/6-62.7%) of CD4+ cells produced IFNγ in response to CMV peptides when cultured in IL-4/IL-7 and IL-15/IL-6, respectively (p=0.46). In summary, compared with IL-4/IL-7, cultures in IL-15/IL-6 produced a comparable number of CMV reactive CD3+ VSTs that were more skewed towards a CD8+ phenotype.

VSTs are effector memory in phenotype. The extent of T cell differentiation has been suggested to influence the persistence of adoptively transferred T cells [27, 28]. The surface phenotype of VSTs expanded by in vitro culture in IL-15/IL-6 and IL-4/IL-7 was characterized to identify the proportion of cells expressing different combinations of T cell memory markers in FIG. 7. Pre-culture CD3+ T cells comprised on average 32% naïve/stem cell memory cells, 30.4% effector memory cells, 22% central memory cells and 15.4% terminal effectors. Ten-day VST products had a preponderance of effector-memory CD3+ cells representing 72.3% and 76.9% of cells grown in IL-4/IL-7 and IL-15/IL-6, respectively. Terminal effector cells lacking both CCR7 and CD45RO, represented 11.3% in IL-4/IL-7, and 14.3% in IL-15/IL-6. Central memory cells represented a minority of cells after culture, 9.3% and 6.6% in IL-4/IL-7 and IL-15/IL-6 cultures, respectively. There was a greater frequency of naïve cells in IL-4/IL-7 cultures (7.0%) as compared to culture in IL-15/IL-6 (2.3%). Both culture conditions substantially reduced the frequency of naïve cells as compared to pre-culture frequencies (32%). The memory phenotype of antigen specific cells was also compared with antigen non-responsive cells. CD3+ IFNγ+ cells had a predominantly effector memory phenotype, while a small number of naïve cells (4.2% in IL-4/IL-7 and 1.7% in IL-15/IL-6) remained within the IFN-γ negative (antigen non-reactive) fraction, suggesting that culture in IL-4/IL-7 was preserving a subset of naïve cells within the final product.

Process development time substantially reduced by using the IQue. Process development of antigen-specific T cells such as VSTs has largely been limited to testing individual conditions in 24-well plates or Grex-10 devices, which limits the systematic testing of an array of cytokines and growth conditions. Expanding and testing VSTs using such methods requires approximately 20 hours of work and 34 hours of incubation per cytokine condition. In contrast, process development in 96 well plates and a 13 color flow cytometry panel required only 12 hours of work and 8 hours of incubation. To evaluate 40 cytokine conditions would therefore take 2160 hours per sample by existing methods, but only 20 hours using the improved approach disclosed herein (Table 5A).

TABLE 5A
Comparison of traditional process development (PD) in culture vessels, traditional PD in plates, and micro assay using flow cytometry.

| | Traditional | Traditional | Micro assay |
|---|---|---|---|
| | PD in culture vessels | PD in plates | using flow cytometry |
| Culture vessel | Grex-10 | 24-well plate | 96-well plate |
| Sample usage | 15 × 10⁶ | 50 × 10⁶ | 10 × 10⁶ |
| Conditions | 1 | 24 | 48 duplicates |
| per culture vessel | | | |
| Time to Ficoll, | 8 hours culture | 10 hours culture | 8 hours culture |
| culture, and feed | | | |
| Time for analysis- | 4 hours setup | 4 hours setup | Included in |
| ELISPOT | 18 hours incubation | 18 hours incubation | intracellular |
| Phenotype | 2 hours setup | 2 hours setup | Included in |
| | 1 hour analysis | 1 hour analysis | intracellular |
| Cytotoxicity | 4 hours setup | 4 hours setup | Included in |
| | 8 hours incubation | 8 hours incubation | intracellular |
| Viable count | 1 hour setup | 1 hour setup | Included in |
| | | | intracellular |
| Intracellular flow | NA | NA | 4 hours setup |
| | | | 6 hours incubation |
| Total time | 8 hours culture | 10 hours culture | 8 hours culture |
| investment for all | 38 hours analysis | 38 hours analysis | 10 hours analysis |
| conditions | (4416 hours for 96 | (192 hours for 96 | (36 hours for 96 |
| | conditions) | conditions) | conditions) |

Optimized cytokine conditions identified in 96-well plate translates to clinical scale manufacturing. Miniaturized cell cultures may not reliably scale-up in a linear fashion. To test whether this system could predict the phenotype and function of clinical size products, whether IL-15/IL-6 cultures in Grex-10 culture vessels would recapitulate the data from the experiments in 96-well plates was investigated. At least 1×10⁷ cells were seeded with IE1 and pp65 peptide pools in Grex-10 culture vessels with medium and either IL-4/IL-7 at 400 U/mL IL-4/100 ng/mL IL-7 or 10 ng/mL IL-15/100 ng/mL IL-6 (FIG. 8). Cells grown in IL-15/IL-6 produced a mean of 638 f 297 spots per 100,000 cells in response to CMV peptides, while cells cultured in IL-4 and IL-7 produced a mean of 555 f 230 spots per 100,000 added cells in response to CMV peptide pool re-stimulation. The CMV response was antigen specific, as cells produced less than 10 spots on average in response to either actin or no peptide controls per 100,000 added cells. This demonstrated that cells grown in IL-15/IL-6 were functionally equivalent to cells grown in IL-4/IL-7 when cultured to clinical scale and that the high throughput screening method can reliably optimize product development.

Discussion Here, a high throughput flow cytometric assay to rapidly and efficiently evaluate growth of viral-specific T cells from donor PBMCs in multiple cytokine combinations was described. Among the combinations tested, superior and comparable expansion and T cell effector function for cells cultured in IL-4/IL-7 and IL-15/IL-6 was identified. Culture with IL-4/IL-7 favored expansion of CD4+ T cells, at the expense of CD8+ T cells, while culture with IL-15/IL-6 expanded both CD8+ and CD4+ T cells. It was subsequently confirmed that the IL-15/IL-6 cytokine growth condition was equivalent to IL-4/IL-7 by IFNγ at clinical scales.

This flow cytometry approach allowed the evaluation of 40 cytokine combinations per plate using only 1×10⁷ cells. Promising cytokine combinations were re-investigated with additional replicates in subsequent experiments, ultimately using only 3×10⁷ total PBMCs to measure 90 total cytokine combinations. Importantly, both the culture conditions and the functional assay were modular by design, allowing for simple exchange of new cytokine combinations into the culture layout as inferior conditions were removed, as well as introducing new flow cytometric markers into the functional assay. This flexibility helps process development through speed, replicate reproducibility, and efficient use of a limited starting product. The approach generates a large amount of data necessitating some restrictions on the parameters selected to study. Determining the optimal growth conditions for T cell expansion, secretion of IFNγ and TNFα, and central/effector memory status were therefore selected. Cytokine combinations of IL-6, IFNα, and IL21 not including IL-15 were excluded because they induced little CD3+ proliferation when compared with combinations of IL-4 and IL-7. When comparing IL-7 and IL-4 in combination with other cytokines, it was found that IL-7 but not IL-4 promoted VST growth. This is consistent with observations that IL-4 promotes cell survival but only supports growth of naïve T cells [5, 6, 29].

It was also found that IL-6 improved cell expansion in combination with IL-15 without modifying effector function. These results are consistent with knockout mouse experiments showing that IL-6 reduces the threshold for TCR signaling in CD8+ T cells [30], promoting memory T cell expansion in response to antigen specific peptide re-stimulation. The requirement for including IL-15 or IL-7 for memory T cell expansion is not unexpected as both receptors share homology with IL2 and use the common γ-chain and its associated Jak/STAT signaling proteins [31-33]. Recombinant IL-7 has been used clinically to expand T cell subsets in cases of lymphopenia [34, 35], and was included with IL-4 for its pro-survival benefits for T cells [36].

The results here showed that culture in IL-15 and IL-6 supports robust antigen specific CD4+ T cell expansion. This is in contrast to earlier studies suggesting that culture in IL-15 was inferior to culture in IL-4/IL-7 because of a lack of antigen specific CD4+ T cell expansion—despite superior total cell expansion—and excessive CD56+NK cell growth [36]. Only a small (median of 6.6%) growth of NK cells was identified in culture with either IL-15/IL-6 or IL-15 alone. Production of an efficacious mix of viral specific CD4+ and CD8+ is a needed for T cell therapy products to enhance the cytotoxic CD8+ T cell response with “help” provided by anti-viral CD4+ T cells in the form of immune activation, recruitment, and inhibition of viral replication [37]. For CMV infections, CD8+ T cells responses correlate with resolution of disease after HSCT [38], while addition of CMV specific CD4+ T cells has also been demonstrated to help CD8+ T cell responses for some HSCT patients [39], and has been suggested to support CD8+ cell persistence [40]. While the data here show a bias towards expanding CD4+ T cells in products cultured with IL-4/IL-7, multiple clinical trials have used VST cells cultured in IL-4/IL-7 to treat ongoing viral infections, including EBV related post-transplant lymphoproliferative disorder (PTLD) [41-44]. These successes may represent the relative abundance of antigen specific memory T cells expanded by the memory VST protocol. Nevertheless, the combination of IL-15/IL-6 may provide a more balanced ratio of antigen specific CD4+ to CD8+ T cells during polyclonal expansion of T cell products against not only viral specific antigens, but also other targets, including tumor-associated antigens.

In conclusion, this high throughput plate-based flow cytometric assay was shown to be able to effectively and reliably measure T cell growth, function, and phenotype to optimize VST product development. The data here show that IL-15/IL-6 is equivalent to IL-4/IL-7 in GMP culture conditions. The modular nature of the assay facilitates future investigations to optimize culture conditions with 3 or 4 cytokine combinations using IL-15/IL-6 and IL-4/IL-7 as a baseline.

### Example 2

IL-18 Function with Cytokines

An experiment was performed with two samples of lymphocytes from subjects, isolating the lymphocytes and culturing the lymphocytes with IL12 or IL18 in combination with IL-15, IL-6, IL-4, and IL-7. While no or limited improvement in T cell effector numbers were stimulated with IL18 or IL12 for CD3 expansion or extra IFNγ production by the T cells. A combination of IL18 with IL-6 or the combination of IL12 with IL-6 did not expand CD3+ T cells at all. A combination of IL18 with IL-15, IL-4, or IL-7 or IL-12 with IL-15, IL-4, or IL-7 did expand CD3+ T cell populations. Both data points suggest that IL-18 with IL-15 may expand effector T cell population but at least that substitution of IL18 or IL12 did not improve the expansion or effector cytokine production.

### Example 3

### SARS-CoV-2 Specific T Cells Predominantly Recognize Conserved Regions of Membrane Protein

The first report of T cell responses to SARS-CoV-2 structural proteins with the identification of immunodominant viral epitopes in conserved regions is presented herein. SARS-CoV-2-specific T cells (CSTs) were expanded from the peripheral blood of 23 convalescent donors by stimulation with peptides spanning the SARS-CoV-2 envelope, membrane, nucleocapsid, and spike antigens. Following in vitro expansion using a GMP-compliant methodology (designed to allow the rapid translation of SARS-CoV-2 T cell therapies to the clinic), membrane, spike, and nucleocapsid peptides elicited IFN-7 production, in 12 (52%), 6 (26%), and 4 (17%) convalescent donors (respectively), and in none of 11 unexposed controls. Multiple novel CD4-restricted epitopes were identified within membrane protein, which induced polyfunctional T cell responses critical for the development of effective vaccine and T cell therapies.

Introduction. SARS-CoV-2, a novel coronavirus first reported in December 2019 from Wuhan, China, is responsible for the ongoing pandemic of coronavirus disease 2019 (COVID-19)[46]. The adaptive immune response to SARS-CoV-2 remains ill-defined, and there is an urgent need to fill this gap in knowledge in order to enable the development of effective vaccines and therapies. Though antibody responses to the spike and nucleocapsid proteins are well described [45, 47], the characterization of T cell response to SARS-CoV-2 is still limited. Three studies have reported that both CD4+ and CD8+ T-cell responses to SARS-CoV-2 are detectable in convalescent patients, but were somewhat confounded by background detection in unexposed individuals [48-50]. Recent studies have also reported that immunocompromised patients are at high risk of severe disease as well as infected individuals who develop lymphocytopenia, suggesting that T cell immunity is essential for overcoming COVID-19 [51, 52]. Studies of the related virus SARS-CoV demonstrated that T cells recognizing viral epitopes within SARS-CoV structural proteins were integral in viral clearance, and remained detectable for up to 4 years after exposure [53, 54].

Here, the first identification of immunodominant T cell epitopes within conserved regions of SARS-CoV-2 structural proteins is presented. These predominately comprise MHC-II-restricted CD4+ T cell responses, similar to those observed in response to other respiratory viruses. By defining the T cell response to SARS-CoV-2 in concert with humoral immunity, this study advances our understanding of the overall adaptive immune response to SARS-CoV-2, facilitating the development of both adoptive T cell therapies and of effective vaccines for the treatment of individuals at risk of infection.

Materials and Methods

Donors. Peripheral blood mononuclear cells (PBMCs) from volunteers, both healthy and those with presumed or documented COVID-19 infection, were obtained from Children's National Hospital (Washington, D.C.) under informed consent approved by the Institutional Review Board in accordance with the Declaration of Helsinki.

Generation of SARS-CoV-2-specific T cells (CSTs). Evaluated T cell products included SARS-CoV-2 specific T cells, manufactured from PBMCs of seropositive and seronegative volunteers. VSTs were produced using a rapid expansion protocol previously described. Briefly, PBMCs were pulsed with overlapping peptide pools encompassing viral antigens (1 μg/15×10⁶ PBMCs) for 30 minutes at 37° C. Peptide libraries of 15-mers with 11 amino acids overlaps encompassing the spike, membrane, nucleocapsid, and envelope proteins were generated (A&A peptide, San Diego, Calif., USA) from the SARS-CoV-2 reference sequence (NC_045512.2). After incubation, cells were resuspended with IL-4 (400 IU/ml; R&D Systems, Minneapolis, Minn.) and IL-7 (10 ng/ml; R&D Systems) in CTL media consisting of 45% RPMI (GE Healthcare, Logan, Utah), 45% Click's medium (Irvine Scientific, Santa Ana, Calif.), 10% fetal bovine serum, and supplemented with 2 mM GlutaMax (Gibco, Grand Island, N.Y.). Cytokines were replenished on day 7. On day 10, cells were harvested and evaluated for antigen specificity and functionality.

IFN-γ Enzyme-Linked Immunospot (ELISpot) Assay. Antigen specificity of T cells was measured by IFN-γ ELISpot (Millipore, Burlington, Mass.). T cells were plated at 1×10⁵/well with no peptide, actin (control), or each of the individual SARS-CoV-2 pepmixes (200 ng/peptide/well). Plates were sent for IFN-γ spots forming cells (SFC) counting (Zellnet Consulting, Fort Lee, N.J.).

Flow Cytometry. VSTs were stained with fluorophore-conjugated antibodies against CD4, CD8, TCRαβ, TCRγδ, and CD56. (Miltenyi Biotec, Bergisch Gladbach, Germany; BioLegend, San Diego, Calif.). All samples were acquired on a CytoFLEX cytometer (Beckman Coulter, Brea, Calif.) Intracellular cytokine staining was performed as follows: 1×10⁶ VSTs were plated in a 96-well plate and stimulated with pooled pepmixes or individual peptides (200 ng/peptide/well) or actin (control) in the presence of brefeldin A (Golgiplug; BD Biosciences, San Jose, Calif.) and CD28/CD49d (BD Biosciences) for 6 hours. T cells were fixed, permeabilized with Cytofix/Cytoperm solution (BD Biosciences) and stained with IFNγ, TNFα, and IL-2 antibodies (Miltenyi Biotec). Data was analyzed with FlowJo X (FlowJo LLC, Ashland, Oreg.; hypertext transfer protocol secure://bearmail.cnmc.org/owa/#).

Luciferase Immunoprecipitation Systems (LIPS) for Measurement of SARS-CoV-2 Antibodies. Testing for antibodies to spike and nucleocapsid proteins were performed using a LIPS assay as recently described [45]. Briefly, plasma samples were incubated with spike and nucleocapsid proteins fused to Gaussia and Renilla luciferase, respectively, protein A/G beads were added, the mixture was washed, coelenterazine substrate (Promega) was added, and luciferase activity was measured in light units with a Berthold 165 LB 960 Centro microplate luminometer. Antibody levels were reported as the geometric mean level (GML) with 95% confidence interval (CI). Cut-off limits for determining positive antibodies in the SARS-CoV-2-infected samples were based on the mean plus three standard deviations of the serum values derived from uninfected blood donor controls or by receiver operator characteristics (ROC) analysis. For some of the data percentages for categorical variables, mean and range, geometric mean plus 95% CI were used to describe the data. Wilcoxon signed rank were used for statistical analysis.

SARS-CoV-2 Antigenic Peptides

Sequences of SARS-CoV-2 antigenic peptides are provided in the following Table 5B.

TABLE 5B

| VIRAL | | MHC |
|---|---|---|
| PROTEIN | AA SEQUENCE | RESTRICTION |
| Nucleocapsid | ILLNKHIDH | A*02:01 |
| | (SEQ ID NO: 14, | |
| | residues 351-359) | |
| | MEVTPSGTWL | HLA-B*40:01 |
| | (SEQ ID NO: 14, | |
| | residues 322-331) | |
| | GMSRIGMEV | HLA-A*02:01 |
| | (SEQ ID NO: 14, | |
| | residues 316-324) | |
| | ILLNKHIDA | HLA-A*02:01 |
| | (SEQ ID NO: 14, | |
| | residues 351-359 | |
| | ALNTPKDHI | HLA-A*02:01 |
| | (SEQ ID NO: 14, | |
| | residues 137-146) | |
| | LALLLLDRL | HLA-A*02:01 |
| | (SEQ ID NO: 14, | |
| | residues 219-227) | |
| | LLLDRLNQL | HLA-A*02:01 |
| | (SEQ ID NO: 14, | |
| | residues 221-230) | |
| | LQLPQGTTL | HLA-A*02:01 |
| | (SEQ ID NO: 14, | |
| | residues 159-167) | |
| Spike | GAALQIPFAMQMAYRF | HLA- |
| protein | (SEQ ID NO: 11, | DRA*01:01, |
| | residues 891- | HLADRB1* |
| | 906) | |
| | MAYRFNGIGVTQNVLY | HLA-DRB1*04:01 |
| | (SEQ ID NO: 11, | |
| | residues 902- | |
| | 917) | |
| | QLIRAAEIRASANLAATK | HLA-DRB1*04:01 |
| | (SEQ ID NO: 11, | |
| | residues 1011- | |
| | 1028) | |
| | FIAGLIAIV | HLA-A*02:01 |
| | (SEQ ID NO: 11, | |
| | residues 1221- | |
| | 1228) | |
| | ALNTLVKQL | HLA-A*02:01 |
| | (SEQ ID NO: 11, | |
| | residues 958- | |
| | 966) | |
| | LITGRLQSL | HLA-A2 I |
| | (SEQ ID NO: 11, | |
| | residues 996- | |
| | 1004) | |
| | NLNESLIDL | HLA-A*02:01 |
| | (SEQ ID NO: 11, | |
| | residues 1192- | |
| | 1200) | |
| | QALNTLVKQLSSNFGAI | HLA-DRB1* |
| | (SEQ ID NO: 11, | 04:01 |
| | residues 957-973) | |
| | RLNEVAKNL | HLA-A*02:01 |
| | (SEQ ID NO: 11, | |
| | residues 1185- | |
| | 1193) | |
| | VLND1LSRL | HLA-A*02:01 |
| | (SEQ ID NO: 11, | |
| | residues 976- | |
| | 984) | |
| | VVFLHVTYV | HLA-A*02:01 |
| | (SEQ ID NO: 11, | |
| | residues 1060- | |
| | 1068) | |
| Membrane | LRGHLRIAGHHLGRC | HLA-DRB4, |
| protein | SEQ ID NO: 13, | HLA-DRB5 |
| | residues 145- | |
| | 159) | |
| | LRIAGHHLGRCDIKD | HLA-DPB1 |
| | SEQ ID NO: 13, | |
| | residues 149- | |
| | 163) | |
| | SRTLSYYKLGASQRV | HLA-DRB1, |
| | SEQ ID NO: 13, | HLA-DRB4, |
| | residues 173- | HLA-DRB5, |
| | 187) | HLA-DQB1, |
| | | HLA-DPA1, |
| | | HLA-DPB1 |
| | SYYKLGASQRVAGDS | HLA-DRB1, |
| | SEQ ID NO: 13, | HLA-DRB3, |
| | residues 177- | HLA-DRB5, |
| | 191) | HLA-DQA1, |
| | | HLA-DQB1 |

The epitopes contained within the peptides described in Table 5B and the other tables herein may be employed in the methods for culturing T cells disclosed herein or for treatment or prevention of disease using T cells recognizing these epitopes.

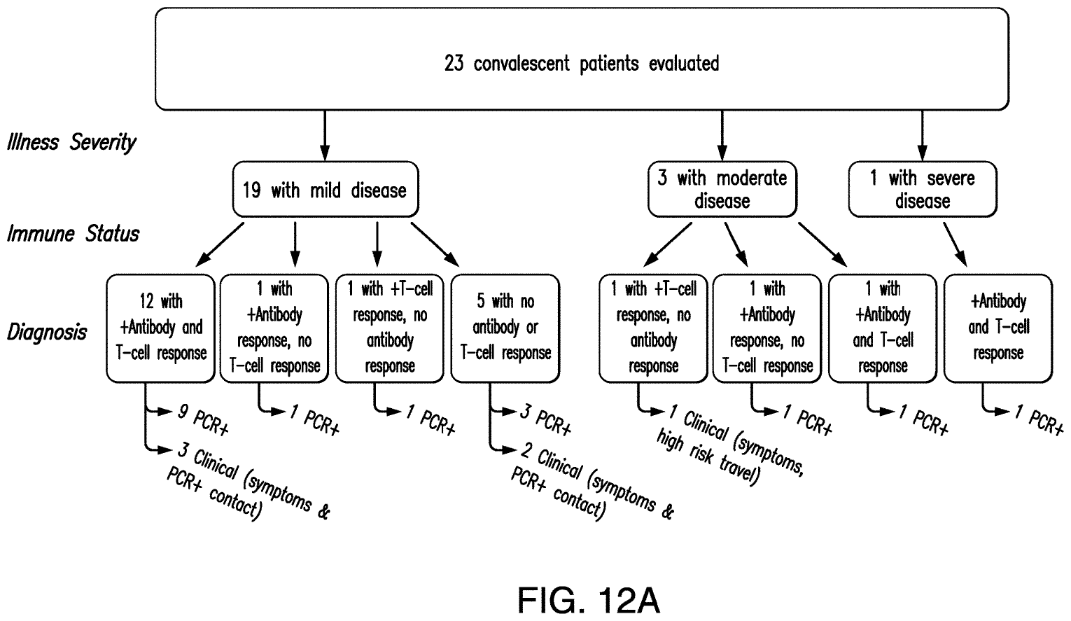

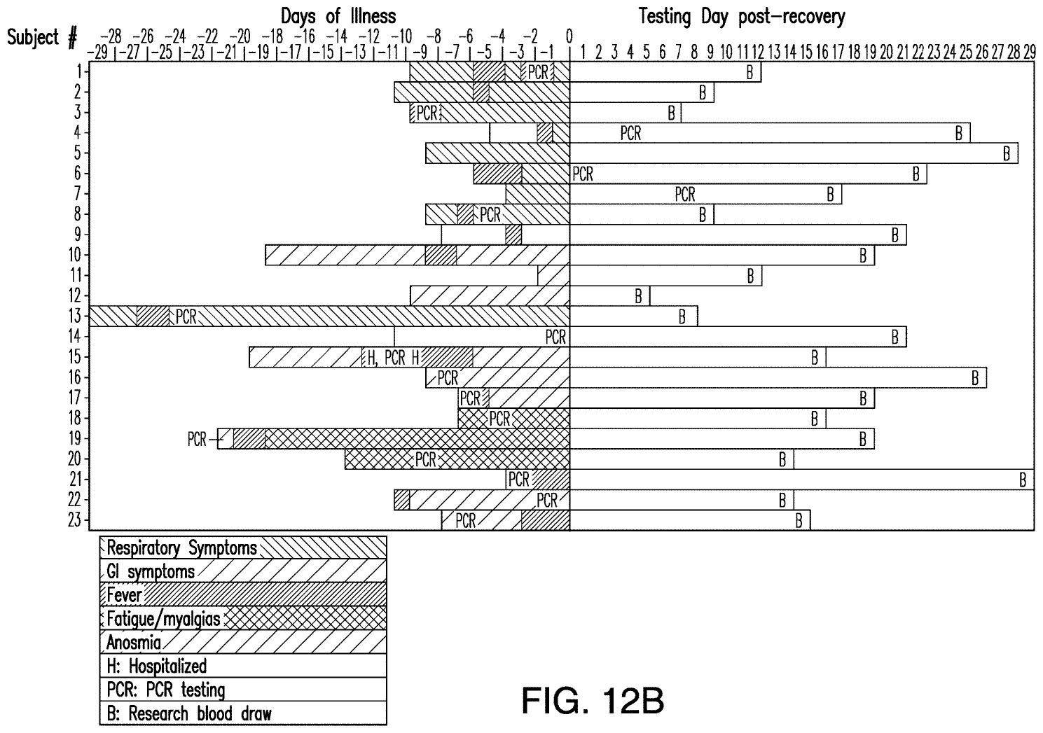

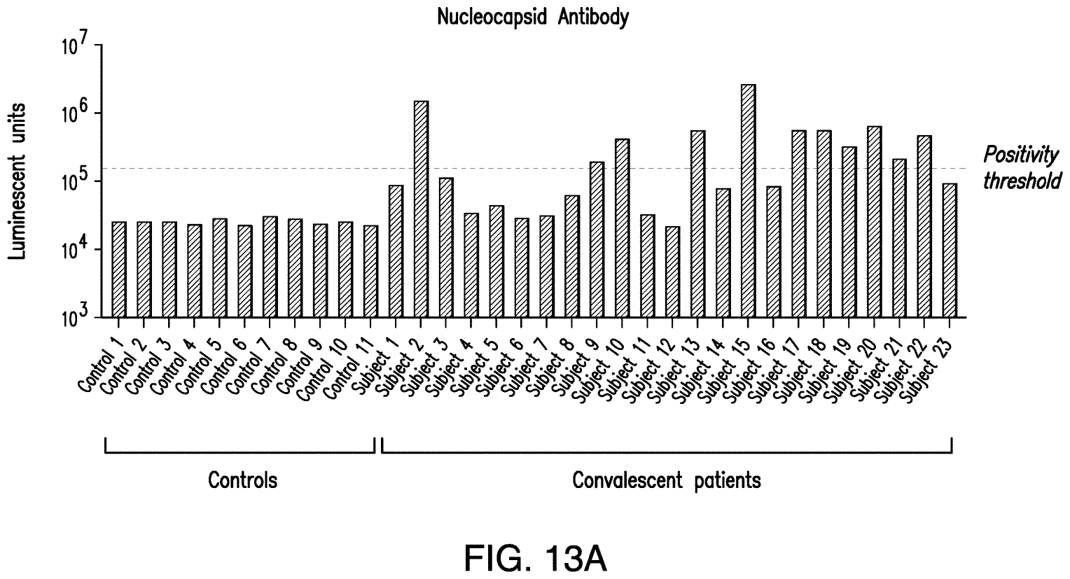

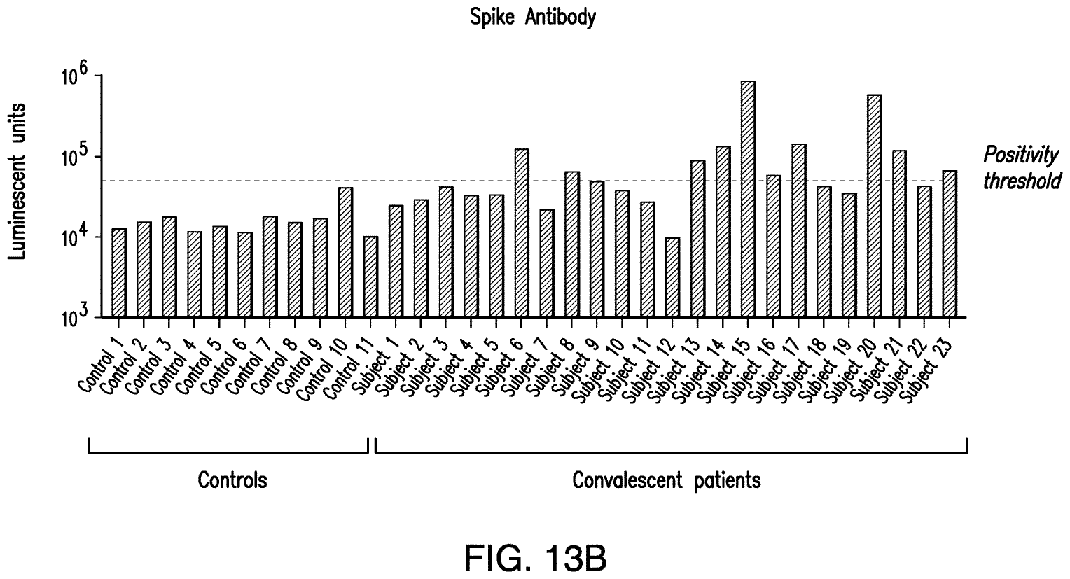

Results. Twenty-three convalescent donors from the eastern and midwestern US with recent COVID-19 (17 PCR proven and 6 probable due to symptoms and positive contacts) were evaluated at a median time of 29 days after symptom onset (range 17-41). Median donor age was 35 years. Most patients had mild disease (83%), whereas 3 had moderate disease and one had severe disease based on WHO classification, with a median of 10 days of illness (Table 6 and FIGS. 12A-12B). Antibody responses were detected in 16 of 23 convalescent donors (14/23 to spike protein, and 11/23 to nucleocapsid protein, FIG. 13A-B), and none of 11 control subjects.

TABLE 6
Convalescent patient demographics.

| Description | Value | |
|---|---|---|
| Median age in years (range) | 35 | (21-69) |
| Male gender | 12 | (52%) |

Disease Severity

| | | |
|---|---|---|
| Mild | 19 | (83%) |
| Moderate | 3 | (13%) |
| Severe | 1 | (4%) |

Symptoms

| | | |
|---|---|---|
| Fever | 15 | (65%) |
| Respiratory symptoms | 17 | (74%) |
| GI symptoms | 4 | (17%) |
| Fatigue | 5 | (22%) |
| Anosmia | 6 | (26%) |

| | | |
|---|---|---|
| Median length of symptoms, days (range) | 10 | (2-30) |
| Need for Hospitalization | 1 | (4%) |

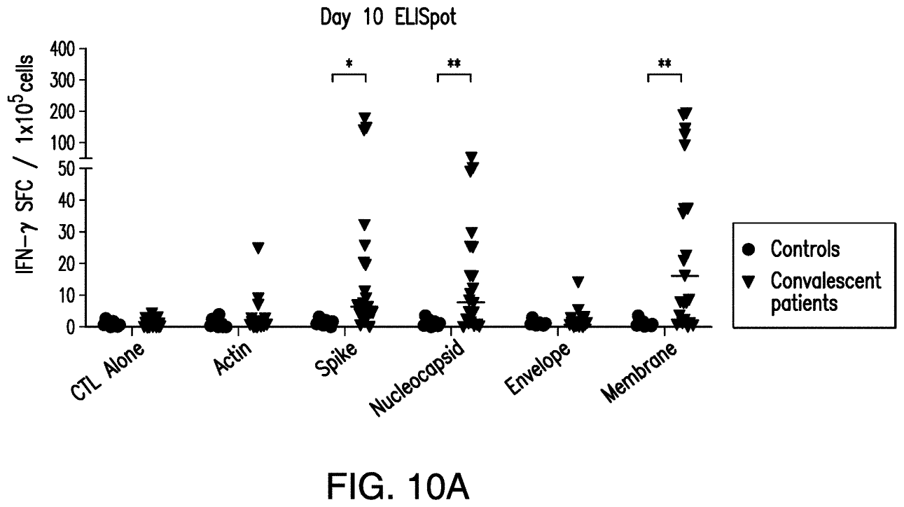

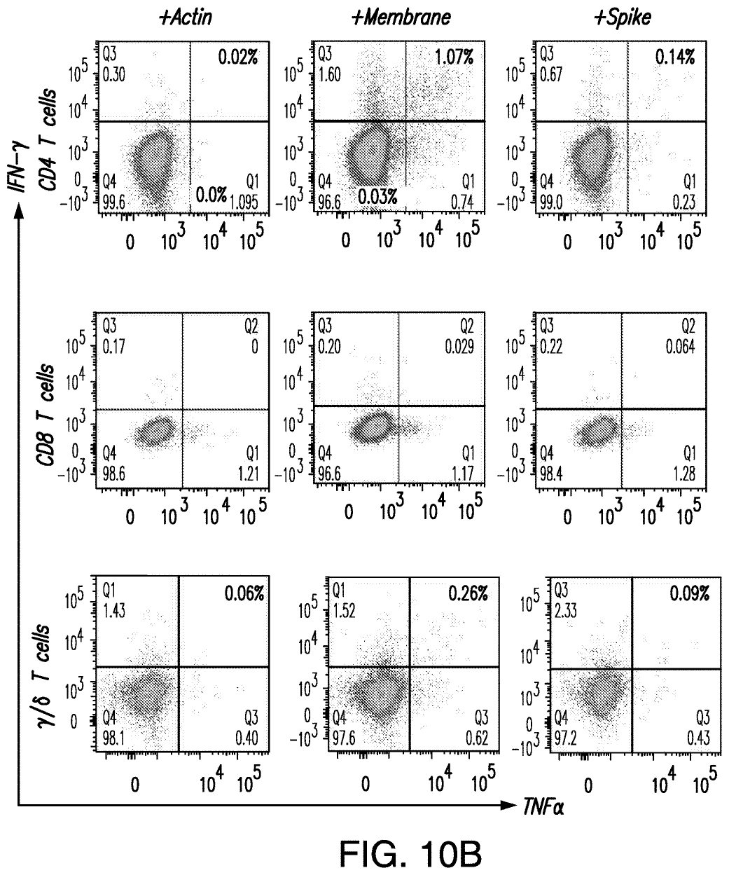

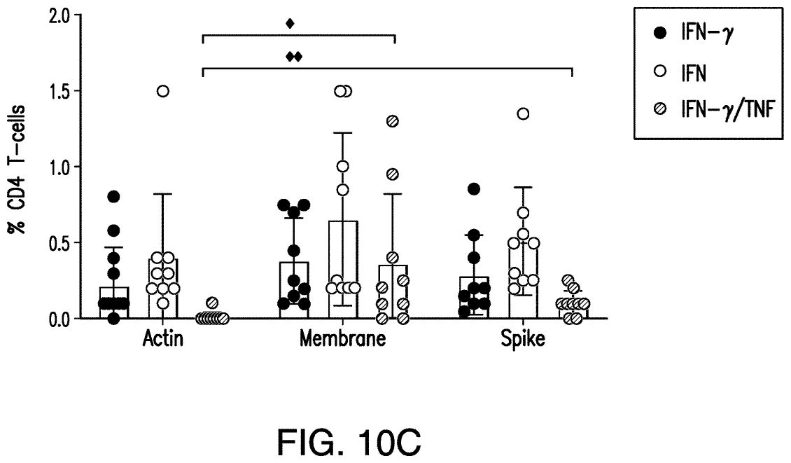

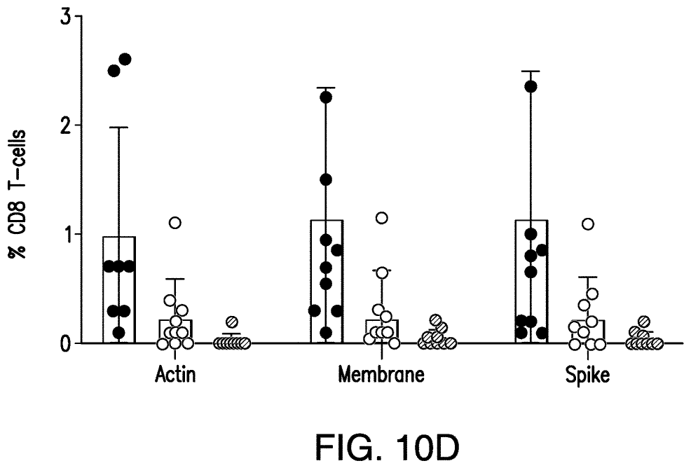

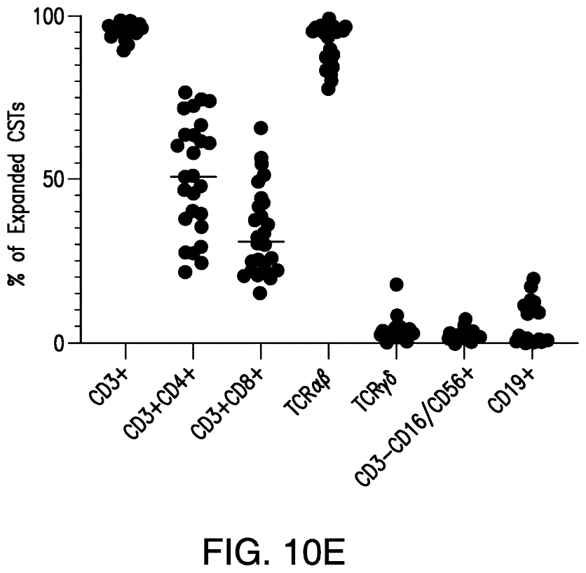

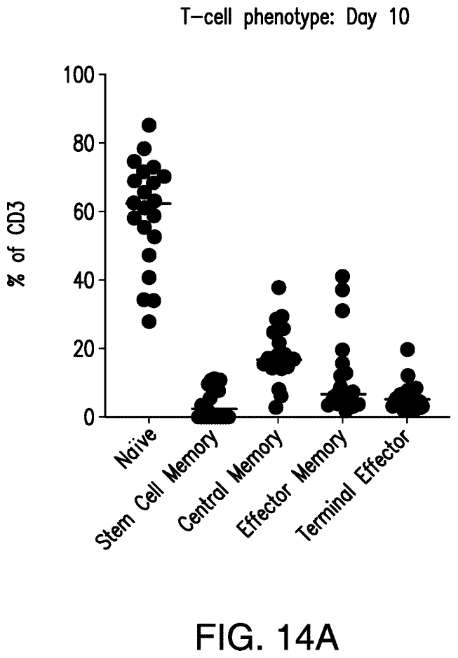

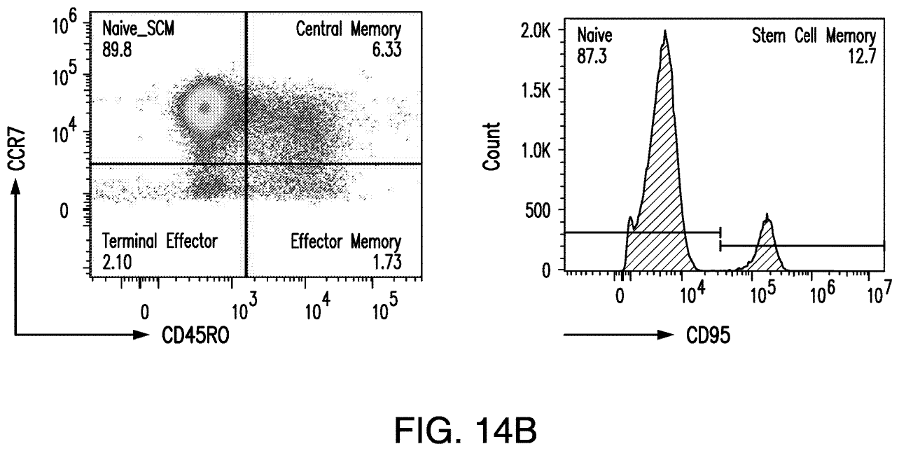

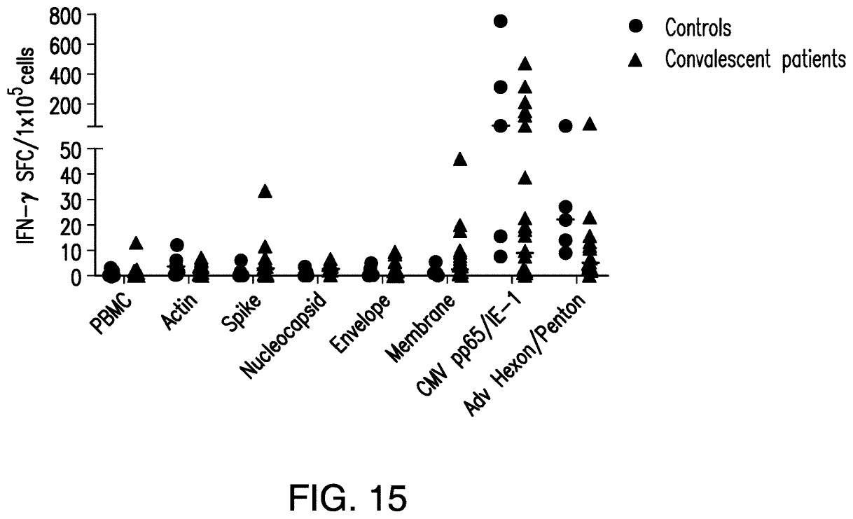

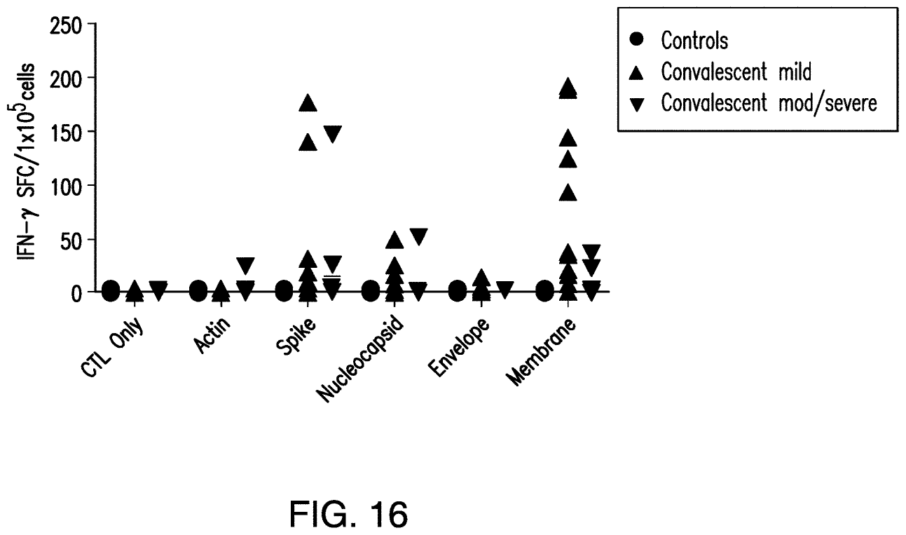

Following stimulation and expansion of CSTs, specific activity against SARS-CoV-2 structural proteins were detectable in 16 of 23 convalescent donors and in none of the control subjects (FIG. 10A) via IFN-γ ELISpot. Convalescent donors responded to membrane (12/23, p=0.003 versus controls), spike (6/23, p=0.021 versus controls), and nucleocapsid (4/23, p=0.002 versus controls) proteins. Expanded cells were predominantly CD4+ T-cells (FIG. 10B), including central memory and effector memory subsets (FIG. 14A-B). Responses to spike and membrane proteins were confirmed to be predominantly CD4 restricted in 9/9 tested patients (FIG. 10B-D), with significant elevations in IFNγ/TNFα-expressing populations targeting membrane and spike proteins (p=0.047 and p=0.008 in comparison to actin, respectively). Non-amplified responses to viral antigens were detectable from PBMCs in only 2 of 27 patients (FIG. 15). Five convalescent donors had no detectable T cell or antibody responses (FIG. 12A-B). Two donors had antibody responses without detectable T cell responses, and two donors had T cell responses without accompanying antibody responses as observed with other infections such as EBV and HSV [55, 56]. A significant association was noted between presence of an antibody response and T cell response to any of the evaluated antigens in convalescent patients (p=0.02 via Pearson Chi-squared test). Although there was no detectable correlation between disease severity and the magnitude of T cell or antibody responses (FIG. 16), 6 of the 9 patients with sub-optimal immune responses had mild disease.

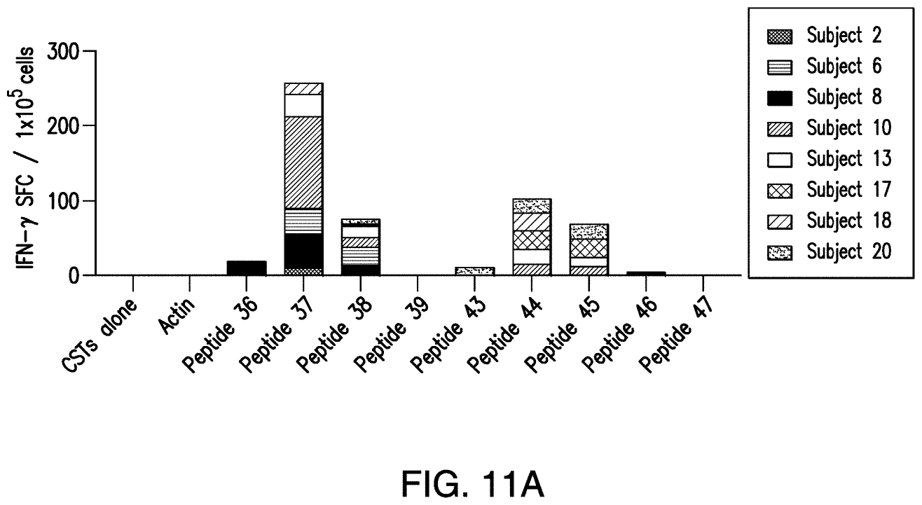

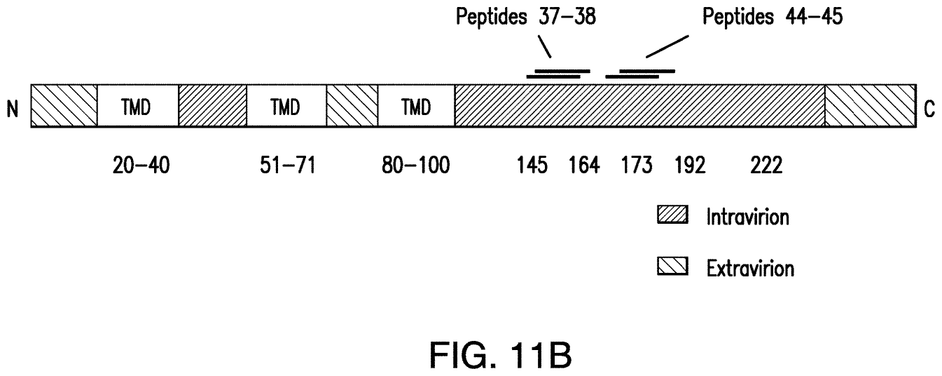

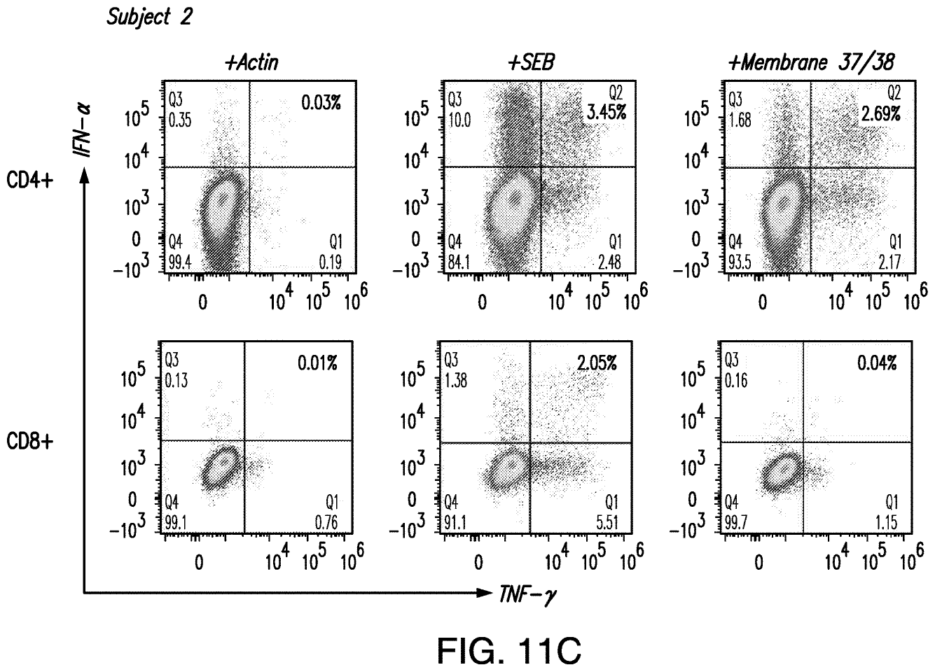

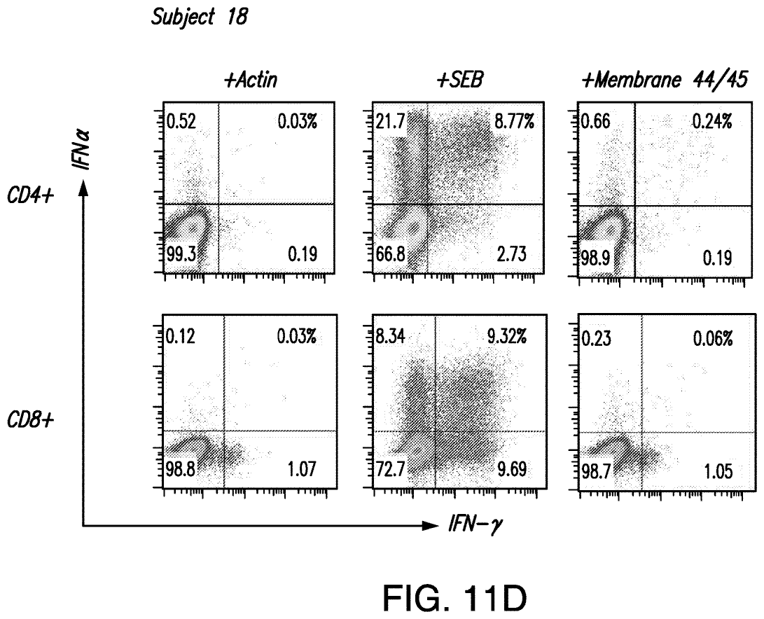

Epitope mapping of the membrane protein, the most abundant structural protein in coronaviruses, yielded multiple epitopes at the C-terminal domain (FIGS. 11A-11B). Two epitopes at AA 144-163 were exclusively CD4-restricted (FIG. 11C), and predicted to be restricted by HLA-DR11 by in silico analysis (Table 7) [57, 58]. Similarly, epitopes at AA 173-192 were recognized by 5 patients, and also confirmed to be CD4-restricted (FIG. 11C). These epitopes all lie within the C-terminal domain which is located inside the virion and on intracellular membranes of infected cells that is a conserved region within all known strains of SARS-CoV2 [56].

TABLE 7
Identified epitopes in membrane protein and predicted HLA restrictions.

| LRGHLRIA | | | | | | | | | | |
|---|---|---|---|---|---|---|---|---|---|---|
| GHHLGRC | | | | | | | | | | |
| (SEQ ID | | | | | | | | | | |
| NO: 13, | | | | | | | | | | |
| residues | | | HLA- | HLA- | HLA- | HLA- | HLA- | HLA- | HLA- | HLA- |
| 145-159) | 145-159 | Subject | DRB1 | DRB3 | DRB4 | DRB5 | DQA1 | DQB1 | DPA1 | DPB1 |
| | | 257 | 07:01, | 02:02 | [symbol] | | 02:01, | 03:01, | 01:03, | 03:01, |
| | | | 11:04 | | | | 05:01 | 03:03 | 02:01 | 03:03 |
| | | 262 | 07:01, | 02:02 | [symbol] | | 02:01, | 02:02, | 01:03, | 04:01, |
| | | | 11:01 | | | | 05:01 | 03:01 | 03:01 | 11:01 |
| | | 265 | 11:04, | 02:02 | [symbol] , | [symbol] | 01:03, | 03:01, | 01:03, | 02:01, |
| | | | 15:02 | | [symbol] | | 05:01 | 06:01 | 02:01 | 14:01 |
| LRIAGHHL | 149-163 | 270 | 11:04, | 02:02 | | | 01:02, | 03:01, | 01:03, | 04:01, |
| GRCDIKD | | | 15:01 | | | | 05:01 | 06:02 | 02:01 | 14:01 |
| (SEQ ID | | | | | | | | | | |
| NO: 13, | | | | | | | | | | |
| residues | | | | | | | | | | |
| 149-163) | | | | | | | | | | |
| | | 257 | 07:01, | 02:02 | 01:03 | | 02:01, | 03:01, | 01:03, | 03:01, |
| | | | 11:04 | | | | 05:01 | 03:03 | 02:01 | 03:03 |
| SRTLSYY | 173-187 | 265 | 11:04, | 02:02 | 01:01, | 01:02 | 01:03, | 03:01, | 01:03, | [symbol] |
| KLGASQRV | | | 15:02 | | 01:03 | | 05:01 | 06:01 | 02:01 | 14:01 |
| (SEQ ID | | | | | | | | | | |
| NO: 13, | | | | | | | | | | |
| residues | | | | | | | | | | |
| 173-187) | | | | | | | | | | |
| | | 262 | [symbol] | 02:02 | [symbol] | | 02:01, | 02:02, | [symbol] , | [symbol] |
| | | | 11:01 | | | | 05:01 | [symbol] | 03:01 | 11:01 |
| | | 265 | 11:04, | 02:02 | [symbol] , | 01:02 | 01:03, | [symbol] , | 01:03, | [symbol] |
| | | | [symbol] | | [symbol] | | 05:01 | 06:01 | 02:01 | 14:01 |
| | | 269 | 12:01, | 02:02 | [symbol] | 01:01 | 01:02, | [symbol] , | 01:03, | [symbol] |
| | | | [symbol] | | | | 05:01 | 05:01 | 01:03 | 04:01 |
| | | 270 | 11:04, | 02:02 | | | 01:02, | [symbol] | 01:03, | [symbol] |
| | | | [symbol] | | | | 05:01 | 06:02 | [symbol] | 14:01 |
| SYYKLGA | 177-191 | 273 | 03:02, | 01:62 | [symbol] , | [symbol] | 01:02, | [symbol] | 02:02, | 01:01, |
| SQRVAGDS | | | [symbol] | | [symbol] | | 04:01 | 05:02 | 02:02 | 01:01 |
| (SEQ ID | | | | | | | | | | |
| NO: 13, | | | | | | | | | | |
| residues | | | | | | | | | | |
| 177-191) | | | | | | | | | | |
| | | 262 | 07:01, | [symbol] | 01:01 | | [symbol] | 02:02, | 01:03, | 04:01, |
| | | | [symbol] | | | | 05:01 | 03:01 | 03:01 | 11:01 |
| | | 269 | 12:01, | [symbol] | 01:01 | [symbol] | 01:02, | 03:01, | 01:03, | 02:01, |
| | | | 15:01 | | | | 05:01 | 05:01 | 01:03 | 04:01 |
| LRGHLRIA | 145-159 | 273 | 03:02, | 01:62 | 01:01, | 02:02 | [symbol] , | [symbol] | 02:02, | 01:01, |
| GHHLGRC | | | [symbol] | | 01:03 | | [symbol] | [symbol] | 02:02 | 01:01 |
| (SEQ ID | | | | | | | | | | |
| NO: 13, | | | | | | | | | | |
| residues | | | | | | | | | | |
| 145-159) | | | | | | | | | | |

Bold: strong binder (<2).
Bold italic: Weak binder (2-10)

Discussion. Advancing knowledge of the immune response to SARS-CoV-2 is critical at the current juncture, in order to guide candidate vaccine studies as well as to identify novel therapeutic targets for T cell therapies. Multiple studies have focused on the antibody response following COVID-19, but T-cell responses are known to endure for years in response to SARS-CoV and MERS-CoV [54, 60]. This study presents the first demonstration that T-cell responses targeting multiple structural proteins of SARS-CoV-2 are common following recovery from COVID-19, and target multiple viral structural proteins with identified class II restricted epitope “hot spots.” Membrane, spike, and nucleocapsid proteins showed a clear hierarchy of immunodominance, and were associated with significant increases in IFNγ/TNFα producing CD4⁺ T-cell populations. Though the role and biologic significance of these cell populations in combating SARS-CoV-2 is not fully clear, the importance of polyfunctional CD4 T-cell responses are well-documented in many other respiratory viruses. The efficacy of adoptive, predominantly MHC class II-restricted, T-cell therapies targeting adenovirus in immunocompromised patients is a prime example of the importance of T-cell immunity for clearance of respiratory viruses [61].

Multiple regions within the highly conserved C-terminal domain of the membrane protein elicited CD4-restricted responses that were shared by multiple individuals. In silico analysis suggested restriction of these epitopes through HLA-DR11, DR7, DQ3, and DQ7 which are present in roughly 50% of the population [62].

Overall, 70% of the evaluated convalescent donors had demonstrable T-cell responses to one or more structural viral proteins, and an association was detected between the presence of antibody and T-cell responses. Interestingly, not all convalescent donors had detectable humoral and cellular responses, and some incongruous responses were noted. In patients who recovered from SARS-CoV, disease severity was noted to correlate with the magnitude of CD4 T-cell response [54], and it is possible that the magnitude of cellular immune responses may similarly relate to severity in subjects with COVID-19.

Recent studies of unexpanded peripheral blood samples identified both CD4 and CD8-restricted responses to viral structural proteins in convalescent donors, albeit at low levels [48-50]. However, these studies also demonstrated low level responses in unexposed donors, which were postulated to be due to cross-reactivity with common circulating coronaviruses. In contrast, the approach of expanding T cells ex vivo, prior to measurement, presented here yielded robust responses in most infected individuals, alongside a complete absence of T-cell responses in unexposed control subjects (in agreement with antibody responses). Although rare T-cell populations recognizing SARS-CoV-2 proteins in virus-naïve donors cannot be definitively ruled out, the absence of such responses in this study, even following ex vivo expansion with immunodominant antigens, calls into question whether there is meaningful cross-reactive adaptive immunity to SARS-CoV-2 in unexposed individuals. Larger, longitudinal studies of cellular response to other coronaviruses and their possible cross-reactivities with SARS-CoV-2 will therefore be necessary to address this possibility.

Limitations of this study include the sample size, and the tendency toward mild illness in the subjects. However, as the vast majority of the convalescent donors had uncomplicated disease, the data suggests that T-cell and humoral responses measured here represent an effective adaptive immune response to SARS-CoV-2. Subjects were not tested longitudinally, and therefore the absence of T-cell responses in 30% of subjects may relate to the timing of T-cell responses following primary infection. Evaluation was limited to structural viral proteins, given their described immunodominance in related coronaviruses, but it is possible that T-cell responses to non-structural proteins may also occur. Lastly, a maladaptive immune response is highly suspected to be the cause of hyper-inflammatory complications such as multisystemic inflammatory syndrome in children [63], and an understanding of the role of adaptive and innate responses in patients with inflammatory complications will be critical in determining the characteristics of an effective and enduring adaptive immune response to SARS CoV-2. This consideration also affects whether adoptive T-cell immunotherapy may be safe and beneficial for immunocompromised patients with COVID-19.

In summary, it is demonstrated for the first time that the majority of convalescent patients have T-cell immunity to SARS-CoV-2 structural proteins, principally CD4+ responses to conserved regions of the membrane protein. The immunodominance of the membrane protein therefore has important implications for vaccine development to elicit cellular immune responses, as most current vaccine candidates are focused exclusively on the spike protein to elicit neutralizing antibody. It is also shown for the first time that ex vivo expansion of coronavirus-specific T-cells (CST) is feasible using a GMP-compliant methodology which will allow the rapid translation of this novel therapeutic to the clinic. Future studies will therefore evaluate whether “off the shelf” adoptive T-cell immunotherapy using this novel CST product may emerge as a useful treatment modality in high-risk patients with COVID-19, as it has been effectively shown for the treatment of other respiratory viruses [64].

### Example 4

### IL-15/IL-6 Function, IL-15/IL-7 Function, and IL-7/IL-4 Function

Results. Using the methods and materials disclosed in Example 1, the following data were observed.

Representative data examining high throughput analysis of cytokine combinations. Data were collated from individual plates to evaluate the growth and function of cells under different cytokine conditions. A representative set of data from one sample is collected. For this sample, it was found that IL-15 supported survival of CD3+ T cells when used at the highest dose of 10 ng/mL in combination with other cytokines including IL-6, IL21, or IFNα. Culture of PBMCs without additional cytokine did not support T cell growth, with a mean of only 2432 viable CD3+ cells recovered. Culture of PBMCs in IL-15 alone induced substantial T cell growth, with an average of 54,788 viable CD3+ T cells recovered the top dilution of 10 ng/mL of IL-15, representing a 22.5-fold increase over wells without cytokine. Decreasing the concentration of IL-15 substantially reduced the expansion of CD3+ T cells for this sample, with an average of 11,333 viable CD3+ T cells (4.7-fold increase over no cytokine) recovered in wells cultured with 2 ng/mL IL-15, and only 4555 viable CD3+ T cells recovered in wells cultured with 0.4 ng/mL IL-15 (1.9-fold increase over no cytokine).

Addition of IL-6 to wells containing IL-15 did not appear to substantially alter CD3+ T cell expansion, with an average of 63,814 viable CD3+ T cells recovered in wells with 100 ng/mL IL-6+10 ng/mL IL-15 (26.2-fold increase over no cytokine). Dilution of IL-6 did not appear to consistently alter the recovery of CD3+ T cells, as one set of replicates remained above 60,000 cells while the second set of replicates slightly decreased to 18,151 cells in this sample. Addition of IFNα inhibited CD3+ expansion, with only 18,747 viable CD3+ T cells on average recovered from wells incubated with 10,000 U/mL of IFNα (7.7-fold increase). Reducing the IFNα restored CD3+ expansion, increasing the recovered cell count to 54,487 cells on average (22.4-fold increase) at the lowest dilution of IFNα, suggesting IFNα was having an inhibitory effect on the outgrowth of viral specific CD3+ T cells. Addition of high concentrations of IL21 decreased CD3 expansion, as 44,462 viable CD3+ T cells (18.3-fold increase) were recovered on average in wells cultured with 100 ng/mL of IL21 and 10 ng/mL of IL-15. This increased to 59,799 cells on average (24.6-fold increase) as IL21 was diluted to 4 ng/mL. CD3+ T cell expansion was also significantly reduced in wells with lower concentrations of IL-15 regardless of the presence of additional cytokines IL-6, IL21, or IFNα. Also, culture with IL-6, IL21, or IFNα alone was not sufficient to stimulate T cell expansion in the absence of IL-15. CD3+ T cell expansion in the presence of IL-4 and IL-7 (400 U/mL and 10 ng/mL) was less than seen with cells cultured in IL-15 for this sample, with an average of 29,998 CD3+ cells recovered (12.3-fold increase over no cytokine controls). Lower concentrations of IL-4+IL-7 reduced the expansion of CD3+ T cells, as 7837 cells were recovered on average (3.2-fold increase) in culture with 16 U/mL IL-4 and 0.4 ng/mL IL-7. Intermixing IL-6, IL21, and IFNα did not produce appreciable CD3+ expansion, with the greatest expansion found in wells cultured in IL21 and IL-6 (7002 cells for 100 ng/mL each; a 2.9-fold increase), however this amount was still substantially below wells cultured in the presence of IL-15 or IL-4/IL-7.

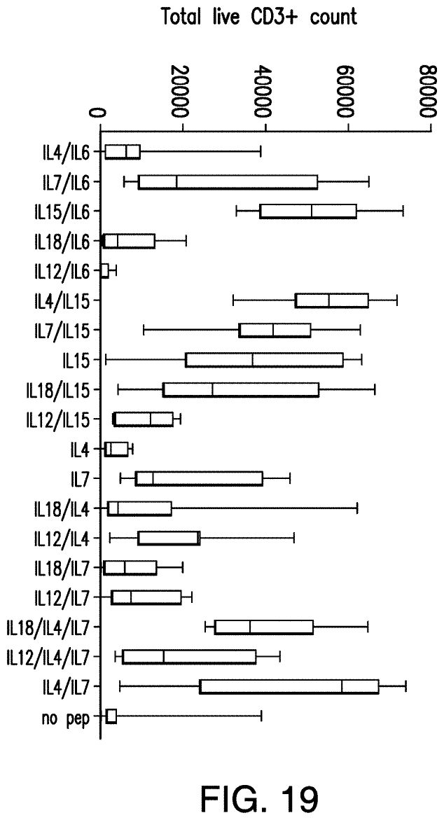

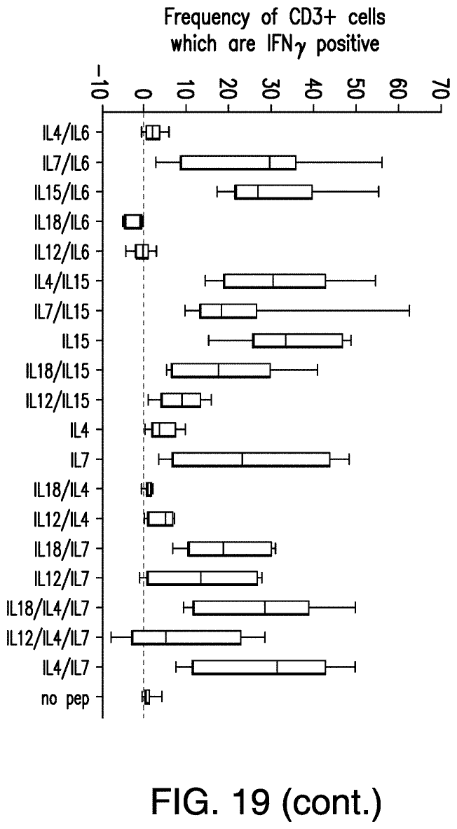

The combination of IL-15 (10 ng/mL) and IL-6 (100 ng/mL) was selected for further investigation based on the favorable expansion of CD3+ T cells and their cytokine production as seen in four of the samples tested compared with other cytokine combinations (FIG. 17). The plate layout was further modified to investigate additional replicates of IL-15 and IL-6, new cytokine combinations with IL12 and IL18 while removing IL21 and IFNα cytokines, and new combinations of 11-4, IL-15, IL-6, and IL-7 with each other (FIG. 19). These added investigations identified that IL12 was strongly inhibitory for CD3+ T cell expansion and production of IFNγ, while IL18 was less inhibitory. Addition of IL-15 or IL-7 was sufficient for expansion of CD3+ T cells, and that the original selection of IL-15+IL-6 remained superior when compared with other combinations. Overall, culture in a combination of IL-15/IL-6 consistently promoted CD3+ T cell expansion and IFNγ production to levels similar to culture in IL-4/IL-7.

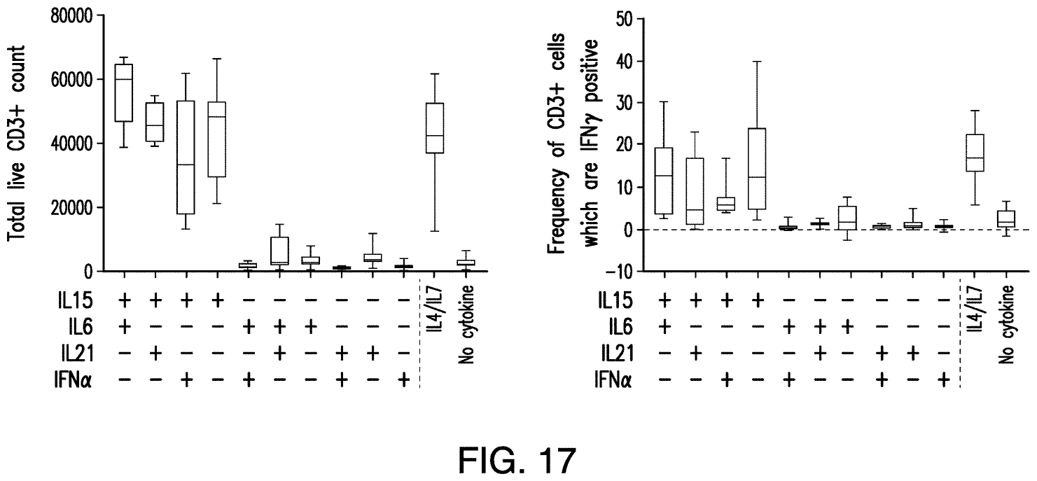

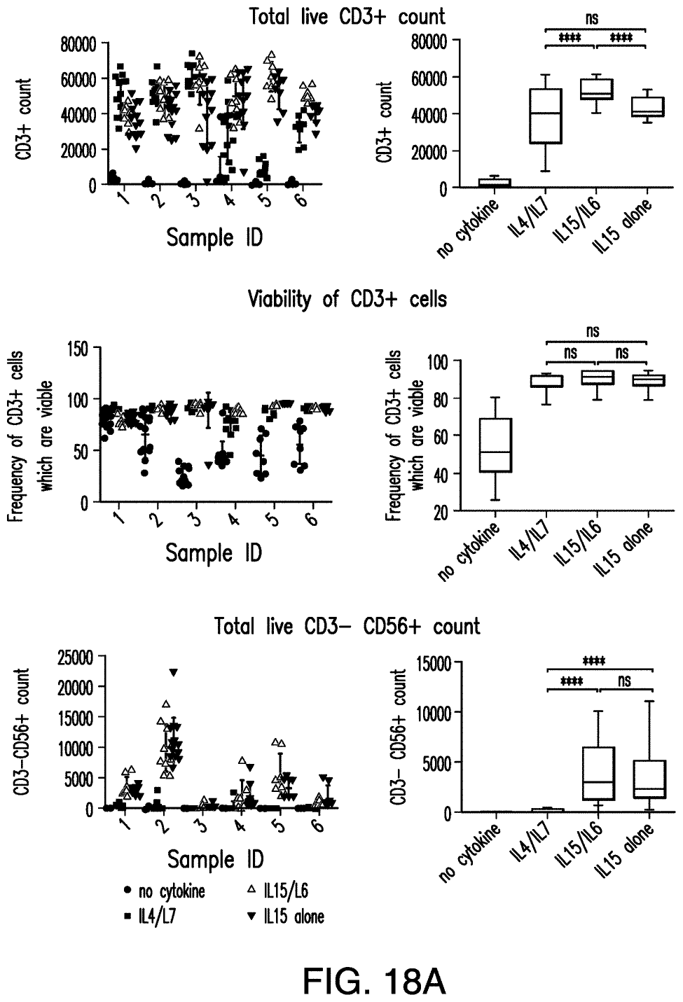

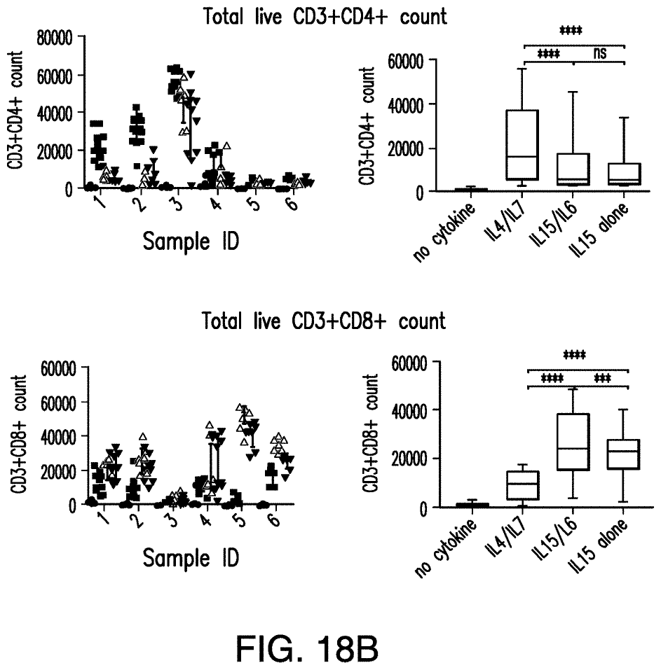

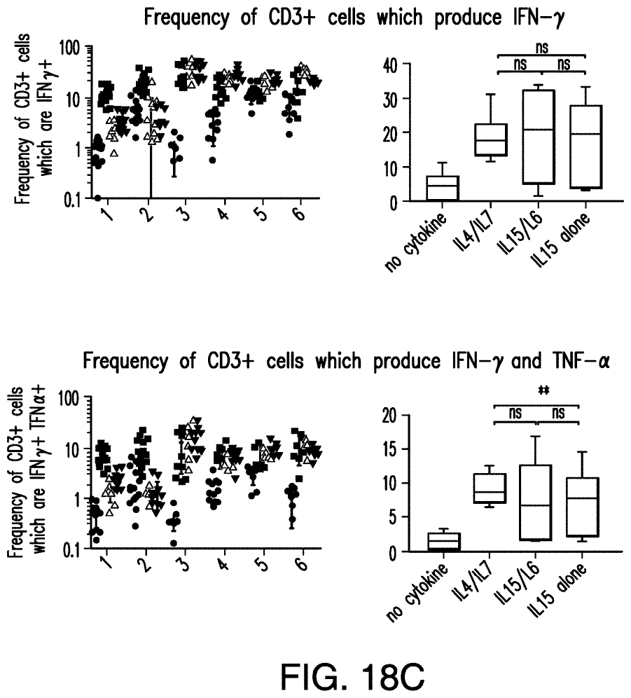

Selective cytokine culture imparts bias on the ratio of CD4 vs CD8 cells in viral specific T cell products. All replicates were averaged combining IL-15 and IL-6 cytokines (n=52 wells) and compared them with growth in IL-4 and IL-7 (400 U/mL and 10 ng/mL; n=68 wells), IL-15 alone (n=68 wells), and no cytokine controls (n=68 wells) for the six independent CMV reactive patient samples (FIG. 18). Overall, culture in IL-15 and IL-6 expanded VST cells was comparable to that seen when cells were cultured in IL-4 and IL-7. Culture in IL-15/IL-6 expanded 18.8-fold more CD3+ cells on average as compared with no cytokine controls (FIG. 18A; 52,146 cells vs 2775 cells). Culture in IL-4/IL-7 expanded 14.0-fold more CD3+ cells on average compared with no cytokine controls (FIG. 18A; 38,838 vs 2755 cells). Viability of CD3+ cells on average was not significantly different between wells containing IL-15/IL-6 or IL-4/IL-7, averaging 90% and 89%, respectively. NK cells were present in cultures expanded with IL-15+IL-6, averaging 3878 cells, which was 6.2% of total cells recovered (FIG. 18A). Less than 300 NK cells were recovered on average from wells containing IL-4/IL-7, representing 0.6% of total cells recovered.

Interestingly, a strong bias was identified in the ratio of CD4+ to CD8+ cells in the final product depending on the cytokines used for initial culture. Culture in IL-15 and IL-6 favored outgrowth of CD8+ T cells compared with culture in IL-4 and IL-7, which favored outgrowth of CD4+ T cells. Culturing cells in IL-4/IL-7 expanded 81% more CD3+CD4+ T cells than when cells were cultured in IL-15/IL-6 (FIG. 18A; 21,205 vs 11,696 CD4+ cells). Culturing cells in IL-15/IL-6 more than doubled the expansion of CD3+CD8+ cells compared with IL-4/IL-7, with 176% more CD8+ cells on average in IL-15/IL-6 vs IL-4/IL-7 (FIG. 18A; 26,412 vs 9551 CD3+CD8+ cells). The viability of CD4+ and CD8+ cells was also not significantly different comparing culture in IL-15/IL-6 and IL-4/IL-7 (data not shown; CD4 viability p=0.4381; CD8 viability p=0.1033), indicating the different cytokine combinations were stimulating outgrowth of CD4+ vs CD8+ cells rather than preserving the selective survival of individual subsets. This substantial difference in the ratio of helper vs. cytotoxic cells within the final cell product demonstrates a potential avenue for purposeful skewing of cell therapy products which was previously unrecognized.

Both culture conditions expanded CD3+ T cells which produced IFNγ in response to CMV peptide re-stimulation. An average of 24% of CD8+ T cells produced IFNγ in response to antigenic peptide in wells cultured in the highest concentration of IL-15+IL-6, while approximately 30% of CD8+ T cells cultured in the highest concentration of IL-4+IL-7 recognized CMV peptide (FIG. 18C). Over 30% of CD4+ T cells also recognized CMV peptide when cultured in IL-15+IL-6, as compared with under 20% of CD4+ T cells cultured in the highest concentration of IL-4+IL-7. The proportion of multi-cytokine producing cells was investigated as evidence that these cells offer superior protection against viral infection as compared with single cytokine producing cells. An average of 4.0% of CD3+ cells cultured in IL-15/IL-6 produced both IFNγ and TNFα in response to CMV peptide, while 8.0% of cells cultured in IL-4/IL-7 produced both IFNγ and TNFα in response to CMV peptide (FIG. 18C). Overall, our analysis identified that culture in IL-15 and IL-6 produced an equivalent number of CMV reactive CD3+ VSTs which were less skewed towards a CD4+ phenotype as compared with culture in IL-4 and IL-7.

T cells produced as cellular therapy products are effector memory in phenotype. The lower frequency of CMV specific CD4+ T cells within IL-4/IL-7 culture indicated IL-4 can be preserving a large fraction of non-specific CD4+ T cells within final products. To investigate this, surface expression of markers associated with T cell memory in addition to intracellular cytokine staining was measured as part of the comprehensive panel. Cells were stained with antibodies specific for CCR7 and CD45RO as primary indicators of memory, along with CD95, CD28, and CD45RA to investigate how the different cytokine combinations affected the evolution of memory in the products.

Pre-culture naïve/stem cell memory cells comprised 32% of CD3+ T cells on average, with effector memory cells comprising the next highest fraction with 30.4% of cells on average. Central memory cells were 22% of the CD3+ population on average, and terminal effectors were the smallest fraction of CD3+ T cells on average (15.4%). Further analysis of CD4+ and CD8+ subsets identified helper and cytotoxic specific staining patterns as shown in one representative sample. For this sample, 44% of CD4+ T cells displayed a naive/stem cell memory phenotype (CCR7+CD45RO−) compared with 13% of CD8+ T cells. More CD4+ T cells were also central memory (37%; CCR7+CD45RO+) than effector memory (18%; CCR7− CD45RO+). CD8+ T cells reversed this pattern, with 27% of cells displaying an effector memory phenotype compared with 9% displaying central memory phenotype. Finally, less than 1% of CD4+ T cells were terminal effectors (CCR7− CD45RO−), while 51% of CD8+ T cells were terminal effectors.

Once cells were cultured for ten days with peptide and cytokines, the majority of viable cells recovered from wells which displayed significant T cell growth were effector memory in surface phenotype. 72.3% of cells grown in IL-4/IL-7 were effector memory on average, compared with 76.9% of cells grown in IL-15/IL-6. The next highest fraction of cells were identified as terminal effector cells lacking both CCR7 and CD45RO, 11.3% in IL-4/IL-7, and 14.3% in IL-15/IL-6. Central memory cells were also low in number after culture, with 9.3% in IL-4/IL-7, and 6.6% in IL-15/IL-6 on average. The only significant difference in phenotype was in the frequency of naive cells, where IL-4/IL-7 had significantly more naive cells (7.0%) compared with IL-15/IL-6 (2.3%).

The memory phenotype of antigen specific cells and antigen non-responsive cells within the same well was determined by IFNγ production in response to peptide. CD3+ IFNγ+ cells were also predominantly effector memory in phenotype, while a small but recurrent fraction of naïve cells were identified within the antigen non-reactive fraction (IFN-7 negative) in cells cultured with IL-4 and IL-7 (4.2%) which was lower than when cells were grown in IL-15 and IL-6 (1.7%). These naïve cells were CD4+, indicating that culture in IL-4/IL-7 was preserving a subset of naïve cells within the final product.

Cells grown in Grex-10 flasks with IL-15/IL-6 produce similar amounts of IFNγ compared to IL-4+IL-7 in antigen specific ELISPOTs. Whether culture of cells with IL-15 and IL-6 within Grex-10 culture vessels would recapitulate the data observed when cultured within 96 well plates was investigated. At least 1×10⁷ cells were seeded in Grex-10 culture vessels with media and either IL-4/IL-7 at 400 U/mL and 100 ng/mL or IL-15/IL-6 at 10 ng/mL and 100 ng/mL plus IE1 and pp65 peptides at 1 μg/mL. Cells grown in IL-15/IL-6 produced an average of 646 spots per 100,000 added cells after re-stimulation with IE1 and pp65 peptide, while cells cultured in IL-4 and IL-7 produced an average of 621 spots per 100,000 added cells in response to CMV peptide re-stimulation. There were no significant differences in the antigen specific responses across the range of ELISPOT conditions when comparing cells grown in IL-4/IL-7 to cells grown in IL-15/IL-6 for the five samples tested. The CMV response was also antigen-specific, as cells produced less than 10 spots on average in response to either actin or no peptide controls per 100,000 added cells. This demonstrated that cells grown in IL-15/IL-6 were functionally equivalent to cells grown in IL-4/IL-7 when cultured within the normal Grex-10 culture vessels, and that cytokine conditions identified within the high throughput screen can be further advanced during process development to clinical stage conditions.

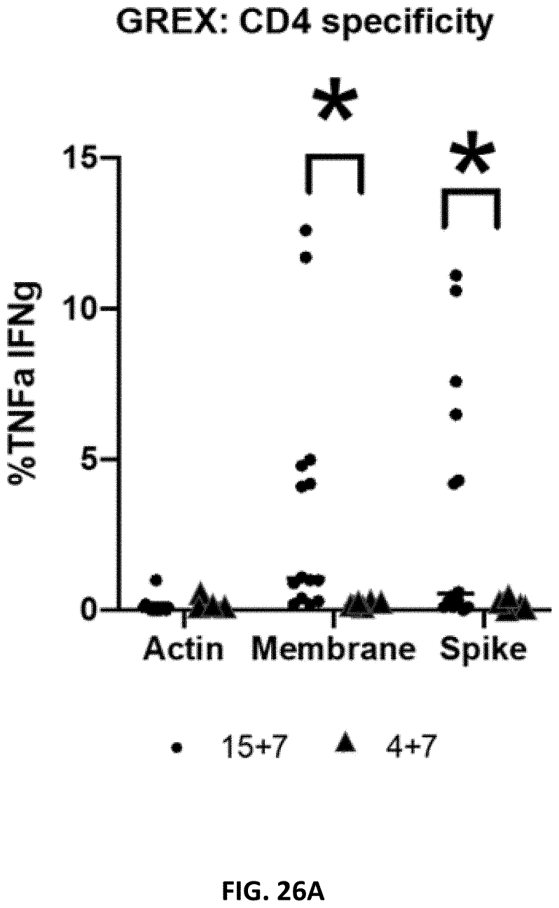

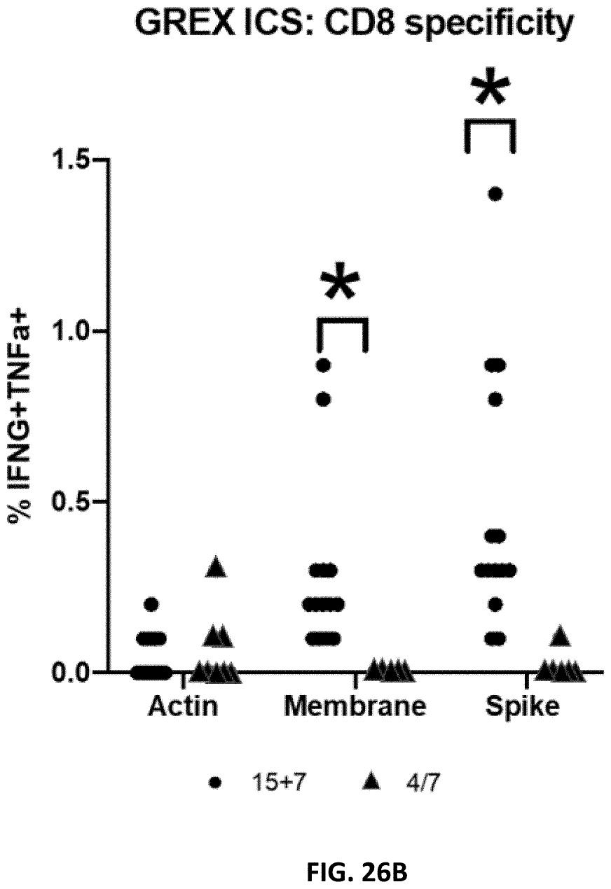

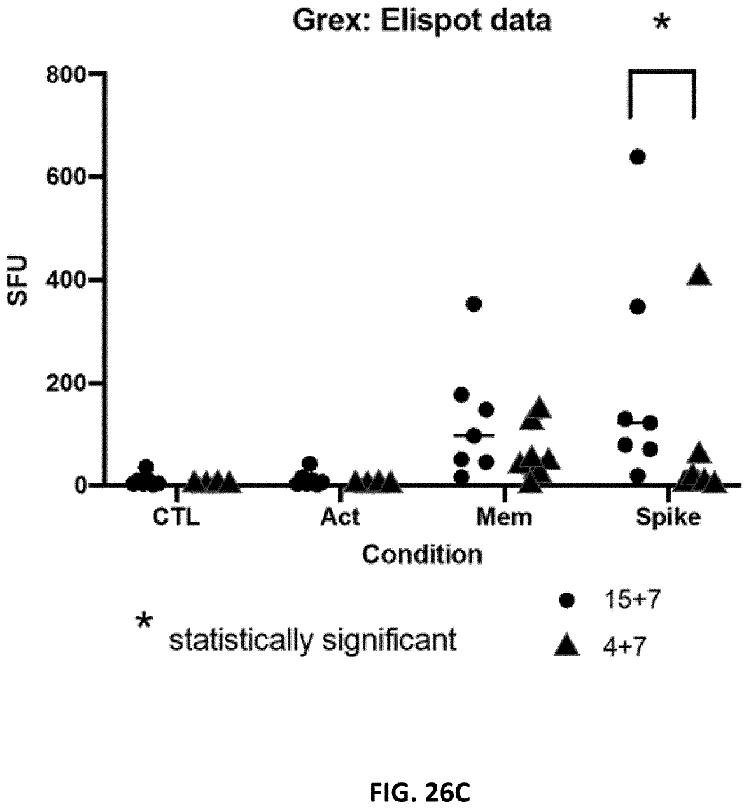

The use of IL-15/IL-7 to maximize specificity (CD4+ and CD8+) and cell expansion compared to IL-4/IL-7 in Grex expansion. Total counts of cell expansion for CD3+, CD4+ or CD8+ T cells were conducted using cytokine conditions: IL-4-2000 U/mL, 1:2000 dilution (200 uL final vol=0.1 uL per well); IL-15-5 ug/mL, 1:1000 dilution (200 uL final vol=0.2 uL per well); IL-7-10 ug/mL, 1:1000 dilution (200 uL final vol=0.2 uL per well); IL-6-100 ug/mL 1:1000 dilution (200 uL final vol=0.2 uL per well) with the combinations of (1). no cytokine, (2). IL-4 and IL-7 (IL-4/7), (3). IL-15, (4). IL-15 and IL-4 (IL-15/4), (5). IL-15 and IL-6 (IL-15/6), and (6). IL-15 and IL-7 (IL-15/7). Expanded in microexpansion format by initially choosing 2 lines with prior CD4+ specificity to membrane and spike proteins, then choosing 3 lines with some faint CD8+ specificity on prior microexpansion. Flow cytometry was performed on Day 10 by comparing to IL-4/7 (standard condition). IL-15/7 showed preserved total CD3+ cell counts. Mildly decreased total CD3+ cell counts were seen in IL-15, IL-15/4, and IL-15/6. IL-15/4 and IL-15/7 showed preserved CD4+ cell counts. Decreased CD4+ cell counts were seen in IL-15 and IL-15/6. Increased CD8+ cell counts were seen in IL-15/6 and IL-15/7. Regarding specificity on flow cytometry for CD4+, IL-15/7 appeared to maximize CD4+ specificity comparing the other combinations. Regarding specificity on flow cytometry for CD8+, IL-15 and IL-15/6 showed highest CD8+ specificity. IL-15/7 showed high specificity in some lines comparing to IL-4/7. Grex expansion was used to validate cell expansion and maximization of specificity of CD4+ and CD8+ by comparing IL-15/7 and IL-4/7. 7 lines were used for the comparison between IL-4/7 and IL-15/7 on expansion and specificity. Expanded in Grex10 Format. Seeded 10-15E6 on day 0. Fed on Day 7. Harvested on Day 10. Flow cytometry and Elispot was performed on Day 10. IL-15/7 has mean fold expansion 3.2×. IL-4/7 has mean fold expansion 1.9×. p=0.08. FIG. 26A-C show ICS (CD4+ and CD8+) and ELISpot specificities. FIG. 26A shows significantly increased CD4+ specificity using IL-15/7 compared to IL-4/7 in Grex validation. FIG. 26B shows increased CD8+ specificity using IL-15/7 compared to IL-4/7 in Grex validation. FIG. 26C shows increased specificity using IL-15/7 compared to IL-4/7 in Grex-ELISpot. In conclusion, IL-15/7 showed increased CD4+ and CD8+ specificity compared to IL-4/7.

Discussion. In this study, a high throughput flow cytometric assay was utilized to evaluate dozens of different cytokine combinations for growth of viral specific T cells from different donor PBMCs. The experiments identified clear preferences for expansion and T cell effector function for cells cultured in IL-4/IL-7, and in IL-15/IL-6. Culture with IL-4/IL-7 favors expansion of CD4+ T cells at the expense of CD8+ T cells, while culture with IL-15/IL-6 expands a both CD8+ and CD4+ T cells. Finally, it was demonstrated that the IL-15/IL-6 cytokine growth condition identified in this high throughput screens was adaptable to normal G-rex 10 cultures, producing cells functionally equivalent to IL-4/IL-7 for IFN-7 production.

These investigations demonstrated the utility of miniaturized flow cytometric assays for evaluating high throughput cell cultures. By utilizing flow cytometry to test for efficacy, it was possible to seed cultures with 1×10⁵ cells per well, totaling 1×10⁷ cells and interrogating up to 40 cytokine combinations per plate. Favorable cytokine combinations were re-investigated with additional replicates in subsequent experiments, ultimately using only 3×10⁷ total PBMCs to measure 90 total cytokine combinations. Importantly, both the culture conditions and the functional assay were modular by design, allowing for simple exchange of new cytokine combinations into the culture layout as inferior conditions were removed, as well as new validated flow cytometric markers into the functional assay. This flexibility clearly benefits process development with its emphasis upon speed, replicate reproducibility, and efficient use of limited starting product.

Of the newly tested cytokine combinations, it was found that unfavorable cytokine combinations became readily apparent within initial screens via a clear deficiency in CD3+ T cell expansion. Cytokine combinations of IL-6, IFNα, and IL21 which did not include IL-15 were all insufficient for CD3+ proliferation for all samples tested as compared with a combination of IL-4 and IL-7. Separate combinations of IL-4 and IL-7 with IL-15, IL-6, IL18, and IL12 for two samples were investigated to determine whether IL-4 or IL-7 promoted growth of memory VSTs in culture. These initial investigations identified that IL-7 was responsible for the growth of VSTs, while IL-4 could not support memory T cell expansion without addition of IL-15 or IL-7. This requirement for memory T cell expansion via stimulation through the IL-15 receptor or IL-7 receptor is not unexpected as both receptors share homology with IL2 via utilizing the common γ-chain and its associated Jak/STAT signaling proteins. The IL-15 receptor is a heterotrimer composed of the IL2Rβ, the common γ-chain, and the specific IL-15Rα, while the IL-7 receptor is a heterodimer consisting of the common γ-chain with IL-7Rα/CD127. Recombinant IL-7 had been previously used clinically to expand T cell subsets in cases of lymphopenia, and was included with IL-4 for its pro-survival benefits for T cells. This previous investigation suggested inclusion of IL-15 was inferior to IL-4/IL-7 because of a lack of CD4+ T cell expansion and excessive CD56+ NK cell growth (37.7%). In contrast, our current data identified a robust CD4+ T cell expansion over six samples coupled with a much smaller but still detectable percentage of NK cells in wells cultured with either IL-15/IL-6 or IL-15 alone, 6.2% on average. Further investigations would be warranted to confirm NK cell growth IL-15/IL-6 was minimal compared to CD3+ T cell growth and to further investigate their safety profile.

It was identified that culture in IL-4 alone for two samples was not sufficient for cell growth-only when IL-4 was co-cultured with IL-7 or IL-15 was there expansion of CD3+ T cells. IL-4 had been described to support T cell survival by inhibiting degradation of anti-apoptotic factors Bcl2 and Bcl-xL, while IL-4 induced proliferation was more limited to naive cells. The data also demonstrated that addition of IL-6 to IL-15 improved the total CD3+ and CD8+ expansion, while showing no significant difference for effector function compared with IL-15 alone. Initial experiments in knockout mice previously demonstrated that IL-6 was not required for survival of naive cells. For CD8+ T cells, signaling via IL-6 has been described to reduce the threshold for TCR signaling in CD8+ T cells, which aligns with the data and would be ideal for promoting memory T cell expansion in response to peptide re-stimulation.

Most intriguingly, the investigation highlights the significant bias cytokines can impart upon the final balance of CD4+ vs CD8+ T cells within polyclonal T cell products. Production of an efficacious mix of viral specific CD4+ and CD8+ has been a focus for T cell therapy products to combine the directly cytotoxic CD8+ T cell response with “help” provided by anti-viral CD4+ T cells through promoting immune activation, recruitment, and inhibition of viral replication. For CMV infections, CD8+ T cells responses correlate with resolution of disease after HSCT, while addition of CMV specific CD4+ T cells has also been demonstrated to help CD8+ T cell responses for some HSCT patients. While the data indicate a clear bias towards expanding CD4+ T cells in products cultured with IL-4/IL-7, multiple clinical trials have used VST cells cultured in IL-4/IL-7 to treat ongoing viral infections, including EBV related post-transplant lymphoproliferative disorder (PTLD). These successes represent the relative abundance of antigen specific memory T cells expanded by the memory VST protocol. Nevertheless, the combination of IL-15/IL-6 can provide a more balanced ratio of antigen specific CD4+ to CD8+ T cells during polyclonal expansion of T cell products against not only viral specific antigens, but also other targets, including tumor associated antigens.

VST products grown in either 96 well plates or G-rex vessels were CCR7− CD45RO+ CD45RA− CD62L−, nominally effector memory, when cultured with either IL-4/IL-7 or IL-15/IL-6. Cytokine combinations which produced a substantial frequency of either central memory cells, or stem cell memory cells were not identified. Culture in IL-4 promoted survival of a naive CD4+ population which was absent in cultures with IL-15/IL-6. While transfer of less differentiated T cells has been indicated to be more favorable for long term reconstitution for cellular therapy against tumors, naive, non-antigen reactive cells would not be expected to contribute to the anti-viral response in the near term. Also, studies demonstrate tumor specific T cell products in patients with durable anti-melanoma responses demonstrated a remarkably plastic surface expression, as initial cell populations transitioned from a CD27ₗₒ CD28ₗₒ CD45RA− CD62L− CCR7− expression profile to a persistent effector memory CD27+ CD28+ CD45RO+ CD45RAᵢₙₜ CCR7− CD62L− profile. Clinical evidence suggests the absolute dose of antigen specific T cells delivered to the patient may be the most critical determinant for the effectiveness of the cell product rather than the specific memory phenotype. Thus, products grown in IL-15/IL-6 would be predicted to be equivalent to products grown in IL-4/IL-7 based on the total growth of CD3+ IFNγ+ cells.

Overall it has been demonstrated that a plate-based flow cytometric assay can effectively measure growth, function, and phenotype in a high throughput fashion for process development. This assay was demonstrated effective for screening 90 different cytokine combinations, rapidly identifying IL-15/IL-6 as equivalent to current GMP culture conditions using IL-4/IL-7. Furthermore, it was demonstrated that culture in IL-15/IL-6 provides an advantage when expanding CD8+ T cells, while culture in IL-4/IL-7 would be advantageous for expansion favoring CD4+ T cells. The modular nature of the assay can promote future investigations.

### Example 5

### SARS-CoV-2 Specific T-Cells Rapidly Expanded for Therapeutic Use, and Target Conserved Regions of the Membrane Protein

T-cell responses to SARS-CoV-2 have been described in recovered patients, and may be important for immunity following infection and vaccination as well as for the development of an adoptive immunotherapy for the treatment of immunocompromised individuals. Here, we demonstrate that SARS-CoV-2-specific T-cells can be expanded from convalescent donors, and recognize immunodominant viral epitopes in conserved regions of membrane, spike, and nucleoprotein. Following in vitro expansion using a GMP-compliant methodology (designed to allow the rapid translation of this novel SARS-CoV-2 T-cell therapy to the clinic), membrane, spike, and nucleocapsid peptides elicited IFN-γ production, in 26 (58%), 11 (24%), and 9 (20%) convalescent donors (respectively), as well as in 2 of 15 unexposed controls. We identified multiple novel polyfunctional CD4-restricted T-cell epitopes within a highly conserved region of membrane protein, which induced polyfunctional T cell responses, which may be critical for the development of effective vaccine and T cell therapies. Hence, our study shows that SARS-CoV-2 directed T-cell immunotherapy targeting structural proteins, most importantly membrane protein, should be feasible for the prevention or early treatment of SARS-CoV-2 infection in immunocompromised patients with blood disorders or after bone marrow transplantation to achieve anti-viral control while mitigating uncontrolled inflammation.

Introduction. The adaptive immune response to SARS-CoV-2 remains ill-defined, and there is an urgent need to fill this gap in knowledge in order to enable the development of effective vaccines and therapies. Antibody responses to the spike and nucleocapsid proteins are well described, and recently the characterization of T cell responses to SARS-CoV-2 predominantly to spike, membrane, and nucleocapsid proteins has also been reported. Recent studies have reported that both CD4+ and CD8⁺ T-cell responses to SARS-CoV-2 are detectable in convalescent patients, as well as in a proportion of unexposed individuals, albeit at lower levels. Recent reports have also suggested that immunocompromised patients may be at high risk of severe and potentially prolonged disease, suggesting that T-cell immunity is essential for overcoming COVID-19. Studies of the related virus SARS-CoV demonstrated that T-cells recognizing viral epitopes within SARS-CoV structural proteins were integral in viral clearance, and remained detectable for >10 years after exposure.

Knowledge of T cell epitopes recognized in other viruses such as EBV, CMV, and adenovirus have successfully led to the development of adoptive immunotherapy with ex vivo expanded virus-specific T cells (VSTs). This approach has been highly successful in preventing or treating viral infections in high risk patients after bone marrow transplant (BMT) with minimal risk of GVHD. To date, over 1,000 patients have been treated internationally in phase I/II protocols using VSTs. Importantly, expansion of VSTs in vivo correlates strongly with antiviral efficacy. Hence, the expansion of such approaches to include SARS-CoV-2 specific T cells may also offer protection from COVID-19 to these vulnerable individuals. Here, we define the immunodominant T-cell epitopes within conserved regions of SARS-CoV-2 structural proteins, including the novel discovery that SARS-CoV-2 specific T cells predominantly recognize regions in the C-terminus of the membrane protein, which represents a critical “hot spot” for CD4-restricted T cell epitopes. We also noted an association between SARS-CoV-2 seropositivity and the breadth of T-cell responses to structural viral proteins in patients who recover from COVID-19. This data suggests that patient who mount an antibody response to SARS-CoV-2 are more likely to have a broader T-cell response following COVID-19, which may have implications for protective immunity in recovered patients. It also provides proof of concept for the rapid generation of GMP-compliant SARS-CoV-2-specific T cell therapeutics, with the potential to prevent or treat COVID-19 in immunocompromised patients with blood disorders and/or after bone marrow transplantation (BMT).

Materials and Methods

Donors. Peripheral blood mononuclear cells (PBMCs) from volunteers, both healthy and those with presumed or documented COVID-19 infection, were obtained from Children's National Hospital (Washington, D.C.) and the National Institute of Health under informed consent approved by the Institutional Review Board of both institutions in accordance with the Declaration of Helsinki.

Generation of SARS-CoV-2-Specific T-Cells

Evaluated T cell products included SARS-CoV-2 specific T cells (CSTs), manufactured from PBMCs of seropositive and seronegative volunteers. VSTs were produced using a rapid expansion protocol previously described. Briefly, PBMCs were pulsed simultaneously with overlapping peptide pools encompassing viral structural proteins (1 μg/15×10⁶ PBMCs) for 30 minutes at 37° C. Peptide libraries of 15-mers with 11 amino acid overlaps encompassing the spike, membrane, nucleoprotein, and envelope proteins were generated (A&A peptide, San Diego, Calif., USA) from the SARS-CoV-2 reference sequence (NC_045512.2). After incubation, cells were resuspended with IL-4 (400 IU/ml; R&D Systems, Minneapolis, Minn.) and IL-7 (10 ng/ml; R&D Systems) in CTL media consisting of 45% RPMI (GE Healthcare, Logan, Utah), 45% Click's medium (Irvine Scientific, Santa Ana, Calif.), 10% fetal bovine serum, and supplemented with 2 mM GlutaMax (Gibco, Grand Island, N.Y.) according to our Good Manufacturing Practice (GMP)-compliant standard operating procedures (SOPs). Cytokines were replenished on day 7. On day 10, cells were harvested and evaluated for antigen specificity and functionality. A subset of samples were re-stimulated with autologous PBMCs that were pulsed with the viral peptide libraries, irradiated at 75 grey, and co-cultured with the CSTs at a ratio of 1:4 (CSTs to PBMCs). These restimulated cells were incubated in IL-4 (400 IU/ml) and IL-7 (10 ng/ml), with cytokines replenished at day 17, and harvested at day 21 for further testing.

Isolation and maintenance of SARS-CoV-2-specific T-cell clones. Membrane and Spike specific T-cells were isolated from frozen VSTs using an IFN-γ capture assay protocol previously described. Briefly, VSTs were thawed, washed in warm X-VIVO-15, and resuspended at a concentration of 1×10⁷ cells/ml. VSTs were stimulated for 3 hours with overlapping peptide pools encompassing viral antigens to Spike and Membrane to a final concentration of 1 μg/ml. T-cells producing IFN-γ in response to this stimulation were enriched using the IFN-γ secretion detection and enrichment kit (Miltenyi cat #130-054-201) in accordance with the manufacturer's instructions. These T-cells were plated at a series of dilutions in 96-well plates with irradiated feeder medium (RPMI 1640 supplemented with 10% FBS, L-glutamine, and PenStrep [R-10] with 1×10⁶ cells/ml 5,000 rad irradiated PBMC+50 U/ml IL-2+10 ng/ml IL-15+0.1 ug/ml each of anti-CD3 (Ultra-LEAF purified Anti-human CD3 antibody clone OKT3, Biolegend, cat 317325) and anti-CD28 (Ultra-LEAF purified Anti-human CD28 antibody clone 28.2, Biolegend, cat 302933). One month later, colonies were selected from the lowest dilution plates with positive wells (<⅓ of wells positive) and screened for responsiveness to Membrane or Spike peptide pools by intracellular cytokine staining for IFN-gamma, TNF-alpha, and degranulation by CD107a staining.

Membrane and Spike-specific T-cell clones were expanded bi-weekly with irradiated feeder medium.

IFN-γ Enzyme-Linked Immunospot (ELISpot) Assay

Antigen specificity of T-cells was measured by IFN-γ ELISpot (Millipore, Burlington, Mass.). T-cells were plated at 1×10⁵/well with no peptide, actin (control), or each of the individual SARS-CoV-2 pepmixes (200 ng/peptide/well). Plates were sent for IFN-γ spots forming cells (SFC) counting (Zellnet Consulting, Fort Lee, N.J.).

Flow Cytometry

VSTs were stained with fluorophore-conjugated antibodies against CD4, CD8, TCRαβ, TCRγδ, CXCR3, CXCR5, CCR6, CD127, CD25, and CD56 (Miltenyi Biotec, Bergisch Gladbach, Germany; BioLegend, San Diego, Calif.). All samples were acquired on a CytoFLEX cytometer (Beckman Coulter, Brea, Calif.). Intracellular cytokine staining was performed as follows: 1×10⁶ VSTs were plated in a 96-well plate and stimulated with pooled pepmixes or individual peptides (200 ng/peptide/well) or actin (control) in the presence of brefeldin A (Golgiplug; BD Biosciences, San Jose, Calif.) and CD28/CD49d (BD Biosciences) for 6 hours. T-cells were fixed, made permeable with Cytofix/Cytoperm solution (BD Biosciences) and stained with IFN-γ and TNF-α, and IL-2 antibodies (Miltenyi Biotec).

For intracellular flow cytometry of T-cell clones, cells were stimulated with Membrane and Spike peptide pools to a concentration of 1 μg/ml, and stained with anti-CD107a PE ((LAMP-1) Antibody Clone H4A3 Biolegend) and incubated at 37° C. 5% CO₂. After 1 hour, 1 μg/ml of brefeldin A was added to each well, and plates were incubated for another 5 hours. Cells were then washed in 2% FBS PBS and surface stained with fluorochrome-conjugated antibodies to CD3-Brilliant Violet 785 clone OKT3, CD4-Alexa Fluor 700 clone RPA-T4, CD8-FITC clone RPA-T8, OX40-Brilliant Violet 711 clone Ber-ACT35 (ACT35), (all from Biolegend), CD69-APC-eFluor 780 clone FN50, as well as fixable aqua viability dye (both from Invitrogen). Cells were fixed, permeabilized using BD Cytofix/Cytoperm solution, and stained with anti-IFN-γ Brilliant Violet 421 clone 4S.B3, anti-TNF-77a PerCP-Cyanine5.5 clone Mab11 (both from Biolegend). Cells were analyzed on an Attune N×T flow cytometer. Data was analyzed with FlowJo X (FlowJo LLC, Ashland, Oreg.).

Epitope Mapping

CSTs were tested for specificity to minipools containing 8-24 peptides spanning the SARS-CoV2 antigens by IFN-γ ELISpot. Cross-reactive pools were analyzed and individual peptides were tested to confirm epitope specificity. In silico predictions of MHC restrictions was performed using MARIA (hypertex transfer protocol://maria.stanford.edu) and NetMHCIIPan (http://www.cbs.dtu.dk/services/NetMHCIIpan-4.0/). To confirm the restricted HLA allele, CSTs were plated at 1×10⁵/well with partially HLA-matched phytohaemagglutinin (PHA)-treated lymphoblasts (PHA-blasts, 25 Gy irradiated) either alone or pulsed with peptide (1 ug/ml) and tested via IFN-γ ELISpot.

Luciferase Immunoprecipitation Systems (LIPS) for Measurement of SARS-CoV-2 Antibodies

Testing for antibodies to spike and nucleocapsid proteins were performed using a LIPS assay as recently described. Briefly, plasma samples were incubated with spike and nucleocapsid proteins fused to Gaussia and Renilla luciferase, respectively, protein A/G beads were added, the mixture was washed, coelenterazine substrate (Promega) was added, and luciferase activity was measured in light units with a Berthold 165 LB 960 Centro microplate luminometer. Antibody levels were reported as the geometric mean level (GML) with 95% confidence interval (CI). Cut-off limits for determining positive antibodies in the SARS-CoV-2-infected samples were based on the mean plus three standard deviations of the serum values derived from uninfected blood donor controls or by receiver operator characteristics (ROC) analysis. For some of the data percentages for categorical variables, mean and range, geometric mean plus 95% CI were used to describe the data. Wilcoxon signed rank were used for statistical analysis.

Multiplex Cytokine Assay

CSTs were plated at 1×10⁵/well in 96-well plates, stimulated with pooled pepmixes (200 ng/peptide/well) or control actin peptide, and incubated 48 hours. Supernatants were harvested and the cytokine profile analysis was performed using the Bio-plex Pro Human 17-plex Cytokine Assay kit (Bio-Rad, Hercules, Calif.), and read on a MAGPIX system (Luminex, Austin, Tex.).

Chromium Release Assay

Phytohaemagglutinin blasts were labeled with Chromium-51 (Perkin Elmer, Waltham, Mass., USA) at 10 μCi per 5×10⁵ cells. CST were co-plated with ⁵¹Cr-labeled, MHC-mismatched irradiated PHA blasts at effector:target ratios between 40:1 and 5:1, and incubated at 37° C. for 4 hours. Maximal release was evaluated by lysis of ⁵¹Cr-labeled targets with Triton-X-100. Supernatants were transferred to lumiplates and read on a MicroBeta2 plate reader (Perkin Elmer). Specific lysis was calculated as follows: (Experimental Counts per minute[CPM]−Background CPM)/(Maximal CPM−Background CPM).

Results

The majority of convalescent patients show antibody responses to SARS-CoV-2. Forty-six convalescent donors from the eastern and midwestern US with presumptive recent COVID-19 (36 PCR proven and 10 presumed positive because they were: (i) symptomatic and in close contact with PCR-positive individuals and/or (ii) positive for SARS CoV-2 antibody testing) were evaluated at a median time of 36 days after symptom onset (range 18-111). Median donor age was 34.5 years (range 20-69). Most patients had mild disease (84%) and 4 were asymptomatic, whereas 3 had moderate disease and one had severe disease based on WHO classification, with a median of 12 days of illness (Table 8). Antibody responses were detected in 33 of the 46 convalescent donors (27/46 to spike protein, and 29/46 to nucleocapsid protein). None of the 15 control subjects had detectable antibody responses.

TABLE 8
Convalescent Patient Demographics (n = 45).

| Description | Value | |
|---|---|---|
| Median age in years (range) | 34 | (20-69) |
| Male gender | 20 | (44%) |

Disease Severity

| | | |
|---|---|---|
| Mild | 37 | (83%) |
| Moderate | 3 | (7%) |
| Severe | 1 | (2%) |
| Asymptomatic | 4 | (9%) |

Symptoms

| | | |
|---|---|---|
| Fever | 24 | (53%) |
| Respiratory symptoms | 37 | (82%) |
| GI symptoms | 8 | (18%) |
| Fatigue | 14 | (42%) |
| Anosmia | 19 | (42%) |

| | | |
|---|---|---|
| Median length of symptoms, days (range) | 12 | (0-30) |
| Need for Hospitalization | 2 | (4%) |

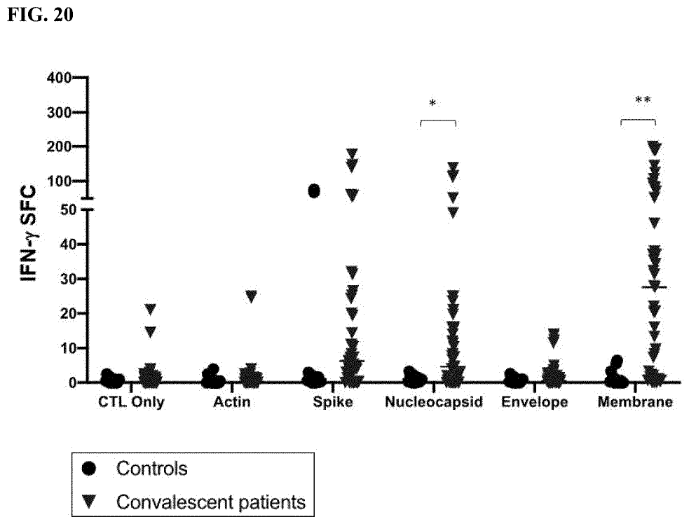

CSTs from convalescent donors are polyfunctional and recognize multiple viral proteins. Following stimulation and expansion of CSTs, specific T cell activity against SARS-CoV-2 structural proteins were detected in 31 of 46 convalescent donors and 2 of 15 control subjects (FIG. 20) via IFN-γ ELISpot. Convalescent donors predominantly responded to membrane (26/45, p=0.000013 versus control subjects), followed by spike (11/45, p=0.78 versus control subjects), and nucleocapsid proteins (9/45, p=0.0015 versus control subjects). Non-amplified responses to SARS-CoV-2 viral antigens were detectable from PBMCs via IFN-γ ELISpot in only 2 of 46 patients and none of 15 controls suggesting that the frequency of the SARS-CoV-2 response is relatively low, consistent with T cell immune responses observed against other respiratory viruses (e.g. adenovirus).

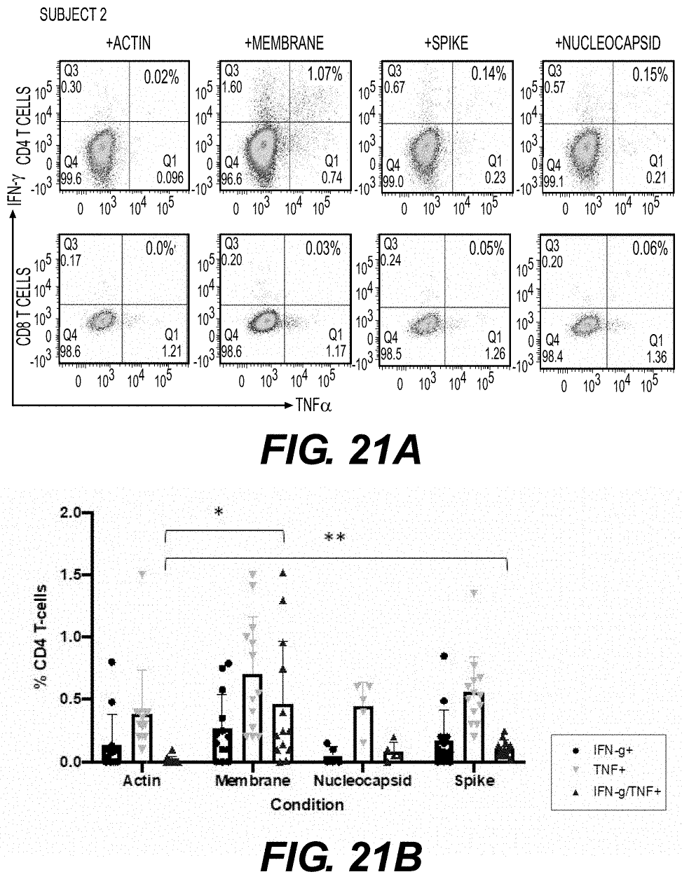

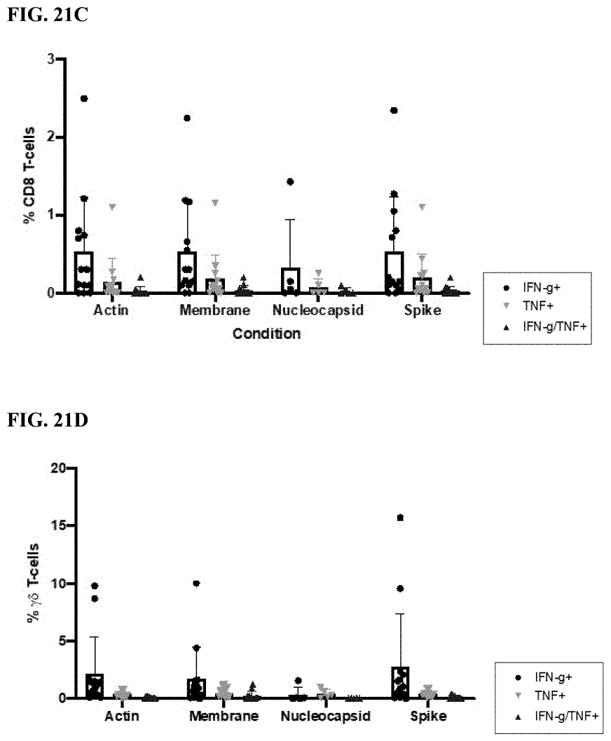

Post-expansion T cells were predominantly CD4⁺, with central memory and effector memory subsets. The predominant CD4+ T cell population was CXCR3⁺CCR6⁻ (mean 42.3% of CD4+ T-cells) consistent with a Th1 population, with minor populations expressing CXCR5⁺/CXCR3⁻ (mean 12.95% of CD4+ T-cells) and CD127⁻/CD25⁺ (mean 15.18% of CD4+ T-cells). These ratios were proportionate to rapidly expanded Virus-specific T cells targeting cytomegalovirus, Epstein-Barr virus, and adenovirus. Responses to spike and membrane proteins were confirmed to be predominantly CD4⁺ restricted in 11/11 tested patients (FIG. 21), with significant elevations in IFN-γ/TNFα-expressing populations targeting membrane and spike proteins (p=0.008 and p=0.0002 in comparison to actin, respectively). Following re-stimulation with viral structural proteins, CSTs produced multiple cytokines, with significant production of IL-1β, IL-2, IL-4, IL-6, IL-7, IL-12, G-CSF, IFN-γ, and TNFα.

CSTs expanded to 18 days following a second stimulation showed a similar pattern of cytokine production, which was not statistically different from the cytokine profile following the first stimulation. Alloreactivity testing of CSTs via ⁵¹Cr release assay showed no lysis of HLA-mismatched PHA blasts by T-cells following 10 or 21 days of expansion. Culture of clonal CST populations by limiting dilution and re-stimulation yielded several CD4+ T-cell clones, which showed polyfunctional cytokine production on peptide restimulation.

In order to assess cross-reactivity, CSTs were tested against peptides corresponding to variant epitopes in circulating SARS-CoV-2 genotypes, and from the HKU1 and OC43 coronaviruses.

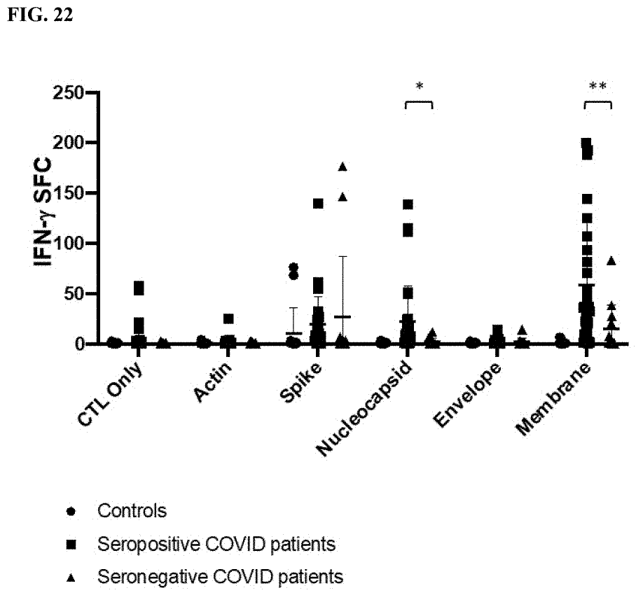

CSTs from seropositive donors recognize a broader array of viral antigens than CSTs derived from donors who lack detectable humoral responses. Of the 46 convalescent patients with history of COVID-19, 25 had demonstrable antibody and T-cell responses to SARS-CoV-2. Seven convalescent donors had no detectable T-cell or antibody responses. Seven donors had antibody responses without detectable T-cell responses, and 6 donors had T-cell responses without accompanying antibody responses, as has been observed with other infections such as EBV and HSV. A significant association was noted between presence of an antibody response and T-cell response to spike protein in convalescent patients (p=0.004 via Pearson Chi-squared test). Additionally, seropositive subjects were also more likely to demonstrate a T-cell response to membrane (p=0.0014) and nucleocapsid proteins (p=0.0029) (FIG. 22). Although there was no detectable correlation between disease severity and the magnitude of T-cell or antibody responses, 14 of the 20 patients who lacked T-cell and/or antibody responses had mild disease, and all 4 asymptomatic donors had incomplete immune responses (3 donors had SARS-CoV-2 T-cell responses only, and 1 donor had detectable SARS-CoV-2 antibody responses only). Evaluation of T-cell responses prior to COVID-19 infection was able to be performed on two subject who had banked cells collected before the pandemic. Subject 4 had mild GI disease, fever, and shortness of breath, and developed a CD4+ T-cell response to Spike protein (which was not detectable pre-illness), but no detectable antibody response to Spike or Nucleocapsid. SARS-CoV-2 immune (humoral and adaptive) responses were absent in the pre-pandemic sample, and post infection (after being confirmed to be PCR+ for SARS-CoV-2), a robust T cell response to spike protein was demonstrated, though this individual did not have an antibody response to Spike. Subject 46 had mild respiratory symptoms, anosmia, and GI symptoms, and developed a CD4+ T-cell response, as well as antibody response to both Spike and Nucleocapsid, both of which were absent two months prior to his illness.

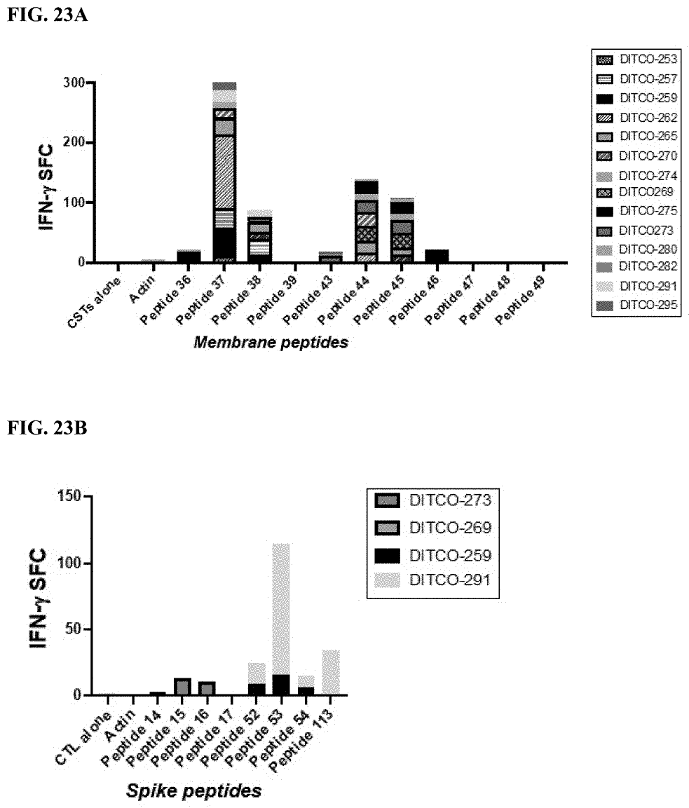

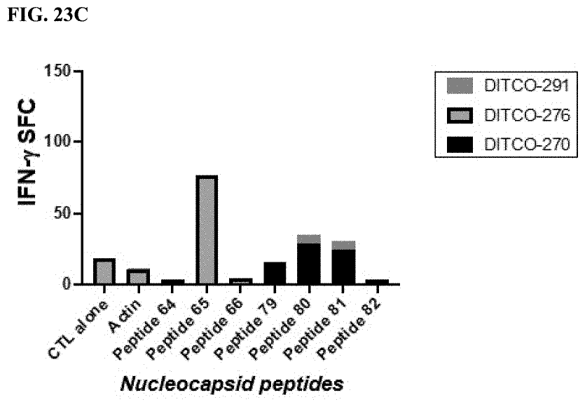

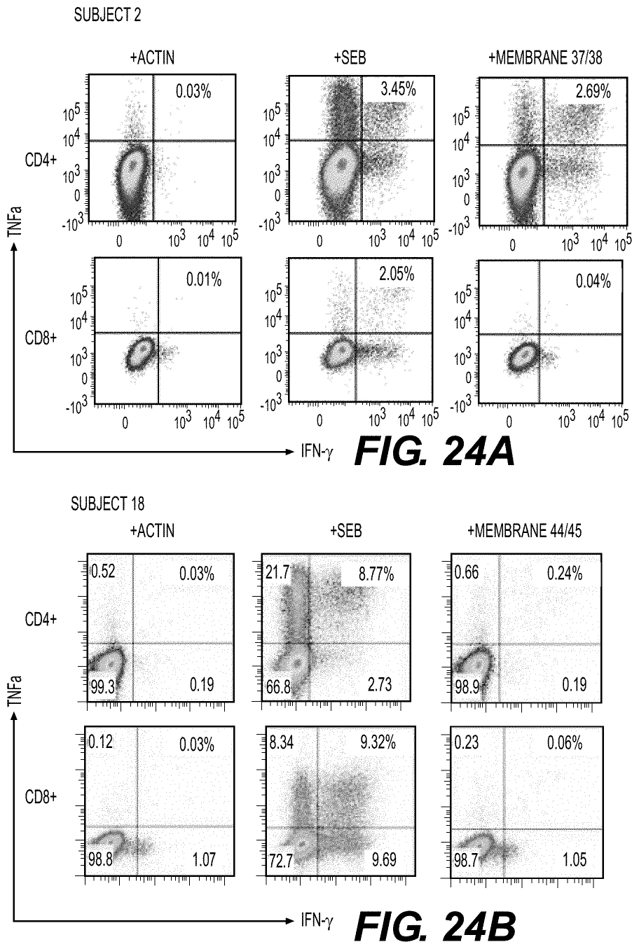

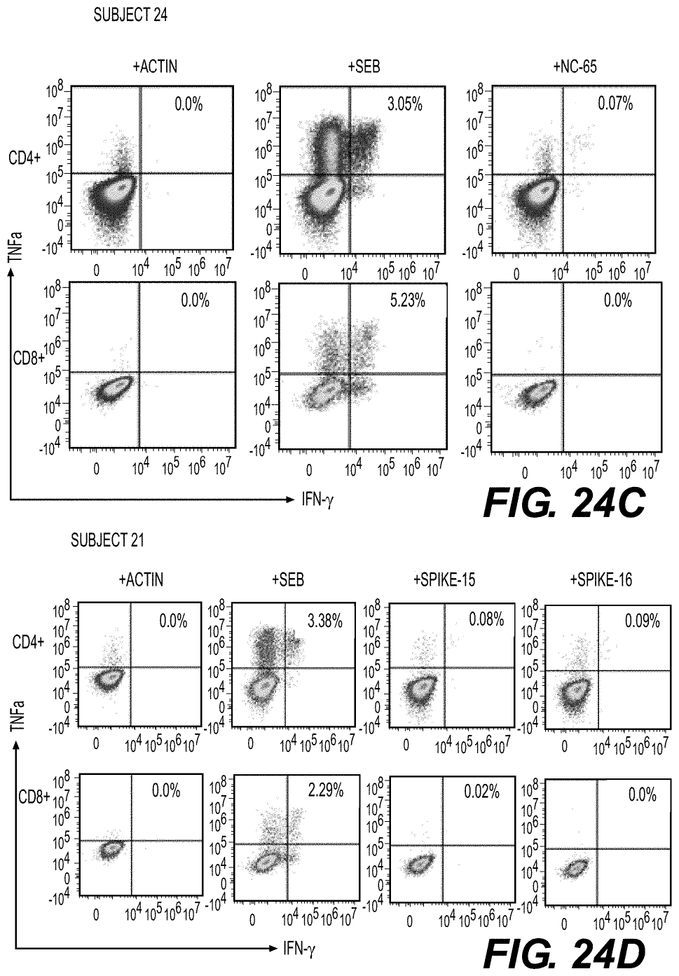

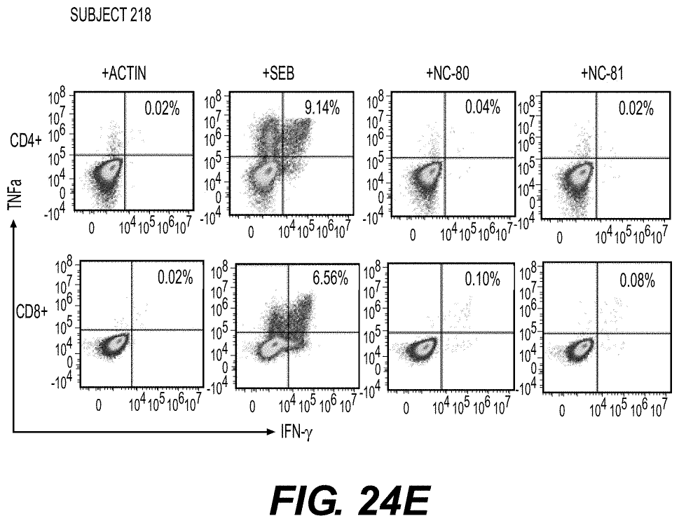

CSTs recognize multiple immunodominant epitopes in membrane, nucleocapsid, and spike proteins. Epitope mapping of the membrane protein yielded multiple epitopes at the C-terminal domain (FIG. 23A). Two epitopes at AA 144-163, were recognized by 8 donors, and were exclusively CD4-restricted (FIG. 24A). Using in silico analysis, the predicted HLA restrictions of these responses were HLA-DRB1*11 and DRB4*01 (Table 9). Similarly, epitopes at AA 173-192 were recognized by 6 donors, and were also confirmed to be CD4-restricted (FIG. 24B). These epitopes lie within the C-terminal domain which is located inside the virion and on intracellular membranes of infected cells that is a conserved region within all known strains of SARS-CoV2. Confirmatory restriction testing using partially-HLA matched cells confirmed that Membrane peptide 37 (AA 145-160) is bound by HLA-DRB1*11:01.

TABLE 9
Identified Class II Epitopes in Membrane, Nucleoprotein, and Spike proteins
and predicted HLA restrictions.
Membrane

| | Amino | | | | | | | | | |
|---|---|---|---|---|---|---|---|---|---|---|
| Peptide | acid | | HLA- | HLA- | HLA- | HLA- | HLA- | HLA- | HLA- | HLA- |
| sequence | location | Subject | DRB1 | DRB3 | DRB4 | DRB5 | DQA1 | DQB1 | DPA1 | DPB1 |
| LRGHLRIA | 145-159 | 257 | 07:01, | 02:02 | [symbol] | | 02:01, | 03:01, | 01:03, | 03:01, |
| GHHLGRC | | | 11:04 | | | | 05:01 | 03:03 | 02:01 | 03:03 |
| (SEQ ID | | | | | | | | | | |
| NO: 13, | | | | | | | | | | |
| residues | | | | | | | | | | |
| 145-159) | | | | | | | | | | |
| | | 262 | 07:01, | 02:02 | [symbol] | | 02:01, | 02:02, | 01:03, | 04:01, |
| | | | 11:01 | | | | 05:01 | 03:01 | 03:01 | 11:01 |
| | | 265 | 11:04, | 02:02 | [symbol] , | [symbol] | 01:03, | 03:01, | 01:03, | 02:01, |
| | | | 15:02 | | [symbol] | | 05:01 | 06:01 | 02:01 | 14:01 |
| | | 270 | 11:04, | 02:02 | | | 01:02, | 03:01, | 01:03, | 04:01, |
| | | | 15:01 | | | | 05:01 | 06:02 | 02:01 | 14:01 |
| LRIAGHHL | 149-163 | 257 | 07:01, | 02:02 | 01:03 | | 02:01, | 03:01, | 01:03, | 03:01, |
| GRCDIKD | | | 11:04 | | | | 05:01 | 03:03 | 02:01 | 03:03 |
| (SEQ ID | | | | | | | | | | |
| NO: 13, | | | | | | | | | | |
| residues | | | | | | | | | | |
| 149-163) | | | | | | | | | | |
| | | 265 | 11:04, | 02:02 | 01:01, | 01:02 | 01:03, | 03:01, | 01:03, | [symbol] |
| | | | 15:02 | | 01:03 | | 05:01 | 06:01 | 02:01 | 14:01 |
| SRTLSYYK | 173-187 | 262 | [symbol] | 02:02 | [symbol] | | 02:01, | 02:02, | [symbol] | [symbol] |
| LGASQRV | | | 11:01 | | | | 05:01 | [symbol] | 03:01 | 11:01 |
| (SEQ ID | | | | | | | | | | |
| NO: 13, | | | | | | | | | | |
| residues | | | | | | | | | | |
| 173-187) | | | | | | | | | | |
| | | 265 | 11:04, | 02:02 | [symbol] , | 01:02 | 01:03, | [symbol] | 01:03, | [symbol] |
| | | | [symbol] | | [symbol] | | 05:01 | 06:01 | 02:01 | 14:01 |
| | | 269 | 12:01, | 02:02 | [symbol] | 01:01 | 01:02, | [symbol] | 01:03, | [symbol] |
| | | | [symbol] | | | | 05:01 | 05:01 | 01:03 | 04:01 |
| | | 270 | 11:04, | 02:02 | | | 01:02, | [symbol] | 01:03, | [symbol] |
| | | | [symbol] | | | | 05:01 | 06:02 | [symbol] | 14:01 |
| | | 273 | 03:02, | 01:62 | [symbol] , | [symbol] | 01:02, | [symbol] | 02:02, | 01:01, |
| | | | [symbol] | | [symbol] | | 04:01 | 05:02 | 02:02 | 01:01 |
| | | 275 | [symbol] , | 01:01 | [symbol] , | | [symbol] , | 02:01, | [symbol] | [symbol] |
| | | | 03:01 | | [symbol] | | [symbol] | [symbol] | [symbol] | [symbol] |
| SYYKLGA | 177-191 | 262 | 07:01, | [symbol] | 01:01 | | [symbol] | 02:02, | 01:03, | 04:01, |
| SQRVAGDS | | | [symbol] | | | | 05:01 | 03:01 | 03:01 | 11:01 |
| (SEQ ID | | | | | | | | | | |
| NO: 13, | | | | | | | | | | |
| residues | | | | | | | | | | |
| 177-191) | | | | | | | | | | |
| | | 269 | 12:01, | [symbol] | 01:01 | [symbol] | 01:02, | 03:01, | 01:03, | 02:01, |
| | | | 15:01 | | | | 05:01 | 05:01 | 01:03 | 04:01 |
| | | 273 | 03:02, | 01:62 | 01:01, | 02:02 | [symbol] | [symbol] | 02:02, | 01:01, |
| | | | 16:02 | | 01:03 | | [symbol] | [symbol] | 02:02 | 01:01 |
| | | 275 | 01:01, | [symbol] | 01:01, | | 01:01, | [symbol] | 01:03, | 02:01, |
| | | | 03:01 | | 01:03 | | 05:01 | 05:01 | 01:03 | 02:01 |
| LGASQRV | 181-195 | 275 | 01:01, | [symbol] | 01:01, | | 01:01, | 02:01, | 01:03, | 02:01, |
| AGDSGFAA | | | 03:01 | | 01:03 | | 05:01 | 05:01 | 01:03 | 02:01 |
| (SEQ ID | | | | | | | | | | |
| NO: 13, | | | | | | | | | | |
| residues | | | | | | | | | | |
| 181-195) | | | | | | | | | | |

Nucleoprotein

| | | | | | | | | | |
|---|---|---|---|---|---|---|---|---|---|
| KPRQKRT | 257-271 | 276 | 04:01, | 01:01 | 01:03 | 03:01, | 03:01, | 02:01, | 14:01, |
| ATKAYNVT | | | 07:01 | | | 05; 05 | 03:02 | 02:01 | 14:01 |
| (SEQ ID | | | | | | | | | |
| NO: 14, | | | | | | | | | |
| residues | | | | | | | | | |
| 257-271) | | | | | | | | | |
| AFFGMSRI | 313-327 | 270 | 11:04, | 02:02 | | 01:02, | 03:01, | 01:03, | 04:01, |
| GMEVTPS | | | 15:01 | | | 05:01 | 06:02 | 02:01 | 14:01 |
| (SEQ ID | | | | | | | | | |
| NO: 14, | | | | | | | | | |
| residues | | | | | | | | | |
| 313-327) | | | | | | | | | |

Spike

| | | | | | | | | | |
|---|---|---|---|---|---|---|---|---|---|
| PFFSNVT | 57-71 | 259 | 03:01, | 01:01 | 01:03 | 01:03, | 02:01, | 01:03, | 04:01, |
| WFHAIHVS | | | 13:01 | | | 05:01 | 06:03 | 01:03 | 04:01 |
| (SEQ ID | | | | | | | | | |
| NO: 11, | | | | | | | | | |
| residues 57- | | | | | | | | | |
| 71) | | | | | | | | | |
| NVTWFHA | 61-75 | 259 | 03:01, | 01:01 | 01:03 | 01:03, | 02:01, | 01:03, | 04:01, |
| IHVSGTNG | | | 13:01 | | | [symbol] | [symbol] | 01:03 | 04:01 |
| (SEQ ID | | | | | | | | | |
| NO: 11, | | | | | | | | | |
| residues 61- | | | | | | | | | |
| 75) | | | | | | | | | |
| SKHTPINL | 205-219 | 291 | 03:01, | 01:01 | [symbol] | [symbol] | [symbol] | 01:03, | 02:01, |
| VRDLPQG | | | [symbol] | | | [symbol] | [symbol] | 01:03 | 03:01 |
| (SEQ ID | | | | | | | | | |
| NO: 11, | | | | | | | | | |
| residues | | | | | | | | | |
| 205-219) | | | | | | | | | |
| PINLVRDL | 209-223 | 273 | [symbol] | 01:62 | 01:01, | [symbol] | 04:02, | 02:02, | 01:01, |
| PQGFSAL | | | 16:02 | | 01:03 | 04:01 | [symbol] | 02:02 | 01:01 |
| (SEQ ID | | | | | | | | | |
| NO: 11, | | | | | | | | | |
| residues | | | | | | | | | |
| 209-233) | | | | | | | | | |
| | | 291 | 03:01, | 01:01 | 01:03 | 03:01, | 02:01, | 01:03, | 02:01, |
| | | | 04:01 | | | 05:01 | 03:02 | 01:03 | 03:01 |
| YNYLYRL | 449-463 | 291 | 03:01, | 01:01 | 01:03 | 03:01, | 02:01, | 01:03, | 02:01, |
| FRKSNLKP | | | 04:01 | | | 05:01 | 03:02 | 01:03 | 03:01 |
| (SEQ ID | | | | | | | | | |
| NO: 13, | | | | | | | | | |
| residues | | | | | | | | | |
| 449-463) | | | | | | | | | |

Bold: strong binder (<2).
Bold italic: Weak binder (2-10)

The epitopes contained within the peptides described in Table 9 and the other tables herein may be employed in the methods for culturing T cells disclosed herein or for treatment or prevention of disease using T cells recognizing these epitopes. Typically, these peptide epitopes are expressed or complexed with HLA molecules capable of presenting the epitopes to the cellular immune system such as the HLA molecules disclosed by the Tables herein.

Epitope mapping of spike protein yielded 3 epitopes (FIG. 23B) within the S1 domain, which were also CD4-restricted (Figure within the S1 domain (FIG. 24D).

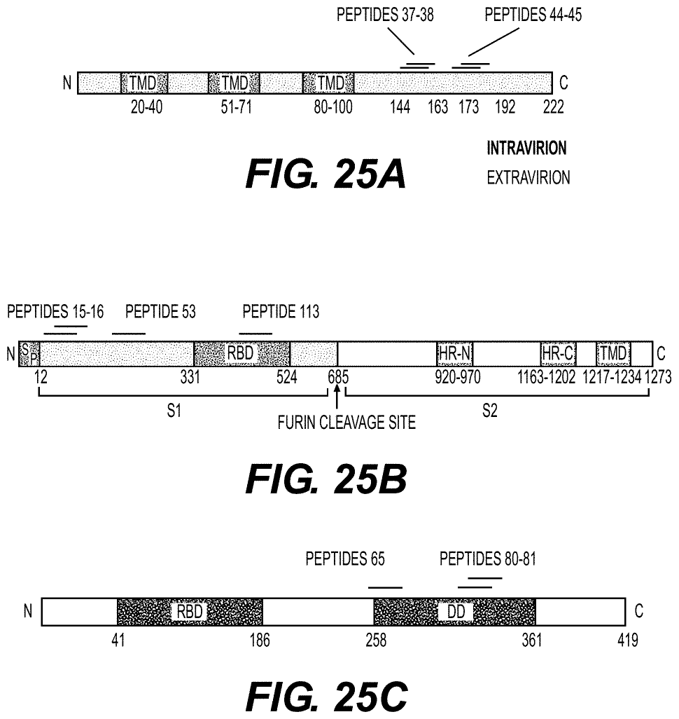

Epitope mapping of nucleoprotein yielded CD4-restricted epitopes at AA 257-271 (FIG. 23C and FIG. 24C), as well as a CD8 restricted epitope at AA 317-335 (FIG. 23C and Table 10). These lie in the dimerization domain, and are also highly conserved within SARS-CoV-2 genotypes (FIG. 25).

TABLE 10
Identified Class I Epitopes in Nucleoprotein and predicted HLA restrictions.

| | Amino | | | | |
|---|---|---|---|---|---|
| Peptide | acid | | | | |
| sequence | location | Subject | HLA-A | HLA-B | HLA-C |
| MSRIGMEV | 317-331 | 270 | | | |
| TPSGTWL | | | 24:02, 26:01 | 40:01, [symbol] | 02:02, 03:04 |
| (SEQ ID | | | | | |
| NO: 14, | | | | | |
| residues | | | | | |
| 513-331) | | | | | |
| GMEVTPSG | 321-335 | 270 | | | |
| [symbol] TWLTYTG | | | 24:02, 26:01 | 40:01, [symbol] | 02:02, 03:04 |
| [symbol] (SEQ ID | | | | | |
| [symbol] NO: 14, | | | | | |
| [symbol] residues | | | | | |
| 321-:335) | | | | | |

strong binder (<2). Bold italic: Weak binder (2-10).
*Predicted B*44:05 peptide: GMEVTPSGTW (SEQ ID NO: 14, residues 321-330) [symbol]
indicates data missing or illegible when filed

The epitopes contained within the peptides described in Table 10 and the other tables herein may be employed in the methods for culturing T cells disclosed herein or for treatment or prevention of disease using T cells recognizing these epitopes. Typically, these peptide epitopes are expressed or complexed with HLA molecules capable of presenting the epitopes to the cellular immune system such as the HLA molecules disclosed by the Tables herein

Discussion. Advancing knowledge of the immune response to SARS-CoV-2 is critical at the current juncture, to not only guide candidate vaccine studies but importantly to identify novel therapeutic targets for the design of a robust therapeutic T cell product for the treatment of immune compromised patients with blood disorders. Multiple studies have focused on the antibody response following COVID-19, but the persistence of antibody is unclear. Comparatively, T-cell responses are known to endure for years in response to SARS-CoV and MERS-CoV. In immunocompromised patients, including those undergoing BMT, viruses represent a significant risk for morbidity. Though to date, relatively few immunocompromised patients have died of COVID-19 relative to the general population, prolonged illness and prolonged viral shedding has also been described, which could increase risk for other patients and staff. Furthermore, even after recovery, this population is likely to be at risk for re-infection due to compromised adaptive responses. Adoptive T-cell immunotherapy may accordingly be beneficial for prevention or early treatment of COVID-19.

In this study, we demonstrate that ex vivo expanded CSTs may be easily generated from convalescent patients, following recovery from COVID-19, and recognize multiple immunodominant epitopes within membrane protein, which represent class II restricted T-cell epitope “hot spots”. Cross-reactivity with corresponding epitopes from circulating coronaviruses further suggest that T-cell recognition of these domains is common. Membrane, spike, and nucleoprotein showed a clear hierarchy of immunodominance, and were associated with significant increases in IFN-γ/TNF-α producing CD4+ T-cell populations. Though the understanding of the role and biologic significance of T-cell populations in combating SARS-CoV-2 remains limited, decreases in activated T-cell populations have been shown to correlate with patient acuity scores. Furthermore, the importance of polyfunctional CD4 T-cell responses are well-documented for anti-viral immunity against other respiratory viruses. Moreover, the efficacy of adoptive, predominantly MHC class II-restricted T-cell therapies targeting adenovirus in immunocompromised patients is a prime example of the potency of T cell therapies for clearance of respiratory viruses in the immune compromised host. Though T-cell immunotherapy targeting RNA viruses has not been attempted, the concept is supported by prior murine RSV studies. Accordingly, CSTs derived from an HSCT donor may be an effective preventative therapy especially for patients undergoing BMT. Further, for patients who lack a donor with immunity to COVID-19, the administration of partially-HLA matched third-party CSTs may be a consideration as an “on-demand: treatment of COVID-19 early in the course of infection to prevent invasive disease, with the goal to reduce the length and severity of illness.

However, the development of a potent “off the shelf” virus specific T cell therapy requires extensive characterization of the T cell products to discover the epitope specificity and HLA restrictions of the virus specific T cells to ensure optimal matching between the virus-specific T cell donor and the recipient. In this study, we showed that multiple regions within the highly conserved C-terminal domain of the membrane protein elicited CD4-restricted responses were shared by CST products generated from multiple individuals. Confirmation of HLA-restriction for Membrane peptide 37 was confirmed to be mediated by HLA-DRB1*11:01, and in silico analysis suggested restriction of these epitopes through HLA-DR11, DR7, DQ3, and DQ7, which are present in roughly 50% of the population. This information is therefore highly useful for the manufacture of a CST bank for clinical use. Moreover, given the increased severity of COVID-19 within minority populations, it is important to determine if there are risk associations with specific HLA types, which would need to be accounted for in candidate vaccines and understanding that these HLA restricted responses will be critical for the development of a third party CST bank to treat the majority of screened high risk patients (including ethnically diverse populations) as we and others have effectively achieved for other “off the shelf” virus specific T cell products. Finally, the demonstration of T-cell responses to described variant epitopes within SARS-CoV-2 suggests that CSTs are likely to have activity against many circulating viral strains in spite of genetic variation.

Overall, CSTs with specificity for one or more viral antigens could be successfully produced from 58% of the evaluated convalescent donors, and an association was detected between SARS-CoV-2 seropositivity and T-cell responses to non-Spike antigens. It is plausible that T-follicular helper cells play a role in this association, and a population of CXCR5+CD4+ T-cells were noted in expanded CSTs. Interestingly, not all convalescent donors had detectable humoral and cellular responses, and many incongruous responses were noted. In particular, those with mild disease and those who were asymptomatic appeared to have a higher rate of seronegativity and/or absent T-cell responses to the targeted antigens, which may implications for long-term protection for convalescent individuals, as well as for donor selection for immunotherapy. This was similarly described in several recent studies of humoral and T-cell responses. In our patients, seroconversion was noted to correlate with the presence of T-cell responses to a broader range of structural proteins. In patients who recovered from SARS-CoV disease, severity was noted to correlate with the magnitude of CD4 T-cell response, and it is possible a similar correlation exists in subjects with COVID-19.

Recent studies evaluating the T cell immune response to SARS-CoV2 in unexpanded peripheral blood samples identified both CD4 and CD8-restricted responses to viral structural proteins in convalescent donors, as well as in a fraction of unexposed subjects. Prior studies have postulated that this may be due to cross-reactivity with common circulating coronaviruses. In our study, we also observed T cell responses to spike proteins in 2 of 15 unexposed control subjects. However, we did not observe any responses to nucleocapsid or membrane proteins, which also paralleled our observation that responses to these proteins were predominantly detected in subjects with confirmed humoral immunity (i.e. seropositivity). Although we cannot definitively rule out rare T-cell populations recognizing non-spike proteins in virus-naïve donors, the absence of these responses in our study, even following ex vivo expansion, suggests that T-cell reactivity in unexposed individuals is more limited than in seropositive convalescent patients, which may reflect the differences in structural proteins in SARS-CoV-2 versus other commonly circulating coronaviruses (Table 11). Larger, longitudinal studies to analyze the cellular response to other coronaviruses and their possible cross-reactivities with SARS-CoV-2 will therefore be necessary to understand the clinical implications of pre-existing T-cell responses to SARS-CoV-2 antigens. Whether T-cell responses in unexposed donors may be effectively harnessed through selection, rapid expansion, or through methods akin to generation of CMV-specific T-cells from naïve donors, will also require study. Nevertheless, given the information currently available, our recommendation would be that seropositivity may not be necessary for the generation of a donor-derived CST product to be given prophylactically in the BMT setting. In contrast, for the development of a third party “off the shelf” CST therapeutic for the treatment of high-risk patients with known infection, our data suggests that utilizing donors with confirmed humoral immunity will enable the generation of broadly antigen and epitope specific therapeutic T cell products.

TABLE 11
Epitope Homology with Other Human Coronaviruses.

| | | | |
|---|---|---|---|
| SARS-CoV-2 | Other | | Amino Acid |
| Epitope | Human | Protein | Sequence |
| Identified | Coronavirus | Name | Alignment |

Membrane

| | | | |
|---|---|---|---|
| LRGHLRIAGHHLGRC | SARS | M protein | LRGHLRIAGHHLGRC |
| SEQ ID NO: 13, | Coronavirus | Membrane | (SEQ ID NO: 484) |
| residues | HKUI | glycoprotein | RGHLRMAGHPLGRC |
| 145-159) | MERS | M protein | (SEQ ID NO: 485) |
| | | | RGHLYIQGVKLG |
| | | | (SEQ ID NO: 486) |
| | | | GHLKIAGMHFGAC |
| | | | (SEQ ID NO: 487) |
| LRIAGHHLGRCDIKD | SARS | Membrane | LRIAGHHLGRCDIKD |
| SEQ ID NO: 13, | Coronavirus | protein | (SEQ ID NO: 488) |
| residues | | | LRMAGHPLGRCDIKD |
| 149-163) | | | (SEQ ID NO 489) |
| SRTLSYYKLGASQRV | SARS | Membrane | SRTLSYYKLGASQRV |
| SEQ ID NO: 13, | Coronavirus | protein | (SEQ ID NO: 490) |
| residues | | | SRTLSYYKLGASQRV |
| 173-187) | | | (SEQ ID NO: 491) |
| SYYKLGASQRVAGDS | SARS | Membrane | SYYKLGASQRVAGDS |
| SEQ ID NO: 13, | Coronavirus | protein | (SEQ ID NO: 492) |
| residues | | | SYYKLGASQRVGTDS |
| 177-191) | | | (SEQ ID NO: 493) |
| LGASQRVAGDSGFAA | SARS | Membrane | LGASQRVAGDSGFAA |
| SEQ ID NO: 13, | Coronavirus | glycoprotein | (SEQ ID NO: 494) |
| residues | NL63 | Orf1a protein | LGASQRVGTDSGFAA |
| 181-195) | | | (SEQ ID NO: 495) |
| | | | LGAS--VTEDVKFAA |
| | | | (SEQ ID NO: 496) |

Nucleoprotien

| | | | |
|---|---|---|---|
| KPRQKRTATKAYNVT | SARS | Nucleocapsid | KPRQKRTATKAYNVT |
| SEQ ID NO: 14, | coronavirus | protein | (SEQ ID NO: 497) |
| residues | OC43 | Nucleocapsid | KPRQKRTATKQYNVT |
| 257-271) | NL63 | protein | (SEQ ID NO: 498) |
| | MERS | Chain A, | KPRQKRSPNK |
| | | nucleoprotein | (SEQ ID NO: 499) |
| | | Nucleocapsid | KPRWKRVPTREENV |
| | | protein | (SEQ ID NO: 500) |
| | | | RHKRVATKSFNV |
| | | | (SEQ ID NO: 501) |
| AFFGMSRIGMEVTPS | SARS | Nucleocapsid | AFFGMSRIGMEVTPS |
| SEQ ID NO: 14, | coronavirus | protein N | (SEQ ID NO: 502) |
| residues | | | AFFGMSRIGMEVTPS |
| 313-327) | | | (SEQ ID NO: 503) |
| MSRIGMEVTPSGTWL | SARS | Nucleocapsid | MSRIGMEVTPSGTWL |
| SEQ ID NO: 14, | coronavirus | protein | (SEQ ID NO: 504) |
| residues | | | MSRIGMEVTPSGTWL |
| 317-331) | | | (SEQ ID NO: 505) |
| GMEVTPSGTWLTYTG | SARS | Nucleocapsid | GMEVTPSGTWLTYTG |
| SEQ ID NO: 14, | coronavirus | protein | (SEQ ID NO: 506) |
| residues | | | GMEVTPSGTWLTY |
| 321-335) | | | (SEQ ID NO: 507) |

Spike

| | | | |
|---|---|---|---|
| PFFSNVTWFHAIHVS | — | — | — |
| SEQ ID NO: 11, | | | |
| residues | | | |
| 57-71) | | | |
| NVTWFHAIHVSGTNG | — | — | — |
| SEQ ID NO: 11, | | | |
| residues | | | |
| 61-75) | | | |
| SKFTTPINLVRDLPQG | OC43 | Replicase | SKHTPINLVRDLPQG |
| SEQ ID NO: 11, | SARS | polvprotein | (SEQ ID NO: 508) |
| residues | coronavirus | lab | PANIV--LPQG |
| 205-219) | | S1 protein | (SEQ ID NO: 509) |
| | | | PIDVVRDLPSG |
| | | | (SEQ ID NO: 510) |
| PINLVRDLPQGFSAL | SARS | Chain A, | PINLVRDLPQGFSAL |
| SEQ ID NO: 11, | coronavirus | Spike | (SEQ ID NO: 511) |
| residues | OC43 | Replicase | PIDVVRDLPSGFNTL |
| 219-223) | | polyprotein | (SEQ ID NO: 512) |
| | | lab | PANIV--LPQG |
| | | | (SEQ ID NO: 513) |
| YNYLYRLFRKSNLKP | SARS | Chain E, | YNYLYRLFRKSNLKP |
| SEQ ID NO: 11, | coronavirus | spike | (SEQ ID NO: 514) |
| residues | | glycoprotein | YNYKYRYLRHGKLRP |
| 449-463) | | | (SEQ ID NO: 515) |

In some embodiments, biological samples may be obtained from subjects recognizing homologous or similar epitopes from other coronaviruses, such as those described above, and used to produce T cells recognizing SARS-CoV-2 or other related coronaviruses.

Limitations of this study include the sample size, the tendency toward mild illness in the subjects, and that not all the subjects were PCR-positive or antibody-positive for SARS-CoV-2. However, as the vast majority of the convalescent donors had uncomplicated disease, our data suggests that T-cell and humoral responses measured here represent an effective adaptive immune response to SARS-CoV-2 which can be effectively harnessed (especially from BMT donors) for the manufacture of CST products for clinical use. We did not evaluate donors longitudinally, and therefore the absence of T-cell responses in 42% of subjects may relate to the timing of T-cell responses following primary infection. We limited evaluation to structural viral proteins, given their described immunodominance in related coronaviruses, but it is possible that T-cell responses to non-structural proteins may have been present, as has been demonstrated in recent studies. The study was also inadequately powered to determine if any correlations exist between clinical severity and recognition of specific T-cell epitopes. However, as all of the evaluated patients survived and recovered without significant inflammatory or thrombotic complications, it is a reasonable assumption that the detected T-cell responses represent beneficial adaptive cellular responses.

We do acknowledge that a maladaptive immune response is highly suspected to be the cause of hyper-inflammatory complications such as multisystemic inflammatory syndrome in children, and an understanding of the role of adaptive and innate responses in patients with inflammatory complications will be critical in determining the characteristics of an effective and enduring adaptive immune response to SARS CoV-2. While this is a consideration when developing adoptive T-cell immunotherapy trials for this disease, the detection of T cells in our cohort of donors, the majority of whom had mild disease suggests that judicious use of CST products, given early in the infection process or given as prophylaxis (e.g. early post BMT) to high-risk patients warrants investigation. Inflammatory complications in patients with COVID-19 have been correlated with elevation of IL-6, IL-10, and IL-13. As other inflammatory complications such as cytokine release syndrome are very rare after virus-specific T-cell therapy, the risk of inflammatory complications after adoptive T-cell therapy for COVID-19, particularly when utilized early and derived from donors who themselves did not have inflammatory disease, are likely low.

In summary, this is the first report to demonstrate that a broadly specific T-cell therapeutic targeting three structural proteins of SARS-CoV-2 can be reliably expanded using GMP-compliant methodologies from the majority of convalescent donors. Moreover, the CST products are principally comprised of CD4⁺ T cells specific for conserved regions of these proteins, and most frequently, the membrane protein. The immunodominance of the membrane protein therefore also has important implications for vaccine development to elicit cellular immune responses, as most current vaccine candidates are focused exclusively on the spike protein to elicit neutralizing antibody. This work now enables the rapid translation of this novel treatment to the clinic. Future studies will therefore evaluate whether patient-specific and/or “off the shelf” adoptive T-cell immunotherapies using this novel CST product will emerge as a safe and useful treatment modality in high-risk patients with COVID-19, as we and others have effectively shown for the treatment of other respiratory viruses especially in the BMT setting.

Those skilled in the art will recognize, or be able to ascertain using no more than routine experimentation, many equivalents to the specific embodiments of the method and compositions described herein. Such equivalents are intended to be encompassed by the following claims.

Embodiments of this technology include, but are not limited to the following.

Embodiment 1. A method of culturing a composition comprising a population of one or a plurality of CD4+ and/or CD8+ T cells, said method comprising, consisting essentially of, or consisting of:
(a) contacting one or a plurality of T cells with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, for a time period sufficient to stimulate growth and proliferation of the one or plurality of CD4+ and/or CD8+ T cells; preferably wherein said two peptides comprise IL-15 and IL-7; and preferably wherein said T cells recognize, or are contacted with, a membrane peptide epitope of SARS-CoV-2

Embodiment 2. The method of embodiment 1 further comprising:
(b1) contacting the one or plurality of T cells with one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1, or one or a plurality of vectors that encode one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1 for a time period sufficient to activate the one or plurality of CD4+ and/or CD8+ T cells against a cell expressing PRAME, Survivin, or WT1, or functional fragments or combinations thereof, or
(b2) contacting the one or plurality of T cells with one or a plurality of viral antigens from a virus of the family Coronaviridae for a time period sufficient to activate the one or plurality of CD4+ and/or CD8+ T cells against a cell expressing the one or plurality of viral antigens.

Embodiment 3. The method of embodiment 1 or 2, wherein the time period sufficient to stimulate growth or proliferation of the one or plurality of CD4+ and/or CD8+ T cells is from about 2 to about 10 days.

Embodiment 4. The method of any of embodiments 1 through 3, wherein the time period sufficient to stimulate growth or proliferation of the one or plurality of CD4+ and/or CD8+ T cells is from about 3 to about 7 days.

Embodiment 5. The method of any of embodiments 1 through 4, wherein the time period sufficient to stimulate growth or proliferation of the one or plurality of CD4+ and/or CD8+ T cells is a time period in which no less than about 5% of CD8+ or CD4+ T cells produce either interferon-gamma or TNF-alpha.

Embodiment 6. The method of any of embodiments 1 through 5, wherein the time period sufficient to stimulate growth or proliferation of the one or plurality of CD4+ and/or CD8+ T cells is a time period in which no less than about 10% of CD8+ or CD4+ T cells produce either interferon-gamma or TNF-alpha.

Embodiment 7. The method of any of embodiments 1 through 6, wherein the time period sufficient to stimulate growth or proliferation of the one or plurality of CD4+ and/or CD8+ T cells is a time period in which no less than about 15% of CD8+ or CD4+ T cells produce either interferon-gamma or TNF-alpha.

Embodiment 8. The method of any of embodiments 1 through 7, wherein the time period sufficient to stimulate growth or proliferation of the one or plurality of CD4+ and/or CD8+ T cells is a time period in which no less than about 20% of CD8+ or CD4+ T cells produce interferon-gamma or TNF-alpha.

Embodiment 9. The method of any of embodiments 1 through 8, wherein the time period sufficient to stimulate growth or proliferation of the one or plurality of CD4+ and/or CD8+ T cells is a time period in which no less than about 22% of CD8+ or CD4+ T cells produce either interferon-gamma or TNF-alpha.

Embodiment 10. The method of any of embodiments 1 through 9, further comprising a step of culturing a composition comprising peripheral blood mononuclear cells (PBMCs) in vitro prior to performing step (a).

Embodiment 11. The method of any of embodiments 1 through 9, further comprising a step of isolating PBMCs from a subject and culturing the PBMCs in vitro prior to performing step (a).

Embodiment 12. The method of any of embodiments 1 through 11, wherein the one or plurality of T cells are isolated from:
(i) a subject diagnosed with or suspected of having cancer,
(ii) a subject diagnosed with or suspected of having COVID-19;
(iii) a subject diagnosed with or suspected of having an infection by a virus of the family Coronaviridae;
(iv) a subject diagnosed with or suspected of having an infection by SARS-CoV-2; or
(v) a subject diagnosed with or suspected of having an infection with an intracellular pathogen including a persistent intracellular pathogen.

Embodiment 13. The method of any of embodiments 1 through 12, wherein the T cells are obtained through an ex vivo expansion of a single population of T cells.

Embodiment 14. The method of any of embodiments 1 through 12, wherein the T cells are obtained through an ex vivo expansion of separate T cell subpopulations, wherein each T cell subpopulation is specific for a single viral antigen.

Embodiment 15. The method of any of embodiments 1 through 14, wherein the one or plurality of T cells are obtained from an allogeneic source.

Embodiment 16. The method of any of embodiments 1 through 14, wherein the one or plurality of T cells are obtained from an autologous source.

Embodiment 17. The method of any of embodiments 1 through 16, wherein the composition further comprises NKT cells and γδ T cells.

Embodiment 18. The method of any of embodiments 1 through 17, wherein the at least two peptides are:
(i) IL-15 and IL-6, or functional fragments or variants thereof;
(ii) IL-15 and IL-7, or functional fragments or variants thereof, or
(iii) IL-7 and IL-4, or functional fragments or variants thereof.

Embodiment 19. The method of any of embodiments 1 through 18, wherein the method is performed in a closed tissue culture system over at least 1, 2, 3, 4 or 5 days.

Embodiment 20. The method of any of embodiments 1 through 19, wherein the method is performed in a closed tissue culture system over from about 5, 6, 7, 8, 9 to about 10 days.

Embodiment 21. The method of any of embodiments 1 through 20, wherein the one or plurality of T cells are naïve cells prior to step (a) and the one or plurality of T cells are CCR7+CD45RO+ after performing step (a).

Embodiment 22. A method of expanding a population of CD8+ memory effector T cells in a composition of cultured cells, said method comprising, consisting essentially of, or consisting of:
(a) contacting one or a plurality of lymphocytes with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, for a time period sufficient to stimulate growth and proliferation of the one or plurality of CD8+ memory effector T cells within the composition of cultured cells; preferably wherein said at least two peptides comprise IL-15 and IL-7; and preferably wherein said CD8+ memory effector T cells recognize, or are contacted with, a peptide epitope of SARS-CoV-2

Embodiment 23. The method of embodiment 22 further comprising:
(b1) contacting the one or plurality of lymphocytes with one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1, or one or a plurality of vectors that encode one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1, for a time period sufficient to activate the one or plurality of CD8+ memory effector T cells against a cell expressing PRAME, Survivin, or WT1, or functional fragments or combinations thereof, or
(b2) contacting the one or plurality of lymphocytes with one or a plurality of viral antigens from a virus of the family Coronaviridae for a time period sufficient to activate the one or plurality of CD8+ memory effector T cells against a cell expressing the one or plurality of viral antigens.

Embodiment 24. The method of embodiment 22 or 23, wherein the time period sufficient to stimulate growth or proliferation of the one or plurality of CD8+ memory effector T cells is a time period sufficient to cause an increase of expression of CCR7 or CD45RO on the T cells after performing step (a).

Embodiment 25. The method of any of embodiments 22 through 24, wherein the time period sufficient to stimulate growth or proliferation of the one or plurality of CD8+ memory effector T cells is a time period sufficient to cause an increase of expression of CCR7 and CD45RO on the T cells after performing step (a).

Embodiment 26. The method of any of embodiments 22 through 25, wherein the time period sufficient to stimulate growth or proliferation of the one or plurality of CD8+ memory effector T cells is a time period sufficient to cause expression of CCR7 and CD45RO after performing step (a) in no less than about 2% of the CD8+ T cells.

Embodiment 27. The method of any of embodiments 22 through 26, wherein the time period sufficient to stimulate growth or proliferation of the one or plurality of CD8+ memory effector T cells is a time period sufficient to cause expression of CCR7 and CD45RO after performing step (a) in no less than about 5% of the CD8+ T cells.

Embodiment 28. The method of any of embodiments 22 through 27, wherein the time period sufficient to stimulate growth or proliferation of the one or plurality of CD8+ memory effector T cells is a time period sufficient to cause expression of CCR7 and CD45RO after performing step (a) in no less than about 10% of the CD8+ T cells.

Embodiment 29. The method of any of embodiments 22 through 28, wherein the time period sufficient to stimulate growth or proliferation of the one or plurality of CD8+ memory effector T cells is a timer period sufficient to cause expression of CCR7 and CD45RO after performing step (a) in no less than 15% of the CD8+ T cells.

Embodiment 30. The method of any of embodiments 22 through 29, wherein the CD8+ T cells secrete IFN-gamma or TNF-alpha.

Embodiment 31. The method of any of embodiments 22 through 30, wherein the CD8+ T cells secrete IFN-gamma and TNF-alpha.

Embodiment 32. The method of any of embodiments 22 through 31, wherein the CD8+ T cells are CD3+ and secrete IFN-gamma or TNF-alpha.

Embodiment 33. The method of any of embodiments 22 through 32, wherein the method further comprises stimulating proliferation of a subpopulation of CD4+ T cells that secrete IFN-gamma or TNF-alpha by exposing the cells simultaneously or sequentially with a polypeptide or nucleic acid encoding a polypeptide of IL-7, or functional fragments or variants thereof, and a polypeptide or nucleic acid encoding a polypeptide of IL-4, or functional fragments or variants thereof.

Embodiment 34. A method of expanding a population of CD4+ memory effector T cells in a composition of cultured lymphocytes, said method comprising, consisting essentially of, or consisting of:
(a) contacting one or a plurality of lymphocytes with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, for a time period sufficient to stimulate growth and proliferation of the one or plurality of CD4+ memory effector T cells within the composition of cultured cells; preferably wherein said at least two peptides comprise IL-15 and IL-7; and preferably wherein said CD4+ memory effector T cells recognize, or are contacted with, a peptide epitope of SARS-CoV-2.

Embodiment 35. The method of embodiment 34 further comprising:
(b1) contacting the one or plurality of lymphocytes with one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1, or one or a plurality of vectors that encode one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1, for a time period sufficient to activate the one or plurality of CD4+ memory effector T cells against a cell expressing PRAME, Survivin, or WT1, functional fragments or combinations thereof, or
(b2) contacting the one or plurality of lymphocytes with one or a plurality of viral antigens from a virus of the family Coronaviridae for a time period sufficient to activate the one or plurality of CD4+ memory effector T cells against a cell expressing the one or plurality of viral antigens.

Embodiment 36. The method of embodiment 34 or 35, wherein the time period sufficient to stimulate growth or proliferation of the one or plurality of CD4+ memory effector T cells is a time period sufficient to cause an increase of expression of CCR7 or CD45RO on the T cells after performing step (a).

Embodiment 37. The method of any of embodiments 34 through 36, wherein the time period sufficient to stimulate growth or proliferation of the one or plurality of CD4+ memory effector T cells is a time period sufficient to cause an increase of expression of CCR7 and CD45RO on the T cells after performing step (a).

Embodiment 38. The method of any of embodiments 34 through 37, wherein the time period sufficient to stimulate growth or proliferation of the one or plurality of CD4+ memory effector T cells is a time period sufficient to cause expression of CCR7 and CD45RO after performing step (a) in no less than about 2% of the CD4+ T cells.

Embodiment 39. The method of any of embodiments 34 through 38, wherein the time period sufficient to stimulate growth or proliferation of the one or plurality of CD4+ memory effector T cells is a time period sufficient to cause expression of CCR7 and CD45RO after performing step (a) in no less than about 5% of the CD4+ T cells.

Embodiment 40. The method of any of embodiments 34 through 39, wherein the time period sufficient to stimulate growth or proliferation of the one or plurality of CD4+ memory effector T cells is a time period sufficient to cause expression of CCR7 and CD45RO after performing step (a) in no less than about 10% of the CD4+ T cells.

Embodiment 41. The method of any of embodiments 34 through 40, wherein the time period sufficient to stimulate growth or proliferation of the one or plurality of CD4+ memory effector T cells is sufficient to cause expression of CCR7 and CD45RO after performing step (a) in no less than 15% of the CD4+ T cells.

Embodiment 42. The method of any of embodiments 34 through 41, wherein the CD4+ T cells secrete IFN-gamma or TNF-alpha.

Embodiment 43. The method of any of embodiments 34 through 42, wherein the CD4+ T cells secrete IFN-gamma and TNF-alpha.

Embodiment 44. The method of any of embodiments 34 through 43, wherein the CD4+ T cells are CD3+ and secrete IFN-gamma or TNF-alpha.

Embodiment 45. The method of any of embodiments 34 through 44, wherein the method further comprises stimulating proliferation of a subpopulation of CD8+ T cells that secrete IFN-gamma or TNF-alpha by exposing the cells simultaneously or sequentially with a polypeptide or nucleic acid encoding a polypeptide of IL-15, or functional fragments or variants thereof, and a polypeptide or nucleic acid encoding a polypeptide of IL-6, or functional fragments or variants thereof.

Embodiment 46. A method of generating, culturing and/or manufacturing CD8+ and/or CD4+ effector memory cells, said method comprising:
(a) contacting one or plurality of lymphocytes with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, for a time period sufficient to stimulate growth and proliferation of the one or plurality of CD8+ and/or CD4+ effector memory cells within the composition of cultured cells; preferably wherein said at least two peptides comprise IL-15 and IL-7; and preferably wherein said CD8+ and/or CD4+ memory effector T cells recognize, or are contacted with, a peptide epitope of SARS-CoV-2.

Embodiment 47. The method of embodiment 46 further comprising:
(b1) contacting the one or plurality of lymphocytes with one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1; or one or a plurality of vectors that encode one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1 for a time period sufficient to activate the one or plurality of CD8+ and/or CD4+ effector memory cells against a cell expressing PRAME, Survivin, or WT1, or functional fragments or combinations thereof, or
(b2) contacting the one or plurality of lymphocytes with one or a plurality of viral antigens from a virus of the family Coronaviridae for a time period sufficient to activate the one or plurality of CD8+ and/or CD4+ effector memory cells against a cell expressing the one or plurality of viral antigens.

Embodiment 48. The method of embodiment 46 or 47 further comprising:
(c) contacting the one or plurality of lymphocytes with one or a plurality of peptides that comprise at least about 70%, 75%, 80%, 85%, 90%, 91%, 92%, 93%, 94%, 95%, 96%, 97%, 98%, 99%, <100%, or 100% sequence identity to one or a plurality of tumor associated antigens.

Embodiment 49. The method of any of embodiments 46 through 48, wherein the one or plurality of lymphocytes are from naïve T cells from a subject.

Embodiment 50. The method of any of embodiments 46 through 48 further comprising isolating PBMCs from a subject.

Embodiment 51. The method of embodiment 50, wherein the step of isolating PBMCs comprising placing a whole blood sample from the subject through an apheresis unit.

Embodiment 52. The method of any of embodiments 46 through 51, further comprising a step of aliquoting PBMCs into a cell culture unit comprising a plurality of vessels prior to exposing the cells to the cytokines, wherein each of the vessels comprises a volume defined by at least one base surface and at least one sidewall.

Embodiment 53. A T cell composition comprising from about 2% to about 23% of CD4+ and/or CD8+ T cells manufactured by any of the methods of embodiments 1 through 21 and embodiments 69 through 72 or CD4+ and/or CD8+ memory effector T cells manufactured by any of methods of embodiments 22 through 52 and embodiments 73 through 84; preferably wherein said CD4+ and/or CD8+ memory effector T cells recognize, or are contacted with, a peptide epitope of SARS-CoV-2.

Embodiment 54. An isolated composition comprising lymphocytes, wherein the lymphocytes comprise:
(i) from about 2% to about 25% of CD4+ memory effector T cells; and/or
(ii) from about 2% to about 25% of CD8+ memory effector T cells.

Embodiment 55. A tissue culture system comprising a composition comprising lymphocytes and tissue culture media, wherein the lymphocytes comprise:
(i) from about 2% to about 25% of CD4+ memory effector T cells; and/or
(ii) from about 2% to about 25% of CD8+ memory effector T cells.

Embodiment 56. The tissue culture system of embodiment 55, further comprising NKT cells.

Embodiment 57. The tissue culture system of embodiment 55 or 56, wherein the system further comprises at least one cell culture surface area onto which the lymphocytes adhere, a tissue culture media reservoir in closed fluid communication with the at least one cell culture surface area by one or more media lines and a pump in operable connection to the reservoir and configured to create pressure in the media lines sufficient to generate a rate of flow of tissue culture media over the cell culture surface area.

Embodiment 58. A method of inducing an antigen-specific immune response against a viral antigen, the method comprising:
(a) contacting one or a plurality of lymphocytes with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, for a time period sufficient to stimulate growth and proliferation of one or a plurality of CD4+ and/or CD8+ T cells within a composition of cultured cells; preferably wherein said at least two peptides comprise IL-15 and IL-7; and preferably wherein said CD8+ memory effector T cells recognize, or are contacted with, a peptide epitope of SARS-CoV-2.

Embodiment 59. The method of embodiment 58 further comprising:
(b1) contacting the one or plurality of lymphocytes with one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1, or one or a plurality of vectors that encode one or a plurality of peptides that comprise at least about 70%, 75%, 80%, 85%, 90%, 91%, 92%, 93%, 94%, 95%, 96%, 97%, 98%, 99%, <100%, or 100% sequence identity to PRAME, Survivin, or WT1 for a time period sufficient to activate the one or plurality of CD4+ and/or CD8+ T cells against a cell expressing PRAME, Survivin, or WT1, or functional fragments or combinations thereof.

Embodiment 60. The method of embodiment 58 or 59 further comprising a step of contacting the one or plurality of lymphocytes with one or a plurality of viral antigens.

Embodiment 61. The method of embodiment 60, wherein the viral antigens are:
(i) peptides comprising at least about 70%, 75%, 80%, 85%, 90%, 91%, 92%, 93%, 94%, 95%, 96%, 97%, 98%, 99%, <100%, or 100% sequence identity to any one or combination of sequences provided in FIG. 9, or any functional fragment or variant thereof,
(ii) nucleic acids encoding an amino acid sequence that comprises at least about 70%, 75%, 80%, 85%, 90%, 91%, 92%, 93%, 94%, 95%, 96%, 97%, 98%, 99%, <100%, or 100% sequence identity to any one or combination of sequences provided in FIG. 9, or any functional fragment or variant thereof, or
(iii) any combination of (i) and (ii).

Embodiment 62. The method of any of embodiments 58 through 61, wherein the viral antigens are selected from one or a combination of EBV, HHV, CMV, BKV, or HPIV.

Embodiment 63. The method of embodiment 60, wherein the viral antigens are:
(i) from a virus of the family Coronaviridae;
(v) from a coronavirus; or
(vi) from SARS-CoV-2.

Embodiment 64. A method of selectively growing CD8+ and/or CD4+ memory effector T cells from a cell composition comprising naïve T cells, said method comprising, consisting essentially of, or consisting of:
(a) contacting one or a plurality of lymphocytes comprising the naïve T cells with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, IL-6, IL-7, or IL-4, or functional fragments or variants thereof, for a time period sufficient to stimulate growth and proliferation of the CD8+ and/or CD4+ memory effector T cells within the composition of cultured cells; preferably wherein said at least two peptides comprise IL-15 and IL-7; and preferably wherein said CD8+ memory effector T cells recognize, or are contacted with, a membrane peptide epitope of SARS-CoV-2.

Embodiment 65. The method of embodiment 64, wherein, if growth and proliferation of a population of CD8+ memory effector T cells is stimulated, the step of contacting one or plurality of lymphocytes comprises: contacting one or plurality of lymphocytes comprising the naïve T cells with at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, or IL-6, or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-18, IL-15, or IL-6, or functional fragments or variants thereof, for a time period sufficient to stimulate growth and proliferation of the population of CD8+ memory effector T cells within the composition of cultured cells; or wherein, if growth and proliferation of a population of CD4+ memory effector T cells is stimulated, the step of contacting one or plurality of lymphocytes comprises: contacting one or plurality of lymphocytes comprising the naïve T cells with at least two peptides that comprise at least about 70%, 75%, 80%, 85%, 90%, 91%, 92%, 93%, 94%, 95%, 96%, 97%, 98%, 99%, <100%, or 100% sequence identity to IL-7 and IL-4, or functional fragments or variants thereof, or one or a plurality of vectors that encode at least two peptides that comprise at least about 70%, 75%, 80%, 85%, 90%, 91%, 92%, 93%, 94%, 95%, 96%, 97%, 98%, 99%, <100%, or 100% sequence identity to IL-7 and IL-4, or functional fragments or variants thereof, for a time period sufficient to stimulate growth and proliferation of the population of memory effector T cells within the composition of cultured cells.

Embodiment 66. The method of embodiment 64 or 65 further comprising a step of contacting the one or plurality of lymphocytes or the one or plurality of CD8+ and/or CD4+ memory effector T cells with one or a plurality of viral antigens.

Embodiment 67. The method of embodiment 64 or 65 further comprising a step of contacting the one or plurality of lymphocytes or the one or plurality of CD8+ and/or CD4+ memory effector T cells with one or a plurality of viral antigens for a time period sufficient to induce an antigen-specific immune response against the one or plurality of viral antigens.

Embodiment 68. The method of either of embodiments 64 or 65 further comprising a step of contacting the one or plurality of lymphocytes or the one or plurality of CD8+ and/or CD4+ memory effector T cells with one or a plurality of viral antigens for a time period sufficient to induce an antigen-specific immune response against the one or plurality of viral antigens as measured by the number of CD3+ T cells that secrete interferon-gamma after exposure to the one or plurality of viral antigens.

Embodiment 69. The method of any of embodiments 1 through 21, wherein the at least two peptides comprise at least about 70%, 75%, 80%, 85%, 90%, 91%, 92%, 93%, 94%, 95%, 96%, 97%, 98%, 99%, <100%, or 100% sequence identity to SEQ ID NO: 1, SEQ ID NO: 3, SEQ ID NO: 5, SEQ ID NO: 7 or SEQ ID NO: 9.

Embodiment 70. The method of any of embodiments 1 through 21 and embodiment 69, wherein the one or plurality of CD4+ and/or CD8+ T cells are specific for one or a plurality of viral antigens from Coronaviridae.

Embodiment 71. The method of embodiment 70, wherein the one or plurality of viral antigens is a SARS-CoV-2 antigen.

Embodiment 72. The method of embodiment 70 or 71, wherein the one or plurality of viral antigens comprise at least about 70%, 75%, 80%, 85%, 90%, 91%, 92%, 93%, 94%, 95%, 96%, 97%, 98%, 99%, <100%, or 100% sequence identity to SEQ ID NO: 11, SEQ ID NO: 12, SEQ ID NO: 13, and/or SEQ ID NO: 14, or functional fragments or variants thereof.

Embodiment 73. The method of any of embodiments 22 through 33, wherein the at least two peptides comprise at least about 70%, 75%, 80%, 85%, 90%, 91%, 92%, 93%, 94%, 95%, 96%, 97%, 98%, 99%, <100%, or 100% sequence identity to SEQ ID NO: 1, SEQ ID NO: 3, SEQ ID NO: 5, SEQ ID NO: 7 or SEQ ID NO: 9.

Embodiment 74. The method of any of embodiments 22 through 33 and embodiment 73, wherein the one or plurality of CD4+ and/or CD8+ T cells are specific for one or a plurality of viral antigens from Coronaviridae.

Embodiment 75. The method of embodiment 74, wherein the viral antigen is a SARS-CoV-2 antigen.

Embodiment 76. The method of embodiment 74 or 75, wherein the one or plurality of viral antigens comprise at least about 70%, 75%, 80%, 85%, 90%, 91%, 92%, 93%, 94%, 95%, 96%, 97%, 98%, 99%, <100%, or 100% sequence identity to SEQ ID NO: 11, SEQ ID NO: 12, SEQ ID NO: 13, and/or SEQ ID NO: 14, or functional fragments or variants thereof.

Embodiment 77. The method of any of embodiments 34 through 45, wherein the at least two peptides comprise at least about 70%, 75%, 80%, 85%, 90%, 91%, 92%, 93%, 94%, 95%, 96%, 97%, 98%, 99%, <100%, or 100% sequence identity to SEQ ID NO: 1, SEQ ID NO: 3, SEQ ID NO: 5, SEQ ID NO: 7 or SEQ ID NO: 9.

Embodiment 78. The method of any of embodiments 34 through 45 and embodiment 77, wherein the one or plurality of CD4+ and/or CD8+ T cells are specific for one or a plurality of Coronaviridae viral antigens.

Embodiment 79. The method of embodiment 78, wherein the one or plurality of antigens are SARS-CoV-2 antigens.

Embodiment 80. The method of embodiment 78 or 79, wherein the one or plurality of viral antigens comprise at least about 70%, 75%, 80%, 85%, 90%, 91%, 92%, 93%, 94%, 95%, 96%, 97%, 98%, 99%, <100%, or 100% sequence identity to SEQ ID NO: 11, SEQ ID NO: 12, SEQ ID NO: 13, and/or SEQ ID NO: 14, or functional fragments or variants thereof.

Embodiment 81. The method of any of embodiments 46 through 52, wherein the at least two peptides comprise at least about 70%, 75%, 80%, 85%, 90%, 91%, 92%, 93%, 94%, 95%, 96%, 97%, 98%, 99%, <100%, or 100% sequence identity to SEQ ID NO: 1, SEQ ID NO: 3, SEQ ID NO: 5, SEQ ID NO: 7 or SEQ ID NO: 9.

Embodiment 82. The method of any of embodiments 46 through 52 and embodiment 81, wherein the one or plurality of CD4+ and/or CD8+ T cells are specific for one or a plurality of viral antigens from a coronavirus.

Embodiment 83. The method of embodiment 82, wherein the coronavirus is SARS-CoV-2.

Embodiment 84. The method of embodiment 82 or 83, wherein the one or plurality of viral antigens comprise at least about 70%, 75%, 80%, 85%, 90%, 91%, 92%, 93%, 94%, 95%, 96%, 97%, 98%, 99%, <100%, or 100% sequence identity to SEQ ID NO: 11, SEQ ID NO: 12, SEQ ID NO: 13, and/or SEQ ID NO: 14, or functional fragments or variants thereof.

Embodiment 85. nA method of generating, culturing and/or manufacturing one or a plurality of CD4+ and/or CD8+ T cells specific to one or a plurality of viral antigens, the method comprising, consisting essentially of, or consisting of:
(a) contacting one or a plurality of lymphocytes with at least two cytokines chosen from IL-18, IL-15, IL-6, IL-7, and IL-4, or functional fragments or variants thereof, for a time period sufficient to stimulate growth and proliferation of the one or plurality of CD4+ and/or CD8+ T cells within a composition of cultured cells; and
(b) contacting the one or plurality of lymphocytes with the one or plurality of viral antigens for a time period sufficient to activate the one or plurality of CD4+ and/or CD8+ T cells against a cell expressing the one or plurality of viral antigens; preferably wherein said at least two peptides comprise IL-15 and IL-7; and preferably wherein said CD8+ memory effector T cells recognize, or are contacted with, a peptide epitope of SARS-CoV-2.

Embodiment 86. The method of embodiment 85, wherein the one or plurality of viral antigens are from a virus of the family Coronaviridae.

Embodiment 87. The method of embodiment 85 or 86, wherein the one or plurality of viral antigens are from a coronavirus.

Embodiment 88. The method of any of embodiments 85 through 87, wherein the one or plurality of viral antigens are from SARS-CoV-2.

Embodiment 89. The method of any of embodiments 85 through 88, wherein the one or plurality of viral antigens comprise at least about 70%, 75%, 80%, 85%, 90%, 91%, 92%, 93%, 94%, 95%, 96%, 97%, 98%, 99%, <100%, or 100% sequence identity to SEQ ID NO: 11, SEQ ID NO: 12, SEQ ID NO: 13, and/or SEQ ID NO: 14, or functional fragments or variants thereof.

Embodiment 90. The method of any of embodiments 85 through 89, wherein:
(i) the cytokine IL-15 comprises at least about 70%, 75%, 80%, 85%, 90%, 91%, 92%, 93%, 94%, 95%, 96%, 97%, 98%, 99%, <100%, or 100% sequence identity to SEQ ID NO: 1;
(ii) the cytokine IL-7 comprises at least about 70%, 75%, 80%, 85%, 90%, 91%, 92%, 93%, 94%, 95%, 96%, 97%, 98%, 99%, <100%, or 100% sequence identity to SEQ ID NO: 3;
(iii) the cytokine IL-6 comprises at least about 70%, 75%, 80%, 85%, 90%, 91%, 92%, 93%, 94%, 95%, 96%, 97%, 98%, 99%, <100%, or 100% sequence identity to SEQ ID NO: 5;
(iv) the cytokine IL-4 comprises at least about 70%, 75%, 80%, 85%, 90%, 91%, 92%, 93%, 94%, 95%, 96%, 97%, 98%, 99%, <100%, or 100% sequence identity to SEQ ID NO: 7; and/or
(v) the cytokine IL-18 comprises at least about 70%, 75%, 80%, 85%, 90%, 91%, 92%, 93%, 94%, 95%, 96%, 97%, 98%, 99%, <100%, or 100% sequence identity to SEQ ID NO: 9.

Embodiment 91. The method of any of embodiments 85 through 90, wherein the time period sufficient to stimulate growth and proliferation of the one or plurality CD4+ and/or CD8+ T cells and/or the time period sufficient to activate the one or plurality of CD4+ and/or CD8+ T cells against a cell expressing the one or plurality of viral antigens is from about 2 to about 10 days.

Embodiment 92. The method of any of embodiments 85 through 91, wherein the time period sufficient to stimulate growth and proliferation of the one or plurality of CD4+ and/or CD8+ T cells and/or the time period sufficient to activate the one or plurality of CD4+ and/or CD8+ T cells against a cell expressing the one or plurality of viral antigens is from about 3 to about 7 days.

Embodiment 93. The method of any of embodiments 85 through 92, wherein the at least two cytokines are:
(i) IL-15 and IL-6, or functional fragments or variants thereof;
(ii) IL-15 and IL-7, or functional fragments or variants thereof, or
(iii) IL-7 and IL-4, or functional fragments or variants thereof.

Embodiment 94. The method of any of embodiments 85 through 93, wherein no less than about 2% of the one or plurality of CD4+ and/or CD8+ T cells are CCR7+CD45RO+.

Embodiment 95. The method of any of embodiments 85 through 94, wherein no less than about 5% of the one or a plurality of CD4+ and/or CD8+ T cells are CCR7+CD45RO+.

Embodiment 96. The method of any of embodiments 85 through 95, wherein no less than about 10% of the one or plurality of CD4+ and/or CD8+ T cells are CCR7+CD45RO+.

Embodiment 97. The method of any of embodiments 85 through 96, wherein no less than about 1, 2, 5, 10 or 15% of the one or plurality of CD4+ and/or CD8+ T cells are CCR7+CD45RO+.

Embodiment 98. The method of any of embodiments 85 through 97, wherein the one or plurality of CD4+ and/or CD8+ T cells secrete IFN-gamma or TNF-alpha.

Embodiment 99. A T cell composition comprising from about 2%, 5%, 10%, 15%, 20% to about 23% of CD4+ and/or CD8+ T cells manufactured by any of the methods of embodiments 85 through 98.

Embodiment 100. A pharmaceutical composition comprising, consisting essentially of, or consisting of:
(i) the one or plurality of CD4+ and/or CD8+ T cells generated by any of the methods of embodiments 85 through 98 or the T cell composition of embodiment 99; and
(ii) a pharmaceutically acceptable carrier.

Embodiment 101. A method of preventing and/or treating viral infection in a subject in need thereof, said method comprising, consisting essentially of, or consisting of administering a pharmaceutically effective amount of the one or plurality of CD4+ and/or CD8+ T cells generated by any of the methods of embodiments 85 through 98, the T cell composition of embodiment 99, or the pharmaceutical composition of embodiment 100 to the subject in need thereof.

Embodiment 102. The method of embodiment 101, wherein the viral infection is a Coronaviridae infection.

Embodiment 103. The method of embodiment 101 or 102, wherein the viral infection is coronavirus infection.

Embodiment 104. The method of any of embodiments 101 through 103, wherein the viral infection is a SARS-CoV-2 of COVID-19 infection.

Embodiment 105. The method of embodiment 84, wherein the one or plurality of viral antigens comprise one or plurality of viral epitopes chosen from Table A, Table B, and/or Table C.

Embodiment 106. A T cell composition comprising T cells manufactured by the method of embodiment 105.

Embodiment 107. The method of embodiment 89, wherein the one or plurality of viral antigens comprise one or plurality of viral epitopes chosen from Table A, Table B, and/or Table C.

Embodiment 108. A T cell composition comprising T cells manufactured by the method of embodiment 107.

### REFERENCES

1. S. A. Rosenberg, et al. Adoptive cell transfer as personalized immunotherapy for human cancer, Science 348(6230) (2015) 62-8.
2. U. Gerdemann, et al. Safety and clinical efficacy of rapidly-generated trivirus-directed T cells as treatment for adenovirus, EBV, and CMV infections after allogeneic hematopoietic stem cell transplant, Mol Ther 21(11) (2013) 2113-21.
3. U. Gerdemann, et al. Generation of multivirus-specific T cells to prevent/treat viral infections after allogeneic hematopoietic stem cell transplant, J Vis Exp (51) (2011).
4. P. J. Hanley, et al. Expansion of T cells targeting multiple antigens of cytomegalovirus, Epstein-Barr virus and adenovirus to provide broad antiviral specificity after stem cell transplantation, Cytotherapy 13(8) (2011) 976-86.
5. A. Vella, et al. Interleukin 4 (IL-4) or IL-7 prevents the death of resting T cells: stat6 is probably not required for the effect of IL-4, The Journal of experimental medicine 186(2) (1997) 325-30.
6. A. T. Vella, et al. Cytokine-induced survival of activated T cells in vitro and in vivo, Proceedings of the National Academy of Sciences of the United States of America 95(7) (1998) 3810-5.
7. J. Hu-Li, et al. B cell stimulatory factor 1 (interleukin 4) is a potent costimulant for normal resting T lymphocytes, The Journal of experimental medicine 165(1) (1987) 157-72.
8. O. Boyman, et al. Cytokines and T-cell homeostasis, Current opinion in immunology 19(3) (2007) 320-6.
9. F. Melchionda, et al. Adjuvant IL-7 or IL-15 overcomes immunodominance and improves survival of the CD8+ memory cell pool, The Journal of clinical investigation 115(5) (2005) 1177-87.
10. J. Jin, M. et al. Simplified method of the growth of human tumor infiltrating lymphocytes in gas-permeable flasks to numbers needed for patient treatment, J Immunother 35(3) (2012) 283-92.
11. A. Kimura, et al. IL-6: regulator of Treg/Th17 balance, European journal of immunology 40(7) (2010) 1830-5.
12. T. J. Fry, et al. Interleukin-7: master regulator of peripheral T-cell homeostasis?, Trends in immunology 22(10) (2001) 564-71.
13. L. A. Geiselhart, et al. IL-7 administration alters the CD4:CD8 ratio, increases T cell numbers, and increases T cell function in the absence of activation, J Immunol 166(5) (2001) 3019-27.
14. J. T. Tan, et al. IL-7 is critical for homeostatic proliferation and survival of naïve T cells, Proceedings of the National Academy of Sciences of the United States of America 98(15) (2001) 8732-7.
15. R. Zeng, et al. Synergy of IL-21 and IL-15 in regulating CD8+ T cell expansion and function, The Journal of experimental medicine 201(1) (2005) 139-48.
16. K. A. Casey, et al. IL-21 promotes differentiation of naïve CD8 T cells to a unique effector phenotype, J Immunol 178(12) (2007) 7640-8.
17. Y. Li, M. et al. IL-21 influences the frequency, phenotype, and affinity of the antigen-specific CD8 T cell response, J Immunol 175(4) (2005) 2261-9.
18. C. M. Rooney, et al. Infusion of cytotoxic T cells for the prevention and treatment of Epstein-Barr virus-induced lymphoma in allogeneic transplant recipients, Blood 92(5) (1998) 1549-55.
19. C. M. Bollard, et al. Cytotoxic T lymphocyte therapy for Epstein-Barr virus+ Hodgkin's disease, The Journal of experimental medicine 200(12) (2004) 1623-33.
20. P. J. Hanley, et al. CMV-specific T cells generated from naïve T cells recognize atypical epitopes and may be protective in vivo, Sci Transl Med 7(285) (2015) 285ra63.
21. A. Papadopoulou, et al. Activity of broad-spectrum T cells as treatment for AdV, EBV, CMV, BKV, and HHV6 infections after HSCT, Sci Transl Med 6(242) (2014) 242ra83.
22. R. Geyeregger, et al. Short-term in-vitro expansion improves monitoring and allows affordable generation of virus-specific T-cells against several viruses for a broad clinical application, PloS one 8(4) (2013) e59592.
23. M. D. Keller, et al. T-cell receptor sequencing demonstrates persistence of virus-specific T cells after antiviral immunotherapy, British journal of haematology 187(2) (2019) 206-218.
24. M. D. Keller, et al. Virus specific T-cell Therapies for Patients with Primary Immune Deficiency, Blood (2020).
25. A. M. Leen, et al. Multicenter study of banked third-party virus-specific T cells to treat severe viral infections after hematopoietic stem cell transplantation, Blood 121(26) (2013) 5113-23.
26. A. M. Leen, et al. Monoculture-derived T lymphocytes specific for multiple viruses expand and produce clinically relevant effects in immunocompromised individuals, Nature medicine 12(10) (2006) 1160-6.
27. Y. D. Mahnke, et al. The who's who of T-cell differentiation: human memory T-cell subsets, European journal of immunology 43(11) (2013) 2797-809.
28. D. H. Busch, et al. Role of memory T cell subsets for adoptive immunotherapy, Seminars in immunology 28(1) (2016) 28-34.
29. J. Geginat, et al. Cytokine-driven proliferation and differentiation of human naïve, central memory, and effector memory CD4(+) T cells, The Journal of experimental medicine 194(12) (2001) 1711-9.
30. J. Gagnon, et al. IL-6, in synergy with IL-7 or IL-15, stimulates TCR-independent proliferation and functional differentiation of CD8+ T lymphocytes, J Immunol 180(12) (2008) 7958-68.
31. F. Carrette, et al. IL-7 signaling and CD127 receptor regulation in the control of T cell homeostasis, Seminars in immunology 24(3) (2012) 209-17.
32. T. J. Fry, et al. The many faces of IL-7: from lymphopoiesis to peripheral T cell maintenance, J Immunol 174(11) (2005) 6571-6.
33. Y. Tagaya, et al. IL-15: a pleiotropic cytokine with diverse receptor/signaling pathways whose expression is controlled at multiple levels, Immunity 4(4) (1996) 329-36.
34. S. A. Rosenberg, et al. IL-7 administration to humans leads to expansion of CD8+ and CD4+ cells but a relative decrease of CD4+ T-regulatory cells, J Immunother 29(3) (2006) 313-9.
35. C. Sportes, et al. Administration of rhIL-7 in humans increases in vivo TCR repertoire diversity by preferential expansion of naive T cell subsets, The Journal of experimental medicine 205(7) (2008) 1701-14.
36. U. Gerdemann, et al. Rapidly generated multivirus-specific cytotoxic T lymphocytes for the prophylaxis and treatment of viral infections, Mol Ther 20(8) (2012) 1622-32.
37. A. J. Sant, et al. Revealing the role of CD4(+) T cells in viral immunity, The Journal of experimental medicine 209(8) (2012) 1391-5.
38. P. Reusser, et al. Cytotoxic T-lymphocyte response to cytomegalovirus after human allogeneic bone marrow transplantation: pattern of recovery and correlation with cytomegalovirus infection and disease, Blood 78(5) (1991) 1373-80.
39. H. Einsele, E et al. Infusion of cytomegalovirus (CMV)-specific T cells for the treatment of CMV infection not responding to antiviral chemotherapy, Blood 99(11) (2002) 3916-22.
40. S. R. Riddell, et al. Cytotoxic T cells specific for cytomegalovirus: a potential therapy for immunocompromised patients, Rev Infect Dis 13 Suppl 11 (1991) S966-73.
41. A. Houghtelin, et al. Virus-Specific T Cells for the Immunocompromised Patient, Frontiers in immunology 8 (2017) 1272.
42. K. M. Harris, et al. Virus-Specific T Cells: Current and Future Use in Primary Immunodeficiency Disorders, J Allergy Clin Immunol Pract 7(3) (2019) 809-818.
43. S. Naik, et al. Adoptive immunotherapy for primary immunodeficiency disorders with virus-specific T lymphocytes, The Journal of allergy and clinical immunology 137(5) (2016) 1498-1505 el.
44. L. P. McLaughlin, et al. Adoptive T Cell Therapy for Epstein-Barr Virus Complications in Patients With Primary Immunodeficiency Disorders, Frontiers in immunology 9 (2018) 556.
45. Burbelo, P. D. et al. Detection of Nucleocapsid Antibody to SARS-CoV-2 is More Sensitive than Antibody to Spike Protein in COVID-19 Patients. J Infect Dis Pre-print (2020).
46. Zhu, N. et al. A Novel Coronavirus from Patients with Pneumonia in China, 2019. N Engl J Med 382, 727-733, doi:10.1056/NEJMoa2001017 (2020).
47. Long, Q. X. et al. Antibody responses to SARS-CoV-2 in patients with COVID-19. Nat Med, doi:10.1038/s41591-020-0897-1 (2020).
48. Grifoni, A. et al. Targets of T cell responses to SARS-CoV-2 coronavirus in humans with COVID-19 disease and unexposed individuals. Cell In press (2020).
49. Braun, J. et al. Presence of SARS-CoV-2-reactive T cells in COVID-19 patients and healthy donors. medRxiv (2020).
50. Thieme, C. J. et al. The SARS-CoV-2 T-cell immunity is directed against the spike, membrane, and nucleocapsid protein and associated with COVID 19 severity. medRxiv (2020).
51. Dai, M. et al. Patients with cancer appear more vulnerable to SARS-COV-2: a multicenter study during the COVID-19 outbreak. Cancer Discov, doi:10.1158/2159-8290.CD-20-0422 (2020).
52. Liang, W. et al. Cancer patients in SARS-CoV-2 infection: a nationwide analysis in China. Lancet Oncol 21, 335-337, doi:10.1016/S1470-2045(20)30096-6 (2020).
53. Yang, L. T. et al. Long-lived effector/central memory T-cell responses to severe acute respiratory syndrome coronavirus (SARS-CoV) S antigen in recovered SARS patients. Clin Immunol 120, 171-178, doi:10.1016/j.clim.2006.05.002 (2006).
54. Li, C. K. et al. T cell responses to whole SARS coronavirus in humans. J Immunol 181, 5490-5500, doi:10.4049/jimmunol.181.8.5490 (2008).
55. Savoldo, B. et al. Generation of EBV-specific CD4+ cytotoxic T cells from virus naive individuals. J Immunol 168, 909-918, doi:10.4049/jimmunol.168.2.909 (2002).
56. Posavad, C. M. et al. Detailed characterization of T cell responses to herpes simplex virus-2 in immune seronegative persons. J Immunol 184, 3250-3259, doi:10.4049/jimmunol.0900722 (2010).
57. Reynisson, B. et al. Improved Prediction of MHC II Antigen Presentation through Integration and Motif Deconvolution of Mass Spectrometry MHC Eluted Ligand Data. J Proteome Res, doi:10.1021/acs.jproteome.9b00874 (2020).
58. Chen, B. et al. Predicting HLA class II antigen presentation through integrated deep learning. Nat Biotechnol 37, 1332-1343, doi:10.1038/s41587-019-0280-2 (2019).
59. Chang, T. J. et al. Genomic Analysis and Comparative Multiple Sequence of SARS-CoV2. J Chin Med Assoc, doi:10.1097/JCMA.0000000000000335 (2020).
60. Zhao, J. et al. Recovery from the Middle East respiratory syndrome is associated with antibody and T-cell responses. Sci Immunol 2, doi:10.1126/sciimmunol.aan5393 (2017).
61. O'Reilly, R. J., Prockop, S., Hasan, A. N., Koehne, G. & Doubrovina, E. Virus-specific T-cell banks for ‘off the shelf’ adoptive therapy of refractory infections. Bone Marrow Transplant, doi:10.1038/bmt.2016.17 (2016).
62. Klitz, W. et al. New HLA haplotype frequency reference standards: high-resolution and large sample typing of HLA DR-DQ haplotypes in a sample of European Americans. Tissue Antigens 62, 296-307, doi:10.1034/j.1399-0039.2003.00103.x (2003).
63. Riphagen, S., Gomez, X., Gonzalez-Martinez, C., Wilkinson, N. & Theocharis, P. Hyperinflammatory shock in children during COVID-19 pandemic. Lancet, doi:10.1016/S0140-6736(20)31094-1 (2020).
64. Keller, M. D. & Bollard, C. M. Virus-specific T-cell therapies for patients with primary immune deficiency. Blood 135, 620-628, doi:10.1182/blood.2019000924 (2020).

1. A method for culturing CD4+ and/or CD8+ T cells from a biological sample that comprises T cells, which recognize at least one viral peptide epitope or other epitope, comprising:
contacting T cells from the biological sample with at least one peptide epitope and with a combination of at least two of IL-4 (SEQ ID NO: 7), IL-6 (SEQ ID NO: 5), IL-7 (SEQ ID NO: 3), IL-15 (SEQ ID NO: 1), or IL-18 (SEQ ID NO: 9) cytokines, with fragments thereof, or with variants thereof, for a time and under conditions sufficient to stimulate growth or proliferation of the CD4+ and/or CD8+ T cells; thereby producing a culture containing CD4+ and/or CD8+ T cells;
wherein said IL-4, IL-6, IL-7, IL-15, or IL-18 cytokine variants have at least 90% sequence similarity to the amino acid sequences of IL-4 (SEQ ID NO: 7), IL-6 (SEQ ID NO: 5), IL-7 (SEQ ID NO: 3), IL-15 (SEQ ID NO: 1), or IL-18 (SEQ ID NO: 9).

2-3. (canceled)

4. The method of claim 1, wherein said contacting comprises contacting the T cells with IL-6 and IL-15 under conditions favoring outgrowth of CD8+ T cells compared to outgrowth of CD4+ T cells, and, optionally isolating CD8+ central memory T cells expressing CD45RO, CCR7 and L-selectin; or isolating CD8+ effector memory T cells expressing CD45RO without expressing CCR7 or L-selectin from other cells in the culture.

5. The method of claim 1, wherein said contacting comprises contacting the T cells with IL-7 and IL-15 under conditions favoring outgrowth or increased specificity of CD4+ T cells and/or outgrowth of or increased specificity of CD8+ cells compared to outgrowth of, or specificity of CD4+ and/or CD8+ when the T cells are contacted with IL-4 and IL-7, and, optionally isolating CD4+ central memory T cells expressing CD45RO, CCR7 and L-selectin; isolating CD4+ effector memory T cells expressing CD45RO without expressing CCR7 or L-selectin; or isolating CD8+ effector memory T cells expressing CD45RO without expressing CCR7 or L-selectin.

6. The method of claim 1, wherein said contacting comprises contacting the T cells with IL-4 and IL-7 under conditions favoring outgrowth of CD4+ T cells compared to the outgrowth of CD8+ T cells, and, optionally isolating CD4+ central memory T cells expressing CD45RO, CCR7 and L-selectin; or isolating CD4+ effector memory T cells expressing CD45RO without expressing CCR7 or L-selectin.

7. The method of claim 1, wherein the T cells recognize at least one SARS-CoV-2 epitope from Spike (S), Membrane (M), Envelope (E) or Nucleocapsid (N) protein.

8. (canceled)

9. The method of claim 1, wherein the at least one peptide epitope is present on at least one of the group of peptides described by SEQ ID NOS: 50 to 515.

10. The method of claim 1, wherein the biological sample is obtained from a subject infected with SARS-CoV-2, obtained from a subject previously infected with SARS-CoV-2, obtained from a subject previously immunized with at least one epitope of a SARS-CoV-2 protein, or obtained from a subject who has previously been administered T cells that recognize a SARS-CoV-2 epitope.

11-17. (canceled)

18. The method of claim 1, further comprising viably storing, banking, or cryopreserving the T cells or the cultured T cells and, optionally, identifying the HLA backgrounds of the stored cells.

19. A method for preventing or treating a disease, disorder, or condition comprising administering to a subject in need thereof the T cells produced by the method of claim 1.

20. The method of claim 19, wherein the T cells comprise expanded CD4+ memory T cells.

21. The method of claim 19, wherein the T cells comprise expanded CD8+ memory T cells.

22. The method of claim 19, wherein the disease, disorder of condition is an infection by SARS-CoV-2.

23. (canceled)

24. The method of claim 19, wherein the T cells are autologous to the subject.

25. The method of claim 19, wherein the T cells are allogeneic to the subject.

26. The method of claim 19, wherein the T cells are allogeneic to the subject and share at least one HLA class 1 or HLA class 2 antigen or allele with the subject.

27. The method of claim 19, wherein the T cells are allogeneic to the subject and share at least four HLA class 1 or HLA class 2 antigens or alleles with the subject.

28. The method of claim 19, that comprises administering CD8+ memory effector T cells which recognize a SARS-CoV-2 peptide epitope or other viral epitope to a subject in need thereof or to a subject deficient in therapeutically effective numbers of said CD8+ memory effector T cells.

29. The method of claim 19, that comprises administering CD4+ memory effector T cells which recognize a SARS-CoV-2 peptide epitope or other viral epitope to a subject in need thereof or to a subject deficient in therapeutically effective numbers of said CD4+ memory effector T cells.

30. The method of claim 19, that comprises administering CD8+ and CD4+ memory effector T cells which recognize a SARS-CoV-2 peptide epitope or other viral epitope to a subject in need thereof or to a subject deficient in therapeutically effective numbers of said CD4+ and CD8+ memory effector T cells.

31. A composition comprising:
A(i) an artificial culture medium for T cells, (ii) cytokines consisting essentially of IL-4 and IL-7, and (iii) isolated T cells or precursor T cells, wherein said cytokines are present in an amount sufficient to increase the numbers of CD4+ T cells compared to the numbers of CD4+ T cells in said artificial culture medium in combination with IL-6 and IL-15 instead of IL-4 and IL-7; or wherein said cytokines are present in an amount sufficient to increase numbers of CD3+ T cells compared to numbers of CD3+ T cells in said artificial culture medium containing no added cytokines; or
B(i) an artificial culture medium for T cells, B(ii) cytokines consisting essentially of IL-6 and IL-15, and B (iii) isolated T cells or precursor T cells, wherein said cytokines are present in an amount sufficient to increase the numbers of CD8+ T cells compared to the numbers of CD8+ T cells in said artificial culture medium in combination with IL-4 and IL-7 instead of IL-6 and IL-15; or wherein said cytokines are present in an amount sufficient to increase numbers of CD3+ T cells compared to numbers of CD3+ T cells in said artificial culture medium containing no added cytokines; or wherein said cytokines are present in an amount sufficient to increase numbers of NK cells compared to numbers of NK cells in said artificial culture medium containing in combination with IL-4 and IL-7 instead of IL-IL-6 and IL-15; or
C(i) an artificial culture medium for T cells, C(ii) cytokines consisting essentially of IL-7 and IL-15, and C (iii) isolated T cells or precursor T cells, wherein said cytokines are present in an amount sufficient to increase the numbers of CD4+ T cells compared to numbers of CD4+ T cells in said artificial culture medium containing IL-6 and IL-15, or compared to IL-4 and IL-7, instead of IL-7 and IL-15; or wherein said IL-7 and IL-15 cytokines are present in an amount sufficient to increase expansion of CD3+ T cells compared to expansion of CD3+ T cells in said artificial culture medium for T cells containing no added cytokines.

32-39. (canceled)
